(19) United States
(12) Patent Application Publication
(10) Pub. No.: US 2011/0044895 A1
(43) Pub. Date: Feb. 24, 2011

(54) METHODS AND COMPOSITIONS FOR REDUCING STEMNESS IN ONCOGENESIS

(76) Inventors: David A. Berry, Brookline, MA (US); Eric J. Devroe, Malden, MA (US); Noubar B. Afeyan, Lexington, MA (US); Brett Chevalier, Malden, MA (US); Sashank K. Reddy, Boston, MA (US)

Correspondence Address: Maria Laccotripe Zacharakis PH.D,JD, Mccarter & Elglish, LLP, 265 Franklin Street, Boston, MA, 02110, US

(21) Appl. No.: 12/852,973

(22) Filed: Aug. 9, 2010

Related U.S. Application Data
(63) Continuation of application No. 12/171,923, filed on Jul. 11, 2008, now abandoned.
(60) Provisional application No. 60/949,409, filed on Jul. 12, 2007.

(51) Int. Cl. A61K 51/00; C12N 5/09; A61K 39/395; A61K 31/7105; A61K 49/00; C12N 5/095; A61K 31/7088; A61P 35/00

(52) U.S. Cl. 424/1.11; 435/375; 424/130.1; 514/44 A; 424/9.2

(57) ABSTRACT

The invention provides methods and compositions for reducing the number of cancer stem cells in a mixed population of differentiated cells (for example, cancer cells) and cancer stem cells. The cancer stem cells, if present, can be more resistant to traditional drug-based therapies and can provide a source for new, differentiated cancer cells associated with the development of drug-resistance and more aggressive phenotypes. When combined with traditional cancer therapies, for example, drug-based therapies, the methods and compositions of the invention provide a more effective way for treating cancer and can provide a model system for developing new cancer therapies and new treatment modalities.

### CROSS REFERENCE TO RELATED APPLICATION

This application is a continuation application of U.S. patent application Ser. No. 12/171,923, filed Jul. 11, 2008, which claims the benefit of and priority to U.S. Provisional Application No. 60/949,409, filed on Jul. 12, 2007. The entire contents of each of which are incorporated herein in their entirety by this reference

### FIELD OF INVENTION

The field of the invention is cell biology, molecular biology and oncology. More particularly, the field relates to methods and compositions for reducing sternness during oncogenesis.

### BACKGROUND

Cancer is one of the most significant health conditions facing individuals in both developed and developing countries. The National Cancer Institute has estimated that in the United States alone, one in three people will be afflicted with cancer during their lifetime. Moreover, approximately 50% to 60% of people afflicted with cancer will eventually succumb to the disease. Although significant progress has been made in the early detection and treatment of certain cancers, other cancers have been more difficult to detect and/or treat.

To date, typical therapies include surgery, chemotherapy, radiation therapy, hormone therapy, and immunotherapy. However, each of these therapies have certain disadvantages, which include, for example, complications that result from surgery or drug-based therapies, lack of short term or long term efficacy, and toxicities that can occur, for example, due to non-specific adverse effects on normal cells and tissues. In general, conventional drug-based therapies and regimens have been designed to target rapidly proliferating cells (i.e., differentiated cancer cells that comprise the bulk of a cancer). As a result, cells that do not proliferate as quickly as normal cells may not respond, or may be less likely to respond, to a given treatment regime.

It has been reported that cancers include both differentiated, rapidly proliferating cells and also more slowly replicating cells, for example, cancer stem cells, as described, for example, in U.S. Patent Application Publication Nos. US2006/0083682A1 and US2008/0118418A1. It is believed that the slower replicating stem-like cells, which may be less susceptible to conventional drug-based therapies, may be causes of clinical relapses or recurrences that can occur during and after treatment.

Accordingly, there is still an ongoing need for new methods and compositions that reduce the number of cancer stem cells.

### SUMMARY OF THE INVENTION

It is believed that cancer stem cells, which represent a small fraction of cancer cells, are particularly resistant to treatment with one or more anti-cancer agents. As a result, even though initial treatment may be successful, the residual cancer stem cells can be the source of new, and potentially more aggressive and/or resistant cancer cells. Accordingly, the invention provides methods and compositions for reducing the number of cancer stem cells so that concurrent or subsequent cancer therapy is more effective.

In one aspect, the invention provides a method of facilitating cell death of cancer stem cells or differentiated cells with a propensity for forming cancer stem cells in a mixed population of cancer stem cells and differentiated cells. The method comprises the steps of (a) inhibiting the formation of cancer stem cells in the initial mixed population, and optionally inhibiting the maintenance of the cancer stem cells in the initial mixed population, thereby to produce a second population of cells with fewer cancer stem cells or differentiated cells with a propensity for forming cancer stem cells than the initial mixed population, and (b) inducing cell death of the differentiated cells in the second population of cells. It is understood that step (b) can occur after or contemporaneously with step (a).

In certain embodiments, an agent used to inhibit the formation of cancer stem cells and/or to inhibit the maintenance of the cancer stem cell directly reduces the expression or activity of a transcription factor, for example, a transcription factor selected from the group consisting of Oct4, Sox2, Klf2, Klf4, Klf5, Tbx3, Nanog, ESRRB and REST. Furthermore the targeted transcription factor can also include Foxc1, Foxc2, Goosecoid, Sip1, Snail1, Snail2, TCF3, and Twist. The agent may include, for example, a protein, such as, an antibody, a nucleic acid, such as, an anti-sense RNA or a small interfering RNA (siRNA), or a small molecule, or a combination thereof.

In another aspect, the invention provides a method of reducing the number of cancer stem cells or differentiated cells with a propensity for forming cancer stem cells in a mixed population of cancer stem cells and differentiated cells. The method comprises exposing the mixed population of cancer stem cells and differentiated cells to at least two different agents that directly reduce the expression or activity of two transcription factors that modulate the formation of cancer stem cells from one or more of the differentiated cells and/or modulate the maintenance of the cancer stem cells, thereby to reduce the number of cancer stem cells or differentiated stem cells with a propensity for forming cancer stem cells in the mixed population.

It is understood, the method contemplates exposing the cells to the two agents simultaneously or one after the other. The method also contemplates exposing the cells to at least three, four, five or six different agents, either simultaneously or one after the other.

In one embodiment, the transcription factor that modulates the formation of cancer stem cells and/or modulates the maintenance of cancer stem cells is selected from the group consisting of Oct4, Sox2, Klf2, Klf4, Klf5, Tbx3, Nanog, ESRRB and REST. Other exemplary transcription factors include, for example, Foxc1, Foxc2, Goosecoid, Sip1, Snail1, Snail2, TCF3, and Twist. The agents that modulate the activity of such transcription factors can include, for example, a protein, such as, an antibody, a nucleic acid, such as, an anti-sense RNA or an siRNA, a small molecule, or a combination thereof.

In another aspect, the invention provides a method of reducing the relative number of cancer stem cells or differentiated cells with a propensity for forming cancer stem cells in a mixed population of cancer stem cells and differentiated cells. The method comprises exposing the mixed population of cancer stem cells and differentiated cells with a combination of a first agent that inhibits the formation of cancer stem cells from one or more of the differentiated cells and a second agent that inhibits the maintenance of the cancer stem cells, thereby to reduce the relative number of cancer stem cells or differentiated cells with a propensity for forming cancer stem cells in the mixed population.

It is understood, however, that depending upon the targets chosen, the first agent and the second agent may both inhibit the formation of cancer stem cells from one or more differentiated cells and inhibit the maintenance of the cancer stem cells. It is understood that the expression or activity of certain transcription factors, for example, a transcription factor selected from the group consisting of Oct4, Sox2, Klf2, Klf4, Klf5, Tbx3, Nanog, ESRRB and REST can have such effects. Other exemplary transcription factors include Foxc1, Foxc2, Goosecoid, Sip1, Snail1, Snail2, TCF3, and Twist. The first and second agents can be a protein, for example, an antibody, a nucleic acid, for example, an anti-sense RNA or an siRNA, or a small molecule, or a combination thereof.

In another aspect, the invention provides a method of treating cancer in a mammal. The method comprises administering to the mammal in need thereof an effective amount of at least two agents (for example, two three, four, five or six agents) that inhibit the formation of cancer stem cells from differentiated cells and/or inhibit the maintenance of cancer stem cells, thereby to treat the cancer in the mammal.

In certain embodiments, the agent that inhibits the formation of cancer stem cells or inhibits the maintenance of cancer stem cells directly reduces the expression or activity of a transcription factor, for example, a transcription factor selected from the group consisting of Oct4, Sox2, Klf2, Klf4, Klf5, Tbx3, Nanog, ESRRB, REST, Foxc1, Foxc2, Goosecoid, Sip1, Snail1, Snail2, TCF3, and Twist. The agent can include, for example, a protein, such as, an antibody, a nucleic acid, such as, an anti-sense RNA or an siRNA, or a small molecule.

In such an approach, the method can include administering to the mammal at least two agents that inhibit the formation of cancer stem cells. The method can include at least two agents that inhibit the maintenance of cancer stem cells. Alternatively, the method can comprise administering a combination of an agent that inhibits the formation of cancer stem cells and a separate agent that inhibits the maintenance of cancer stem cells.

In another aspect, the invention provides a method of treating cancer in a mammal. The method comprises administering to the mammal an effective amount one or more agents that directly reduce the expression or activity of Oct4, Sox2, Klf2, Klf4, Klf5, Tbx3, Nanog, ESRRB, REST, Foxc1, Foxc2, Goosecoid, Sip1, Snail1, Snail2, TCF3, and Twist, thereby to ameliorate one or more symptoms of the cancer. The agent or agents can include, for example, a protein, such as, an antibody, a nucleic acid, such as, an anti-sense RNA or an siRNA, or a small molecule.

In another aspect, the invention provides a method of treating cancer in a mammal, the method comprising administering to the mammal an effective amount of one or more agents that directly reduce the expression or activity of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist disposed with an encapsulation vehicle. The agent or agents can include, for example, a protein, such as, an antibody, a nucleic acid, such as, an anti-sense RNA or an siRNA, or a small molecule.

The encapsulation vehicle, for example, a liposome, cell, or particle (for example, a nanoparticle) can be conjugated, via standard conjugation techniques, to a targeting molecule, which can be a molecule that binds a cell surface molecule found on the surface of a cancer cell or a cancer stem cell. Exemplary targeting molecules include, for example, an antibody that binds specifically to a cell surface molecule present on cancer cells or cancer stem cells, a ligand of a cell surface molecule found on cancer cells or cancer stem cells, or an aptamer that binds a cell surface molecule found on cancer cells or cancer stem cells.

In another aspect, the invention provides a composition comprising (a) a plurality of agents (for example, two, three, four or five agents) that directly reduce the expression or activity of Oct4, Sox2, Klf2, Klf4, Klf5, Tbx3, Nanog, ESRRB, and REST; and (b) a pharmaceutically acceptable carrier. The agents can be selected from the group consisting of a protein, for example, an antibody, a nucleic acid, for example, an anti-sense RNA or an siRNA, and a small molecule, or a combination thereof.

In another aspect, the invention provides a composition comprising (a) a plurality of agents that directly reduce the expression or activity of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist; and (b) a pharmaceutically-acceptable delivery vehicle, wherein the delivery vehicle contains one or more moieties that target and bind surface molecules on a cancer cell or a cancer stem cell. The agents can be selected from the group consisting of a protein, for example, an antibody, a nucleic acid, for example, an anti-sense RNA or an siRNA, and a small molecule, or a combination thereof.

These and other aspects and advantages of the invention will become apparent upon consideration of the following figures, detailed description, and claims.

### BRIEF DESCRIPTION OF FIGURES

The invention can be more completely understood with reference to the following drawings, in which:

FIG. 1 is a schematic representation showing a first transition from a differentiated cell into a cancer stem cell and a second transition from a cancer stem cell into a differentiated cell, together with an agent that inhibits the transition from a differentiated cell into a cancer stem cell, an agent that inhibits the maintenance of the cancer stem cell state, and an agent that enhances differentiation of a cancer stem cell into a differentiated cell;

FIG. 2 shows three exemplary approaches for reducing the number of cancer stem cells in a mixed population of differentiated cells (boxes) and cancer stem cells (circles). In accordance with the teachings of the invention, existing cancer stem cells are stimulated to become differentiated cells (stars). Viable cells are denoted by solid lines, and dead cells are denoted by dashed lines. The dashed line surrounding the boxes, circles and stars represents an outline of a tumor or the remnants of a tumor. FIG. 2A shows an approach where a mixed population of cells is exposed to (i) one or more agents that inhibit differentiated cells from becoming cancer stem cells and/or inhibit maintenance of cancer stem cells (i.e., stemness reducing agents) and (ii) one or more anti-neoplastic agents that kill the originally differentiated cells (including the differentiated cells that had a propensity to becomes stem cells) and the differentiated cells that were originally cancer stem cells. FIG. 2B shows an approach where a mixed population of cells is exposed to one or more stemness reducing agents and then the differentiated cells are exposed to one or more anti-neoplastic agents that kill the originally differentiated cells and the differentiated cells that were originally cancer stem cells. FIG. 2C shows an approach where the mixed population of cells are exposed to one or more stemness reducing agents. The mixed cell population then is exposed to the same or similar stemness reducing agents together with to one or more anti-neoplastic agents that kill the originally differentiated cells and the differentiated cells that were originally cancer stem cells.

### DETAILED DESCRIPTION

The oncogenesis and progression of cancer has been associated with the development of cells with increased stemness. As used herein and with reference to a mammalian cell, for example, a human or non-human cell, the term “stemness” is understood to mean the ability of a cell to self-renew and to generate an additional, phenotypically distinct cell type.

Cancer stem cells have been reported to constitute a small fraction, for example, 0.1% to 10%, of all cancer cells in a tumor. It is believed that cancer cells having stem cell-like characteristics may, under certain circumstances, be the critical initiating cells in the genesis of cancer as well as in the progression of cancer by evolving cells with phenotypes distinct from previous generations. Stem-like cells, namely cancer stem cells, because of their slow growth and replication, are thought to be the hardest cells to eradicate in a cancer. The residual cancer stem cells can then facilitate the replication of an entire cancer following the elimination of all other cells. Following treatment, there may be a period of remission followed by a period of recurrence. Nevertheless, by inhibiting a stem-like phenotype, such cells can be eliminated, thereby preventing or reducing the possibility of a cancer from recurring. Furthermore, treatment with stemness-reducing agents reduces the number of cells with stem cell like qualities and as a result reduces the likelihood of adaption (resistance) when a cell is exposed to an anti-cancer agent.

The invention, therefore, provides methods and compositions for reducing or eliminating cancer stem cells either alone or in a mixed population of differentiated cells. As a result, the invention not only provides new approaches for treating cancer but also provides model systems for developing therapeutic agents, combinations of therapeutic agents and treatment regimens that ultimately can be used for treating cancer.

The term “stem cell” as used herein refers to a cell that (i) is capable of self-renewal, and (ii) is capable of generating an additional phenotypically distinct cell type. The term “differentiated cell” as used herein refers to a cell with a distinct phenotype that is incapable of producing cells with a distinctly different phenotype.

The term “cancer cell” as used herein refers to a cell capable of producing a neoplasm. A neoplasm can be malignant or benign, and is present after birth. Cancer cells have acquired one or more of the “hallmarks of cancer” defined by Hanahan and Weinberg (CELL 100:57-70, 2000) including: i) self-sufficiency in growth signals, ii) insensitivity to anti-growth signals, iii) evasion of apoptosis, iv) ability to promote sustained angiogenesis, v) ability to invade tissues and metastasize, and vi) ability for limitless replicative potential. It is understood that the acquisition of any of these hallmarks may result form genetic mutation(s) and/or epigenetic mechanisms.

The term “cancer stem cell” as used herein refers to a cell that exhibits at least one hallmark of cancer, and is capable of generating at least one additional, phenotypically distinct cell type. Furthermore, cancer stem cells are capable of both asymmetric and symmetric replication. It is appreciated that a cancer stem cell may result from differentiated cancer cells that acquire stemness traits and/or stem cells that acquire phenotypes associated with cancer cells. It is further appreciated that, under certain circumstances, cancer stem cells can reconstitute non-stromal cell types within a tumor.

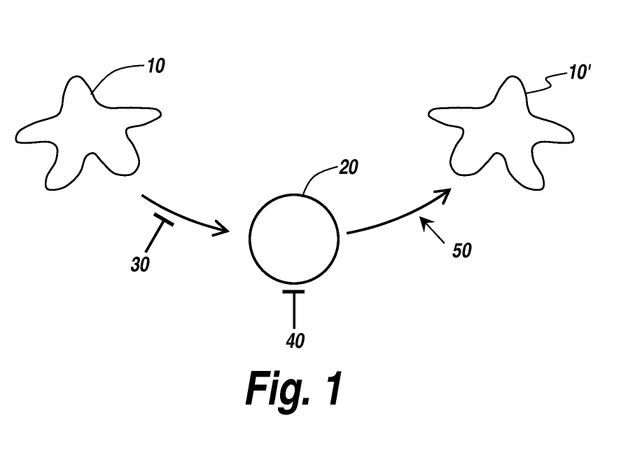

The invention is based, in part, upon the reduction of stem-like cells, namely, cancer stem cells, which can provide the basis for producing new, differentiated cancer cells by the process of asymmetric replication. It is understood that the reduction in stemness, which can occur on a cell-by-cell basis or on a population basis, can be facilitated by one or more approaches shown in FIG. 1.

In particular, FIG. 1 shows a first transition from a differentiated cell 10 to a cancer stem cell 20, and a second transition from the cancer stem cell 20 to a differentiated cell 10′. It is understood that the differentiated cell 10′ can be phenotypically the same as, or phenotypically different from, the original differentiated cell 10. It is understood that the reduction in stemness can be facilitated by one or more agents that include (i) an agent 30 that inhibits the transition of a differentiated cell 10 (a differentiated cell with a propensity to form a cancer stem cell) into cancer stem cell 20, (ii) an agent 40 that inhibits the maintenance of a cancer stem cell 20, and (iii) an agent 50 that enhances the differentiation of cancer stem cell 20 into differentiated cells 10′.

It is understood that there is considerable overlap between the agents, as many of the targets for the agents, in particular, certain transcription factors, are involved in both inducing the transition of differentiated cells into cancer stem cells and in maintaining the sternness phenotype of cancer stem cells. It is understood that the practice of the invention can include using two or more agents (for example, two, three, four, five or six agents or more) to reduce the number of cancer stem cells in a population.

The invention provides a method of facilitating cell death of cancer stem cells or differentiated cells with a propensity for forming cancer stem cells in a mixed population of cancer stem cells and differentiated cells. The method comprises the steps of (a) exposing the mixed population of cancer stem cells and differentiated cells to an effective amount of one or more agents that inhibit the formation of cancer stem cells from one or more of the differentiated cells thereby to produce a second population of cells with fewer cancer stem cells or differentiated cells with a propensity for forming cancer stem cells, and (b) exposing the second population of cells to an effective amount of an anti-neoplastic agent, for example, a chemotherapeutic agent, that causes cell death of the differentiated cells in the second population of cells. It is understood that step (b) can occur after or contemporaneously with step (a).

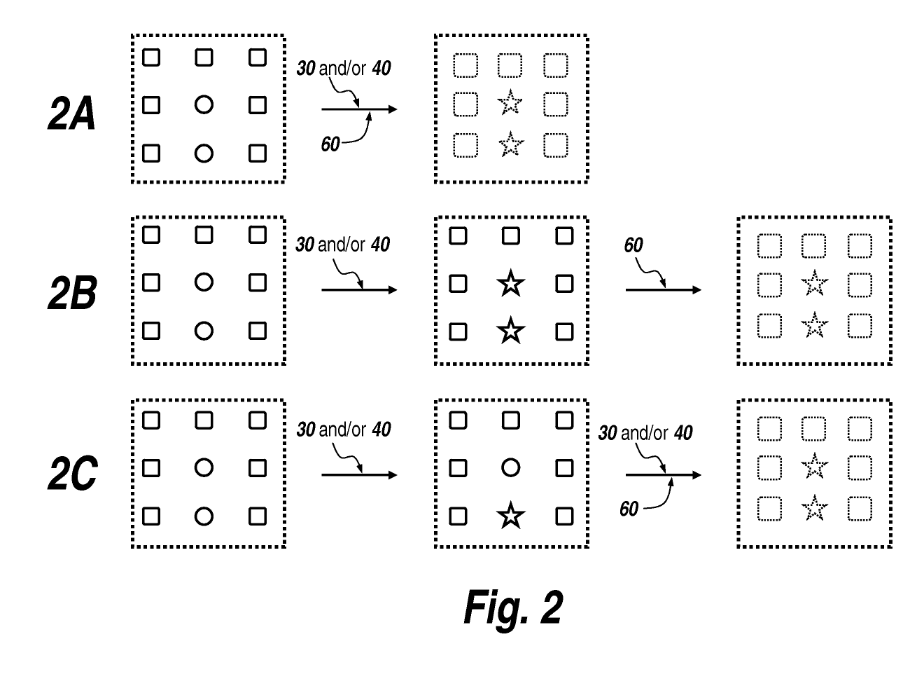

The reduction in stemness in a mixed population of differentiated cells and cancer stem cells can be facilitated by a number of approaches, as shown in FIG. 2. Differentiated cells are denoted by boxes, cancer stem cells are denoted by circles and differentiated cells that originated from stem cells are denoted by stars. Viable cells are denoted by solid lines, and dead cells are denoted by dashed lines. The dashed line surrounding the cells denotes a tumor or the space where a tumor used to exist.

FIG. 2A shows an approach where a mixed population of cells is simultaneously exposed to (i) one or more stemness reducing agents that stop differentiated cells from becoming cancer stem cells and/or inhibit the maintenance of cancer stem cells (e.g., one or more agents 30, one or more agents 40, or a combination of agents 30 and 40) and (ii) one or more anti-neoplastic agents 60 that kill the originally differentiated cells (including the differentiated cells that had a propensity to becomes stem cells) and the differentiated cells that were originally cancer stem cells.

FIG. 2B shows a sequential approach where mixed population of cells is initially exposed to one or more stemness reducing agents (30 and/or 40). Thereafter, the differentiated cells are exposed to one or more anti-neoplastic agents 60 that kill the originally differentiated cells and the differentiated cells that were originally cancer stem cells.

FIG. 2C shows a sequential approach where the mixed population of cells is exposed to one or more stemness reducing agents (30 and/or 40). The mixed cell population then is exposed to the same or similar stemness reducing agents together with one or more anti-neoplastic agents 60 that kill the originally differentiated cells and the differentiated cells that were originally cancer stem cells.

The invention also provides a method of reducing the number of cancer stem cells or differentiated cells with a propensity for forming cancer stem cells in a mixed population of cancer stem cells and differentiated cells. The method comprises exposing the mixed population of cancer stem cells and differentiated cells to at least two different agents that directly reduce the expression or activity of two transcription factors that modulate the formation of cancer stem cells from one or more of the differentiated cells and/or modulate the maintenance of the cancer stem cells thereby to reduce the number of cancer stem cells or differentiated stem cells with a propensity for forming cancer stem cells in the mixed population.

The invention also provides a method of reducing the relative number of cancer stem cells or differentiated cells with a propensity for forming cancer stem cells in a mixed population of cancer stem cells and differentiated cells. The method comprises exposing the mixed population of cancer stem cells and differentiated cells with a combination of a first agent that inhibits the formation of cancer stem cells from one or more of the differentiated cells and a second agent that inhibits the maintenance of the cancer stem cells thereby to reduce the relative number of cancer stem cells or differentiated cells with a propensity for forming cancer stem cells in the mixed population.

### A. Active Agents

It is understood that a variety of active agents, either alone or in combination, can be used in the practice of the methods described herein, and are discussed in the following sections.

With respect to the agents described herein, the terms “modulate” and “modulation” refer to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit” or “reduce” as used herein refer to any inhibition, reduction, decrease, suppression, downregulation, or prevention in expression or activity and include partial or complete inhibition of gene expression or gene product activity. Partial inhibition can imply a level of expression or activity that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression or activity. The terms “activate” or “induce” are used herein to refer to any activation, induction, increase, stimulation, or upregulation in expression or activity and include partial activation of gene expression or gene product activity, such as, for example, an increase of at least 5%, at least 10%, at least 20%, at least 40%, at least 60%, at least 80%, at least 100%, at least 150%, at least 200%, of the expression or activity in the absence of the agonist.

The term “gene product” as used herein means an RNA (for example, a messenger RNA (mRNA)) or protein that is encoded by the gene. The term “expression” is used herein to mean the process by which a polypeptide is produced from DNA. The process involves the transcription of the gene into mRNA and the translation of this mRNA into a polypeptide. Depending on the context in which used, “expression” may refer to the production of mRNA, protein, or both.

(a) Stem Cell or Stemness Reducing Agents

Because certain transcription factors are upregulated in cancer stem cells versus differentiated cells, the transition of differentiated cells into cancer stem cells and/or the maintenance of stem cells can be modulated by exposing the cells to an antagonist that directly reduces the expression or activity of such transcription factors. An agent acts “directly” when the agent (either alone or in combination with one or more other agents) itself specifically modulates the expression or activity of a target molecule, for example, a transcription factor described herein, at the level of the expression of the gene encoding the target molecule or the gene product. As a result, such agents can inhibit the production of cancer stem cells and/or can stimulate, induce or promote the differentiation of cancer stem cells into differentiated cells.

Exemplary transcription factors involved in the induction and/or maintenance of cancer stem cells, which represent targets for blocking the transition of differentiated cells into cancer stem cells and/or the maintenance of cancer stem cells, include: Oct4 (NM_{—}002701 (DNA: SEQ ID NO: 1; Protein: SEQ ID NO: 2), NP_{—}002692 (SEQ ID NO: 2), NM_{—}203289 (DNA: SEQ ID NO: 3; Protein: SEQ ID NO: 4), NP_{—}976034 (SEQ ID NO: 4)), Sox2 (NM_{—}003106 (DNA: SEQ ID NO: 5; Protein: SEQ ID NO: 6), NP_{—}003097 (SEQ ID NO: 6)), Klf4 (NM_{—}004235 (DNA: SEQ ID NO: 7; Protein: SEQ ID NO: 8), NP_{—}004226 (SEQ ID NO: 8)), Nanog (NM_{—}024865 (DNA: SEQ ID NO: 9; Protein: SEQ ID NO: 10), NP_{—}079141 (SEQ ID NO: 10)), c-Myc (NM_{—}002467 (DNA: SEQ ID NO: 11; Protein: SEQ ID NO: 12), NP_{—}002458 (SEQ ID NO: 12)), Klf5 (NM_{—}001730 (DNA: SEQ ID NO: 13; Protein: SEQ ID NO: 14), NP_{—}001721 (SEQ ID NO: 14)), Klf2 (NM_{—}016270 (DNA: SEQ ID NO: 15; Protein: SEQ ID NO: 16), NP_{—}057354 (SEQ ID NO: 16)), and ESRRB (NM_{—}004452 (DNA: SEQ ID NO: 17; Protein: SEQ ID NO: 18), NP_{—}004443 (SEQ ID NO: 18)), REST (NM_{—}005612 (DNA: SEQ ID NO: 19; Protein: SEQ ID NO: 20), NP_{—}005603 (SEQ ID NO: 20)), and Tbx3 (NM_{—}005996 (DNA: SEQ ID NO: 21; Protein: SEQ ID NO: 22), NP_{—}005987 (SEQ ID NO: 22)).

A full length nucleotide sequence encoding and a protein sequence defining a first variant of Oct4 appear in SEQ ID NO: 1 and 2, respectively. A full length nucleotide sequence encoding and a protein sequence defining a second variant of Oct4 appears in SEQ ID NO: 3 and 4, respectively. A full length nucleotide sequence encoding and a protein sequence defining Sox2 appear in SEQ ID NO: 5 and 6, respectively. A full length nucleotide sequence encoding and a protein sequence defining Klf4 appear in SEQ ID NO: 7 and 8, respectively. A full length nucleotide sequence encoding and a protein sequence defining Nanog appear in SEQ ID NO: 9 and 10, respectively. A full length nucleotide sequence encoding and a protein sequence defining c-myc appear in SEQ ID NO: 11 and 12, respectively. A full length nucleotide sequence encoding and a protein sequence defining Klf5 appear in SEQ ID NO: 13 and 14, respectively. A full length nucleotide sequence encoding and a protein sequence defining Klf2 appear in SEQ ID NO: 15 and 16, respectively. A full length nucleotide sequence encoding and a protein sequence defining ESRRB appear in SEQ ID NO: 17 and 18, respectively. A full length nucleotide sequence encoding and a protein sequence defining REST appear in SEQ ID NO: 19 and 20, respectively. A full length nucleotide sequence encoding and a protein sequence defining TBX3 appear in SEQ ID NO: 21 and 22, respectively.

Additionally, exemplary transcription factors involved in the induction and/or maintenance of cancer stem cells, which represent targets for blocking the transition of differentiated cells into cancer stem cells and/or the maintenance of cancer stem cells, include Foxc1 (NM_{—}001453 (DNA: SEQ ID NO: 23; Protein: SEQ ID NO: 24), NP_{—}001444 (SEQ ID NO: 24)), Foxc2 (NM_{—}005251 (DNA: SEQ ID NO: 25; Protein: SEQ ID NO: 26), NP_{—}005242 (SEQ ID NO: 26)), Goosecoid (NM_{—}173849 (DNA: SEQ ID NO: 27; Protein: SEQ ID NO: 28), NP_{—}776248 (SEQ ID NO: 28)), Sip1 (NM_{—}001009183 (DNA: SEQ ID NO: 29; Protein: SEQ ID NO: 30), NP_{—}001009183 (SEQ ID NO: 30)), Snail1 (NM_{—}005985 (DNA: SEQ ID NO: 31; Protein: SEQ ID NO: 32), NP_{—}005976 (SEQ ID NO: 32)), Snail2 (NM_{—}003068 (DNA: SEQ ID NO: 33; Protein: SEQ ID NO: 34), NP_{—}003059 (SEQ ID NO: 34)), TCF3 (NM_{—}003200 (DNA: SEQ ID NO: 35; Protein: SEQ ID NO: 36), NP_{—}003191 (SEQ ID NO: 36)), and Twist (NM_{—}000474 (DNA: SEQ ID NO: 37; Protein: SEQ ID NO: 38), NP_{—}000465 (SEQ ID NO: 38)).

A full length nucleotide sequence encoding and a protein sequence defining Foxc1 appear in SEQ ID NO: 23 and 24, respectively. A full length nucleotide sequence encoding and a protein sequence defining Foxc2 appear in SEQ ID NO: 25 and 26, respectively. A full length nucleotide sequence encoding and a protein sequence defining Goosecoid appear in SEQ ID NO: 27 and 28, respectively. A full length nucleotide sequence encoding and a protein sequence defining Sip1 appear in SEQ ID NO: 29 and 30, respectively. A full length nucleotide sequence encoding and a protein sequence defining Snail1 appear in SEQ ID NO: 31 and 32, respectively. A full length nucleotide sequence encoding and a protein sequence defining Snail2 appear in SEQ ID NO: 33 and 34, respectively. A full length nucleotide sequence encoding and a protein sequence defining TCF3 appear in SEQ ID NO: 35 and 36, respectively. A full length nucleotide sequence encoding and a protein sequence defining Twist appear in SEQ ID NO: 37 and 38, respectively.

These targets can be inhibited (e.g., by inhibiting their transcription, their translation, or their post-translation levels or activity) separately or in combination. For example, inhibitors of two, three, four, five, six, seven, or more of these transcription factors can be used concurrently, sequentially, or otherwise in combination to discourage the induction and/or maintenance of stemness. Accordingly, it is contemplated that the practice of the invention may involve the use of a single inhibitor, for example, an inhibitor of Oct4 or an inhibitor of Sox2. However, it is also contemplated that the practice of the invention may involve the use of a combination of different inhibitors, for example, an inhibitor of Oct4 combined (used either together or sequentially) and an inhibitor of Sox2.

It is understood that various combinations of inhibitors can include inhibitors as set forth in TABLE 1. In TABLE 1, where an inhibitor of Klf4 is included, inhibitors of Klf2 and/or Klf5 can replace or supplement the Klf4 inhibitor.

TABLE 1

| Oct4 | Sox2 | Klf4 | Nanog | c-Myc | ESRRB | REST | Tbx3 |
|---|---|---|---|---|---|---|---|
| X | X | | | | | | |
| X | | X | | | | | |
| X | | | X | | | | |
| X | | | | X | | | |
| X | | | | | X | | |
| X | | | | | | X | |
| X | | | | | | | X |
| | X | X | | | | | |
| | X | | X | | | | |
| | X | | | X | | | |
| | X | | | | X | | |
| | X | | | | | X | |
| | X | | | | | | X |
| | | X | X | | | | |
| | | X | | X | | | |
| | | X | | | X | | |
| | | X | | | | X | |
| | | X | | | | | X |
| | | | X | X | | | |
| | | | X | | X | | |
| | | | X | | | X | |
| | | | X | | | | X |
| | | | | X | X | | |
| | | | | X | | X | |
| | | | | X | | | X |
| | | | | | X | X | |
| | | | | | X | | X |
| | | | | | | X | X |
| X | X | X | | | | | |
| X | | X | X | | | | |
| X | | | X | X | | | |
| X | | | | X | X | | |
| X | | | | | X | X | |
| X | | | | | | X | X |
| X | X | | X | | | | |
| X | X | | | X | | | |
| X | X | | | | X | | |
| X | X | | | | | X | |
| X | X | | | | | | X |
| X | | X | | X | | | |
| X | | X | | | X | | |
| X | | X | | | | X | |
| X | | X | | | | | X |
| X | | | X | | X | | |
| X | | | X | | | X | |
| X | | | X | | | | X |
| X | | | | X | | X | |
| X | | | | X | | | X |
| X | | | | | X | | X |
| | X | X | X | | | | |
| | X | X | | X | | | |
| | X | X | | | X | | |
| | X | X | | | | X | |
| | X | X | | | | | X |
| | X | | X | X | | | |
| | X | | X | | X | | |
| | X | | X | | | X | |
| | X | | X | | | | X |
| | X | | | X | X | | |
| | X | | | X | | X | |
| | X | | | X | | | X |
| | X | | | | X | X | |
| | X | | | | X | | X |
| | X | | | | | X | X |
| | | X | X | X | | | |
| | | X | X | | X | | |
| | | X | X | | | X | |
| | | X | X | | | | X |
| | | X | | X | X | | |
| | | X | | X | | X | |
| | | X | | X | | | X |
| | | X | | | X | X | |
| | | X | | | X | | X |
| | | X | | | | X | X |
| | | | X | X | X | | |
| | | | X | X | | X | |
| | | | X | X | | | X |
| | | | X | | X | X | |
| | | | X | | X | | X |
| | | | X | | | X | X |
| | | | | X | X | X | |
| | | | | X | X | | X |
| | | | | X | | X | X |
| | | | | | X | X | X |
| X | X | X | X | | | | |
| X | X | X | | X | | | |
| X | X | X | | | X | | |
| X | X | X | | | | X | |
| X | X | X | | | | | X |
| X | X | | X | X | | | |
| X | X | | X | | X | | |
| X | X | | X | | | X | |
| X | X | | X | | | | X |
| X | X | | | X | X | | |
| X | X | | | X | | X | |
| X | X | | | X | | | X |
| X | X | | | | X | X | |
| X | X | | | | X | | X |
| X | X | | | | | X | X |
| X | | X | X | X | | | |
| X | | X | X | | X | | |
| X | | X | X | | | X | |
| X | | X | X | | | | X |
| X | | X | | X | X | | |
| X | | X | | X | | X | |
| X | | X | | X | | | X |
| X | | X | | | X | X | |
| X | | X | | | X | | X |
| X | | X | | | | X | X |
| X | | | X | X | X | | |
| X | | | X | X | | X | |
| X | | | X | X | | | X |
| X | | | X | | X | X | |
| X | | | X | | X | | X |
| X | | | X | | | X | X |
| X | | | | X | X | X | |
| X | | | | X | X | | X |
| X | | | | X | | X | X |
| X | | | | | X | X | X |
| | X | X | X | X | | | |
| | X | X | X | | X | | |
| | X | X | X | | | X | |
| | X | X | X | | | | X |
| | X | X | | X | X | | |
| | X | X | | X | | X | |
| | X | X | | X | | | X |
| | X | X | | | X | X | |
| | X | X | | | X | | X |
| | X | X | | | | X | X |
| | X | | X | X | X | | |
| | X | | X | X | | X | |
| | X | | X | X | | | X |
| | X | | X | | X | X | |
| | X | | X | | X | | X |
| | X | | X | | | X | X |
| | X | | | X | X | X | |
| | X | | | X | X | | X |
| | X | | | X | | X | X |
| | X | | | | X | X | X |
| | | X | X | X | X | | |
| | | X | X | X | | X | |
| | | X | X | X | | | X |
| | | X | X | | X | X | |
| | | X | X | | X | | X |
| | | X | X | | | X | X |
| | | X | | X | X | X | |
| | | X | | X | X | | X |
| | | X | | X | | X | X |
| | | X | | | X | X | X |
| | | | X | X | X | X | |
| | | | X | X | X | | X |
| | | | X | X | | X | X |
| | | | X | | X | X | X |
| | | | | X | X | X | X |
| X | X | X | X | X | | | |
| X | X | X | X | | X | | |
| X | X | X | X | | | X | |
| X | X | X | X | | | | X |
| X | X | X | | X | X | | |
| X | X | X | | X | | X | |
| X | X | X | | X | | | X |
| X | X | X | | | X | X | |
| X | X | X | | | X | | X |
| X | X | X | | | | X | X |
| X | X | | X | X | X | | |
| X | X | | X | X | | X | |
| X | X | | X | X | | | X |
| X | X | | X | | X | X | |
| X | X | | X | | X | | X |
| X | X | | X | | | X | X |
| X | X | | | X | X | X | |
| X | X | | | X | X | | X |
| X | X | | | X | | X | X |
| X | X | | | | X | X | X |
| X | | X | X | X | X | | |
| X | | X | X | X | | X | |
| X | | X | X | X | | | X |
| X | | X | X | | X | X | |
| X | | X | X | | X | | X |
| X | | X | X | | | X | X |
| X | | X | | X | X | X | |
| X | | X | | X | X | | X |
| X | | X | | X | | X | X |
| X | | X | | | X | X | X |
| X | | | X | X | X | X | |
| X | | | X | X | X | | X |
| X | | | X | X | | X | X |
| X | | | X | | X | X | X |
| X | | | | X | X | X | X |
| | X | X | X | X | X | | |
| | X | X | X | X | | X | |
| | X | X | X | X | | | X |
| | X | X | X | | X | X | |
| | X | X | X | | X | | X |
| | X | X | X | | | X | X |
| | X | X | | X | X | X | |
| | X | X | | X | X | | X |
| | X | X | | X | | X | X |
| | X | X | | | X | X | X |
| | X | | X | X | X | X | |
| | X | | X | X | X | | X |
| | X | | X | X | | X | X |
| | X | | X | | X | X | X |
| | X | | | X | X | X | X |
| | | X | X | X | X | X | |
| | | X | X | X | X | | X |
| | | X | X | X | | X | X |
| | | X | X | | X | X | X |
| | | X | | X | X | X | X |
| | | | X | X | X | X | X |

It is understood that the combinations of targeted transcription factors listed in TABLE 1 can also include one or more of the transcription factors selected from the group consisting of Klf2, Klf5, Foxc1, Foxc2, Goosecoid, Sip1, Snail1, Snail2, TCF3, and Twist.

Agents that inhibit the expression or activity of a stemness inducing transcription factor and/or a stemness maintaining transcription factor include, but are not limited to, nucleic acids, polypeptides, and small molecule drugs (e.g., small molecules having a molecular weight of less than 1 kDa). Additionally, the agent may be a metabolite, a carbohydrate, a lipid, or any other molecule that binds or interacts with a gene product of one or more of the foregoing transcription factors.

Furthermore it is contemplated that in the case of a cocktail of inhibitors it is possible that the inhibitors can include a combination of one or more nucleic acids (one or more of which may be directed to a particular transcription factor gene or may be directed to different transcription factor genes), one or more proteins, and/or one or more small molecules.

Exemplary nucleic acid-based modulators include, but are not limited to, RNAs, DNAs, and PNAs. Exemplary RNAs include, for example, antisense RNA, short interfering RNA (siRNA), short hairpin RNA (shRNA), and microRNA (miRNA). In addition, it is contemplated that RNA and DNA aptamers can be used in the practice of the invention.

In certain embodiments, the agent is a siRNA specific to one or more genes encoding a stemness inducing transcription factor and/or a sternness maintenance transcription factor. Exemplary synthetic siRNAs include 21 nucleotide RNAs chemically synthesized using methods known in the art (e.g., Expedite RNA phophoramidites and thymidine phosphoramidite (Proligo, Germany)). Synthetic oligonucleotides preferably are deprotected and gel-purified using methods known in the art (see, e.g., Elbashir et al. (2001) GENES DEV. 15: 188-200). Longer RNAs may be transcribed from promoters, such as T7 RNA polymerase promoters, known in the art. A single RNA target, placed in both possible orientations downstream of an in vitro promoter, can transcribe both strands of the target to create a dsRNA oligonucleotide of the desired target sequence.

The resulting siRNAs can be delivered as multiple siRNAs with each siRNA targeting one or more genes. Alternatively, multiple siRNAs can be used to target a target gene (see, for example, U.S. Patent Application Publication No. US2005/0197313, which describes a system for delivering multiple siRNAs to target multiple versions of the same gene). Alternatively, a single siRNA can be used to target multiple genes.

The following sections provide exemplary siRNAs that can be used to reduce the expression of certain transcription factors, including, Oct4, Sox2, Klf4, Nanog, c-Myc, Klf5, Klf2, ESRRB, REST, TBX3, Foxc1, Foxc2, Goosecoid, Sip1, Snail1, Snail2, TCF3, and Twist. It is understood that siRNAs often have extra bases added (UU) for overhangs that are not explicitly placed on the sequences presented. Furthermore, the siRNAs presented in Tables 2-19 represent single stranded RNAs, however, it is understood that complementary RNA sequences may also be useful in the practice of the invention. In addition, it is understood that the siRNAs shown in the Tables 2-19 or RNA sequences complementary thereto maybe delivered using conventional delivery techniques known to those skilled in the art. Alternatively, longer RNA sequences, or double stranded DNA sequences that encode at least the sequences noted in Tables 2-19 can be delivered using conventional techniques known to those skilled in the art.

Exemplary siRNAs for Oct4 are shown below in TABLE 2. The location of each siRNA relative to the target is identified where the term “ORF” denotes the open reading frame and the term “UTR” denotes the untranslated region.

TABLE 2

| | REGION IN | START | |
|---|---|---|---|
| SEQUENCE | TARGET | POSITION | SEQ ID NO. |
| AGGAGAAGCUGGAGCAAAA | ORF | 431 | 39 |
| CCGUGAAGCUGGAGAAGGA | ORF | 416 | 40 |
| GAGUCGGGGUGGAGAGCAA | ORF | 353 | 41 |
| AGAAGGAGAAGCUGGAGCA | ORF | 428 | 42 |
| AAGGAGAAGCUGGAGCAAA | ORF | 430 | 43 |
| GUGCCGUGAAGCUGGAGAA | ORF | 413 | 44 |
| GUGAAGCUGGAGAAGGAGA | ORF | 418 | 45 |
| UGGAGAAGGAGAAGCUGGA | ORF | 425 | 46 |
| GAAGGAGAAGCUGGAGCAA | ORF | 429 | 47 |
| GAGCAAAACCCGGAGGAGU | ORF | 442 | 48 |
| AGAAAGAACUCGAGCAAUU | ORF | 482 | 49 |
| AGGAGAAGCUGGAGCAAAA | ORF | 431 | 50 |
| CAUCAAAGCUCUGCAGAAA | ORF | 468 | 51 |
| GCAGAAAGAACUCGAGCAA | ORF | 480 | 52 |
| CCGUGAAGCUGGAGAAGGA | ORF | 416 | 53 |
| GAGGCAACCUGGAGAAUUU | ORF | 779 | 54 |
| GGAGAUAUGCAAAGCAGAA | ORF | 708 | 55 |
| GCUUCAAGAACAUGUGUAA | ORF | 632 | 56 |
| CGAAAGAGAAAGCGAACCA | ORF | 742 | 57 |
| GGGAGGAGCUAGGGAAAGA | 3′ UTR | 1174 | 58 |
| GGAUUAAGUUCUUCAUUCA | 3′ UTR | 1221 | 59 |
| CAGAAGGGCAAGCGAUCAA | ORF | 901 | 60 |
| GGGACACAGUAGAUAGACA | 3′ UTR | 1377 | 61 |
| GUAGAUAGACACACUUAAA | 3′ UTR | 1385 | 62 |
| GAGUCGGGGUGGAGAGCAA | ORF | 353 | 63 |
| ACAUCAAAGCUCUGCAGAA | ORF | 467 | 64 |
| UCAAAGCUCUGCAGAAAGA | ORF | 470 | 65 |
| GGGUGGAGGAAGCUGACAA | ORF | 674 | 66 |
| AGAGAAAGCGAACCAGUAU | ORF | 746 | 67 |
| CAAUGAUGCUCUUGAUUUU | 3′ UTR | 1315 | 68 |
| CCAAGCUCCUGAAGCAGAA | ORF | 503 | 69 |
| GAGAUAUGCAAAGCAGAAA | ORF | 709 | 70 |
| CUAAGGAAGGAAUUGGGAA | 3′ UTR | 1240 | 71 |
| CAGUAGAUAGACACACUUA | 3′ UTR | 1383 | 72 |
| UUGCCAAGCUCCUGAAGCA | ORF | 500 | 73 |
| AGAAGUGGGUGGAGGAAGC | ORF | 668 | 74 |
| AGAAGGAGAAGCUGGAGCA | ORF | 428 | 75 |
| AAGGAGAAGCUGGAGCAAA | ORF | 430 | 76 |
| GCAGAAGUGGGUGGAGGAA | ORF | 666 | 77 |
| GCCCGAAAGAGAAAGCGAA | ORF | 739 | 78 |
| UGAGAGGCAACCUGGAGAA | ORF | 776 | 79 |
| AGGGGAGGAGCUAGGGAAA | 3′ UTR | 1172 | 80 |
| GGGAUUAAGUUCUUCAUUC | 3′ UTR | 1220 | 81 |
| GUGCCGUGAAGCUGGAGAA | ORF | 413 | 82 |
| GAACCGAGUGAGAGGCAAC | ORF | 768 | 83 |
| AGAAGGAUGUGGUCCGAGU | ORF | 863 | 84 |
| UAAGGAAGGAAUUGGGAAC | 3′ UTR | 1241 | 85 |
| GUGAAGCUGGAGAAGGAGA | ORF | 418 | 86 |
| UGGAGAAGGAGAAGCUGGA | ORF | 425 | 87 |
| CUGCAGUGCCCGAAACCCA | ORF | 802 | 88 |
| GAAGGAGAAGCUGGAGCAA | ORF | 429 | 89 |
| AGCUUGGGCUCGAGAAGGA | ORF | 851 | 90 |
| GAGCAAAACCCGGAGGAGU | ORF | 442 | 91 |
| GAAAGAACUCGAGCAAUUU | ORF | 483 | 92 |
| GCCAGAAGGGCAAGCGAUC | ORF | 899 | 93 |
| UGGUUGGAGGGAAGGUGAA | 3′ UTR | 1293 | 94 |
| AGUAGAUAGACACACUUAA | 3′ UTR | 1384 | 95 |
| CAGAAAGAACUCGAGCAAU | ORF | 481 | 96 |
| AGAAAGAACUCGAGCAAUU | ORF | 226 | 97 |
| CAUCAAAGCUCUGCAGAAA | ORF | 212 | 98 |
| GCAGAAAGAACUCGAGCAA | ORF | 224 | 99 |
| GAGGCAACCUGGAGAAUUU | ORF | 523 | 100 |
| GGGAAGGUAUUCAGCCAAA | ORF | 324 | 101 |
| GGAGAUAUGCAAAGCAGAA | ORF | 452 | 102 |
| GCUUCAAGAACAUGUGUAA | ORF | 376 | 103 |
| CGAAAGAGAAAGCGAACCA | ORF | 486 | 104 |
| GGGAGGAGCUAGGGAAAGA | 3′ UTR | 918 | 105 |
| GGAUUAAGUUCUUCAUUCA | 3′ UTR | 965 | 106 |
| CAGAAGGGCAAGCGAUCAA | ORF | 645 | 107 |
| GGGACACAGUAGAUAGACA | 3′ UTR | 1121 | 108 |
| GUAGAUAGACACACUUAAA | 3′ UTR | 1129 | 109 |
| ACAUCAAAGCUCUGCAGAA | ORF | 211 | 110 |
| CUGAAGCAGAAGAGGAUCA | ORF | 255 | 111 |
| UCAAAGCUCUGCAGAAAGA | ORF | 214 | 112 |
| AGAGGAUCACCCUGGGAUA | ORF | 265 | 113 |
| GGGUGGAGGAAGCUGACAA | ORF | 418 | 114 |
| CGUGCAGGCCCGAAAGAGA | ORF | 476 | 115 |
| GUGCAGGCCCGAAAGAGAA | ORF | 477 | 116 |
| AGAGAAAGCGAACCAGUAU | ORF | 490 | 117 |
| CAAUGAUGCUCUUGAUUUU | 3′ UTR | 1059 | 118 |
| CCAAGCUCCUGAAGCAGAA | ORF | 247 | 119 |
| GAGAUAUGCAAAGCAGAAA | ORF | 453 | 120 |
| CUAAGGAAGGAAUUGGGAA | 3′ UTR | 984 | 121 |
| CAGUAGAUAGACACACUUA | 3′ UTR | 1127 | 122 |
| UUGCCAAGCUCCUGAAGCA | ORF | 244 | 123 |
| AGAAGUGGGUGGAGGAAGC | ORF | 412 | 124 |
| GCAGAAGUGGGUGGAGGAA | ORF | 410 | 125 |
| GCCCGAAAGAGAAAGCGAA | ORF | 483 | 126 |
| UGAGAGGCAACCUGGAGAA | ORF | 520 | 127 |
| AGGGGAGGAGCUAGGGAAA | 3′ UTR | 916 | 128 |
| GGGAUUAAGUUCUUCAUUC | 3′ UTR | 964 | 129 |
| GGUUCUAUUUGGGAAGGUA | ORF | 314 | 130 |
| GAACCGAGUGAGAGGCAAC | ORF | 512 | 131 |
| AGAAGGAUGUGGUCCGAGU | ORF | 607 | 132 |
| UAAGGAAGGAAUUGGGAAC | 3′ UTR | 985 | 133 |
| GUUCUAUUUGGGAAGGUAU | ORF | 315 | 134 |
| CUGCAGUGCCCGAAACCCA | ORF | 546 | 135 |
| GAGGAUCACCCUGGGAUAU | ORF | 266 | 136 |
| AGGAUCACCCUGGGAUAUA | ORF | 267 | 137 |
| AGCUUGGGCUCGAGAAGGA | ORF | 595 | 138 |
| GCCAGAAGGGCAAGCGAUC | ORF | 643 | 139 |
| GAAAGAACUCGAGCAAUUU | ORF | 227 | 140 |
| UGGUUGGAGGGAAGGUGAA | 3′ UTR | 1037 | 141 |
| AGUAGAUAGACACACUUAA | 3′ UTR | 1128 | 142 |
| UGGGAUAUACACAGGCCGA | ORF | 277 | 143 |
| UUGGGAAGGUAUUCAGCCA | ORF | 322 | 144 |
| UCUUCAGGAGAUAUGCAAA | ORF | 446 | 145 |
| GGGAAUGGGUGAAUGACAU | 5′ UTR | 17 | 146 |
| AUUGAUAACUGGUGUGUUU | ORF | 150 | 147 |
| GGAAAGGGGAGAUUGAUAA | ORF | 139 | 148 |
| CUUGAAUCCCGAAUGGAAA | ORF | 125 | 149 |
| GUGAACAGGGAAUGGGUGA | 5′ UTR | 10 | 150 |
| GAGUCAGUGAACAGGGAAU | 5′ UTR | 4 | 151 |
| GAACAGGGAAUGGGUGAAU | 5′ UTR | 12 | 152 |
| UGGAAAGGGGAGAUUGAUA | ORF | 138 | 153 |
| UUACAAGUCUUCUGCCUUU | ORF | 175 | 154 |
| ACAGGGAAUGGGUGAAUGA | 5′ UTR | 14 | 155 |
| UCUUGAAUCCCGAAUGGAA | ORF | 124 | 156 |
| GGUUAUUUCUAGAAGUUAG | 5′ UTR | 45 | 157 |
| GACAUUUGUGGGUAGGUUA | 5′ UTR | 31 | 158 |
| AGGGAAUGGGUGAAUGACA | 5′ UTR | 16 | 159 |
| ACACGUAGGUUCUUGAAUC | ORF | 114 | 160 |
| GGAGAUUGAUAACUGGUGU | ORF | 146 | 161 |
| AGAAAGAACUCGAGCAAUU | ORF | 226 | 162 |
| CAUCAAAGCUCUGCAGAAA | ORF | 212 | 163 |
| GCAGAAAGAACUCGAGCAA | ORF | 224 | 164 |
| GAGGCAACCUGGAGAAUUU | ORF | 523 | 165 |
| GGGAAGGUAUUCAGCCAAA | ORF | 324 | 166 |
| GGAGAUAUGCAAAGCAGAA | ORF | 452 | 167 |
| GGGAAUGGGUGAAUGACAU | 5′ UTR | 17 | 168 |
| GCUUCAAGAACAUGUGUAA | ORF | 376 | 169 |
| AUUGAUAACUGGUGUGUUU | ORF | 150 | 170 |
| CGAAAGAGAAAGCGAACCA | ORF | 486 | 171 |
| GGGAGGAGCUAGGGAAAGA | 3′ UTR | 918 | 172 |
| GGAUUAAGUUCUUCAUUCA | 3′ UTR | 965 | 173 |
| CAGAAGGGCAAGCGAUCAA | ORF | 645 | 174 |
| GGGACACAGUAGAUAGACA | 3′ UTR | 1121 | 175 |
| GUAGAUAGACACACUUAAA | 3′ UTR | 1129 | 176 |
| GGAAAGGGGAGAUUGAUAA | ORF | 139 | 177 |
| CUUGAAUCCCGAAUGGAAA | ORF | 125 | 178 |
| ACAUCAAAGCUCUGCAGAA | ORF | 211 | 179 |
| GUGAACAGGGAAUGGGUGA | 5′ UTR | 10 | 180 |
| UCAAAGCUCUGCAGAAAGA | ORF | 214 | 181 |
| CUGAAGCAGAAGAGGAUCA | ORF | 255 | 182 |
| GAGUCAGUGAACAGGGAAU | 5′ UTR | 4 | 183 |
| AGAGGAUCACCCUGGGAUA | ORF | 265 | 184 |
| GGGUGGAGGAAGCUGACAA | ORF | 418 | 185 |
| CGUGCAGGCCCGAAAGAGA | ORF | 476 | 186 |
| GUGCAGGCCCGAAAGAGAA | ORF | 477 | 187 |
| GAACAGGGAAUGGGUGAAU | 5′ UTR | 12 | 188 |
| AGAGAAAGCGAACCAGUAU | ORF | 490 | 189 |
| CAAUGAUGCUCUUGAUUUU | 3′ UTR | 1059 | 190 |
| UGGAAAGGGGAGAUUGAUA | ORF | 138 | 191 |
| CCAAGCUCCUGAAGCAGAA | ORF | 247 | 192 |
| GAGAUAUGCAAAGCAGAAA | ORF | 453 | 193 |
| CUAAGGAAGGAAUUGGGAA | 3′ UTR | 984 | 194 |
| CAGUAGAUAGACACACUUA | 3′ UTR | 1127 | 195 |
| UUACAAGUCUUCUGCCUUU | ORF | 175 | 196 |
| UUGCCAAGCUCCUGAAGCA | ORF | 244 | 197 |
| AGAAGUGGGUGGAGGAAGC | ORF | 412 | 198 |
| ACAGGGAAUGGGUGAAUGA | 5′ UTR | 14 | 199 |
| UCUUGAAUCCCGAAUGGAA | ORF | 124 | 200 |
| GGUUAUUUCUAGAAGUUAG | 5′ UTR | 45 | 201 |
| GACAUUUGUGGGUAGGUUA | 5′ UTR | 31 | 202 |
| GCAGAAGUGGGUGGAGGAA | ORF | 410 | 203 |
| GCCCGAAAGAGAAAGCGAA | ORF | 483 | 204 |
| UGAGAGGCAACCUGGAGAA | ORF | 520 | 205 |
| AGGGGAGGAGCUAGGGAAA | 3′ UTR | 916 | 206 |
| GGGAUUAAGUUCUUCAUUC | 3′ UTR | 964 | 207 |
| AGGGAAUGGGUGAAUGACA | 5′ UTR | 16 | 208 |
| ACACGUAGGUUCUUGAAUC | ORF | 114 | 209 |
| GGAGAUUGAUAACUGGUGU | ORF | 146 | 210 |

Exemplary siRNAs for Sox2 are shown in TABLE 3.

TABLE 3

| | REGION IN | START | SEQ ID |
|---|---|---|---|
| SEQUENCE | TARGET | POSITION | NO. |
| CCAAGACGCUCAUGAAGAA | ORF | 774 | 211 |
| CGUUCAUCGACGAGGCUAA | ORF | 693 | 212 |
| UCAUGAAGAAGGAUAAGUA | ORF | 783 | 213 |
| UGAUGGAGACGGAGCUGAA | ORF | 438 | 214 |
| CGCUCAUGAAGAAGGAUAA | ORF | 780 | 215 |
| ACGCUCAUGAAGAAGGAUA | ORF | 779 | 216 |
| AUGAAGAAGGAUAAGUACA | ORF | 785 | 217 |
| CAGUACAACUCCAUGACCA | ORF | 1043 | 218 |
| GCUCUUGGCUCCAUGGGUU | ORF | 1133 | 219 |
| CGGAAAACCAAGACGCUCA | ORF | 767 | 220 |
| AGGAGCACCCGGAUUAUAA | ORF | 735 | 221 |
| CCAUGGGUUCGGUGGUCAA | ORF | 1143 | 222 |
| ACAUGAACGGCUGGAGCAA | ORF | 912 | 223 |
| UGACCAGCUCGCAGACCUA | ORF | 1056 | 224 |
| GCUCGCAGACCUACAUGAA | ORF | 1062 | 225 |
| ACCAAGACGCUCAUGAAGA | ORF | 773 | 226 |
| UGAAGAAGGAUAAGUACAC | ORF | 786 | 227 |
| UGCAGGACCAGCUGGGCUA | ORF | 948 | 228 |
| CCACCUACAGCAUGUCCUA | ORF | 1089 | 229 |
| CAGCGCAGAUGCAGCCCAU | ORF | 999 | 230 |
| ACAGUUACGCGCACAUGAA | ORF | 900 | 231 |
| UGGAAACUUUUGUCGGAGA | ORF | 662 | 232 |
| GUGAACCAGCGCAUGGACA | ORF | 884 | 233 |
| CUGCAGUACAACUCCAUGA | ORF | 1040 | 234 |
| GGAGCACCCGGAUUAUAAA | ORF | 736 | 235 |
| AGACGCUCAUGAAGAAGGA | ORF | 777 | 236 |
| GCAACGGCAGCUACAGCAU | ORF | 927 | 237 |
| UGGCAUGGCUCUUGGCUCC | ORF | 1126 | 238 |
| ACCAGCGCAUGGACAGUUA | ORF | 888 | 239 |
| UGAGCGCCCUGCAGUACAA | ORF | 1032 | 240 |
| CAUGAAGAAGGAUAAGUAC | ORF | 784 | 241 |
| GCACAUGAACGGCUGGAGC | ORF | 910 | 242 |
| CACAUGAACGGCUGGAGCA | ORF | 911 | 243 |
| UGGAGCAACGGCAGCUACA | ORF | 923 | 244 |
| AGACCUACAUGAACGGCUC | ORF | 1068 | 245 |
| UGGUCAAGUCCGAGGCCAG | ORF | 1155 | 246 |
| UCGACGAGGCUAAGCGGCU | ORF | 699 | 247 |
| GCACCCGGAUUAUAAAUAC | ORF | 739 | 248 |
| AGUGGAAACUUUUGUCGGA | ORF | 660 | 249 |
| CUGCGAGCGCUGCACAUGA | ORF | 716 | 250 |
| AGAAAGAAGAGGAGAGAGA | 5′ UTR | 104 | 251 |
| GUGCAAAAGAGGAGAGUAA | 3′ UTR | 1444 | 252 |
| AGACUAGGACUGAGAGAAA | 5′ UTR | 90 | 253 |
| AAAGAAGAGGAGAGAGAAA | 5′ UTR | 106 | 254 |
| AUGCACAGUUUGAGAUAAA | 3′ UTR | 2458 | 255 |
| GGAAAGAAAGCUACGAAAA | 3′ UTR | 1710 | 256 |
| UAGAAUAAGUACUGGCGAA | 3′ UTR | 2058 | 257 |
| CCAAGACGCUCAUGAAGAA | ORF | 774 | 258 |
| GUAUAGAUCUGGAGGAAAG | 3′ UTR | 1697 | 259 |
| CCAUGAAAUUACUGUGUUU | 3′ UTR | 2238 | 260 |
| AGAAGAGAGUGUUUGCAAA | 5′ UTR | 43 | 261 |
| AAAGAAAGGGAGAGAAGUU | 5′ UTR | 122 | 262 |
| GCAAAUGACAGCUGCAAAA | 3′ UTR | 1531 | 263 |
| AGAUAAACAUGGCAAUCAA | 3′ UTR | 1870 | 264 |
| AAGAGGAGAGAGAAAGAAA | 5′ UTR | 110 | 265 |
| GCACAGUUUGAGAUAAAUA | 3′ UTR | 2460 | 266 |
| GAGAAGAGAGUGUUUGCAA | 5′ UTR | 42 | 267 |
| GGAGAGAGAAAGAAAGGGA | 5′ UTR | 114 | 268 |
| AGAAAGAAAGGGAGAGAAG | 5′ UTR | 120 | 269 |
| UGAGAGAGAUCCUGGACUU | 3′ UTR | 1610 | 270 |
| AGGAAAGAAAGCUACGAAA | 3′ UTR | 1709 | 271 |
| GCUGAGAAUUUGCCAAUAU | 3′ UTR | 1907 | 272 |
| CCUUAUAACAGGUACAUUU | 3′ UTR | 2416 | 273 |
| GAAGAGAGUGUUUGCAAAA | 5′ UTR | 44 | 274 |
| AGAAGAGGAGAGAGAAAGA | 5′ UTR | 108 | 275 |
| GCAAAAGAGGAGAGUAAGA | 3′ UTR | 1446 | 276 |
| UGAAAUAUGGACACUGAAA | 3′ UTR | 2485 | 277 |
| CGUUCAUCGACGAGGCUAA | ORF | 693 | 278 |
| AGAGAAAGAAAGGGAGAGA | 5′ UTR | 118 | 279 |
| UCAUGAAGAAGGAUAAGUA | ORF | 783 | 280 |
| AAGAAACAGCAUGGAGAAA | 3′ UTR | 1461 | 281 |
| CCGCGAUGCCGACAAGAAA | 3′ UTR | 1584 | 282 |
| GGAGAGGCUUCUUGCUGAA | 3′ UTR | 1933 | 283 |
| GAAUCAGUCUGCCGAGAAU | 3′ UTR | 2370 | 284 |
| UAAGAAACAGCAUGGAGAA | 3′ UTR | 1460 | 285 |
| UUGUAUAGAUCUGGAGGAA | 3′ UTR | 1695 | 286 |
| UGAUGGAGACGGAGCUGAA | ORF | 438 | 287 |
| GGUAGGAGCUUUGCAGGAA | 3′ UTR | 1753 | 288 |
| GGACAGUUGCAAACGUGAA | 3′ UTR | 1976 | 289 |
| AAUAAGUACUGGCGAACCA | 3′ UTR | 2061 | 290 |
| AGGUUGACACCGUUGGUAA | 3′ UTR | 2165 | 291 |
| GAGAAAGAAAGGGAGAGAA | 5′ UTR | 119 | 292 |
| CAGGAGUUGUCAAGGCAGA | 5′ UTR | 25 | 293 |
| CGCUCAUGAAGAAGGAUAA | ORF | 780 | 294 |
| AAGAGGAGAGUAAGAAACA | 3′ UTR | 1450 | 295 |

Exemplary siRNAs for Klf4 are shown in TABLE 4.

TABLE 4

| | REGION IN | START | |
|---|---|---|---|
| SEQUENCE | TARGET | POSITION | SEQ ID NO. |
| GGAGAGAGACCGAGGAGUU | ORF | 580 | 296 |
| CAGAGGAGCCCAAGCCAAA | ORF | 1429 | 297 |
| GGACGGCUGUGGAUGGAAA | ORF | 1592 | 298 |
| GGGAGAAGACACUGCGUCA | ORF | 391 | 299 |
| CCUUCAACCUGGCGGACAU | ORF | 853 | 300 |
| CAGAAUUGGACCCGGUGUA | ORF | 919 | 301 |
| UGGGCAAGUUCGUGCUGAA | ORF | 979 | 302 |
| GGUCAUCAGCGUCAGCAAA | ORF | 1040 | 303 |
| GGCAAAACCUACACAAAGA | ORF | 1512 | 304 |
| UGACCAGGCACUACCGUAA | ORF | 1630 | 305 |
| CCAGAGGAGCCCAAGCCAA | ORF | 1428 | 306 |
| CCUUACACAUGAAGAGGCA | ORF | 1717 | 307 |
| CGGGAAGGGAGAAGACACU | ORF | 385 | 308 |
| CCAAAGAGGGGAAGACGAU | ORF | 1443 | 309 |
| UUACACAUGAAGAGGCAUU | ORF | 1719 | 310 |
| CCGAGGAGUUCAACGAUCU | ORF | 589 | 311 |
| GAGAGACCGAGGAGUUCAA | ORF | 583 | 312 |
| GCGGCAAAACCUACACAAA | ORF | 1510 | 313 |
| AACCCACACAGGUGAGAAA | ORF | 1556 | 314 |
| GGACUUUAUUCUCUCCAAU | ORF | 617 | 315 |
| GCACGUGCCCCAAGAUCAA | ORF | 1117 | 316 |
| GGAGAAGACACUGCGUCAA | ORF | 392 | 317 |
| AGAUCAAGCAGGAGGCGGU | ORF | 1129 | 318 |
| GUUCCCAUCUCAAGGCACA | ORF | 1531 | 319 |
| CAGAUGAACUGACCAGGCA | ORF | 1621 | 320 |
| AGACCGAGGAGUUCAACGA | ORF | 586 | 321 |
| GUGCUGAAGGCGUCGCUGA | ORF | 990 | 322 |
| CGGUCAUCAGCGUCAGCAA | ORF | 1039 | 323 |
| AAGCAGGUGCCCCGAAUAA | ORF | 409 | 324 |
| AAUUGGACCCGGUGUACAU | ORF | 922 | 325 |
| AAACCUACACAAAGAGUUC | ORF | 1516 | 326 |
| AGGCACUACCGUAAACACA | ORF | 1635 | 327 |
| GAGAAGACACUGCGUCAAG | ORF | 393 | 328 |
| GGUGAGAAACCUUACCACU | ORF | 1566 | 329 |
| UCAACGAUCUCCUGGACCU | ORF | 598 | 330 |
| GCGGGAAGGGAGAAGACAC | ORF | 384 | 331 |
| CCCUGGGUCUUGAGGAAGU | ORF | 1255 | 332 |
| CCGAUCAGAUGCAGCCGCA | ORF | 1360 | 333 |
| GCAUGCCAGAGGAGCCCAA | ORF | 1423 | 334 |
| CAAAGAGUUCCCAUCUCAA | ORF | 1525 | 335 |
| UCAACCUGGCGGACAUCAA | ORF | 856 | 336 |
| GGAAAAGGACCGCCACCCA | ORF | 1471 | 337 |
| ACACAAAGAGUUCCCAUCU | ORF | 1522 | 338 |
| UGAGAAACCUUACCACUGU | ORF | 1568 | 339 |
| GACCAGGCACUACCGUAAA | ORF | 1631 | 340 |
| GGCCAGAAUUGGACCCGGU | ORF | 916 | 341 |
| CCGUCGGUCAUCAGCGUCA | ORF | 1035 | 342 |
| GCCCCAAGAUCAAGCAGGA | ORF | 1123 | 343 |
| GCCAAAGAGGGGAAGACGA | ORF | 1442 | 344 |
| GUGAGAAACCUUACCACUG | ORF | 1567 | 345 |
| GGAGAGAGACCGAGGAGUU | ORF | 580 | 346 |
| UGUUAGAAGAAGAGGAAGA | 3′ UTR | 2166 | 347 |
| AGGAAGAAAUUCAGGUACA | 3′ UTR | 2178 | 348 |
| UAGAAGAAGAGGAAGAAAU | 3′ UTR | 2169 | 349 |
| AGAAGAAGAGGAAGAAAUU | 3′ UTR | 2170 | 350 |
| CAGAGGAGCCCAAGCCAAA | ORF | 1429 | 351 |
| GAAGAAGGAUCUCGGCCAA | 5′ UTR | 180 | 352 |
| GGACGGCUGUGGAUGGAAA | ORF | 1592 | 353 |
| GACUGGAAGUUGUGGAUAU | 3′ UTR | 2019 | 354 |
| GAUGUUAGAAGAAGAGGAA | 3′ UTR | 2164 | 355 |
| AGAAAUUCAGGUACAGAAA | 3′ UTR | 2128 | 356 |
| GAUCAACAUUUAUGACCUA | 3′ UTR | 2332 | 357 |
| GGGAGAAGACACUGCGUCA | ORF | 391 | 358 |
| GCACUACAAUCAUGGUCAA | 3′ UTR | 1842 | 359 |
| CCACACUGCCAGAAGAGAA | 3′ UTR | 1764 | 360 |
| CCAGAAGAGAAUUCAGUAU | 3′ UTR | 1772 | 361 |
| AAGAAGAGGAAGAAAUUCA | 3′ UTR | 2172 | 362 |
| AAGUAUGCCUUAAGCAGAA | 3′ UTR | 2446 | 363 |
| GGAUAUCAGGGUAUAAAUU | 3′ UTR | 2032 | 364 |
| AGUCUUGGUUCUAAAGGUA | 3′ UTR | 2236 | 365 |
| CUGCAUACUUUGACAAGGA | 3′ UTR | 2285 | 366 |
| CCUUCAACCUGGCGGACAU | ORF | 853 | 367 |
| CUAAAUCCGACUUGAAUAU | 3′ UTR | 1972 | 368 |
| GAAUAUUCCUGGACUUACA | 3′ UTR | 1985 | 369 |
| CAGAAUUGGACCCGGUGUA | ORF | 919 | 370 |
| UGGGCAAGUUCGUGCUGAA | ORF | 979 | 371 |
| GGUCAUCAGCGUCAGCAAA | ORF | 1040 | 372 |
| CAGAAGAGAAUUCAGUAUU | 3′ UTR | 1773 | 373 |
| CUACAAUCAUGGUCAAGUU | 3′ UTR | 1845 | 374 |
| UCAUCUUGUGAGUGGAUAA | 3′ UTR | 1874 | 375 |
| GUGAGUGGAUAAUCAGGAA | 3′ UTR | 1881 | 376 |
| GAGGAAUCCAAAAGACAAA | 3′ UTR | 1904 | 377 |
| CUUGAAUAUUCCUGGACUU | 3′ UTR | 1982 | 378 |
| GGUGAGUCUUGGUUCUAAA | 3′ UTR | 2232 | 379 |
| GGCAAAACCUACACAAAGA | ORF | 1512 | 380 |
| UGACCAGGCACUACCGUAA | ORF | 1630 | 381 |
| GAAGGAGCCCAGCCAGAAA | 3′ UTR | 1823 | 382 |
| GAGUGGAUAAUCAGGAAAA | 3′ UTR | 1883 | 383 |
| CUAUAUAGUUCCUUGCCUU | 3′ UTR | 2478 | 384 |
| CCAGAGGAGCCCAAGCCAA | ORF | 1428 | 385 |
| CCUUACACAUGAAGAGGCA | ORF | 1717 | 386 |
| UCUAAAUCCGACUUGAAUA | 3′ UTR | 1971 | 387 |
| AGAGGAAGAAAUUCAGGUA | 3′ UTR | 2176 | 388 |
| CGGGAAGGGAGAAGACACU | ORF | 385 | 389 |
| CCAAAGAGGGGAAGACGAU | ORF | 1443 | 390 |
| UUACACAUGAAGAGGCAUU | ORF | 1719 | 391 |
| GGAGGGAAGACCAGAAUUC | 3′ UTR | 2067 | 392 |
| GUUAGAAGAAGAGGAAGAA | 3′ UTR | 2167 | 393 |
| AAGAAAUUCAGGUACAGAA | 3′ UTR | 2181 | 394 |
| GCAUACUUUGACAAGGAAA | 3′ UTR | 2287 | 395 |

Exemplary siRNAs for Nanog are shown in TABLE 5.

TABLE 5

| | REGION IN | START | SEQ ID |
|---|---|---|---|
| SEQUENCE | TARGET | POSITION | NO. |
| CUAUUGAGGUAAAGGGUUA | 3′ UTR | 1844 | 396 |
| GAGUAUGGUUGGAGCCUAA | 3′ UTR | 1286 | 397 |
| GGUAAAGGGUUAAGCUGUA | 3′ UTR | 1851 | 398 |
| GAAUCUAACCUCAAGAAUA | 3′ UTR | 1747 | 399 |
| AGAAAGAGGUCUCGUAUUU | 3′ UTR | 1948 | 400 |
| CUAUAACUGUGGAGAGGAA | ORF | 936 | 401 |
| UGACAUGAGUACUGCUUUA | 3′ UTR | 1979 | 402 |
| CAGCAGACCACUAGGUAUU | ORF | 1048 | 403 |
| UCUAAGAGGUGGCAGAAAA | ORF | 664 | 404 |
| GCAUGCAGUUCCAGCCAAA | ORF | 968 | 405 |
| GGGAAGGCCUUAAUGUAAU | ORF | 1028 | 406 |
| UUGGAUAUCUUUAGGGUUU | 3′ UTR | 1727 | 407 |
| CGUAUUUGCUGCAUCGUAA | 3′ UTR | 1960 | 408 |
| UCUAGAGACUCCAGGAUUU | 5′ UTR | 9 | 409 |
| CAGAGAAGAGUGUCGCAAA | ORF | 455 | 410 |
| GGAUCUUCCUGGAGAAAAU | 3′ UTR | 1339 | 411 |
| AGAGAAGAGUGUCGCAAAA | ORF | 456 | 412 |
| AAGACAAGGUCCCGGUCAA | ORF | 479 | 413 |
| AUGAUAGAUUUCAGAGACA | ORF | 548 | 414 |
| GGGGAAGGCCUUAAUGUAA | ORF | 1027 | 415 |
| GGAAGGCCUUAAUGUAAUA | ORF | 1029 | 416 |
| GUGCUAAUCUUUGUAGAAA | 3′ UTR | 1934 | 417 |
| GGAACAGUCCCUUCUAUAA | ORF | 923 | 418 |
| UCUCAUGGAGGGUGGAGUA | 3′ UTR | 1272 | 419 |
| GCAUCCGACUGUAAAGAAU | ORF | 262 | 420 |
| UUCCAGAACCAGAGAAUGA | ORF | 643 | 421 |
| AAAUCUAAGAGGUGGCAGA | ORF | 661 | 422 |
| CCUGAAGACGUGUGAAGAU | ORF | 1120 | 423 |
| CGAGUGUUUCAAUGAGUAA | 3′ UTR | 2063 | 424 |
| CCACCAGUCCCAAAGGCAA | ORF | 422 | 425 |
| CACCAGUCCCAAAGGCAAA | ORF | 423 | 426 |
| GAUAGAUUUCAGAGACAGA | ORF | 550 | 427 |
| GCAACCAGACCCAGAACAU | ORF | 836 | 428 |
| CUAAACUACUCCAUGAACA | ORF | 1096 | 429 |
| GAGCCUAAUCAGCGAGGUU | 3′ UTR | 1297 | 430 |
| CAAUGAUAGAUUUCAGAGA | ORF | 546 | 431 |
| GCUACAAACAGGUGAAGAC | ORF | 620 | 432 |
| GCAAUGGUGUGACGCAGAA | ORF | 701 | 433 |
| GGAACAAUCAGGCCUGGAA | ORF | 908 | 434 |
| CUUGGAAGCUGCUGGGGAA | ORF | 1014 | 435 |
| GAUUUGUGGGCCUGAAGAA | ORF | 297 | 436 |
| AAGAAACAGAAGACCAGAA | ORF | 496 | 437 |
| CCAGAACCAGAGAAUGAAA | ORF | 645 | 438 |
| AACCAGAGAAUGAAAUCUA | ORF | 649 | 439 |
| AACAACUGGCCGAAGAAUA | ORF | 682 | 440 |
| GUAAUACAGCAGACCACUA | ORF | 1042 | 441 |
| UCUUUAGGGUUUAGAAUCU | 3′ UTR | 1734 | 442 |
| GUAAAGGGUUAAGCUGUAA | 3′ UTR | 1852 | 443 |
| CCCAAUUUCUUGAUACUUU | 5′ UTR | 87 | 444 |
| GUCAAGAAACAGAAGACCA | ORF | 493 | 445 |

Exemplary siRNAs for c-Myc are shown in TABLE 6.

TABLE 6

| | REGION IN | START | SEQ ID |
|---|---|---|---|
| SEQUENCE | TARGET | POSITION | NO. |
| GGAACAAGAAGAUGAGGAA | ORF | 1331 | 446 |
| GAGGAUAUCUGGAAGAAAU | ORF | 708 | 447 |
| ACACAAACUUGAACAGCUA | ORF | 1853 | 448 |
| GCGACGAGGAGGAGAACUU | ORF | 643 | 449 |
| GAGAAUGUCAAGAGGCGAA | ORF | 1623 | 450 |
| GAGAACAGUUGAAACACAA | ORF | 1840 | 451 |
| ACACAAUGUUUCUCUGUAA | 3′ UTR | 2138 | 452 |
| AACAAGAAGAUGAGGAAGA | ORF | 1333 | 453 |
| AAGAAGAUGAGGAAGAAAU | ORF | 1336 | 454 |
| UCAGAGGCUUGGCGGGAAA | 5′ UTR | 87 | 455 |
| UGUAGUAAUUCCAGCGAGA | 5′ UTR | 169 | 456 |
| AGGGAGAUCCGGAGCGAAU | 5′ UTR | 264 | 457 |
| GGGUCAAGUUGGACAGUGU | ORF | 1540 | 458 |
| CGAGAACAGUUGAAACACA | ORF | 1839 | 459 |
| GGAAGAAAUUCGAGCUGCU | ORF | 718 | 460 |
| ACAAGAAGAUGAGGAAGAA | ORF | 1334 | 461 |
| CGAUGUUGUUUCUGUGGAA | ORF | 1355 | 462 |
| ACACAGAAUUUCAAUCCUA | 3′ UTR | 2205 | 463 |
| GGGAUCGCGCUGAGUAUAA | 5′ UTR | 119 | 464 |
| CUGCUUAGACGCUGGAUUU | 5′ UTR | 514 | 465 |
| AGGAGGAACAAGAAGAUGA | ORF | 1327 | 466 |
| AGGAAGAAAUCGAUGUUGU | ORF | 1345 | 467 |
| AGAGGAGGAACGAGCUAAA | ORF | 1663 | 468 |
| GGAACUAUGACCUCGACUA | ORF | 598 | 469 |
| AAGAGGACUUGUUGCGGAA | ORF | 1816 | 470 |
| GACGAGAACAGUUGAAACA | ORF | 1837 | 471 |
| CUAACUCGCUGUAGUAAUU | 5′ UTR | 160 | 472 |
| GCGAGGAUAUCUGGAAGAA | ORF | 706 | 473 |
| GCUUGUACCUGCAGGAUCU | ORF | 1093 | 474 |
| GGAAGAAAUCGAUGUUGUU | ORF | 1346 | 475 |
| CGUCCAAGCAGAGGAGCAA | ORF | 1784 | 476 |
| CCACGAAACUUUGCCCAUA | 5′ UTR | 352 | 477 |
| CCGCCAAGCUCGUCUCAGA | ORF | 991 | 478 |
| CAGAGAAGCUGGCCUCCUA | ORF | 1006 | 479 |
| CAAGAAGAUGAGGAAGAAA | ORF | 1335 | 480 |
| CCACACAUCAGCACAACUA | ORF | 1477 | 481 |
| CCAGAGGAGGAACGAGCUA | ORF | 1661 | 482 |
| GCGGAAACGACGAGAACAG | ORF | 1829 | 483 |
| GUUUCAAAUGCAUGAUCAA | 3′ UTR | 1951 | 484 |
| ACUUACAACACCCGAGCAA | 5′ UTR | 397 | 485 |
| CUGAGGAGGAACAAGAAGA | ORF | 1324 | 486 |
| ACUGCGACGAGGAGGAGAA | ORF | 640 | 487 |
| CGAGGAUAUCUGGAAGAAA | ORF | 707 | 488 |
| AGGAUAUCUGGAAGAAAUU | ORF | 709 | 489 |
| CGACGAGACCUUCAUCAAA | ORF | 929 | 490 |
| ACUCUGAGGAGGAACAAGA | ORF | 1321 | 491 |
| GAGGGAUCGCGCUGAGUAU | 5′ UTR | 117 | 492 |
| GCUCAUUUCUGAAGAGGAC | ORF | 1805 | 493 |
| GCAGCGACUCUGAGGAGGA | ORF | 1315 | 494 |
| GCGACUCUGAGGAGGAACA | ORF | 1318 | 495 |

Exemplary siRNAs for Klf5 are shown in TABLE 7.

TABLE 7

| | REGION IN | START | SEQ ID |
|---|---|---|---|
| SEQUENCE | TARGET | POSITION | NO. |
| ACAAAUAGCCAUUGAACAA | 3′ UTR | 2167 | 496 |
| AGGUAAUUCCUUAGAGAUA | 3′ UTR | 2130 | 497 |
| GUGCAGUACUGUUGGUUAA | 3′ UTR | 2697 | 498 |
| CCAAAGGGCAGAAUAAAUA | 3′ UTR | 2912 | 499 |
| GAUGUGAAAUGGAGAAGUA | ORF | 590 | 500 |
| CUAUAAUUCCAGAGCAUAA | ORF | 635 | 501 |
| GCACAAAAGUUUAUACCAA | ORF | 1463 | 502 |
| GGGCAGAAUAAAUAAGCAA | 3′ UTR | 2917 | 503 |
| CAGAGAUGCUCCAGAAUUU | ORF | 1271 | 504 |
| UGGAAGAGCGGAAGAGUUU | 5′ UTR | 139 | 505 |
| UAACCAAAGGGCAGAAUAA | 3′ UTR | 2909 | 506 |
| GAAGAAGAAUGGAUUGUAU | 3′ UTR | 2070 | 507 |
| ACUGAAGAGCUUAAAGAUA | 3′ UTR | 2505 | 508 |
| UGAAACAAUUCCAGGGCAU | ORF | 1148 | 509 |
| ACAAUAAGCUAAACGCAAU | 3′ UTR | 2552 | 510 |
| CCUAACUAUUCCUGUGUAA | 3′ UTR | 1751 | 511 |
| UGAACAAAUGUGUGGGUUU | 3′ UTR | 2179 | 512 |
| GCUGUAUAGUUGUAGAAUU | 3′ UTR | 3262 | 513 |
| UCCCAGAGACCGUGCGUAA | ORF | 781 | 514 |
| AGAUACAAUAGAAGGAGUA | ORF | 1396 | 515 |
| GGGAGUGUGUGCAGCGUUU | 3′ UTR | 1989 | 516 |
| AGUUCAACCUCUUACAAUA | 3′ UTR | 2539 | 517 |
| GUAAAUAGAUGACAAACGA | 3′ UTR | 3099 | 518 |
| GCUCCAGAGGUGAACAAUA | ORF | 922 | 519 |
| GGGUCUUAAUUGAAAUGAA | 3′ UTR | 2947 | 520 |
| CUCCAGAGGUGAACAAUAU | ORF | 923 | 521 |
| CAAGAAAACCACAACUAAA | 3′ UTR | 1792 | 522 |
| UCUUUAGAGGGAAGGAAUA | 3′ UTR | 2395 | 523 |
| CUGAAGAGCUUAAAGAUAG | 3′ UTR | 2506 | 524 |
| ACACAGUGAGACACAGUAA | 3′ UTR | 2746 | 525 |
| GGAAACACACCUACAUGAA | 3′ UTR | 3158 | 526 |
| GCAAACAGCUGUAUAGUUG | 3′ UTR | 3255 | 527 |
| UGAGAGAAUGAGAUGUUUA | 3′ UTR | 3284 | 528 |
| GUCCAGACAAGAUGUGAAA | ORF | 580 | 529 |
| CAAGAUGUGAAAUGGAGAA | ORF | 587 | 530 |
| UCCUAUAAUUCCAGAGCAU | ORF | 633 | 531 |
| CACUGACACUGAAGGGUUA | ORF | 696 | 532 |
| CAGUAUACCUGGCAAUUCA | 3′ UTR | 2149 | 533 |
| AAUCAUUUCUUUAGAGGGA | 3′ UTR | 2388 | 534 |
| GUUCAACCUCUUACAAUAA | 3′ UTR | 2540 | 535 |
| UUACAGUGCAGUUUAGUUA | 3′ UTR | 2776 | 536 |
| GUGUCUGCCUUUAAAUAUA | 3′ UTR | 2814 | 537 |
| UAACACACAUCAAGACAGA | ORF | 797 | 538 |
| ACAUCCAACCUGUCAGAUA | ORF | 1382 | 539 |
| AAGAAUGGAUUGUAUGUCA | 3′ UTR | 2074 | 540 |
| GUAGGUAAUUCCUUAGAGA | 3′ UTR | 2128 | 541 |
| ACAUAUAUGAGUUGCCUAU | 3′ UTR | 2211 | 542 |
| CAAAUCAGCUUUAUAGGUU | 3′ UTR | 2258 | 543 |
| ACACUUACAGUUAGGAUUU | 3′ UTR | 2341 | 544 |
| CUUUAGAGGGAAGGAAUAA | 3′ UTR | 2396 | 545 |

Exemplary siRNAs for Klf2 are shown in TABLE 8.

TABLE 8

| | REGION IN | START | SEQ ID |
|---|---|---|---|
| SEQUENCE | TARGET | POSITION | NO. |
| UGUGAUGCCUUGUGAGAAA | 3′ UTR | 1453 | 546 |
| GUAUAUAGUGACUGACAAA | 3′ UTR | 1516 | 547 |
| GGCAAGACCUACACCAAGA | ORF | 922 | 548 |
| UGGAGCUGCUGGAGGCCAA | ORF | 833 | 549 |
| GCGGCAAGACCUACACCAA | ORF | 920 | 550 |
| GGUAUUUAUUGGACCCAGA | 3′ UTR | 1231 | 551 |
| UAGAGAGACAGGUGGGCAU | 3′ UTR | 1552 | 552 |
| GCACCGACGACGACCUCAA | ORF | 191 | 553 |
| UACUGUACAUAGAGAGACA | 3′ UTR | 1543 | 554 |
| UGUAUAUAGUGACUGACAA | 3′ UTR | 1515 | 555 |
| CCAAACUGUGACUGGUAUU | 3′ UTR | 1218 | 556 |
| UGCUGGAGGCCAAGCCAAA | ORF | 839 | 557 |
| CAGCGUGGCUACAGAGGGU | 3′ UTR | 1265 | 558 |
| AGACCUACACCAAGAGUUC | ORF | 926 | 559 |
| ACUAGAGGAUCGAGGCUUG | 3′ UTR | 1436 | 560 |
| GUAUUACUGUACAUAGAGA | 3′ UTR | 1539 | 561 |
| GUACAUAGAGAGACAGGUG | 3′ UTR | 1547 | 562 |
| AUUACUGUACAUAGAGAGA | 3′ UTR | 1541 | 563 |
| UGGGCUACCUGGUUCGUUU | 3′ UTR | 1574 | 564 |
| GGUGAGAAGCCCUACCACU | ORF | 976 | 565 |
| GCUGGAAGUUUGCGCGCUC | ORF | 1013 | 566 |
| AUUUAUUGGACCCAGAGAA | 3′ UTR | 1234 | 567 |
| GGGUCUCCCUCGAUGACGA | 3′ UTR | 1280 | 568 |
| UCGAUGACGACGACGACGA | 3′ UTR | 1289 | 569 |
| GGGAAAAGACCACGAUCCU | 3′ UTR | 1348 | 570 |
| ACCGAAAGCACACGGGCCA | ORF | 1052 | 571 |
| UCCCAAACUGUGACUGGUA | 3′ UTR | 1216 | 572 |
| ACCAAGAGUUCGCAUCUGA | ORF | 934 | 573 |
| CCAAGAGUUCGCAUCUGAA | ORF | 935 | 574 |
| CCCAAACUGUGACUGGUAU | 3′ UTR | 1217 | 575 |
| UGAUGCCUUGUGAGAAAUA | 3′ UTR | 1455 | 576 |
| ACGACGACCUCAACAGCGU | ORF | 197 | 577 |
| CUGCUGGAGGCCAAGCCAA | ORF | 838 | 578 |
| GUUCGCAUCUGAAGGCGCA | ORF | 941 | 579 |
| GUGAGAAGCCCUACCACUG | ORF | 977 | 580 |
| UCACGCGCCACUACCGAAA | ORF | 1040 | 581 |
| CUGCACAUGAAACGGCACA | ORF | 1129 | 582 |
| UUUAUUGGACCCAGAGAAC | 3′ UTR | 1235 | 583 |
| AGAGAGACAGGUGGGCAUU | 3′ UTR | 1553 | 584 |
| ACACCAAGAGUUCGCAUCU | ORF | 932 | 585 |
| AAACUGUGACUGGUAUUUA | 3′ UTR | 1220 | 586 |
| GGCACAGCGUGGCUACAGA | 3′ UTR | 1261 | 587 |
| UGUCUGAGCUGCUGCGACC | ORF | 359 | 588 |
| CCUUCGGUCUCUUCGACGA | ORF | 737 | 589 |
| GCAAACGCACCGCCACUCA | ORF | 881 | 590 |
| GCGUGGCUACAGAGGGUCU | 3′ UTR | 1267 | 591 |
| GAUCGAGGCUUGUGAUGCC | 3′ UTR | 1443 | 592 |
| GCCUUAAUUUGUACUGUCU | 3′ UTR | 1477 | 593 |
| UUGUACUGUCUGCGGCAUU | 3′ UTR | 1485 | 594 |

Exemplary siRNAs for ESRRB are shown in TABLE 9.

TABLE 9

| | REGION IN | START | |
|---|---|---|---|
| SEQUENCE | TARGET | POSITION | SEQ ID NO. |
| GCGUCAAACUGCAGGGCAA | ORF | 1570 | 595 |
| CAGAGUGCCUGGAUGGAAA | ORF | 1164 | 596 |
| UGGAGAUGCUGGAGGCCAA | ORF | 1612 | 597 |
| UGGUGUACGCUGAGGACUA | ORF | 1231 | 598 |
| ACAAGAAGCUCAAGGUGGA | ORF | 1327 | 599 |
| UGACCAAGAUUGUCUCAUA | ORF | 961 | 600 |
| CCAUGUACAUCGAGGAUCU | ORF | 1396 | 601 |
| CACCAGGAGGCCAGGGAAA | 3′ UTR | 2009 | 602 |
| CGGACAAGCUCUAUGCCAU | ORF | 997 | 603 |
| CAAGCAGGGAUCAGAGCAA | 3′ UTR | 1907 | 604 |
| UCCCUGGGCUGGUGAAUAA | 5′ UTR | 191 | 605 |
| CAGAGGUGAUCCAGUGAUU | 5′ UTR | 271 | 606 |
| GUGGAAGAGAAAUGAGCUU | 5′ UTR | 133 | 607 |
| CCAUCAAGUGCGAGUACAU | ORF | 598 | 608 |
| CGUCAAACUGCAGGGCAAA | ORF | 1571 | 609 |
| GGACAUUGCCUCUGGCUAC | ORF | 656 | 610 |
| AGCUCAAGGUGGAGAAGGA | ORF | 1333 | 611 |
| AGGUGGAGAAGGAGGAGUU | ORF | 1339 | 612 |
| ACGAGGCACUGCAGGACUA | ORF | 1447 | 613 |
| CUCCCAAGGAUGAAAGAAU | 3′ UTR | 1844 | 614 |
| CAAGAGCAGCUUAGAGGAU | ORF | 1825 | 615 |
| GGAAAGCAUCUCUGGCUCA | 3′ UTR | 2023 | 616 |
| ACCGAGAGCUUGUGGUCAU | ORF | 1078 | 617 |
| GCAGGUACAAGAAGCUCAA | ORF | 1321 | 618 |
| GAGAAGGAGGAGUUUGUGA | ORF | 1344 | 619 |
| CAGCACUUCUAUAGCGUCA | ORF | 1557 | 620 |
| GGGCGGAAGUCCUGAUGGU | 3′ UTR | 2155 | 621 |
| CAAGAUUGUCUCAUACCUA | ORF | 965 | 622 |
| CAUCGAGGAUCUAGAGGCU | ORF | 1403 | 623 |
| GCACUUCUAUAGCGUCAAA | ORF | 1559 | 624 |
| CAGCAUGUGCAUUUCCUAA | ORF | 1713 | 625 |
| GAGGAUCUCCCAAGGAUGA | ORF | 1838 | 626 |
| AGAGAAAUGAGCUUGGCUU | 5′ UTR | 138 | 627 |
| GAGCUUGGCUUGCAACUCA | 5′ UTR | 146 | 628 |
| CUUUGAGGCCAGAGGUGAU | 5′ UTR | 262 | 629 |
| UGGAGAAGGAGGAGUUUGU | ORF | 1342 | 630 |
| UCGAGGAUCUAGAGGCUGU | ORF | 1405 | 631 |
| UGAAAGAAUGUCAAGCCAU | 3′ UTR | 1854 | 632 |
| AAUGAGAGAGGCAGGCAGA | 3′ UTR | 1972 | 633 |
| GGGACAUUGCCUCUGGCUA | ORF | 655 | 634 |
| CCAAGGGAACAUUGAGUAC | ORF | 728 | 635 |
| GCGCCUUGAUCGAGUGCGU | ORF | 854 | 636 |
| AUACCUGAGCUUACAAAUU | ORF | 920 | 637 |
| CCUGGCAGACCGAGAGCUU | ORF | 1070 | 638 |
| CCACCAAGAGGCAGCAUGU | ORF | 1702 | 639 |
| AUGAAAGAAUGUCAAGCCA | 3′ UTR | 1853 | 640 |
| GAGAAAUGAGCUUGGCUUG | 5′ UTR | 139 | 641 |
| CCAAAAUGGUGUCCAGAAC | 5′ UTR | 244 | 642 |
| GGACUAUCCAAGGGAACAU | ORF | 721 | 643 |
| CUUCAUGAAAUGCCUCAAA | ORF | 812 | 644 |

Exemplary siRNAs for REST are shown in TABLE 10.

TABLE 10

| | REGION IN | START | |
|---|---|---|---|
| SEQUENCE | TARGET | POSITION | SEQ ID NO. |
| GCAAAGUGGAGGAGAAUAA | ORF | 2035 | 645 |
| GGAUGUGGCUGGAAAGAAA | ORF | 1712 | 646 |
| GGAAAUUGAUGAAGAUGAA | ORF | 3356 | 647 |
| CAACACAGGUGAAGGAAAU | ORF | 2996 | 648 |
| CCACAAGAAUCUAGCAGAA | ORF | 3462 | 649 |
| GGAGGAAACAUUUAAGAAA | ORF | 1135 | 650 |
| GAUCAGAACACAAGAGAGA | ORF | 3201 | 651 |
| GUGCAGAGAAGCAGGCAAA | ORF | 919 | 652 |
| ACAGCAAAGUGGAGGAGAA | ORF | 2032 | 653 |
| CCAUAGAGGUGGUCCAGAA | ORF | 2650 | 654 |
| CCAUGAAGGAAGUGACCUA | ORF | 3386 | 655 |
| GGGAAAAGAUUACAGCAAA | ORF | 3560 | 656 |
| AAAAGAAGGUAGAAAGCAA | ORF | 1978 | 657 |
| GGUAGAAAGCAAAUCCAAA | ORF | 1985 | 658 |
| CCACAGAGGCGGUUCAGAA | ORF | 2197 | 659 |
| UCAGAAAGUAGGAGCAGAA | ORF | 3029 | 660 |
| GGGCAGGAGUAAUGAAACU | ORF | 3630 | 661 |
| UGAAGAGUCUGCUGAUAUA | ORF | 626 | 662 |
| GGCAAGAGCUCGAAGACCA | ORF | 798 | 663 |
| GGAAGAGAGUGCAGAGAAG | ORF | 911 | 664 |
| CUUCUAAAGGAAAGUGUAA | ORF | 2895 | 665 |
| GAGAAGAGGCAUCAGGAGA | ORF | 2965 | 666 |
| GGUGAAACUUUAAAUGGUA | ORF | 3138 | 667 |
| AGAUAGUGAAGAAGGAGAA | ORF | 602 | 668 |
| UGAAGAAGGAGAAGGACUU | ORF | 608 | 669 |
| CCAGAUAUUUACAGUUCAA | ORF | 738 | 670 |
| GAGCGGAGGACAAAGGCAA | ORF | 784 | 671 |
| UAACAGAGGUGAAAGAGAU | ORF | 1834 | 672 |
| ACAGGAAGCAAUUCAGAAA | ORF | 1863 | 673 |
| AGGAAGUGCCAAAGGGUGA | ORF | 2014 | 674 |
| GAAGGAGCCUGUUCAGAUA | ORF | 2570 | 675 |
| AGUCUAACAUGCAGAGUGA | ORF | 2815 | 676 |
| UCUAACAUGCAGAGUGAAA | ORF | 2817 | 677 |
| CCUUAUUGAAGUUGGCUUA | ORF | 2855 | 678 |
| CAGUAACAGAGGUGAAAGA | ORF | 1831 | 679 |
| GGAAGUGACCUAAGUGACA | ORF | 3393 | 680 |
| GUGAUUACCUGGUCGGUGA | ORF | 535 | 681 |
| GAGUAUCACUGGAGGAAAC | ORF | 1125 | 682 |
| AGGAGAACGCCCAUAUAAA | ORF | 1244 | 683 |
| GAUGAGGAAUCUUCAACAA | ORF | 1953 | 684 |
| GCCAAAGGGUGACAGCAAA | ORF | 2021 | 685 |
| AGAAGGAACCUGUUGAGAA | ORF | 2137 | 686 |
| GAGCAGAAGAGGCAGAUGA | ORF | 3040 | 687 |
| AAAGAAAAGUAGUCGGAGA | 5′ UTR | 272 | 688 |
| AAGAACAGUUUGUGCAUCA | ORF | 859 | 689 |
| GCUACAAUACUAAUCGAUA | ORF | 1012 | 690 |
| AAACAAUGGAUGUCUCAAA | ORF | 1600 | 691 |
| AAUCAGUAACAGAGGUGAA | ORF | 1828 | 692 |
| GUGCAUACAGGAAGCAAUU | ORF | 1857 | 693 |

Exemplary siRNAs for Tbx3 are shown in TABLE 11.

TABLE 11

| | REGION IN | START | |
|---|---|---|---|
| SEQUENCE | TARGET | POSITION | SEQ ID NO. |
| GGAAAUGGCCGAAGAGAAA | ORF | 1823 | 694 |
| CGAGAAAGAGGGAGAGGAA | 5′ UTR | 448 | 695 |
| AGAAAGAGGGAGAGGAAGA | 5′ UTR | 450 | 696 |
| GUAAAUAGGUGGAAUAUGA | 3′ UTR | 4073 | 697 |
| CAACAACAUUUCAGACAAA | ORF | 1594 | 698 |
| GAAUAUGAAUGCUUGGAAA | 3′ UTR | 4084 | 699 |
| AAGAAGAGGUGGAGGACGA | ORF | 1251 | 700 |
| AGGACAAGGAAGAGAGAGA | 5′ UTR | 194 | 701 |
| AGGGAGAGGAAGACAGAUA | 5′ UTR | 456 | 702 |
| GUGCCUGCCUAUAGAGAUA | 3′ UTR | 4544 | 703 |
| CCGAAAUGCCAAAGAGGAU | ORF | 1497 | 704 |
| UGGAAAUGGCCGAAGAGAA | ORF | 1822 | 705 |
| CUUGUAAAUAGGUGGAAUA | 3′ UTR | 4070 | 706 |
| GAGAGAUGGUUUAAAGACA | 3′ UTR | 4589 | 707 |
| GGAGAAGAGCCCAGCAAGA | 5′ UTR | 219 | 708 |
| CCGAAGAAGAGGUGGAGGA | ORF | 1248 | 709 |
| CUGCAUACCAGAAUGAUAA | ORF | 1749 | 710 |
| GGACAAGUGAACACAUUAA | 3′ UTR | 3560 | 711 |
| GCACUUUGUCGGAUAUAAA | 3′ UTR | 3185 | 712 |
| GAGAUGGUUUAAAGACAAA | 3′ UTR | 4591 | 713 |
| CCAUGGAGCCCGAAGAAGA | ORF | 1239 | 714 |
| GCUGAUGACUGUCGUUAUA | ORF | 1436 | 715 |
| CAUCGAACCUCAAAGAUUU | ORF | 1989 | 716 |
| CGGACUCCCUCGAGAGAAU | 3′ UTR | 3267 | 717 |
| AGUGAGACUAUUAGACAAA | 3′ UTR | 4026 | 718 |
| AGAGAUGGUUUAAAGACAA | 3′ UTR | 4590 | 719 |
| GGUGGAUGGUGGCUGGUAA | ORF | 1470 | 720 |
| CCAGCGAACUGCAGAGCAU | ORF | 3057 | 721 |
| GCGCCUGGACACAGAUUUA | 5′ UTR | 152 | 722 |
| CCAGCGAGAAAGAGGGAGA | 5′ UTR | 444 | 723 |
| CCAACAACAUUUCAGACAA | ORF | 1593 | 724 |
| GCAAAAGGUUUCCGGGACA | ORF | 1802 | 725 |
| AGAGAAUGUGCUAGAGACA | 3′ UTR | 3279 | 726 |
| GGUAGGAGUUCCAACAUUU | 3′ UTR | 3386 | 727 |
| CCAAUGACAUCUUGAAACU | ORF | 1674 | 728 |
| GGACACAGAUUUAGGAAGC | 5′ UTR | 158 | 729 |
| CGACUAUGUUUGCUGAUUU | 5′ UTR | 713 | 730 |
| GUGCAUUAGUUGUGAUUUC | 5′ UTR | 798 | 731 |
| AAAGGGAAGGAGUGGGCAA | 3′ UTR | 3891 | 732 |
| CCUGGAGGCUAAAGAACUU | ORF | 1282 | 733 |
| CCAUGAGGGUGUUUGAUGA | ORF | 1866 | 734 |
| CCGUGCACUUUGUCGGAUA | 3′ UTR | 3181 | 735 |
| GGAUUUAAAGGGAAGGAGU | 3′ UTR | 3885 | 736 |
| AAGUGAGACUAUUAGACAA | 3′ UTR | 4025 | 737 |
| GACAAAUUCAUGAAGGUAU | 3′ UTR | 4604 | 738 |
| GUGUUAUAGUUGUUGAUGA | 3′ UTR | 4628 | 739 |
| ACGCAGGGCUGGAGUGUCU | 5′ UTR | 573 | 740 |
| CCAUUUAAAGUGAGAUGUU | ORF | 1367 | 741 |
| CAAAGAGGAUGUACAUUCA | ORF | 1506 | 742 |
| ACAUCGAACCUCAAAGAUU | ORF | 1988 | 743 |

Exemplary siRNAs for Foxc1 are shown in TABLE 12.

TABLE 12

| | REGION IN | START | |
|---|---|---|---|
| SEQUENCE | TARGET | POSITION | SEQ ID NO. |
| GGGAAUAGUAGCUGUCAAA | ORF | 1573 | 744 |
| CCAGAUAUGCACAGAUAAA | 3′ UTR | 2757 | 745 |
| CCAGAUAACACGUAAGUUU | 3′ UTR | 1967 | 746 |
| GGCCAGAUAUGCACAGAUA | 3′ UTR | 2755 | 747 |
| UGUAAAUAACCCAGGAAAU | 3′ UTR | 3188 | 748 |
| CCUCAAAGCCGAACUAAAU | 3′ UTR | 1668 | 749 |
| AGAAGAAGGACGCGGUGAA | ORF | 524 | 750 |
| ACAGAUUGGAGUUGGCAUA | 3′ UTR | 2623 | 751 |
| GGAGAUGGCGAUUUGAUUA | 3′ UTR | 3257 | 752 |
| AGGCAACACUUAAGCAGUA | 3′ UTR | 3355 | 753 |
| UGAAGGACAAGGAGGAGAA | ORF | 539 | 754 |
| GGACCAAACGCCAGAAAGU | 3′ UTR | 2200 | 755 |
| CGGUGAAGGACAAGGAGGA | ORF | 536 | 756 |
| GCCAGAAAGUGUUCCCAAA | 3′ UTR | 2209 | 757 |
| GAUUGGAGUUGGCAUAUAA | 3′ UTR | 2626 | 758 |
| GGUUGGAAAGGGAUAUUUA | 3′ UTR | 2980 | 759 |
| GGAAAGGGAUAUUUAAUCU | 3′ UTR | 2984 | 760 |
| CGGGAAUAGUAGCUGUCAA | ORF | 1572 | 761 |
| CGAGAGGAGCAGAACAUUU | 3′ UTR | 3081 | 762 |
| GAUCAUUGUUAAAGGAUUG | 3′ UTR | 3400 | 763 |
| AGGCAAAAUCGAAACUAAA | 3′ UTR | 1724 | 764 |
| GAGUUGGCAUAUAAACAAA | 3′ UTR | 2631 | 765 |
| AUUCAUUAUCUUAGGGUGA | 3′ UTR | 3214 | 766 |
| AGGACGCGGUGAAGGACAA | ORF | 530 | 767 |
| CUAAAUAAACAAACCCGUA | 3′ UTR | 1894 | 768 |
| ACAGCAAAAUCUUGGUUUA | 3′ UTR | 1930 | 769 |
| GGAGUUGGCAUAUAAACAA | 3′ UTR | 2630 | 770 |
| GGGACUGUGCGGCCAGAUA | 3′ UTR | 2745 | 771 |
| GGCGAGAGGAGCAGAACAU | 3′ UTR | 3079 | 772 |
| CCCUCAAAGCCGAACUAAA | 3′ UTR | 1667 | 773 |
| AGGAACCCAUCAAGGCAAA | 3′ UTR | 1712 | 774 |
| CAUCAAGGCAAAAUCGAAA | 3′ UTR | 1719 | 775 |
| GGGAAACUGUAUUAAUCUU | 3′ UTR | 2284 | 776 |
| UGGAGAAACCCUCUGACUA | 3′ UTR | 2486 | 777 |
| AGUUAAACCUAGGGGACAA | 3′ UTR | 3147 | 778 |
| GCUCCUAUCUAGAGGCAAC | 3′ UTR | 3343 | 779 |
| GAACAACUCUCCAGUGAAC | ORF | 1554 | 780 |
| GGACAGUGUUACUCCAGAU | 3′ UTR | 1954 | 781 |
| CCUCUCACCUGUAAGAUAU | 3′ UTR | 2050 | 782 |
| AGUUGGAUGUCGUGGACCA | 3′ UTR | 2187 | 783 |
| GGAGAAACCCUCUGACUAG | 3′ UTR | 2487 | 784 |
| GGUCUAGGGUGGUUUCUUU | 3′ UTR | 3101 | 785 |
| UUGUAAAUAACCCAGGAAA | 3′ UTR | 3187 | 786 |
| GGGAGAUGGCGAUUUGAUU | 3′ UTR | 3256 | 787 |
| CGAUUUGAUUACAGACGUU | 3′ UTR | 3265 | 788 |
| AGUAAUUGCUGUUGCUUGU | 3′ UTR | 3370 | 789 |
| GCUGUUGCUUGUUGUCAAA | 3′ UTR | 3377 | 790 |

Exemplary siRNAs for Foxc2 are shown in TABLE 13.

TABLE 13

| | REGION IN | START | |
|---|---|---|---|
| SEQUENCE | TARGET | POSITION | SEQ ID NO. |
| AGAAGAAGGUGGUGAUCAA | ORF | 623 | 791 |
| CCAAGGAGGCCGAGAAGAA | ORF | 611 | 792 |
| GCUUCAGCGUGGAGAACAU | ORF | 806 | 793 |
| CCGAGAAGAAGGUGGUGAU | ORF | 620 | 794 |
| GAGAAGAAGAUCACCUUGA | ORF | 268 | 795 |
| CGCCUAAGGACCUGGUGAA | ORF | 197 | 796 |
| CCUACGACUGCACGAAAUA | ORF | 1484 | 797 |
| UGUCCAAGGAGAAGGAGGA | ORF | 518 | 798 |
| AGAAGAAGAUCACCUUGAA | ORF | 269 | 799 |
| AGGUGGUGAUCAAGAGCGA | ORF | 629 | 800 |
| GAGAAGAAGGUGGUGAUCA | ORF | 622 | 801 |
| CCAACGUGCGGGAGAUGUU | ORF | 1343 | 802 |
| CAGAAUUACUACCGGGCUG | ORF | 64 | 803 |
| GGGAGAACAAGCAGGGCUG | ORF | 329 | 804 |
| ACCUGAGCGAGCAGAAUUA | ORF | 53 | 805 |
| CCGAGAAGAAGAUCACCUU | ORF | 266 | 806 |
| UGAGCGAGCAGAAUUACUA | ORF | 56 | 807 |
| GCGCCUAAGGACCUGGUGA | ORF | 196 | 808 |
| CCUACCUGAGCGAGCAGAA | ORF | 50 | 809 |
| AAGAAGGUGGUGAUCAAGA | ORF | 625 | 810 |
| CAGUGCAGCAUGCGAGCGA | ORF | 988 | 811 |
| CGGCCCAGCAGCAAACUUU | ORF | 1322 | 812 |
| UGGAGAACAUCAUGACCCU | ORF | 815 | 813 |
| CGGGAGAACAAGCAGGGCU | ORF | 328 | 814 |
| CUGGCUUCAGCGUGGAGAA | ORF | 803 | 815 |
| GGAUUGAGAACUCGACCCU | ORF | 1379 | 816 |
| GUCCCAGGUGAGUGGCAAU | ORF | 1404 | 817 |
| AAGAUCACCUUGAACGGCA | ORF | 274 | 818 |
| GUGCAGCAUGCGAGCGAUG | ORF | 990 | 819 |
| UCCUACGACUGCACGAAAU | ORF | 1483 | 820 |
| CUAAGGACCUGGUGAAGCC | ORF | 200 | 821 |
| AGAUCACCUUGAACGGCAU | ORF | 275 | 822 |
| CCAAGGAGAAGGAGGAGCG | ORF | 521 | 823 |
| GCCGAGAAGAAGGUGGUGA | ORF | 619 | 824 |
| CAGCUGCCCUACAGAUCCA | ORF | 1432 | 825 |
| ACAUCAUGACCCUGCGAAC | ORF | 821 | 826 |
| AGUCCCAGGUGAGUGGCAA | ORF | 1403 | 827 |
| CUACCUGAGCGAGCAGAAU | ORF | 51 | 828 |

Exemplary siRNAs for Goosecoid are shown in TABLE 14.

TABLE 14

| | REGION IN | START | |
|---|---|---|---|
| SEQUENCE | TARGET | POSITION | SEQ ID NO. |
| GGAGAAGAGGGAAGAGGAA | ORF | 873 | 829 |
| GCGGAGAAGUGGAACAAGA | ORF | 832 | 830 |
| CAUCAGAGGAGUCGGAGAA | ORF | 812 | 831 |
| AGAGGGAAGAGGAAGGUAA | ORF | 878 | 832 |
| GAGGGAAGAGGAAGGUAAA | ORF | 879 | 833 |
| GGAACGAGGAGCUGUAAAU | 3′ UTR | 1032 | 834 |
| ACAAUAAAGUGAUGGCGAU | 3′ UTR | 1168 | 835 |
| CGAAGGACUUGCACAGACA | 3′ UTR | 959 | 836 |
| AUAAAGUGAUGGCGAUGUA | 3′ UTR | 1171 | 837 |
| UGACAGUACAAUAAAGUGA | 3′ UTR | 1161 | 838 |
| AGUCGGAGAACGCGGAGAA | ORF | 821 | 839 |
| AGGAGAAAGUGGAGGUCUG | ORF | 749 | 840 |
| CGGCAGAAGCGGUCCUCAU | ORF | 796 | 841 |
| CGGAGAAGAGGGAAGAGGA | ORF | 872 | 842 |
| GCCAAAUGGAGGCGGCAGA | ORF | 784 | 843 |
| UUACCUAACUCGAAGGACU | 3′ UTR | 949 | 844 |
| CGAGAAAGAGGAACGAGGA | 3′ UTR | 1023 | 845 |
| AGAGGAACGAGGAGCUGUA | 3′ UTR | 1029 | 846 |
| GAAAGAGGAACGAGGAGCU | 3′ UTR | 1026 | 847 |
| ACGAGGAGCUGUAAAUAGU | 3′ UTR | 1035 | 848 |
| GGAAAGUGCACCUCCGCGA | ORF | 731 | 849 |
| GCGAGGAGAAAGUGGAGGU | ORF | 746 | 850 |
| CGGAGAACGCGGAGAAGUG | ORF | 824 | 851 |
| GAGGAAGGUAAAAGCGAUU | ORF | 886 | 852 |
| GGUAAAAGCGAUUUGGACU | ORF | 892 | 853 |
| AAGUGGAGGUCUGGUUUAA | ORF | 755 | 854 |
| AGACAGACGAUGCUACUUU | 3′ UTR | 973 | 855 |
| AAUUAAGGGUGACAGUACA | 3′ UTR | 1152 | 856 |
| AAGGGUGACAGUACAAUAA | 3′ UTR | 1156 | 857 |
| AAAGUGAUGGCGAUGUAAA | 3′ UTR | 1173 | 858 |
| GCUACAACAACUACUUCUA | ORF | 383 | 859 |
| ACAACUACUUCUACGGGCA | ORF | 389 | 860 |
| GAACGAGGAGCUGUAAAUA | 3′ UTR | 1033 | 861 |
| AUUAAGGGUGACAGUACAA | 3′ UTR | 1153 | 862 |
| GUGGAGGUCUGGUUUAAGA | ORF | 757 | 863 |
| ACGCGGAGAAGUGGAACAA | ORF | 830 | 864 |
| UCGAAGGCGUCACCGGAGA | ORF | 859 | 865 |
| AAAUUAAGGGUGACAGUAC | 3′ UTR | 1151 | 866 |
| AAGUGAUGGCGAUGUAAAA | 3′ UTR | 1174 | 867 |
| CCGCCAGCAUGUUCAGCAU | ORF | 152 | 868 |
| AAAGUGGAGGUCUGGUUUA | ORF | 754 | 869 |
| CCAAAUGGAGGCGGCAGAA | ORF | 785 | 870 |
| AGAACGCGGAGAAGUGGAA | ORF | 827 | 871 |
| GAGAAGUGGAACAAGACGU | ORF | 835 | 872 |
| CGAAGGCGUCACCGGAGAA | ORF | 860 | 873 |
| AGGAACGAGGAGCUGUAAA | 3′ UTR | 1031 | 874 |
| UAAGGGUGACAGUACAAUA | 3′ UTR | 1155 | 875 |
| GCUGCAAGGACUCGGUGUU | ORF | 197 | 876 |

Exemplary siRNAs for Sip1 are shown in TABLE 15.

TABLE 15

| | REGION IN | START | |
|---|---|---|---|
| SEQUENCE | TARGET | POSITION | SEQ ID NO. |
| AAAUGAAAGUCCUGGAAUA | ORF | 511 | 877 |
| GAAGAAGGCUGGAAGAAAU | ORF | 443 | 878 |
| ACAUAGAAGUCACUGGAAA | ORF | 379 | 879 |
| GGAACUGGCUGGUUUGAAA | ORF | 34 | 880 |
| AGUAAUUGGUUUGGAGAAA | ORF | 617 | 881 |
| UAACUAGUGUCUUGGAAUA | ORF | 594 | 882 |
| GGCCUUAGCAUCAGAAUUA | 3′ UTR | 1216 | 883 |
| GUUCAUAGUCAGCAAUAAA | 3′ UTR | 1261 | 884 |
| CAUAGAAGUCACUGGAAAU | ORF | 380 | 885 |
| GAAUAUGGGUUGAUUUGAA | 3′ UTR | 958 | 886 |
| CCAAAGAAGUUGAAAAGGA | ORF | 239 | 887 |
| UGAAGAAGGCUGGAAGAAA | ORF | 442 | 888 |
| CAAAGAAGUUGAAAAGGAA | ORF | 240 | 889 |
| GGAAGCAAAGUGUGAAUAU | ORF | 255 | 890 |
| GAGUAAUUGGUUUGGAGAA | ORF | 616 | 891 |
| GAGCGGAACUGGCUGGUUU | ORF | 30 | 892 |
| GAAGAUGGCUUUAUGCUUU | ORF | 657 | 893 |
| UCUCAGGGAUAGAAGAUAU | 3′ UTR | 818 | 894 |
| CAGCCUAACUCUGAGGAAA | 3′ UTR | 849 | 895 |
| GCAACAAGUGGCACAGUUU | ORF | 334 | 896 |
| GGACCAGCCACAAAUGAAA | ORF | 500 | 897 |
| UCUUGGAAUAUCUGAGUAA | ORF | 603 | 898 |
| CAACACAUCUUCAACACUA | 3′ UTR | 893 | 899 |
| GCGACUUGACGGAAGGUUU | ORF | 123 | 900 |
| GAACAAACAUAGAAGUCAC | ORF | 373 | 901 |
| GAUGAAGAAGGCUGGAAGA | ORF | 440 | 902 |
| CGACAGAAUGUGAACAAAC | ORF | 362 | 903 |
| GACAGAAUGUGAACAAACA | ORF | 363 | 904 |
| UAAUUGGUUUGGAGAAAGA | ORF | 619 | 905 |
| UCAGAUUGAUACUCAGAAU | 3′ UTR | 943 | 906 |
| CAGAUUGAUACUCAGAAUA | 3′ UTR | 944 | 907 |
| CCGCAGUGGAAGAGUUGAU | ORF | 78 | 908 |
| AAGAAGGCUGGAAGAAAUU | ORF | 444 | 909 |
| UGAAAGUCCUGGAAUAGAU | ORF | 514 | 910 |
| GUAACUAGUGUCUUGGAAU | ORF | 593 | 911 |
| GGAAGAUGGCUUUAUGCUU | ORF | 656 | 912 |
| GCAAGAAGGUGCUCUGAAG | ORF | 734 | 913 |
| AGCCUAACUCUGAGGAAAA | 3′ UTR | 850 | 914 |
| GGAAAAUCCCACUCAGUUU | 3′ UTR | 993 | 915 |
| GGCAAUGUGUUCAUAGUCA | 3′ UTR | 1253 | 916 |
| AAGGAAGCAAAGUGUGAAU | ORF | 253 | 917 |
| GUUGGAUAGUAAUGUGACA | ORF | 406 | 918 |
| AUGAAGAAGGCUGGAAGAA | ORF | 441 | 919 |
| AGACUUUACUCCAGAAUUG | ORF | 637 | 920 |
| AGAAUUGGGAAGAUGGCUU | ORF | 649 | 921 |
| CCUUAGCAUCAGAAUUAAA | 3′ UTR | 1218 | 922 |
| AAAUUGACCCAAAGAAGUU | ORF | 231 | 923 |
| GACCCAAAGAAGUUGAAAA | ORF | 236 | 924 |
| GAAGCAAAGUGUGAAUAUU | ORF | 256 | 925 |
| CCCAACACUUCAAUGGCAA | ORF | 313 | 926 |

Exemplary siRNAs for Snail1 are shown in TABLE 16.

TABLE 16

| | REGION IN | START | |
|---|---|---|---|
| SEQUENCE | TARGET | POSITION | SEQ ID NO. |
| GCUUUGAGCUACAGGACAA | 3′ UTR | 1176 | 927 |
| GGACAAAGGCUGACAGACU | 3′ UTR | 1189 | 928 |
| GAAAAGGGACUGUGAGUAA | 3′ UTR | 1452 | 929 |
| AGAUGAGGACAGUGGGAAA | ORF | 346 | 930 |
| ACUCAGAUGUCAAGAAGUA | ORF | 759 | 931 |
| GGACUUUGAUGAAGACCAU | 3′ UTR | 1006 | 932 |
| GUGACUAACUAUGCAAUAA | 3′ UTR | 1297 | 933 |
| CCUGGGAGGAAGAUGUUUA | 3′ UTR | 1558 | 934 |
| GCAAAUACUGCAACAAGGA | ORF | 537 | 935 |
| AAUACUGCAACAAGGAAUA | ORF | 540 | 936 |
| GAGUGGUUCUUCUGCGCUA | 5′ UTR | 14 | 937 |
| GCUACAGGACAAAGGCUGA | 3′ UTR | 1183 | 938 |
| AAAUACUGCAACAAGGAAU | ORF | 539 | 939 |
| UCAAGAAGUACCAGUGCCA | ORF | 768 | 940 |
| CACAGGACUUUGAUGAAGA | 3′ UTR | 1002 | 941 |
| GCAAUUUAACAAUGUCUGA | 3′ UTR | 1435 | 942 |
| UCUCUGAGGCCAAGGAUCU | ORF | 495 | 943 |
| CGGCCUAGCGAGUGGUUCU | 5′ UTR | 5 | 944 |
| GAUGUGUCUCCCAGAACUA | 3′ UTR | 1517 | 945 |
| GGGCCUGGGAGGAAGAUGU | 3′ UTR | 1555 | 946 |
| UUUUAAAGGUACACUGGUA | 3′ UTR | 1580 | 947 |
| CGAAAGGCCUUCAACUGCA | ORF | 521 | 948 |
| CCCACAGGACUUUGAUGAA | 3′ UTR | 1000 | 949 |
| UUAAAGGUACACUGGUAUU | 3′ UTR | 1582 | 950 |
| GAAAGGCCUUCAACUGCAA | ORF | 522 | 951 |
| ACAAAGGCUGACAGACUCA | 3′ UTR | 1191 | 952 |
| CUCCACGAGGUGUGACUAA | 3′ UTR | 1286 | 953 |
| GAGUAAUGGCUGUCACUUG | 3′ UTR | 1465 | 954 |
| AAUCGGAAGCCUAACUACA | ORF | 110 | 955 |
| GCGAGCUGCAGGACUCUAA | ORF | 129 | 956 |
| CCACAAGCACCAAGAGUCC | ORF | 823 | 957 |
| CAGGACAAAGGCUGACAGA | 3′ UTR | 1187 | 958 |
| ACAAGGAACCCUCAGGCCA | 3′ UTR | 1265 | 959 |
| CAGAUGAGGACAGUGGGAA | ORF | 345 | 960 |
| GAAUGUCCCUGCUCCACAA | ORF | 810 | 961 |
| ACUUUGAUGAAGACCAUUU | 3′ UTR | 1008 | 962 |
| GGCCUGUCUGCGUGGGUUU | 3′ UTR | 1129 | 963 |
| GGGCAAUUUAACAAUGUCU | 3′ UTR | 1433 | 964 |
| UUUAAAGGUACACUGGUAU | 3′ UTR | 1581 | 965 |
| AGGUACACUGGUAUUUAUA | 3′ UTR | 1586 | 966 |
| CAAAUACUGCAACAAGGAA | ORF | 538 | 967 |
| GAACCUGCGGGAAGGCCUU | ORF | 618 | 968 |
| AGACCCACUCAGAUGUCAA | ORF | 753 | 969 |
| AAGCCUAACUACAGCGAGC | ORF | 116 | 970 |
| UCAGAUGAGGACAGUGGGA | ORF | 344 | 971 |
| GCUCGAAAGGCCUUCAACU | ORF | 518 | 972 |
| AUGCACAUCCGAAGCCACA | ORF | 581 | 973 |
| CCACUCAGAUGUCAAGAAG | ORF | 757 | 974 |
| GGCCAUUUCUGUGGAGGGA | 3′ UTR | 1073 | 975 |
| AGUAAUGGCUGUCACUUGU | 3′ UTR | 1466 | 976 |

Exemplary siRNAs for Snail2 are shown in TABLE 17.

TABLE 17

| | REGION IN | START | |
|---|---|---|---|
| SEQUENCE | TARGET | POSITION | SEQ ID NO. |
| CAUUAGUGAUGAAGAGGAA | ORF | 479 | 977 |
| GGACACACAUACAGUGAUU | ORF | 230 | 978 |
| GGCUAGAUUGAGAGAAUAA | 3′ UTR | 1211 | 979 |
| GAACAGUAUUGCUUUGUAA | 3′ UTR | 1337 | 980 |
| CAAAUAAAGUCCAAAGGCA | 3′ UTR | 1030 | 981 |
| CUGUAGUGCUUUAAAGUAU | 3′ UTR | 1495 | 982 |
| AAGAAAUACCAGUGCAAAA | ORF | 879 | 983 |
| AUGGCUAGAUUGAGAGAAU | 3′ UTR | 1209 | 984 |
| UUGUAUAGUUGAUGAGUCA | 3′ UTR | 1827 | 985 |
| AAAUAAAGUCCAAAGGCAU | 3′ UTR | 1031 | 986 |
| CCUGAAGACUUGUGAAAUC | 3′ UTR | 1929 | 987 |
| CUUCAUGAUUAGUACCAAA | 3′ UTR | 2046 | 988 |
| UAAAGAAAUACCAGUGCAA | ORF | 877 | 989 |
| GUAUAGACACACACACAUA | 3′ UTR | 1080 | 990 |
| GCUGAUGGCUAGAUUGAGA | 3′ UTR | 1205 | 991 |
| UGUAAUAGGAUUUCCCAUA | 3′ UTR | 1363 | 992 |
| CCACAAAUGCAAUAAUACA | 3′ UTR | 1782 | 993 |
| GAACAAAACACAGGAGAAU | 3′ UTR | 1543 | 994 |
| UCGUAAAGGAGCCGGGUGA | 5′ UTR | 4 | 995 |
| ACACACACCCACAGAGAGA | 3′ UTR | 1112 | 996 |
| GAGAUGUUGUCUAUAGCUA | 3′ UTR | 1897 | 997 |
| CAUUGAAGCUGAAAAGUUU | ORF | 530 | 998 |
| AAUAAAGUCCAAAGGCAUU | 3′ UTR | 1032 | 999 |
| AGAGAGAGCUGCAAGAGCA | 3′ UTR | 1126 | 1000 |
| GCUGCAAGAGCAUGGAAUU | 3′ UTR | 1133 | 1001 |
| AGAACAAAACACAGGAGAA | 3′ UTR | 1542 | 1002 |
| GAAUGAGUUCUGUAUGAAA | 3′ UTR | 1876 | 1003 |
| UGAUGAAGAGGAAAGACUA | ORF | 485 | 1004 |
| AAUACUGUGACAAGGAAUA | ORF | 649 | 1005 |
| GCACAAACAUGAGGAAUCU | ORF | 932 | 1006 |
| UUGAAUGAGUUCUGUAUGA | 3′ UTR | 1874 | 1007 |
| AAACUGAGAUGUUGUCUAU | 3′ UTR | 1892 | 1008 |
| CCAAACCACUGUACAAAGA | 3′ UTR | 2060 | 1009 |
| ACACACAUACAGUGAUUAU | ORF | 232 | 1010 |
| GUGAUGAAGAGGAAAGACU | ORF | 484 | 1011 |
| GUAAAUACUGUGACAAGGA | ORF | 646 | 1012 |
| CCACAGAGAGAGAGCUGCA | 3′ UTR | 1120 | 1013 |
| AUAUAUUUGCUGAUGGCUA | 3′ UTR | 1197 | 1014 |
| GCUCCUUCCUGGUCAAGAA | ORF | 172 | 1015 |
| GAAACUGAGAUGUUGUCUA | 3′ UTR | 1891 | 1016 |
| AUAAACAACCUGAAGACUU | 3′ UTR | 1921 | 1017 |
| AACCUGAAGACUUGUGAAA | 3′ UTR | 1927 | 1018 |
| AAGCCAAACUACAGCGAAC | ORF | 210 | 1019 |
| CAGAGAGAGAGCUGCAAGA | 3′ UTR | 1123 | 1020 |
| GAUGGGAAUAAGUGCAAAA | 3′ UTR | 1714 | 1021 |
| UUUCAAAUGCAUACCACAA | 3′ UTR | 1769 | 1022 |
| UGUAUGAAACUGAGAUGUU | 3′ UTR | 1886 | 1023 |
| CCUCACUGCAACAGAGCAU | ORF | 810 | 1024 |
| CAAUCAAUGUUUACUCGAA | 3′ UTR | 978 | 1025 |
| GAAGCCAAAUGACAAAUAA | 3′ UTR | 1018 | 1026 |

Exemplary siRNAs for TCF3 are shown in TABLE 18.

TABLE 18

| | REGION IN | START | |
|---|---|---|---|
| SEQUENCE | TARGET | POSITION | SEQ ID NO. |
| AGAAGGAGGACGAGGAGAA | ORF | 1532 | 1027 |
| GCAUAGAAUUCAAACGAGA | 3′ UTR | 4136 | 1028 |
| CCGGAUCACUCAAGCAAUA | ORF | 1054 | 1029 |
| AGAUCAAGCGGGAGGAGAA | ORF | 1517 | 1030 |
| AAACAAAACCUGAAAGCAA | 3′ UTR | 2334 | 1031 |
| ACUCGGAGGAGGAGAAGAA | ORF | 1568 | 1032 |
| GGGCACAUGUGAAAGGUAU | ORF | 1984 | 1033 |
| CCUGAAAGCAAGCAACAAA | 3′ UTR | 2342 | 1034 |
| GGGAGGAGAAGGAGGACGA | ORF | 1526 | 1035 |
| GCACCAGCCUCAUGCACAA | ORF | 1394 | 1036 |
| ACACUUUGUCAGAGAAGAA | 3′ UTR | 2365 | 1037 |
| AGGAGAAGAAGGAGCUGAA | ORF | 1577 | 1038 |
| AAAUUGUGCCUAAGCGAAA | 3′ UTR | 2478 | 1039 |
| CAGACGAGGACGAGGACGA | ORF | 1619 | 1040 |
| UAGCAAUAAACGUGACAUU | 3′ UTR | 4370 | 1041 |
| GUUCGGAGGUUCAGGUCUU | ORF | 162 | 1042 |
| CGGAGGAGGAGAAGAAGGA | ORF | 1571 | 1043 |
| GAAACGGCGAGAAGAGGAA | ORF | 1884 | 1044 |
| GCAUAUGUUUUGUAAGCAA | 3′ UTR | 2609 | 1045 |
| AGAGUAAGAUAGAAGACCA | ORF | 1199 | 1046 |
| GCGCGAGGAGGAAGAAACA | 3′ UTR | 4163 | 1047 |
| CUACAGUGGGCUAGGGCGA | ORF | 1473 | 1048 |
| ACAUACACUUUGUCAGAGA | 3′ UTR | 2361 | 1049 |
| UCUAAAGCCACCAGCAAAU | 3′ UTR | 2463 | 1050 |
| CUGUGUGGUCCAAGGGCAA | 3′ UTR | 3393 | 1051 |
| UGUCAGGUGUGGUUGGAGA | ORF | 1907 | 1052 |
| AAACAUACACUUUGUCAGA | 3′ UTR | 2359 | 1053 |
| CAGACAAGGAGCUCAGUGA | ORF | 65 | 1054 |
| GGGGAAGGGACGUCAGCAA | 3′ UTR | 2952 | 1055 |
| GGAGGAAGAAACAGCAGUU | 3′ UTR | 4169 | 1056 |
| GCAAUAAACGUGACAUUUU | 3′ UTR | 4372 | 1057 |
| CGGCCUGCAGAGUAAGAUA | ORF | 1191 | 1058 |
| AGGAGAAGGAGGACGAGGA | ORF | 1529 | 1059 |
| CUUCUAAAGCCACCAGCAA | 3′ UTR | 2461 | 1060 |
| CCAUUACACCAGAGGGCCA | 3′ UTR | 3284 | 1061 |
| AUGGUAGAUGCAAGGGAAA | 3′ UTR | 3905 | 1062 |
| UAGAAGACCACCUGGACGA | ORF | 1208 | 1063 |
| CCAGCGAGAUCAAGCGGGA | ORF | 1511 | 1064 |
| GCAAAUUGUGCCUAAGCGA | 3′ UTR | 2476 | 1065 |
| GUGCCUAAGCGAAAUAUUU | 3′ UTR | 2483 | 1066 |
| GAUGAAAAUUAGCAAGGAU | 3′ UTR | 2554 | 1067 |
| UCCACGGCCUGCAGAGUAA | ORF | 1187 | 1068 |
| CUGCAGAGUAAGAUAGAAG | ORF | 1195 | 1069 |
| AGGAAAAGGUGUCAGGUGU | ORF | 1898 | 1070 |
| CAUUGCAUUUCUUGAUCAA | 3′ UTR | 2690 | 1071 |
| GGGACUGUCUUGGGUUUAA | 3′ UTR | 3606 | 1072 |
| GAGCAGAGGUGAACGGUGG | ORF | 869 | 1073 |
| UCAGUGACCUCCUGGACUU | ORF | 77 | 1074 |
| UGAACCAGCCGCAGAGGAU | ORF | 32 | 1075 |
| GCAACAAAACAUACACUUU | 3′ UTR | 2353 | 1076 |

Exemplary siRNAs for Twist are shown in TABLE 19.

TABLE 19

| | REGION IN | START | |
|---|---|---|---|
| SEQUENCE | TARGET | POSITION | SEQ ID NO. |
| GGAAAUUAGAAGAGCAAAA | 3′ UTR | 1095 | 1077 |
| CAGAGGAACUAUAAGAACA | 3′ UTR | 1393 | 1078 |
| GGAUCAAACUGGCCUGCAA | 3′ UTR | 1433 | 1079 |
| GGUAACAAUCAGAGGAACU | 3′ UTR | 1384 | 1080 |
| GCAAAACCAUAGUCAGUUA | 3′ UTR | 1448 | 1081 |
| GGACAAGCUGAGCAAGAUU | ORF | 771 | 1082 |
| UUGGAAAUUAGAAGAGCAA | 3′ UTR | 1093 | 1083 |
| CCUCGGACAAGCUGAGCAA | ORF | 767 | 1084 |
| CCGGAGACCUAGAUGUCAU | 3′ UTR | 991 | 1085 |
| GAUAGAAGUCUGAACAGUU | 3′ UTR | 1228 | 1086 |
| AUUGAGGACCCAUGGUAAA | 3′ UTR | 1544 | 1087 |
| CCGACGACAGCCUGAGCAA | ORF | 389 | 1088 |
| AGGAAGAGCCAGACCGGCA | ORF | 413 | 1089 |
| GAGCAAAAUCCAAAUUCAA | 3′ UTR | 1106 | 1090 |
| GAUCAAACUGGCCUGCAAA | 3′ UTR | 1434 | 1091 |
| GCAAAUAGAUCCGGUGUCU | 3′ UTR | 1565 | 1092 |
| GUGUCUAAAUGCAUUCAUA | 3′ UTR | 1578 | 1093 |
| GAGAGAUGAUGCAGGACGU | 5′ UTR | 347 | 1094 |
| UGAGCAACAGCGAGGAAGA | ORF | 401 | 1095 |
| UCGGACAAGCUGAGCAAGA | ORF | 769 | 1096 |
| AGACUCUGGAGCUGGAUAA | 3′ UTR | 1043 | 1097 |
| CUCUGGAGCUGGAUAACUA | 3′ UTR | 1046 | 1098 |
| UAAAAGAGAAAGCGAGACA | 3′ UTR | 1150 | 1099 |
| ACGAGGAGCUGCAGACGCA | ORF | 659 | 1100 |
| UGUCAUUGUUUCCAGAGAA | 3′ UTR | 1004 | 1101 |
| GAAAGGAAAGGCAUCACUA | 3′ UTR | 1343 | 1102 |
| GACGACAGCCUGAGCAACA | ORF | 391 | 1103 |
| GCAAGAAGUCUGCGGGCUG | ORF | 575 | 1104 |
| CUUGGAAAUUAGAAGAGCA | 3′ UTR | 1092 | 1105 |
| AUUCAAAGAAACAGGGCGU | 3′ UTR | 1119 | 1106 |
| CCACUGAAAGGAAAGGCAU | 3′ UTR | 1338 | 1107 |
| AUGGUAACAAUCAGAGGAA | 3′ UTR | 1382 | 1108 |
| GUAACAAUCAGAGGAACUA | 3′ UTR | 1385 | 1109 |
| AAUCAGAGGAACUAUAAGA | 3′ UTR | 1390 | 1110 |
| UAUUGAGGACCCAUGGUAA | 3′ UTR | 1543 | 1111 |
| CCUGAGCAACAGCGAGGAA | ORF | 399 | 1112 |
| CAACAGCGAGGAAGAGCCA | ORF | 405 | 1113 |
| ACAGCGAGGAAGAGCCAGA | ORF | 407 | 1114 |
| GAGAAGGAGAAAAUGGACA | 3′ UTR | 1018 | 1115 |
| UAGAAGAGCAAAAUCCAAA | 3′ UTR | 1101 | 1116 |
| UUUAAAAGAGAAAGCGAGA | 3′ UTR | 1148 | 1117 |
| GGUAAAAUGCAAAUAGAUC | 3′ UTR | 1557 | 1118 |
| GCACCCAGUCGCUGAACGA | ORF | 710 | 1119 |
| CGGACAAGCUGAGCAAGAU | ORF | 770 | 1120 |
| CAUUGUUUCCAGAGAAGGA | 3′ UTR | 1007 | 1121 |
| UUCCAGAGAAGGAGAAAAU | 3′ UTR | 1013 | 1122 |
| AGGAGAAAAUGGACAGUCU | 3′ UTR | 1022 | 1123 |
| CUGCAAAACCAUAGUCAGU | 3′ UTR | 1446 | 1124 |
| GGAGAAAAUGGACAGUCUA | 3′ UTR | 1023 | 1125 |
| AGGCAUCACUAUGGACUUU | 3′ UTR | 1351 | 1126 |

In addition to nucleic acid base modulators, it is contemplated that protein based modulators can be used in the practice of the invention, which can include, for example, antibodies, adzymes, protein-based aptamers, and therapeutic polypeptides.

It is contemplated that antibodies can be used in the practice of the invention. The antibodies preferably specifically bind and inactivate or reduce the activity of one or more of the transcription factors described herein, including, for example, Oct4 (protein sequence—SEQ ID NO: 2 or 4), Sox2 (protein sequence—SEQ ID NO: 6), Klf2 (protein sequence—SEQ ID NO: 16), Klf4 (protein sequence—SEQ ID NO: 8), Klf5 (protein sequence—SEQ ID NO: 14), Nanog (protein sequence—SEQ ID NO: 10), Tbx3 (protein sequence—SEQ ID NO: 22), ESRRB (protein sequence—SEQ ID NO: 18), REST (protein sequence—SEQ ID NO: 20), c-Myc (protein sequence—SEQ ID NO: 12), Foxc1 (protein sequence—SEQ ID NO: 24), Foxc2 (protein sequence—SEQ ID NO: 26), Goosecoid (protein sequence—SEQ ID NO: 28), Sip1 (protein sequence—SEQ ID NO: 30), Snail1 (protein sequence—SEQ ID NO: 32), Snail2 (protein sequence—SEQ ID NO: 34), Tcf3 (protein sequence—SEQ ID NO: 36), and Twist (protein sequence—SEQ ID NO: 38).

It is understood that each antibody directed to a stemness inducing or maintaining transcription factor can be an intact antibody, for example, a monoclonal antibody, an antigen binding fragment of an antibody, or a biosynthetic antibody binding site. Antibody fragments include Fab, Fab′, (Fab′)₂ or Fv fragments. The antibodies and antibody fragments can be produced using conventional techniques known in the art. A number of biosynthetic antibody binding sites are known in the art and include, for example, single Fv or sFv molecules, described, for example, in U.S. Pat. Nos. 5,091,513, 5,132,405, and 5,476,786. Other biosynthetic antibody binding sites include bispecific or bifunctional binding proteins, for example, bispecific or bifunctional antibodies, which are antibodies or antibody fragments that bind at least two different antigens. For example, bispecific binding proteins can bind both Oct4 and Sox2. Methods for making bispecific antibodies are known in art and, include, for example, by fusing hybridomas or by linking Fab′ fragments. See, e.g., Songsivilai et al. (1990) CLIN. EXP. IMMUNOL. 79: 315-325; Kostelny et al. (1992) J. IMMUNOL. 148: 1547-1553.

It is understood that antibodies to each of the foregoing transcription factors are available commercially and may be used in the practice of the invention. For example, anti-Oct4 antibodies (as denoted by their respective catalog number) are available commercially, as ab19857, ab27985, ab18976, ab53028, ab52014, ab27449, ab59545, ab60127, all of which are available from Abcam (Cambridge, Mass., USA); sc-8628, sc-5279, sc-9081, sc-8629, sc-25401, and sc-8630, all of which are available from Santa Cruz Biotechnology (Santa Cruz, Calif., USA); MAB4305, MAB4401 and AB3209, all of which are available from Millipore (Billerica, Mass., USA); 611202 and 611203 which are available from BD Transduction Laboratories (San Jose, Calif., USA); 560186, 560253, 560217, 560307 and 560306, all of which are available from BD Pharmingen (San Diego, Calif., USA); AF1754 and MAB1759 which are available from R&D Systems (Minneapolis, Minn., USA); O5402-09 available from US Biological (Swampscott, Mass., USA); and 14-5841 available from eBioscience (San Diego, Calif., USA).

Anti-Sox2 antibodies (as denoted by their respective catalog number) are available, for example, as ab15830 available from Abcam (Cambridge, Mass., USA).

Anti-Klf4 antibodies (as denoted by their respective catalog number) are available, for example, as ab26648, ab21949, ab34814, ab56542, and ab58358, all of which are available from Abcam (Cambridge, Mass., USA); IMG-3231 available from Imgenex (San Diego, Calif., USA); AB4138 available from Millipore (Billerica, Mass., USA); sc-20691, sc12538 and sc-1905, all of which are available from Santa Cruz Biotechnology (Santa Cruz, Calif., USA); K1891-41 available from US Biological (Swampscott, Mass., USA); and 42-4100 available from Invitrogen (Carlsbad, Calif., USA).

Anti-Nanog antibodies (as denoted by their respective catalog numbers) are available, for example, as ab21603, ab21624, ab62734, ab14959, and ab7102, all of which are available from Abcam (Cambridge, Mass., USA); 14-5768 and 14-5769 which are available from eBioscience (San Diego, Calif., USA); A300-397A and A300-398A which are available from Bethyl Laboratories (Montgomery, Tex., USA); AB5731, AB9220, and MAB10091, all of which are available from Millipore (Billerica, Mass., USA); RHF773 available from Antigenix America (Huntington Station, N.Y., USA); sc-33759, sc-81961, sc-30329, sc-33760, sc30331, and sc-30328, all of which are available from Santa Cruz Biotechnology (Santa Cruz, Calif., USA); 500-P236 available from PeproTech (Rocky Hill, N.J., USA); and AF1997, MAB1997, and AF2729, all of which are available from R&D Systems (Minneapolis, Minn., USA).

Anti-c-Myc antibodies (as denoted by their respective catalog numbers) are available, for example, as ab32, ab56, ab39688, ab32072, ab51156, ab19233, ab51154, ab39686, ab62928, ab19234, ab11917, ab17356, ab10825, ab10827, ab10910, ab1430, ab31430, ab31426, ab19312, ab64478, ab28058, ab17767, ab27027, ab47004, ab12213, ab14286, ab17355, ab63560, ab28056, ab19235, and ab10826, all of which are available from Abcam (Cambridge, Mass., USA); 14-6755, 14-6785, and 14-6784, all of which are available from eBioscience (San Diego, Calif., USA); A190-103A, A190-104A, A190-105A, A190-203A, A190-204A, and A190-205A, all of which are available from Bethyl Laboratories (Montgomery, Tex., USA); MAB8864, MAB8865, CBL439, CBL430, CBL434, AB3252, and AB3419, all of which are available from Millipore (Billerica, Mass., USA); MCA1929, MCA574T, and MCA2200GA, all of which are available from AbD Serotec (Raleigh, N.C., USA); sc-70463, sc-70469, sc-70464, sc-70461, sc-70458, sc-70468, sc70465, sc-56632, sc-70466, sc-70467, sc-70470, sc-70462, sc-53854, sc-70459, sc-70460, sc-40, sc-47694, sc-789, sc-788, sc-42, sc-41, sc-56633, sc-56634, sc-764, sc-56505, and sc-53183, all of which are available from Santa Cruz Biotechnology (Santa Cruz, Calif., USA); C0035-21A, C0035-35, C0036-06, C0035-09, C0035-30, C0035-04, C0035-07A, C0035-07E, C0035-07F, C0035-07G, C0035-07H, and C0035-09A, all of which are available from US Biological (Swampscott, Mass., USA); and 13-2500, 13-2511, A21280, and A21281, all of which are available from Invitrogen (Carlsbad, Calif., USA).

Anti-Klf2 antibodies (as denoted by their respective catalog numbers) are available, for example, as ab17008 and ab28526 which are available from Abcam (Cambridge, Mass., USA); AB4137 available from Millipore (Billerica, Mass., USA); and H00010365-A01 available from Abnova (Walnut, Calif., USA).

Anti-Klf5 antibodies (as denoted by their respective catalog numbers) are available, for example, as ab24331 available from Abcam (Cambridge, Mass., USA); AF3758 available R&D Systems (Minneapolis, Minn., USA); H00000688-A01 and H00000688-M01 which are available from Abnova (Walnut, Calif., USA).

Anti-ESRRB antibodies (as denoted by their respective catalog numbers) are available, for example, as ab12987 and ab12986 which are available from Abcam (Cambridge, Mass., USA); sc-56831, sc-8974, sc-6822, sc-6820, sc-56832, and sc-6821, all of which are available from Santa Cruz Biotechnology (Santa Cruz, Calif., USA); PP-H6705-00 and PP-H6707-00 which are available from R&D Systems (Minneapolis, Minn., USA).

Anti-REST antibodies (as denoted by their respective catalog numbers) are available, for example, as ab28018, ab43684, ab52849, ab52850, and ab21635, all of which are available from Abcam (Cambridge, Mass., USA); sc-15118, sc-15120, and sc-25398, all of which are available from Santa Cruz Biotechnology (Santa Cruz, Calif., USA); and 07-579 and AB15548 which are available from Millipore (Billerica, Mass., USA).

Anti-TBX3 antibodies (as denoted by their respective catalog numbers) are available, for example, as ab58264, ab66306, and ab21756, all of which are available from Abcam (Cambridge, Mass., USA); sc-101166, sc-17871, sc-17872, sc-31656, sc-48781, and sc-31657, all of which are available from Santa Cruz Biotechnology (Santa Cruz, Calif., USA); MAB10089 available from Millipore (Billerica, Mass., USA); and AF4509 available from R&D Systems (Minneapolis, Minn., USA).

Anti-Foxc1 antibodies (as denoted by their respective catalog numbers) are available, for example, as ab5079 and ab24067 which are available from Abcam (Cambridge, Mass., USA); sc-21396 and sc-21394 which are available from Santa Cruz Biotechnology (Santa Cruz, Calif., USA); and H00002296-M02, H00002296-M05, and H00002296-M09, all of which are available from Abnova (Walnut, Calif., USA).

Anti-Foxc2 antibodies (as denoted by their respective catalog numbers) are available, for example, as ab5060, ab55004, and ab24340, all of which are available from Abcam (Cambridge, Mass., USA); sc-31732, sc-31733, sc-28704, sc21397, sc-31734, and sc-101044, all of which are available from Santa Cruz Biotechnology (Santa Cruz, Calif., USA); and H00002303-M01, H00002303-M02, H00002303-M03, H00002303-M04, H00002303-M05, and H00002303-M08, all of which are available from Abnova (Walnut, Calif., USA).

Anti-Goosecoid antibodies (as denoted by their respective catalog numbers) are available, for example, as ab58352, available from Abcam (Cambridge, Mass., USA); sc-81964 and sc-22234 which are available from Santa Cruz Biotechnology (Santa Cruz, Calif., USA); AF4086 available R&D Systems (Minneapolis, Minn., USA), H00145258-B01, H00145258-A01, H00145258-M01, and H00145258-M03, all of which are available from Abnova (Walnut, Calif., USA).

Anti-Sip1 antibodies (as denoted by their respective catalog numbers) are available, for example, as ab6084, available from Abcam (Cambridge, Mass., USA); sc-33703, sc-57006, and sc-32806, all of which are available from Santa Cruz Biotechnology (Santa Cruz, Calif., USA); H00008487-B01 available from Abnova (Walnut, Calif., USA), and 611256 available from BD Biosciences (San Jose, Calif., USA).

Anti-Snail1 antibodies (as denoted by their respective catalog numbers) are available, for example, as ab17732, ab63568, ab63371, and ab53519, all of which are available from Abcam (Cambridge, Mass., USA); sc-10433, sc-10432, and sc-28199, all of which are available from Santa Cruz Biotechnology (Santa Cruz, Calif., USA); MAB5495 available from Millipore (Billerica, Mass., USA); AF3639 available from R&D Systems (Minneapolis, Minn., USA), H00006615-M10 and H00006615-B02 which are available from Abnova (Walnut, Calif., USA).

Anti-Snail2 antibodies (as denoted by their respective catalog numbers) are available, for example, as ab51772, ab27568, ab38551, ab63119, and ab62589, all of which are available from Abcam (Cambridge, Mass., USA); sc-15391, sc-10436, and sc-10437, all of which are available from Santa Cruz Biotechnology (Santa Cruz, Calif., USA); H00006591-A01, H00006591-A03, H00006591-A04, and H00006591-A05, all of which are available from Abnova (Walnut, Calif., USA).

Anti-TCF3 antibodies (as denoted by their respective catalog numbers) are available, for example, as ab59117, ab66373, ab58270, ab11176, and ab54462, all of which are available from Abcam (Cambridge, Mass., USA); sc-763 and sc-416 which are available from Santa Cruz Biotechnology (Santa Cruz, Calif., USA); and H00006929-M01 available from Abnova (Walnut, Calif., USA).

Anti-Twist antibodies (as denoted by their respective catalog numbers) are available, for example, as ab50887, ab50581, and ab49254, all of which are available from Abcam (Cambridge, Mass., USA); sc-6269, sc-6070, sc-15393, and sc-81417, all of which are available from Santa Cruz Biotechnology (Santa Cruz, Calif., USA).

Under certain circumstances, the antibodies can be conjugated, using conventional conjugation chemistries, to a cytotoxic agent. The cytotoxic agent can be, for example, a nitrogen mustard, gemcitabine, an ethylenimine derivative, an alkyl sulfonate, a nitrosourea, a triazene, a folic acid analog, an anthracycline, a taxane, SN-38, a COX-2 inhibitor, a pyrimidine analog, a purine analog, an antibiotic, an enzyme, an enzyme inhibitor, an epipodophyllotoxin, a platinum coordination complex, a vinca alkaloid, a substituted urea, a methyl hydrazine derivative, an adrenocortical suppressant, a hormone antagonist, taxol, camptothecin, doxorubicin, an alkylating agent, an antimitotic, an antiangiogenic agent, an apoptotic agent, and methotrexate.

The therapeutic polypeptide directed to a stemness modulating transcription factor can be delivered to a subject in need thereof to ameliorate one or more symptoms of cancer. The therapeutic polypeptide can be administered systemically (e.g., by intravenous infusion) or locally (e.g., directly to an organ or tissue, such as the eye or the liver). It is understood that the therapeutic polypeptides (for example, the antibodies described herein) can be used in combination with suitable delivery systems to facilitate entry of the therapeutic polypeptides into a cell, and under certain circumstances into a nucleus of a cell.

In addition to nucleic acid-based and protein-based modulators, it is understood that small molecule-based modulators can be used in the practice of the invention. The small molecule-based modulators inhibit the expression of transcription factors or modulate the activity of transcription factors that (i) modulate the differentiation of differentiated cells into cancer stem cells and/or (ii) modulate the maintenance of cancer stem cells. The small molecules can be synthesized using conventional synthetic chemistries well known in the art (reviewed by Thompson and Ellman, CHEM. REV. 96:555-600, 1996; Beeler et al., CURR. OPIN. CHEM. BIOLOGY 9:277-284, 2005).

In addition to molecules that inhibit the transition of differentiated cells into cancer stem cells or molecules that inhibit the maintenance of stem cells, it is contemplated that such molecules can be combined with the agents that promote the differentiation of cancer stem cells. Such agents include, for example, all trans retinoic acid (RA), dimethyl sulfoxide, vitamin D(3), ciglitazone, troglitazone, pioglitazone, rosiglitazone, 12-0-tetradecanoylphorbol 13-acetate (PMA), hexamethylene-bis-acetamide, nerve growth factor (NGF), TGFβ, butyric acid, cAMP, and vesnarinone (reviewed by Kawamata et al. CURRENT PHARMACEUTICAL DESIGN, 12:379-85, 2006; Yasui et al., PPAR RES. 2008:548919, 2008).

(b) Anti-Cancer Agents

During the practice of the invention, the stemness-reducing agents discussed in the previous section are used to reduce the number of differentiated cells with a propensity to form cancer stem cells and/or to reduce the number of cancer stem cells by inhibiting their ability to maintain stemness. The differentiated cells, including those that have lost the properties of stemness, are exposed to standard anti-cancer agents, for example, chemotherapeutic agents, radioisotopes, and immunomodulators, to reduce the number of differentiated cancer cells.

It is understood that one or more of the stemness reducing agents disclosed herein can be used (for example, delivered to a subject, for example, a human or non-human subject with cancer) in combination with a known chemotherapeutic agent. It is contemplated that prior treatment or concurrent treatment with the stemness reducing agent may reduce the number of cancer stem cells in a particular mixture of cancer stem cells and cancer cells.

Exemplary chemotherapeutic agents useful in the practice of the invention include, for example, Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon α-2a; Interferon α-2b; Interferon α-n1; Interferon α-n3; Interferon β-I a; Interferon γ-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; tumor necrosis factor α (TNF), Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin and Zorubicin Hydrochloride.

Chemotherapeutic agents also can include agents that act on the tumor vasculature and include, for example, tubulin-binding agents, such as combrestatin A4 (Griggs et al., LANCET ONCOL. 2:82, 2001), angiostatin and endostatin (reviewed in Rosen, ONCOLOGIST 5:20, 2000), interferon inducible protein 10 (see, for example, U.S. Pat. No. 5,994,292), and the like.

Chemotherapeutic agents also can include inhibitors of neovascularisation, including, for example, the VEGF inhibitors, bevacizumab (Avastin), ranibizumab (Lucentis), sunitinib (Sutent), sorafenib (Nexavar), axitinib, pazopanib, aflibercept (reviewed in Moreira et al., ANTICANCER AGENTS MED. CHEM. 7:223, 2007; Goh et al., CURR. CANCER DRUG TARGETS 7:743, 2007; Glade-bender et al., EXPERT OPIN. BIOL. THER. 3:263, 2003). Chemotherapeutic agents can also include lysosomal inhibitors, such as, Velcade. Furthermore, chemotherapeutic agents also include retinoic acid, retinoic acid derivatives, and other chemical inducers of differentiation known to those skilled in the art.

Other anti-cancer agents include agents that act on tumor neovasculature including, for example, cytotoxic radionuclides, chemical toxins and protein toxins. The cytotoxic radionuclide or radiotherapeutic isotope preferably is an alpha-emitting isotope such as ²²⁵Ac, ²¹¹At, ²¹²Bi, ²¹³Bi, ₂₁₂Pb, ²²⁴Ra or ²²³Ra. Alternatively, the cytotoxic radionuclide may a beta-emitting isotope including, for example, ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons including, for example, ¹²⁵I, ¹²³I or ⁷⁷Br.

One or more agents modulating stemness can be delivered to mammalian cells using methods known in the art. For example, siRNA delivery vehicles can include poly(beta-amino esters), liposomes (including pH-dependent liposomes, e.g., Auguste et al., J. CONTROL RELEASE, Jun. 12, 2008), lipidoids (Akinc et al., NATURE BIOTECHNOLOGY 26:561, 2008), viruses, etc (see, for example, U.S. Pat. Nos. 5,783,567, 5,942,634, and 7,002,027, and U.S. Patent Application Publication Nos. US2004/0071654, US2006/0073127, US2005/0008617, US2006/0240554).

### C. Methods of Treatment, Formulations, and Modes of Administration

(1) Methods of Treatment

The compositions disclosed herein are useful for treating and preventing cancer cell proliferation and metastasis in a subject (for example, a human or non-human mammal) that has or is at risk of having cancer.

A “subject that has cancer” is a subject that has detectable cancerous cells. The cancer may be malignant or non-malignant. Cancers or tumors include, but are not limited to, biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. Cancers also include cancer of the blood and larynx.

A “subject at risk of having a cancer” is a subject who has a high probability of developing cancer. These subjects include, for instance, subjects having a genetic abnormality, the presence of which has been demonstrated to have a correlative relation to a higher likelihood of developing a cancer and subjects exposed to cancer causing agents such as tobacco, asbestos, or other chemical toxins, or a subject who has previously been treated for cancer and is in apparent remission.

The terms “treating” or “treatment” or “alleviation” or “amelioration” refer to both therapeutic treatment and prophylactic or preventative measures, wherein the object is to prevent or slow down (lessen) the targeted pathologic condition or disorder. A subject is successfully “treated” if, after receiving an effective amount of the active agents described herein, the subject shows observable and/or measurable reduction in or absence of one or more of the following: reduction in the number of cancer cells or absence of the cancer cells; reduction in the tumor size; inhibition (i.e., slow to some extent and preferably stop) of cancer proliferation; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition (i.e., slow to some extent and preferably stop) tumor growth; and/or relief to some extent, of one or more of the symptoms associated with the specific cancer; reduced morbidity and mortality, and improvement in quality of life issues.

The foregoing parameters for assessing successful treatment are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively.

A number of known methods can be used to assess the bulk size of a tumor. Non-limiting examples of such methods include imaging methods (e.g., computed tomography (CT), magnetic resonance imaging (MRI), ultrasound, X-ray imaging, mammography, PET scans, radionuclide scans, bone scans), visual methods (e.g., colonoscopy, bronchoscopy, endoscopy), physical examination (e.g., prostate examination, breast examination, lymph nodes examination, abdominal examination, rectal examination, general palpation), blood tests (e.g., prostate specific antigen (PSA) test, carcinoembryonic antigen (CEA) test, cancer antigen (CA)-125 test, alpha-fetoprotein (AFP), liver function tests), bone marrow analyses (e.g., in cases of hematological malignancies), histopathology, cytology, and flow cytometry.

The agents disclosed herein are delivered to subjects with cancer (i.e., a malignant tumor) or at risk for cancer. When the subject already has a malignancy, the development of stemness may have already occurred. Accordingly, the stemness reducing agents described herein, can be used to inhibit the production of new stem cells and/or prevent the maintenance of stemness. By administering the agents to subjects with cancer, the phenotypic alterations of tumors and tumor cells are reduced, preventing the progression of cancer.

In addition, one or more agents can be administered to a subject with a benign tumor. Benign tumors may present a precursor step in the development of malignancy, such as in colon cancer where polyps are believed to precede the development of malignant colorectal carcinomas. The administration of one or more of the stemness reducing agents to a subject with a benign tumor can prevent the development of stemness and concomitantly the development of malignancy.

In addition, one or more agents can be administered to a subject that has no known tumors. This can occur either after surgical and/or chemical removal of a tumor or where no diagnosis of a tumor has been made. In the case where a tumor has been removed, administration of one or more of the stemness reducing agents can prevent the maintenance and/or development of remnant cancer stem cells and prevent recurrence. By preventing stemness, the development of tumors and cancers can be prevented.

In one embodiment, an effective amount of one or more agents that modulate the expression or activity of one or more of the transcription factors described herein (for example, Oct4, Sox2, Klf2, Klf4, Klf5, Tbx3, Nanog, ESRRB, and REST) are administered to a subject with cancer and in need thereof thereby to ameliorate one or more symptoms of cancer.

Under certain circumstances, the practice of the methods described herein may result in at least a 5%, 10%, 15%, 20%, 30%, 40%, 50%, 60%, 70%, 80%, 90%, 95%, 98% or 99% reduction in the cancer stem cell population and/or at least a 5%, 10%, 15%, 20%, 30%, 40%, 50%, 60%, 70%, 80%, 90%, 95%, 98% or 99% reduction in the cancer cell population.

(2) Formulations

It is contemplated that one or more of the active ingredients (stemness reducing agents and/or anti-cancer agents) can be formulated for administration to a subject. The active ingredients can be formulation alone for sequential administration or may be formulation together for concurrent administration.

For example, a modulator of the expression or activity of one of the transcription factors described herein (for example, Oct4, Sox2, Klf2, Klf4, Klf5, Tbx3, Nanog, ESRRB, and REST) can be formulated with a pharmaceutically-acceptable carrier. Alternatively, a plurality of agents (for example, two, three, four or five agents) that directly reduce the expression or activity of one or more of the transcription factors described herein (for example, Oct4, Sox2, Klf2, Klf4, Klf5, Tbx3, Nanog, ESRRB, and REST) can be formulated with a pharmaceutically-acceptable carrier.

The term “pharmaceutically-acceptable carrier” as used herein means one or more compatible solid or liquid filler, diluents or encapsulating substances which are suitable for administration to a subject. The components of the pharmaceutical compositions also are capable of being commingled with each other, in a manner such that there is no interaction, which would substantially impair the desired pharmaceutical efficiency. Such preparations may routinely contain pharmaceutically acceptable concentrations of salt, buffering agents, preservatives, compatible carriers, adjuvants and optionally other therapeutic ingredients.

The compositions of the invention may be administered as a free base or as a pharmaceutically acceptable salt. Such pharmacologically and pharmaceutically acceptable salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene sulphonic, and benzene sulphonic. Also, pharmaceutically acceptable salts can be prepared as alkaline metal or alkaline earth salts, such as sodium, potassium or calcium salts of the carboxylic acid group.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Suitable liquid or solid pharmaceutical preparation forms are, for example, aqueous or saline solutions for inhalation, microencapsulated, encochleated, coated onto microscopic gold particles, contained in liposomes (including pH-dependent release formulations), lipidoids, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, SCIENCE 249:1527-1533, 1990 and Langer and Tirrell, NATURE, 2004 Apr. 1; 428(6982): 487-92.

The compositions may conveniently be presented in unit dosage form and may be prepared by any of the methods well known in the art of pharmacy. In certain embodiments, the composition that is administered is in powder or particulate form rather than as a solution. Examples of particulate forms contemplated as part of the invention are provided in U.S. Patent Application Publication No. US2002/0128225. In some embodiments, the compositions are administered in aerosol form. In other embodiments, the active compounds may be in powder form for constitution with a suitable vehicle, e.g., sterile pyrogen-free water, before use.

Additionally, in the case of more than one siRNA or in the case of a stemness reducing agent in combination with an anti-cancer agent, for example, a chemotherapeutic agent, the various agents can be combined covalently into a single agent. This entity must be formed such that the two agents retain function. In one embodiment, the first agent is a siRNA, which is bound to a second siRNA. In this embodiment, the two siRNAs preferentially are targeted to different genes. Alternatively, they can target different genetic sequences of a common gene. In one approach, two siRNAs are linked through their 3′ ends, using either a 3′ or 2′ site. The linking agent can be a phosphate, a cholesterol, a therapeutic agent, an ester linker, a triacylglycerol, PEG, PEI, or dextran. Alternatively, the siRNAs can be linked through a shared 5′ phosphate. Linkages can also be made by cleavable agents, such as esters. Upon internalization through the endosome pathway, increased acidity will split the ester leading to a siRNA-aldehyde and siRNA alcohol. The resulting composition can be delivered as is or in an agent including, but not limited to, liposomes (including pH-dependent release formulations) lipidoids, viruses PEI, PEG, PLGA, PEG-PLGA, poly(beta-amino esters), dextrans, β-glucan particles and other nanoparticle delivery agents known in the art.

In addition, the compositions described herein may be formulated as a depot preparation, time-release, delayed release or sustained release delivery system. Such systems can avoid repeated administrations of the compounds of the invention, increasing convenience to the subject and the physician. Such long acting formulations may be formulated with suitable polymeric or hydrophobic materials (for example as an emulsion in an acceptable oil) or ion exchange resins, or as sparingly soluble derivatives, for example, as a sparingly soluble salt. Many types of release delivery systems are available and known to those of ordinary skill in the art. They include polymer based systems such as polylactic and polyglycolic acid, beta-glucan particles, polyanhydrides and polycaprolactone; nonpolymer systems that are lipids including sterols such as cholesterol, cholesterol esters and fatty acids, neutral fats such as mono-, di- and triglycerides or lipidoids; hydrogel release systems; silastic systems; peptide based systems; wax coatings, compressed tablets using conventional binders and excipients, partially fused implants and the like. Specific examples include, but are not limited to: (a) erosional systems in which the agent is contained in a form within a matrix, found in U.S. Pat. Nos. 4,452,775; 4,667,014; and 4,748,034 and 5,239,660 and (b) diffusional systems in which an agent permeates at a controlled rate through a polymer, found in U.S. Pat. Nos. 3,832,253 and 3,854,480. In addition, a pump-based hardware delivery system can be used, some of which are adapted for implantation.

Controlled release can also be achieved with appropriate excipient materials that are biocompatible and biodegradable. These polymeric materials which effect slow release may be any suitable polymeric material for generating particles, including, but not limited to, nonbioerodable/non-biodegradable and bioerodable/biodegradable polymers. Such polymers have been described in great detail in the prior art and include, but are not limited to: β-glucan particles, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulfate sodium salt, poly(methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene poly(ethylene glycol), poly(ethylene oxide), poly(ethylene terephthalate), poly(vinyl alcohols), poly(vinyl acetate, poly vinyl chloride polystyrene, polyvinylpryrrolidone, hyaluronic acid, and chondroitin sulfate. In one embodiment the slow release polymer is a block copolymer, such as poly(ethylene glycol) (PEG)/poly(lactic-co-glycolic acid) (PLGA) block copolymer.

Examples of non-biodegradable polymers include ethylene vinyl acetate, poly(meth)acrylic acid, polyamides, copolymers and mixtures thereof.

Examples of biodegradable polymers include synthetic polymers, for example, beta-glucan particles, polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. The foregoing materials may be used alone, as physical mixtures (blends), or as co-polymers. Preferred polymers are polyesters, polyanhydrides, polystyrenes and blends thereof.

It is understood that the active agents can be administered in an encapsulation vehicle, such as, a liposome, cell, particle, nanoparticle, or any other vehicle capable of encapsuling the agent during delivery and then optionally releasing the active agents at a desired site. Furthermore, the compositions can further include a targeting molecule (see Pridgen et al., NANOMED 2:669-680, 2007; Brannon-Peppas and Blanchette, ADV. DRUG DELIV. REV. 56:1649-1659, 2004). The targeting molecule can be attached to the encapsulation vehicle, the active agent, and additional therapeutic agent, or some combination thereof. A targeting molecule is any molecule or compound which is specific for a particular cell or tissue and which can be used to direct the agents provided herein to a particular cell or tissue. The targeted molecules can be any molecule that is differentially present on a particular cell or in a particular tissue. These molecules can be proteins expressed on the cell surface.

For example, suitable delivery vehicles for siRNAs, protein/peptide agents and/or small molecules include nanoparticles such as extracted yeast cell walls composed of beta-glucans (see, U.S. Patent Application Publication Nos. US2005/0281781 and US2006/0083718) and other forms of polymeric, controlled-release nanoparticles (see U.S. Pat. No. 6,007,845, and U.S. Patent Application Publication Nos. US2005/0037075, US2008/0081074) that can be made to display highly-specific receptor-binding molecules on their exterior (e.g. antibodies, aptamers, etc.) for efficient uptake by targeted cells. Nanoparticles can be engineered to be phagocytosed by macrophages so that, upon the event of tumor necrosis and inflammation subsequent to chemotherapy and/or radiotherapy, nanoparticle containing macrophages can migrate to the inflamed tumor site for drug delivery

Effective amounts of the compositions of the invention are administered to a subject in need of such treatment. Effective amounts are those amounts, which will result in a desired improvement in the condition, disease or disorder or symptoms of the condition, disease or disorder.

Effective doses range from 1 ng/kg to 100 mg/kg body weight, or from 100 ng/kg to 50 mg/kg body weight, or from 1 μg/kg to 10 mg/kg body weight, depending upon the mode of administration. Alternatively, effective doses can range from 3 micrograms to 14 milligrams per 4 square centimeter area of cells. The absolute amount will depend upon a variety of factors (including whether the administration is in conjunction with other methods of treatment, the number of doses and individual patient parameters including age, physical condition, size and weight) and can be determined with routine experimentation. It is preferred, generally, that a maximum dose be used, that is, the highest safe dose according to sound medical judgment.

The time between the delivery of the various active agents can be defined rationally by first principles of the kinetics, delivery, release, agent pharmacodynamics, agent pharmacokinetics, or any combination thereof. Alternatively, the time between the delivery of the various agents can be defined empirically by experiments to define when a maximal effect can be achieved.

(3) Modes of Administration

The mode of administration may be any medically acceptable mode including oral administration, sublingual administration, intranasal administration, intratracheal administration, inhalation, ocular administration, topical administration, transdermal administration, intradermal administration, rectal administration, vaginal administration, subcutaneous administration, intravenous administration, intramuscular administration, intraperitoneal administration, intrasternal, administration, or via transmucosal administration.

The particular mode selected will depend upon the particular active agents selected, the desired results, the particular condition being treated and the dosage required for therapeutic efficacy. The methods of this invention, generally speaking, may be practiced using any mode of administration that is medically acceptable, meaning any mode that produces effective levels of inflammatory response alteration without causing clinically unacceptable adverse effects.

The compositions can be provided in different vessels, vehicles or formulations depending upon the disorder and mode of administration. For example, for oral application, the compounds can be administered as sublingual tablets, gums, mouth washes, toothpaste, candy, gels, films, etc.; for ocular application, as eye drops in eye droppers, eye ointments, eye gels, eye packs, as a coating on a contact lens or an intraocular lens, in contacts lens storage or cleansing solutions, etc.; for topical application, as lotions, ointments, gels, creams, sprays, tissues, swabs, wipes, etc.; for vaginal or rectal application, as an ointment, a tampon, a suppository, a mucoadhesive formulation, etc.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, such as, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

One suitable oral form is a sublingual tablet. A sublingual tablet delivers the composition to the sublingual mucosa. As used herein, “tablet” refers to pharmaceutical dosage forms prepared by compressing or molding. Sublingual tablets are small and flat, for placement under the tongue and designed for rapid, almost instantaneous disintegration and release the composition to the sublingual mucosa, for example, within five minutes.

Oral formulations can also be in liquid form. The liquid can be administered as a spray or drops to the entire oral cavity including select regions such as the sublingual area. The sprays and drops of the present invention can be administered by means of standard spray bottles or dropper bottles adapted for oral or sublingual administration. The liquid formulation is preferably held in a spray bottle, fine nebulizer, or aerosol mist container, for ease of administration to the oral cavity. Liquid formulations may be held in a dropper or spray bottle calibrated to deliver a predetermined amount of the composition to the oral cavity. Bottles with calibrated sprays or droppers are known in the art. Such formulations can also be used in nasal administration.

The compositions can also be formulated as oral gels. As an example, the composition may be administered in a mucosally adherent, non-water soluble gel. The gel is made from at least one water-insoluble alkyl cellulose or hydroxyalkyl cellulose, a volatile nonaqueous solvent, and the composition. Although a bioadhesive polymer may be added, it is not essential. Once the gel is contacted to a mucosal surface, it forms an adhesive film due primarily to the evaporation of the volatile or non-aqueous solvent. The ability of the gel to remain at a mucosal surface is related to its filmy consistency and the presence of non-soluble components. The gel can be applied to the mucosal surface by spraying, dipping, or direct application by finger or swab.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. Medical devices for the inhalation of therapeutics are known in the art. In some embodiments the medical device is an inhaler. In other embodiments the medical device is a metered dose inhaler, diskhaler, Turbuhaler, diskus or a spacer. In certain of these embodiments the inhaler is a Spinhaler (Rhone-Poulenc Rorer, West Malling, Kent). Other medical devices are known in the art and include the following technologies Inhale/Pfizer, Mannkind/Glaxo and Advanced Inhalation Research/Alkermes.

The compounds, when desirable to deliver them systemically, may be administered by injection, e.g., by bolus injection or continuous infusion, via intravenous, subcutaneous, intramuscular, intraperitoneal, intrasternal routes. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

The compounds may also be formulated in rectal or vaginal compositions such as suppositories or retention enemas, e.g., containing conventional suppository bases such as cocoa butter or other glycerides.

The compositions can be administered locally or the compositions can further include a targeting molecule (see Pridgen et al., NANOMED 2:669-680, 2007; Brannon-Peppas and Blanchette, ADV. DRUG DELIV. REV. 56:1649-1659, 2004). The targeting molecule can be attached to the agent and/or the additional therapeutic agent or some combination thereof. A targeting molecule is any molecule or compound which is specific for a particular cell or tissue and which can be used to direct the agents provided herein to a particular cell or tissue. The targeted molecules can be any molecule that is differentially present on a particular cell or in a particular tissue. These molecules can be proteins expressed on the cell surface.

For example, suitable delivery vehicles for siRNAs, protein/peptide agents and/or small molecules include nanoparticles such as extracted yeast cell walls composed of beta-glucans (see, U.S. Patent Application Publication Nos. US2005/0281781 and US2006/0083718) and other forms of polymeric, controlled-release nanoparticles (see U.S. Pat. No. 6,007,845, and U.S. Patent Application Publication Nos. US2005/0037075, US2008/0081074) that can be made to display highly-specific receptor-binding molecules on their exterior (e.g. antibodies, aptamers, etc.) for efficient uptake by targeted cells. Nanoparticles can be engineered to be phagocytosed by macrophages so that, upon the event of tumor necrosis and inflammation subsequent to chemotherapy and/or radiotherapy, nanoparticle containing macrophages can migrate to the inflamed tumor site for drug delivery.

It is understood that one issue that can result from generally inhibiting stemness in an organism is the possibility of reducing naturally occurring stem cells or stem-like cells, which have important homeostatic functions, such as wound healing. As a result, one or more agents that prevent or inhibit maintenance of stemness can be targeted to a particular cell or tissue, using any method known in the art. In a preferred embodiment, agents can be targeted based on the expression of tumor-specific markers. Particular tumors, such as malignant melanomas, express markers found in no other cell in the adult body (Hendrix et al., NAT. REV. CANCER 7:246-255, 2007; Postovit et al., EXPERT OPIN. THER. TARGETS 11:497-505, 2007). Similarly, AML cancer stem cells are known to express CD34 and CD44 (Lapidot et al., NATURE 367:645-648, 1994; Jin et al., NATURE MEDICINE 12:1167-1173, 2006). CD44 is also expressed in breast cancer stem cells (Al-Hajj et al., PROC. NATL. ACAD. SCI. USA 100:3983-3988, 2003). CD133 is expressed in colon cancer stem cells (Ricci-Vitiani et al., NATURE 445:111-115, 2007; O'Brien et al., NATURE 445:106-110, 2007) and brain tumorstem cells (Singh et al., NATURE 432:396-401, 2004). Numerous other examples of markers, especially surface markers, unique to and/or associated with specific cancer cells and/or cancer stem cells are well known in the art (see, for example, Ailles and Weissman, CURRENT OPINION IN BIOTECHNOLOGY 18:460-466, 2007). Using antibodies, aptamers, or other agents that specifically bind a tumor-specific marker, cells expressing the particular tumor-specific markers can be targeted for the delivery of agents. In an alternative approach, targeting can be achieved by local delivery, for example by intra- or circum-tumoral injection.

In another approach, cells can be targeted by the co-expression of tumor antigens and stem-like markers (i.e., markers including but not limited to Oct4, Sox2, Nanog, Stat3, E-ras, c-myc, Klf4, REST, ESRRB, β-catenin, SSEA-1, SSEA-3, SSEA-4, alkaline phosphatase, twist, snail, slug, E47, goosecoid, Foxc1, Foxc2, Sip1, N-cadherin, fibronectin, vimentin, CD34, CD44, CD96, CD133 and others known in the art). Tumor-antigens include Melan-A/MART-1, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100Pme1117, PRAME, NY-ESO-1, brain glycogen phosphorylase, SSX-1, SSX-2 (HOM-MEL-40), SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, Smad family of tumor antigens, lmp-1, EBV-encoded nuclear antigen union(EBNA)-1, and c-erbB-2. Targeting moieties can include, for example, antibodies, aptamers, and other binding moieties known in the art.

### Examples

The invention is further illustrated by the following examples. The examples are provided for illustrative purposes only, and are not to be construed as limiting the scope or content of the invention in any way.

### Example 1

### System for Confirming the Activity of Stemness-Reducing Agents

This example describes an assay for identifying and validating inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist for their ability to reduce stemness in human embryonic stem cells. Human embryonic stem cells express specific markers, including the cell surface markers SSEA-3 and SSEA-4 that correlate highly with their stemness, i.e., undifferentiated state (Draper et al., J. ANAT. 200:249-258, 2002). In vitro immunostaining assays can be used to measure the ability of cells to maintain stemness after treatment with inhibitors of Oct3/4, Sox2, Klf4, Nanog, Tbx3, ESRRB, REST, Snail, Twist, Slug, SIP1, FoxC1, FoxC2, goosecoid and TCF3.

Briefly, human embryonic stem cells, available from the National Stem Cell Bank (Madison, Wis.), are cultured in media and under conditions known in the art and are then exposed to the inhibitors under investigation. The resulting cells are trypsinized, fixed and immunostained using fluorescently-conjugated antibodies to SSEA-3 (ab16286, Abcam, Cambridge, Mass., USA) and SSEA-4 (ab16287, Abcam, Cambridge, Mass., USA). The levels of SSEA-3 and SSEA-4 are measured using flow cytometry, normalized to cell number, and compared to human embryonic stem cells not treated with the inhibitors (control cells). It is contemplated that agents which inhibit the maintenance of stemness (i.e., stemness reducing agents) will result in significantly lower levels of SSEA-3 and SSEA-4 compared to the control.

It is also contemplated that this assay system can be used to screen for and identify many types of inhibitors of stemness including but not limited to: siRNAs, shRNAs, antisense oligonucleotides, antibodies, adzymes, aptamers, proteins, and small molecules.

### Example 2

### Epithelial-Mesenchymal Transition (EMT) Model

This example describes an assay for identifying and validating inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist for their ability to reduce stemness in stem cells created by the induction of the epithelial-mesenchymal transition (EMT).

Cells that have undergone EMT have properties of stem cells including the ability to form mammospheres, tumors in immunocompromized mice and the expression of epithelial stem cell markers including N-cadherin and vimentin (Mani et al., CELL 133:704, 2008). The following in vitro immunostaining assay can be used to measure the ability of cells to undergo EMT, and/or maintain the EMT phenotype, after treatment with inhibitors of Oct3/4, Sox2, Klf4, Nanog, Tbx3, ESRRB, REST, Snail, Twist, Slug, SIP1, FoxC1, FoxC2, goosecoid and TCF3.

Briefly, cultured human mammary epithelial (HMLE) cells are exposed to EMT-inducing agents (e.g. TGF-β, see Mani et al. supra) in the presence of the inhibitors using treatment methods well known in the art and dependent on the physical properties of the inhibitors. The cells are then trypsinized, fixed and immunostained using fluorescently-conjugated antibodies to N-cadherin (ab12221, Abcam, Cambridge, UK) and vimentin (ab49918, Abcam, Cambridge, UK). The levels of N-cadherin and vimentin are measured using flow cytometry, normalized to cell number, and compared to cells treated with mock inhibitors. It is contemplated that agents which inhibit the induction of EMT will produce cells with significantly lower levels of N-cadherin and vimentin compared to control cells.

Alternatively, HMLE cells are first treated with EMT-inducing agents and are then treated with inhibitors and measured as above. It is contemplated that agents which inhibit the maintenance of the EMT phenotype will produce cells with significantly lower levels of N-cadherin and vimentin compared to control cells.

It is also contemplated that this assay system can be used to screen for and identify many types of inhibitors of stemness including but not limited to: siRNAs, shRNAs, antisense oligonucleotides, antibodies, adzymes, aptamers, proteins, and small molecules.

### Example 3

### BPLER Model

This example describes a method for reducing or eliminating cancer stem cells in vitro/ex-vivo using inhibitors of any one of, or a combination of, the transcription factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist.

In a mixed population of stem-like and differentiated cells, cancer initiating potential correlates with the number of cancer stem cells. A robust cell line for evaluating the efficacy of stemness reducing agents is the human breast tissue-derived BPLER cell, which possesses relatively high cancer-initiating potential (Tan et al., CANCER CELL 12:160, 2007). BPLER cells are treated with inhibitors of any one, or a combination of, the transcription factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist using treatment methods well known in the art and dependent on the physical properties of the inhibitors. Treated cells or non-treated control cells then are implanted sub-cutaneously into immunocompromised mice using methods known in the art (Tan et al., supra; McAllister et al., CELL 133:944, 2008). Tumor formation and tumor growth in these mice is monitored over a period of several weeks. It is contemplated that agents capable of reducing stemness will reduce the percentage of cancer stem cells and, as a result, lead to lower incidence of primary tumor formation, fewer metastases, and/or less aggressive tumor growth when compared to controls.

### Example 4

### Acute Myelogenous Leukemia Model

This example describes a method for reducing or eliminating cancer stem cell in an animal model of acute myelogenous leukemia (AML) using inhibitors of any or a combination of the factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist.

AML is the first type of cancer for which the role of cancer stem cells in contributing to tumorigenesis was described (Lapidot et al. (1994), supra). Mouse models of AML can be generated by orthotopic transplantation of stem-like cells from human AML patients into severe combined immunodeficient (SCID) mice. Bone marrow or peripheral blood from human patients are obtained from human volunteers. Fluorescence-activated cell sorting (FACS) is used to purify CD34⁺ CD38⁻ cells, which constitute AML stem-like cells, using CD34 and CD38 antibodies. Between 1×10⁵ and 1×10⁶ cells are injected into the tail veins of sublethally irradiated (400cGy using a ¹³⁷CS source) SCID mice. The mice then are treated with recombinant pro-leukemic cytokines PIXY321 (7 μg) and hMGF (10 μg) on alternating days by intraperitoneal injection. Upon 14 to 30 days of such treatment, mice are additionally treated with vehicle control or with inhibitors of any one of, or a combination of, the transcription factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist.

Leukemia colony forming units (AML-CFU) then are assayed using bone marrow cells from transplanted mice. 2×10⁵ bone marrow cells are plated in 0.9% methylcellulose in the presence of fetal bovine serum (15%), human plasma (15%), hMGF (50 ng/ml), PIXY321 (5 ng/ml), hGM-CSF (1 U/ml), hIL-3 (10 U/ml) and human erythropoietin (2 U/ml). After 7 days in culture, leukemic blast colonies are scored by cytology and chromosomal analysis. The formation of leukemic blast colonies reflects clonal expansion of tumorigenic stem-like cells in AML. It is contemplated that bone marrow cells exposed to stemness-reducing agents will produce statistically significant fewer leukemic blast colonies than vehicle treated controls. It is also contemplated that such result would demonstrate the efficacy of using stemness-reducing agents in the treatment of AML.

Alternatively, a delivery vehicle can be used that targets the stem cells of AML. For example, upon 14 to 30 days of treatment, mice are additionally treated with control or with inhibitors of any one of, or a combination of, the transcription factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 in a beta-glucan particle-based delivery vehicle with antibodies to CD44 conjugated to its surface (Jin et al. (2006), supra). It is contemplated that when the antibodies bind the CD44 receptor of AML stem cells, the vehicle is internalized into the cell and the inhibitors are released. It is contemplated that CD44⁺ AML stem cells exposed to stemness-reducing agents will produce statistically significant fewer leukemic blast colonies than vehicle treated controls. It is also contemplated that such result would demonstrate the efficacy of targeting stemness-reducing agents to cancer stem cells in the treatment of AML.

### Example 5

### Breast Cancer Model

This example describes a method for reducing or eliminating cancer stem cell in an animal model of breast cancer using inhibitors of any of, or a combination of, the factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist.

Mouse models of breast cancer can be generated by the orthotopic transplantation of primary or metastatic breast cancer cells from human breast cancer patients into no obese diabetic/severe combined immunodeficient (NOD/SCID) mice (Al-Hajj et al. (2003), supra). Primary or metastatic tumors specimens obtained from human volunteers then are minced with sterile blades and incubated with ultra-pure collagenase III in medium 199 (200 U/ml) at 37° C. for 3-4 hours. The mixture then is pipetted every 15-20 minutes and the cells are filtered through a 45 micron nylon mesh. The cells then are washed once with RPMI media with FBS (20%) and twice with HBSS. Cells then are sorted twice by FACS to identify breast cancer stem-like cells. In the first sorting, non-stem-like cells are excluded using antibodies against CD2, CD3, CD10, CD16, CD18, CD31, CD64, and CD140b, available from BD Bioscience Pharmingen (San Diego, Calif.). Cells not excluded by these antibodies are referred to as Lineage⁻ cells. The resulting lineage⁻ cells are subjected to a second round of FACS sorting using antibodies against CD44 and CD24 to obtain breast cancer stem-like cells which are CD44⁺CD24^{-/low}Lineage.

To generate the mouse model, eight-week-old female NOD-SCID mice are anesthetized with 0.2 ml of ketamine/xylazine and subsequently treated with etoposide via an intraperitoneal injection (30 mg/kg). Simultaneously, estrogen pellets are placed subcutaneously on the dorsal aspect of the mouse neck. Between 1×10⁴ and 1×10⁵ of the breast cancer stem-like cells are suspended in a 1:1 volumetric mixture of HBSS/Matrigel and injected into mammary fat pads of mice. Nexaban is used to seal the injection site. Mice then are maintained for three weeks before being treated with inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist, singly or in combination. The formation of tumors in mice is assessed nine weeks following injection by either gross palpation or by histopathological methods known to those in the art. It is contemplated that cells treated with stemness-reducing agents will produce statistically significant fewer and/or smaller tumors than vehicle treated controls. It is also contemplated that such result would demonstrate the efficacy of using stemness-reducing agents in the treatment of breast cancer.

Alternatively, mice are treated with inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist, alone or in combination, together with one or more additional chemotherapeutic agents, such as, taxol. The additional chemotherapeutic agent(s) can be physically conjugated to the delivery vehicle, placed inside the delivery vehicle with other agents, or administered separately. It is contemplated that cells treated with stemness-reducing agents in combination with the chemotherapeutic agent(s) will produce statistically significant fewer and/or smaller tumors than vehicle treated controls or with chemotherapeutics alone. It is also contemplated that such result would demonstrate the efficacy of using stemness-reducing agents in combination with chemotherapeutics in the treatment of breast cancer.

Alternatively, mice are treated with cancer stem-cell-targeting vehicles containing inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist, alone or in combination, with an additional chemotherapeutic agent, such as, taxol. The additional chemotherapeutic agent can be physically conjugated to the delivery vehicle, placed inside the delivery vehicle with other agents, or administered separately. The surface of the delivery vehicle is coated with antibodies to breast cancer stem cell markers, e.g. CD44. It is contemplated that once the antibodies bind the CD44 receptor of breast cancer stem cells, the vehicle is internalized and the agents are released inside the cell. It is contemplated that cancer stem cell-targeted treatment with stemness-reducing agents, in combination with the chemotherapeutic agent(s), will produce statistically significant fewer and/or smaller tumors than vehicle treated controls, with chemotherapeutics alone or without targeting. It is also contemplated that such result would demonstrate the efficacy of using targeted sternness-reducing agents in combination with the chemotherapeutic agent(s) in the treatment of breast cancer.

### Example 6

### Brain Cancer Model

This example describes a method for reducing or eliminating cancer stem cell in an animal model of brain cancer using inhibitors of any one of, or a combination of, the transcription factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist.

Mouse models of brain cancer can be generated by the orthotopic transplantation of primary glioblastoma or medulloblastoma cells from human brain cancer patients into NOD/SCID mice (Singh et al., NATURE 432:396-401, 2004). Primary glioblastoma or medulloblastoma tumor specimens obtained from human volunteers are immediately washed and dissociated in oxygenated artificial cerebrospinal fluid (CSF), subjected to enzymatic dissociation, and allowed to recover in TSM media as previously described (Singh et al., CANCER RES. 63:5821-5828, 2003). To isolate brain tumor stem-like cells (BTSCs), cells are labeled with anti-CD133 conjugated microbeads (1 μL CD133/1 microbeads per 1×10⁶ cells) using the Miltenyi Biotec CD133 cell isolation kit (Singh et al., 2003 supra). The samples then are periodically subjected to mechanical and chemical trituration. The purity of CD133⁺ cells, which represent putative BTSCs, can be assayed by flow cytometry with FACSCalibur. Within 16 hours of cell sorting, 5×10³ to 5×10⁴ CD133⁺ BTSCs are resuspended in 10 μL of phosphate buffered saline (PBS) and injected stereotactically into the frontal cortices of anesthetized six to eight-week old NOD-SCID mice. Injection coordinates are 3 mm to the right of midline, 2 mm anterior to the coronal suture, and 3 mm deep.

The mice then are maintained for four to ten weeks before being treated with inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist, either alone or in combination with one another. The formation of tumors is assessed at fourteen weeks following injection by histopathological methods known to those in the art. It is contemplated that cells treated with sternness-reducing agents will produce statistically significant fewer and/or smaller tumors than vehicle treated controls. It is also contemplated that such result would demonstrate the efficacy of using stemness-reducing agents in the treatment of brain cancer.

### Example 7

### Colon Cancer Model

This example describes a method for reducing or eliminating cancer stem cell in an animal model of colon cancer using inhibitors of any or a combination of the factors Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist.

Mouse models of colon cancer can be generated by the orthotopic transplantation of colon cancer cells from human colon cancer patients into SCID mice (Ricci-Vitiani et al., NATURE 445:111-115, 2007; O'Brien et al., NATURE 445:106-110, 2007). Primary colon cancer specimens obtained from human volunteers are immediately washed and subjected to mechanical and enzymatic dissociation. The resulting cells are cultured in serum-free media supplemented with 20 ng/ml EGF and 10 ng/ml FGF-2. Alternatively, cells can be directly separated to purify CD133⁺ colon cancer stem-like cells (CCSCs). This is accomplished 24 to 48 hours after dissociation by labeling tumor cells with CD133/1 microbeads and using magnetic separation with the Miltenyi Biotec CD133 cell isolation kit, available from Miltenyi Biotec (Bergisch Gladbach, Germany). Cells can also be separated by FACS using the CD133/1-phycoeruthrin antibody, available from Miltenyi Biotec using standard protocols known to those in the art.

Cell purity can be confirmed by FACS using CD133/2-phycoerythrin antibodies available from Miltenyi Biotec. The CD133⁺ putative CCSCs are injected subcutaneously into the flanks of 6- to 8-week old SCID mice. The mice then are maintained for 2 to 4 weeks before being treated with inhibitors of Oct4, Sox2, Klf4, Nanog, c-Myc, Klf5, Klf2, ESRRB, REST, TBX3, Foxc1, Foxc2, Goosecoid, Sip1, Snail1, Snail2, Tcf3 and Twist, either alone or in combination. The formation of tumors is assessed after a total of 8 to 10 weeks following injection by histopathological methods well known to those in the art. It is contemplated that cells treated with stemness-reducing agents show statistically significant fewer and/or smaller tumors than vehicle treated controls. It is also contemplated that such result would demonstrate the efficacy of using stemness-reducing agents in the treatment of colon cancer.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed:

1. A method of facilitating cell death of cancer stern cells or differentiated cells with a propensity for forming cancer stem cells in an initial mixed population of cancer stern cells and differentiated cells, the method comprising:
(a) inhibiting the formation of cancer stem cells from one or more differentiated cells in the initial mixed population, and optionally inhibiting the maintenance of the cancer stem cells in the initial mixed population, thereby to produce a second population of cells with fewer cancer stem cells or differentiated cells with a propensity for forming cancer stem cells than the initial mixed population; and
(b) inducing cell death of differentiated cells in the second population of cells.

2. The method of claim 1, wherein step (b) occurs after step (a).

3. The method of claim 1, wherein step (b) occurs contemporaneously with step (a).

4. The method of claim 1 wherein an agent to inhibit the formation of cancer stern cells or to inhibit the maintenance of the cancer stem cells directly reduces the expression or activity of a transcription factor.

5. The method of claim 4, wherein the transcription factor is selected from the group consisting of Oct4, Sox2, Klf2, Klf4, KlfS, Tbx3, Nanog, ESRRB and REST.

6. The method of claim 4, wherein the agent is an antibody, an anti-sense RNA, an siRNA or a small molecule.

7. The method of claim 1 wherein in step (b), an agent used to induce cell death of differentiated cells is an anti-cancer agent.

8. The method of claim 7, wherein the anti-cancer agent is a chemotherapeutic agent.

9. A method of reducing the number of cancer stern cells or differentiated cells with a propensity for forming cancer stem cells in a mixed population of cancer stern cells and differentiated cells, the method comprising:
exposing the mixed population of cancer stem cells and differentiated cells to at least two different agents that directly reduce the expression or activity of two transcription factors that (i) modulate the formation of cancer stern cells from one or more of the
differentiated cells or (ii) modulate maintenance of the cancer stern cells, thereby to reduce the number of cancer stern cells or differentiated stem cells with a propensity for forming cancer stem cells in the mixed population.

10. The method of claim 9, wherein the transcription factor that modulates the formation of cancer stem cells or modulates maintenance of cancer stem cells is selected from the group consisting of Oct4, Sox2, Klf2, Klf4, Klf5, Tbx3, Nanog, ESRRB and REST.

11. The method of e claim 9 or 10 wherein the agent is an antibody, an anti-sense RNA, an siRNA or a small molecule.

12. The method of claim 9 comprising exposing the cells to at least three agents.

13. A method of reducing the relative number of cancer stem cells or differentiated cells with a propensity for forming cancer stern cells in a mixed population of cancer stern cells and differentiated cells, the method comprising:
exposing the mixed population of cancer stem cells and differentiated cells with a combination of a first agent that inhibits the formation of cancer stem cells from one or more of the differentiated cells and a second agent that inhibits the maintenance of the cancer stern cells, thereby to reduce the relative number of cancer stem cells or differentiated cells with a propensity for forming cancer stem cells in the mixed population.

14. The method of claim 13, wherein the combination further comprises a third agent.

15. The method of claim 13, wherein the first agent and the second agent directly reduce the expression or activity of a transcription factor.

16. The method of claim 15, wherein the transcription factor is selected from the group consisting of Oct4, Sox2, Klf2, Klf4, Klf5, Tbx3, Nanog, ESRRI3 and REST.

17. The method of claim 15, wherein the first agent, the second agent, or both the first agent and the second agent is an antibody, an anti-sense RNA, an siRNA or a small molecule.

18. The method of claim 13, wherein the third agent directly reduces the expression or activity of a transcription factor.

19. The method of claim 18, wherein the third agent is an antibody, an anti-sense RNA, an siRNA or a small molecule.

20. A method of treating cancer in a mammal, the method comprising:
administering to the mammal in need thereof an effective amount of at least two agents that inhibit the formation of cancer stern cells from differentiated cells or inhibit the maintenance of cancer stern cells, thereby to treat the cancer in the mammal.

21. The method of claim 20, wherein the agents that inhibit the formation of
cancer stem cells or inhibit the maintenance of cancer stem cells directly reduce the expression or activity of a transcription factor.

22. The method of claim 21, wherein the transcription factor is selected from the group consisting of Oct4, Sox2, Klf2, Klf4, Klf5, Tbx3, Nanog, ESRRI3 and REST.

23. The method of claim 22, wherein the agent is an antibody, an anti-sense RNA, an siRNA or a small molecule.

24. The method of any one of claims 20 23 claim 20 comprising administering to the mammal at least two agents that inhibit the formation of cancer stem cells.

25. The method of any one of claims 20 23 claim 20 comprising administering to the mammal at least two agents that inhibit the maintenance of cancer stem cells.

26. The method of any one of claims 20 23 claim 20 comprising administering a combination of an agent that inhibits the formation of cancer stern cells and an agent that inhibits the maintenance of cancer stern cells.

27. A method of treating cancer in a mammal, the method comprising:
administering to the mammal an effective amount of one or more agents that directly reduce the expression or activity of Oct4, Sox2, Klf2, Klf4, Klf5, Tbx3, Nanog, ESRRB, or REST, thereby to ameliorate one or more symptoms of the cancer.

28. The method of claim 27, wherein the agent is selected from the group consisting of an antibody, an anti-sense RNA, an siRNA or a small molecule.

29. A composition comprising (a) a plurality of agents that directly reduce the expression or activity of Oct4, Sox2, Klf2, Klf4, KlfS, Tbx3, Nanog, ESRRB, or REST; and (b) a pharmaceutically-acceptable carrier.

30. The composition of claim 29, wherein the agents are selected from the group consisting of an antibody, an anti-sense RNA, an siRNA or a small molecule.

31. A method of treating cancer in a mammal, the method comprising administering to the mammal an effective amount of one or more agents that directly reduce the expression or activity of Oct4, Sox2, I(1f4, Nanog, c-Myc, Klf5, Klf2, ESRRB, REST, TBX3, Foxc1, Foxc2, Goosecoid, Sipi, Snail1, Snail2, Tcf3 and Twist disposed within an encapsulation vehicle.

32. The method of claim 31, wherein the encapsulation vehicle is conjugated to a targeting agent.

33. The method of claim 32, wherein the targeting agent is an antibody that binds a cell surface molecule found on cancer cells or cancer stem cells.

34. The method of claim 32, wherein the targeting agent is a ligand of a cell surface molecule found on cancer cells or cancer stem cells.

35. The method of claim 32, wherein the targeting agent is an aptamer to a cell surface molecule found on cancer cells or cancer stem cells.

36. The method of claim 31, wherein the agent is selected from the group consisting of an antibody, an anti-sense RNA, an siRNA or a small molecule.

37. A composition comprising (a) a plurality of agents that directly reduce the expression or activity of Oct4, Sox2, Klf4, Nanog, c-Myc, Klf5, Klf2, ESRRB, REST, TBX3, Foxci, Foxc2, (loosecoid, Sipi, Snail1, Snail2, Tc13 and Twist; and (b) a pharmaceutically-acceptable delivery vehicle; wherein the delivery vehicle contains one or more targeting moieties that bind a surface molecule on a cancer cell or cancer stem cell.

38. The composition of claim 37, wherein the agents are selected from the group consisting of an antibody, an anti-sense RNA, an siRNA or a small molecule.

39. The composition of claim 37, wherein the targeting moiety is an antibody, an aptamer or a ligand to a cell surface molecule present on cancer cells or cancer stem cells.

40. A method of identifying an inhibitor of cancer cell stemness, comprising the steps of:
(a) contacting a mouse containing a population of human cancer cells comprising (i) cancer stem cells and (ii) differentiated cancer cells with a stemness-inhibiting agent; and
(b) determining a reduction in the number of cancer stem cells or an increase in the number of differentiated cancer cells in the mouse,
thereby identifying an inhibitor of cancer cell stemness.

41. The method of claim 40, wherein the mouse is contacted with at least two different agents that directly reduce the expression or activity of two transcription factors selected from the group consisting of Oct4, Sox2, Klf2, Klf4, Klf5, Tbx3, Nanog, ESRRB and REST.

42. The method of claim 40, wherein the cancer cells are metastatic breast cancer cells.

43. The method of claim 40, wherein the agent inhibits the transition of differentiated cells into cancer stem cells.

44. The method of claim 40, wherein the agent inhibits cancer stem cell maintenance.

45. A method of identifying an inhibitor of tumor metastasis, comprising:
(a) administering to a mouse comprising an orthotopic transplantation of human cancer cells an effective amount of an agent that reduces stemness in a cancer cell; and
(b) measuring the formation of metastatic tumors in the mouse,
wherein a reduction in the formation of metastatic tumors in the mouse in the presence of the agent as compared to the absence of the agent identifies the agent as an inhibitor of tumor metastasis.

46. The method of claim 45, further comprising the steps of allowing the human cancer cells to form a primary tumor in the mouse, and removing the primary tumor from the mouse prior to or after the administration of the agent.

47. The method of claim 45, wherein the formation of metastatic tumors in the bone, lung and/or brain is measured.

48. A method of reducing the number of cancer stem cells in a mixed population of cancer stem cells and differentiated cells, comprising:
(a) contacting the mixed population with a first agent that inhibits the formation of cancer stem cells from one or more of the differentiated cells;
(b) contacting the mixed population with a second agent that inhibits the maintenance of the cancer stem cells,
thereby reducing the number of cancer stem cells in the population.

49. The method of claim 48, wherein the first agent and the second agent are contacted with the mixed population simultaneously.

50. The method of claim 48, wherein the first agent and the second agent directly reduce the expression or activity of a transcription factor selected from the group consisting of Oct4, Sox2, Klf2, Klf4, Klf5, Tbx3, Nanog, ESRRB and REST.

51. The method of claim 48, further comprising the step of contacting the mixed population with a third agent comprising an anti-cancer agent.

52. The method of claim 48, further comprising the step of contacting the mixed population with a third agent that promotes cancer stem cell differentiation.

53. The method of claim 48, wherein the mixed population is present in a tumor.

54. The method of claim 48, wherein the mixed population is present in vitro.

55. A method of reducing cell number in a population of cells comprising differentiated cancer cells and undifferentiated cells, the method comprising:
(a) inducing cell death of cancer cells in the population of cells using an anti-cancer agent; and
(b) reducing stemness of undifferentiated cells in the population,
thereby reducing cell number in the population.

56. The method of claim 55, wherein step (b) is performed after step (a).

57. The method of claim 55, further comprising the step of inducing cell death in the cells having decreased stemness in step (b).

58. A method of reducing cell number in a population of cells comprising differentiated cancer cells and undifferentiated cells, the method comprising:
(a) inducing cell death of one or more cancer cells in the population of cells using an anti-cancer agent;
(b) decreasing stemness of one or more undifferentiated cells in the population, thereby inhibiting the formation of cancer stem cells from one or more differentiated cells; and
(c) inducing differentiation of one or more cancer stem cells in the population to form a cancer cell or a mesenchymal cell,
thereby reducing cell number in the population.

59. The method of claim 58, further comprising the step of:
(d) inducing cell death in the cells having reduced stemness in step (b).

60. The method of claim 58, further comprising the step of:
(d) inducing cell death in the cancer cells in step (c).


SEQUENCE LISTING (1126 sequences)

SEQ ID NO: 1 - DNA, 1411 bp, Homo sapiens
ccttcgcaagccctcatttcaccaggcccccggcttggggcgccttccttccccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 2 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - DNA, 1155 bp, Homo sapiens
catgagtcagtgaacagggaatgggtgaatgacatttgtgggtaggttatttctagaagttaggtgggcagcttggaaggcagatgcacttctacagactattccttggggccacacgtaggttcttgaatcccgaatggaaaggggagattgataactggtgtgtttatgttcttacaagtcttctgccttttaaaatccagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaactgaggtgcctgcccttctaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacacttaaaaaaaaaaa

SEQ ID NO: 4 - protein, 265 aa, Homo sapiens
MHFYRLFLGATRRFLNPEWKGEIDNWCVYVLTSLLPFKIQSQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 5 - DNA, 2518 bp, Homo sapiens
ctattaacttgttcaaaaaagtatcaggagttgtcaaggcagagaagagagtgtttgcaaaagggggaaagtagtttgctgcctctttaagactaggactgagagaaagaagaggagagagaaagaaagggagagaagtttgagccccaggcttaagcctttccaaaaaataataataacaatcatcggcggcggcaggatcggccagaggaggagggaagcgctttttttgatcctgattccagtttgcctctctctttttttcccccaaattattcttcgcctgattttcctcgcggagccctgcgctcccgacacccccgcccgcctcccctcctcctctccccccgcccgcgggccccccaaagtcccggccgggccgagggtcggcggccgccggcgggccgggcccgcgcacagcgcccgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatgtgagggccggacagcgaactggaggggggagaaattttcaaagaaaaacgagggaaatgggaggggtgcaaaagaggagagtaagaaacagcatggagaaaacccggtacgctcaaaaagaaaaaggaaaaaaaaaaatcccatcacccacagcaaatgacagctgcaaaagagaacaccaatcccatccacactcacgcaaaaaccgcgatgccgacaagaaaacttttatgagagagatcctggacttctttttgggggactatttttgtacagagaaaacctggggagggtggggagggcgggggaatggaccttgtatagatctggaggaaagaaagctacgaaaaactttttaaaagttctagtggtacggtaggagctttgcaggaagtttgcaaaagtctttaccaataatatttagagctagtctccaagcgacgaaaaaaatgttttaatatttgcaagcaacttttgtacagtatttatcgagataaacatggcaatcaaaatgtccattgtttataagctgagaatttgccaatatttttcaaggagaggcttcttgctgaattttgattctgcagctgaaatttaggacagttgcaaacgtgaaaagaagaaaattattcaaatttggacattttaattgtttaaaaattgtacaaaaggaaaaaattagaataagtactggcgaaccatctctgtggtcttgtttaaaaagggcaaaagttttagactgtactaaattttataacttactgttaaaagcaaaaatggccatgcaggttgacaccgttggtaatttataatagcttttgttcgatcccaactttccattttgttcagataaaaaaaaccatgaaattactgtgtttgaaatattttcttatggtttgtaatatttctgtaaatttattgtgatattttaaggttttcccccctttattttccgtagttgtattttaaaagattcggctctgtattatttgaatcagtctgccgagaatccatgtatatatttgaactaatatcatccttataacaggtacattttcaacttaagtttttactccattatgcacagtttgagataaataaatttttgaaatatggacactgaaaaaaaaaaaaaaaaaa

SEQ ID NO: 6 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 7 - DNA, 2639 bp, Homo sapiens
tcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaacctataatattttatctg

SEQ ID NO: 8 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 9 - DNA, 2098 bp, Homo sapiens
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 10 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 11 - DNA, 2377 bp, Homo sapiens
acccccgagctgtgctgctcgcggccgccaccgccgggccccggccgtccctggctcccctcctgcctcgagaagggcagggcttctcagaggcttggcgggaaaaagaacggagggagggatcgcgctgagtataaaagccggttttcggggctttatctaactcgctgtagtaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgacgatgcccctcaacgttagcttcaccaacaggaactatgacctcgactacgactcggtgcagccgtatttctactgcgacgaggaggagaacttctaccagcagcagcagcagagcgagctgcagcccccggcgcccagcgaggatatctggaagaaattcgagctgctgcccaccccgcccctgtcccctagccgccgctccgggctctgctcgccctcctacgttgcggtcacacccttctcccttcggggagacaacgacggcggtggcgggagcttctccacggccgaccagctggagatggtgaccgagctgctgggaggagacatggtgaaccagagtttcatctgcgacccggacgacgagaccttcatcaaaaacatcatcatccaggactgtatgtggagcggcttctcggccgccgccaagctcgtctcagagaagctggcctcctaccaggctgcgcgcaaagacagcggcagcccgaaccccgcccgcggccacagcgtctgctccacctccagcttgtacctgcaggatctgagcgccgccgcctcagagtgcatcgacccctcggtggtcttcccctaccctctcaacgacagcagctcgcccaagtcctgcgcctcgcaagactccagcgccttctctccgtcctcggattctctgctctcctcgacggagtcctccccgcagggcagccccgagcccctggtgctccatgaggagacaccgcccaccaccagcagcgactctgaggaggaacaagaagatgaggaagaaatcgatgttgtttctgtggaaaagaggcaggctcctggcaaaaggtcagagtctggatcaccttctgctggaggccacagcaaacctcctcacagcccactggtcctcaagaggtgccacgtctccacacatcagcacaactacgcagcgcctccctccactcggaaggactatcctgctgccaagagggtcaagttggacagtgtcagagtcctgagacagatcagcaacaaccgaaaatgcaccagccccaggtcctcggacaccgaggagaatgtcaagaggcgaacacacaacgtcttggagcgccagaggaggaacgagctaaaacggagcttttttgccctgcgtgaccagatcccggagttggaaaacaatgaaaaggcccccaaggtagttatccttaaaaaagccacagcatacatcctgtccgtccaagcagaggagcaaaagctcatttctgaagaggacttgttgcggaaacgacgagaacagttgaaacacaaacttgaacagctacggaactcttgtgcgtaaggaaaagtaaggaaaacgattccttctaacagaaatgtcctgagcaatcacctatgaacttgtttcaaatgcatgatcaaatgcaacctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcctagtatatagtacctagtattataggtactataaaccctaattttttttatttaagtacattttgctttttaaagttgatttttttctattgtttttagaaaaaataaaataactggcaaatatatcattgagccaaaaaaaaaaaaaaaaaaaaa

SEQ ID NO: 12 - protein, 454 aa, Homo sapiens
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 13 - DNA, 3350 bp, Homo sapiens
tagtcgcggggcaggtacgtgcgctcgcggttctctcgcggaggtcggcggtggcgggagcgggctccggagagcctgagagcacggtggggcggggcgggagaaagtggccgcccggaggacgttggcgtttacgtgtggaagagcggaagagttttgcttttcgtgcgcgccttcgaaaactgcctgccgctgtctgaggagtccacccgaaacctcccctcctccgccggcagccccgcgctgagctcgccgacccaagccagcgtgggcgaggtgggaagtgcgcccgacccgcgcctggagctgcgcccccgagtgcccatggctacaagggtgctgagcatgagcgcccgcctgggacccgtgccccagccgccggcgccgcaggacgagccggtgttcgcgcagctcaagccggtgctgggcgccgcgaatccggcccgcgacgcggcgctcttccccggcgaggagctgaagcacgcgcaccaccgcccgcaggcgcagcccgcgcccgcgcaggccccgcagccggcccagccgcccgccaccggcccgcggctgcctccagaggacctggtccagacaagatgtgaaatggagaagtatctgacacctcagcttcctccagttcctataattccagagcataaaaagtatagacgagacagtgcctcagtcgtagaccagttcttcactgacactgaagggttaccttacagtatcaacatgaacgtcttcctccctgacatcactcacctgagaactggcctctacaaatcccagagaccgtgcgtaacacacatcaagacagaacctgttgccattttcagccaccagagtgaaacgactgcccctcctccggccccgacccaggccctccctgagttcaccagtatattcagctcacaccagaccgcagctccagaggtgaacaatattttcatcaaacaagaacttcctacaccagatcttcatctttctgtccctacccagcagggccacctgtaccagctactgaatacaccggatctagatatgcccagttctacaaatcagacagcagcaatggacactcttaatgtttctatgtcagctgccatggcaggccttaacacacacacctctgctgttccgcagactgcagtgaaacaattccagggcatgcccccttgcacatacacaatgccaagtcagtttcttccacaacaggccacttactttcccccgtcaccaccaagctcagagcctggaagtccagatagacaagcagagatgctccagaatttaaccccacctccatcctatgctgctacaattgcttctaaactggcaattcacaatccaaatttacccaccaccctgccagttaactcacaaaacatccaacctgtcagatacaatagaaggagtaaccccgatttggagaaacgacgcatccactactgcgattaccctggttgcacaaaagtttataccaagtcttctcatttaaaagctcacctgaggactcacactggtgaaaagccatacaagtgtacctgggaaggctgcgactggaggttcgcgcgatcggatgagctgacccgccactaccggaagcacacaggcgccaagcccttccagtgcggggtgtgcaaccgcagcttctcgcgctctgaccacctggccctgcatatgaagaggcaccagaactgagcactgcccgtgtgacccgttccaggtcccctgggctccctcaaatgacagacctaactattcctgtgtaaaaacaacaaaaacaaacaaaagcaagaaaaccacaactaaaactggaaatgtatattttgtatatttgagaaaacagggaatacattgtattaataccaaagtgtttggtcattttaagaatctggaatgcttgctgtaatgtatatggctttactcaagcagatctcatctcatgacaggcagccacgtctcaacatgggtaaggggtgggggtggaggggagtgtgtgcagcgtttttacctaggcaccatcatttaatgtgacagtgttcagtaaacaaatcagttggcaggcaccagaagaagaatggattgtatgtcaagattttacttggcattgagtagtttttttcaatagtaggtaattccttagagatacagtatacctggcaattcacaaatagccattgaacaaatgtgtgggtttttaaaaattatatacatatatgagttgcctatatttgctattcaaaattttgtaaatatgcaaatcagctttataggtttattacaagttttttaggattcttttggggaagagtcataattcttttgaaaataaccatgaatacacttacagttaggatttgtggtaaggtacctctcaacattaccaaaatcatttctttagagggaaggaataatcattcaaatgaactttaaaaaagcaaatttcatgcactgattaaaataggattattttaaatacaaaaggcattttatatgaattataaactgaagagcttaaagatagttacaaaatacaaaagttcaacctcttacaataagctaaacgcaatgtcatttttaaaaagaaggacttagggtgtcgttttcacatatgacaatgttgcatttatgatgcagtttcaagtaccaaaacgttgaattgatgatgcagttttcatatatcgagatgttcgctcgtgcagtactgttggttaaatgacaatttatgtggattttgcatgtaatacacagtgagacacagtaattttatctaaattacagtgcagtttagttaatctattaatactgactcagtgtctgcctttaaatataaatgatatgttgaaaacttaaggaagcaaatgctacatatatgcaatataaaatagtaatgtgatgctgatgctgttaaccaaagggcagaataaataagcaaaatgccaaaaggggtcttaattgaaatgaaaatttaattttgtttttaaaatattgtttatctttatttattttgtggtaatatagtaagtttttttagaagacaattttcataacttgataaattatagttttgtttgttagaaaagttgctcttaaaagatgtaaatagatgacaaacgatgtaaataattttgtaagaggcttcaaaatgtttatacgtggaaacacacctacatgaaaagcagaaatcggttgctgttttgcttctttttccctcttatttttgtattgtggtcatttcctatgcaaataatggagcaaacagctgtatagttgtagaattttttgagagaatgagatgtttatatattaacgacaattttttttttggaaaataaaaagtgcctaaaaga

SEQ ID NO: 14 - protein, 457 aa, Homo sapiens
MATRVLSMSARLGPVPQPPAPQDEPVFAQLKPVLGAANPARDAALFPGEELKHAHHRPQAQPAPAQAPQPAQPPATGPRLPPEDLVQTRCEMEKYLTPQLPPVPIIPEHKKYRRDSASVVDQFFTDTEGLPYSINMNVFLPDITHLRTGLYKSQRPCVTHIKTEPVAIFSHQSETTAPPPAPTQALPEFTSIFSSHQTAAPEVNNIFIKQELPTPDLHLSVPTQQGHLYQLLNTPDLDMPSSTNQTAAMDTLNVSMSAAMAGLNTHTSAVPQTAVKQFQGMPPCTYTMPSQFLPQQATYFPPSPPSSEPGSPDRQAEMLQNLTPPPSYAATIASKLAIHNPNLPTTLPVNSQNIQPVRYNRRSNPDLEKRRIHYCDYPGCTKVYTKSSHLKAHLRTHTGEKPYKCTWEGCDWRFARSDELTRHYRKHTGAKPFQCGVCNRSFSRSDHLALHMKRHQN

SEQ ID NO: 15 - DNA, 1655 bp, Homo sapiens
gcccgcccgcgccccgaccagcccggcctcgggcagccactcaccggtgtccccgtccgcgtccttcctccccgggtcccggccatggcgctgagtgaacccatcctgccgtccttctccactttcgccagcccgtgccgcgagcgcggcctgcaggagcgctggccgcgcgccgaacccgagtccggcggcaccgacgacgacctcaacagcgtgctggacttcatcctgtccatggggctggatggcctgggcgccgaggccgccccggagccgccgccgccgcccccgccgcctgcgttctattaccccgaacccggcgcgcccccgccctacagcgcccccgcgggtggcctggtgtctgagctgctgcgacccgagctggatgcgccgctggggcccgcactgcacggccgctttctgctggcgccgcccggccgcctggtcaaggccgagccccctgaagcggacggcggcggcggctacggctgcgcccccgggctgacccgtggaccgcgcggcctcaagcgcgagggcgccccgggcccggcggcttcgtgcatgcgaggtcccgggggccgccccccgccgccgcccgacacaccgccgctcagccccgacggccccgcgcgcctgcccgcgcccggtccgcgcgcctccttcccgccgcctttcggtggccctggtttcggcgcgcccgggcccggcctgcattacgcgccgcctgcgcccccagccttcggtctcttcgacgacgcggccgccgccgcggcagccctgggcctggcgccccccgccgcccgcggtctcctcacgccgcctgcgtccccgctggagctgctggaggccaagccaaagcgcggccgccgctcttggccccgcaaacgcaccgccactcacacctgcagctacgcgggctgcggcaagacctacaccaagagttcgcatctgaaggcgcatctgcgcacgcacacaggtgagaagccctaccactgcaactgggacggctgcggctggaagtttgcgcgctcagacgagctcacgcgccactaccgaaagcacacgggccaccggccattccagtgccatctgtgcgatcgtgccttctcgcgctccgatcacctggcgctgcacatgaaacggcacatgtagccgggacgcccccgcccacctgcgcgcggccgtggcgggtcccacgcgccgggcgcggccccctcccaaactgtgactggtatttattggacccagagaaccgggccgggcacagcgtggctacagagggtctccctcgatgacgacgacgacgacgccaccaccccagcccccgtctgtgactgaaggcccggtgggaaaagaccacgatcctccttgacgagttttgtttttcaaaatggtgcaataatttaagtggcatcttctctcccaccgggtctacactagaggatcgaggcttgtgatgccttgtgagaaataagggccttaatttgtactgtctgcggcattttttataatattgtatatagtgactgacaaatattgtattactgtacatagagagacaggtgggcatttttgggctacctggttcgtttttataagattttgctgggttggttttttttttaattaaaaagttttgcatcttttaaaaaaaa

SEQ ID NO: 16 - protein, 355 aa, Homo sapiens
MALSEPILPSFSTFASPCRERGLQERWPRAEPESGGTDDDLNSVLDFILSMGLDGLGAEAAPEPPPPPPPPAFYYPEPGAPPPYSAPAGGLVSELLRPELDAPLGPALHGRFLLAPPGRLVKAEPPEADGGGGYGCAPGLTRGPRGLKREGAPGPAASCMRGPGGRPPPPPDTPPLSPDGPARLPAPGPRASFPPPFGGPGFGAPGPGLHYAPPAPPAFGLFDDAAAAAAALGLAPPAARGLLTPPASPLELLEAKPKRGRRSWPRKRTATHTCSYAGCGKTYTKSSHLKAHLRTHTGEKPYHCNWDGCGWKFARSDELTRHYRKHTGHRPFQCHLCDRAFSRSDHLALHMKRHM

SEQ ID NO: 17 - DNA, 2193 bp, Homo sapiens
cgggagcccatggagcactgtcctcagagatgcgcaggttaggctcactgtctaggccaggcccaccttagtcactgtggactggcaatggaagctcttcctggacacacctgccctagccctcaccctggggtggaagagaaatgagcttggcttgcaactcagaccattccacggaggcatcctcccttccctgggctggtgaataaaagtttcctgaggtcaaggacttccttttccctgccaaaatggtgtccagaactttgaggccagaggtgatccagtgatttgggagctgcaggtcacacaggctgctcagagggctgctgaacaggatgtcctcggacgacaggcacctgggctccagctgcggctccttcatcaagactgagccgtccagcccgtcctcgggcattgatgccctcagccaccacagccccagtggctcgtccgacgccagcggcggctttggcctggccctgggcacccacgccaacggtctggactcgccacccatgtttgcaggcgccgggctgggaggcaccccatgccgcaagagctacgaggactgtgccagcggcatcatggaggactcggccatcaagtgcgagtacatgctcaacgccatccccaagcgcctgtgcctcgtgtgcggggacattgcctctggctaccactacggcgtggcctcctgcgaggcttgcaaggccttcttcaagaggactatccaagggaacattgagtacagctgcccggccaccaacgagtgcgagatcaccaaacggaggcgcaagtcctgccaggcctgccgcttcatgaaatgcctcaaagtggggatgctgaaggaaggtgtgcgccttgatcgagtgcgtggaggccgtcagaaatacaagcgacggctggactcagagagcagcccatacctgagcttacaaatttctccacctgctaaaaagccattgaccaagattgtctcatacctactggtggctgagccggacaagctctatgccatgcctccccctggtatgcctgagggggacatcaaggccctgaccactctctgtgacctggcagaccgagagcttgtggtcatcattggctgggccaagcacatcccaggcttctcaagcctctccctgggggaccagatgagcctgctgcagagtgcctggatggaaatcctcatcctgggcatcgtgtaccgctcgctgccctatgacgacaagctggtgtacgctgaggactacatcatggatgaggagcactcccgcctcgcggggctgctggagctctaccgggccatcctgcagctggtacgcaggtacaagaagctcaaggtggagaaggaggagtttgtgacgctcaaggccctggccctcgccaactccgattccatgtacatcgaggatctagaggctgtccagaagctgcaggacctgctgcacgaggcactgcaggactacgagctgagccagcgccatgaggagccctggaggacgggcaagctgctgctgacactgccgctgctgcggcagacggccgccaaggccgtgcagcacttctatagcgtcaaactgcagggcaaagtgcccatgcacaaactcttcctggagatgctggaggccaaggcctgggccagggctgactcccttcaggagtggaggccactggagcaagtgccctctcccctccaccgagccaccaagaggcagcatgtgcatttcctaactcccttgccccctcccccatctgtggcctgggtgggcactgctcaggctggataccacctggaggttttccttccgcagagggcaggttggccaagagcagcttagaggatctcccaaggatgaaagaatgtcaagccatgatggaaaatgccccttccaatcagctgccttcacaagcagggatcagagcaactccccggggatccccaatccacgcccttctagtccaacccccctcaatgagagaggcaggcagatctcacccagcactaggacaccaggaggccagggaaagcatctctggctcaccatgtaacatctggcttggagcaagtgggtgttctgcacaccaggcagctgcacctcactggatctagtgttgctgcgagtgacctcacttcagagcccctctagcagagtggggcggaagtcctgatggttggtgtccatgaggtggaag

SEQ ID NO: 18 - protein, 500 aa, Homo sapiens
MSSDDRHLGSSCGSFIKTEPSSPSSGIDALSHHSPSGSSDASGGFGLALGTHANGLDSPPMFAGAGLGGTPCRKSYEDCASGIMEDSAIKCEYMLNAIPKRLCLVCGDIASGYHYGVASCEACKAFFKRTIQGNIEYSCPATNECEITKRRRKSCQACRFMKCLKVGMLKEGVRLDRVRGGRQKYKRRLDSESSPYLSLQISPPAKKPLTKIVSYLLVAEPDKLYAMPPPGMPEGDIKALTTLCDLADRELVVIIGWAKHIPGFSSLSLGDQMSLLQSAWMEILILGIVYRSLPYDDKLVYAEDYIMDEEHSRLAGLLELYRAILQLVRRYKKLKVEKEEFVTLKALALANSDSMYIEDLEAVQKLQDLLHEALQDYELSQRHEEPWRTGKLLLTLPLLRQTAAKAVQHFYSVKLQGKVPMHKLFLEMLEAKAWARADSLQEWRPLEQVPSPLHRATKRQHVHFLTPLPPPPSVAWVGTAQAGYHLEVFLPQRAGWPRAA

SEQ ID NO: 19 - DNA, 3663 bp, Homo sapiens
ggcggcggcggcggcgcggactgggtgcgcggcgcagcgtcctgtgttggaatgtgcggctgccgcgagctcgcggcgcagcagcggagcgagcgccgccgaggcccggggccccagaccctggcggcggctgcggcagccgagacggcagggcgaggcccggaggcctgagcaccctctgcagccccactcctgggccttcttggtccacgacggccccagcacccaactttaccaccctcccccacctctcccccgaaactccagcaacaaagaaaagtagtcggagaaggagcggcgactcagggtcgcccgcccctcctcaccgaggaaggccgaatacagttatggccacccaggtaatggggcagtcttctggaggaggagggctgtttaccagcagtggcaacattggaatggccctgcctaacgacatgtatgacttgcatgacctttccaaagctgaactggccgcacctcagcttattatgctggcaaatgtggccttaactggggaagtaaatggcagctgctgtgattacctggtcggtgaagaaagacagatggcagaactgatgccggttggggataacaacttttcagatagtgaagaaggagaaggacttgaagagtctgctgatataaaaggtgaacctcatggactggaaaacatggaactgagaagtttggaactcagcgtcgtagaacctcagcctgtatttgaggcatcaggtgctccagatatttacagttcaaataaagatcttccccctgaaacacctggagcggaggacaaaggcaagagctcgaagaccaaaccctttcgctgtaagccatgccaatatgaagcagaatctgaagaacagtttgtgcatcacatcagagttcacagtgctaagaaattttttgtggaagagagtgcagagaagcaggcaaaagccagggaatctggctcttccactgcagaagagggagatttctccaagggccccattcgctgtgaccgctgcggctacaatactaatcgatatgatcactatacagcacacctgaaacaccacaccagagctggggataatgagcgagtctacaagtgtatcatttgcacatacacaacagtgagcgagtatcactggaggaaacatttaagaaaccattttccaaggaaagtatacacatgtggaaaatgcaactatttttcagacagaaaaaacaattatgttcagcatgttagaactcatacaggagaacgcccatataaatgtgaactttgtccttactcaagttctcagaagactcatctaactagacatatgcgtactcattcaggtgagaagccatttaaatgtgatcagtgcagttatgtggcctctaatcaacatgaagtaacccgccatgcaagacaggttcacaatgggcctaaacctcttaattgcccacactgtgattacaaaacagcagatagaagcaacttcaaaaaacatgtagagctacatgtgaacccacggcagttcaattgccctgtatgtgactatgcagcttccaagaagtgtaatctacagtatcacttcaaatctaagcatcctacttgtcctaataaaacaatggatgtctcaaaagtgaaactaaagaaaaccaaaaaacgagaggctgacttgcctgataatattaccaatgaaaaaacagaaatagaacaaacaaaaataaaaggggatgtggctggaaagaaaaatgaaaagtccgtcaaagcagagaaaagagatgtctcaaaagagaaaaagccttctaataatgtgtcagtgatccaggtgactaccagaactcgaaaatcagtaacagaggtgaaagagatggatgtgcatacaggaagcaattcagaaaaattcagtaaaactaagaaaagcaaaaggaagctggaagttgacagccattctttacatggtcctgtgaatgatgaggaatcttcaacaaaaaagaaaaagaaggtagaaagcaaatccaaaaataatagtcaggaagtgccaaagggtgacagcaaagtggaggagaataaaaagcaaaatacttgcatgaaaaaaagtacaaagaagaaaactctgaaaaataaatcaagtaagaaaagcagtaagcctcctcagaaggaacctgttgagaagggatctgctcagatggaccctcctcagatggggcctgctcccacagaggcggttcagaaggggcccgttcaggtggagctgccacctcccatggagcatgctcagatggagggtgcccagatacggcctgctcctgacgagcctgttcagatggaggtggttcaggaggggcctgctcagaaggagctgctgcctcccgtggagcctgctcagatggtgggtgcccaaattgtacttgctcacatggagctgcctcctcccatggagactgctcagacggaggttgcccaaatggggcctgctcccatggaacctgctcagatggaggttgcccaggtagaatctgctcccatgcaggtggtccagaaggagcctgttcagatggagctgtctcctcccatggaggtggtccagaaggagcctgttcagatagagctgtctcctcccatggaggtggtccagaaggaacctgttaagatagagctgtctcctcccatagaggtggtccagaaggagcctgttcagatggagttgtctcctcccatgggggtggttcagaaggagcctgctcagagggagccacctcctcccagagagcctccccttcacatggagccaatttccaaaaagcctcctctccgaaaagataaaaaggaaaagtctaacatgcagagtgaaagggcacggaaggagcaagtccttattgaagttggcttagtgcctgttaaagatagctggcttctaaaggaaagtgtaagcacagaggatctctcaccaccatcaccaccactgccaaaggaaaatttaagagaagaggcatcaggagaccaaaaattactcaacacaggtgaaggaaataaagaagcccctcttcagaaagtaggagcagaagaggcagatgagagcctacctggtcttgctgctaatatcaacgaatctacccatatttcatcctctggacaaaacttgaatacgccagagggtgaaactttaaatggtaaacatcagactgacagtatagtttgtgaaatgaaaatggacactgatcagaacacaagagagaatctcactggtataaattcaacagttgaagaaccagtttcaccaatgcttcccccttcagcagtagaagaacgtgaagcagtgtccaaaactgcactggcatcacctcctgctacaatggcagcaaatgagtctcaggaaattgatgaagatgaaggcatccacagccatgaaggaagtgacctaagtgacaacatgtcagagggtagtgatgattctggattgcatggggctcggccagttccacaagaatctagcagaaaaaatgcaaaggaagccttggcagtcaaagcggctaagggagattttgtttgtatcttctgtgatcgttctttcagaaagggaaaagattacagcaaacacctcaatcgccatttggttaatgtgtactatcttgaagaagcagctcaagggcaggagtaatgaaactttgaacaaggtttca

SEQ ID NO: 20 - protein, 1097 aa, Homo sapiens
MATQVMGQSSGGGGLFTSSGNIGMALPNDMYDLHDLSKAELAAPQLIMLANVALTGEVNGSCCDYLVGEERQMAELMPVGDNNFSDSEEGEGLEESADIKGEPHGLENMELRSLELSVVEPQPVFEASGAPDIYSSNKDLPPETPGAEDKGKSSKTKPFRCKPCQYEAESEEQFVHHIRVHSAKKFFVEESAEKQAKARESGSSTAEEGDFSKGPIRCDRCGYNTNRYDHYTAHLKHHTRAGDNERVYKCIICTYTTVSEYHWRKHLRNHFPRKVYTCGKCNYFSDRKNNYVQHVRTHTGERPYKCELCPYSSSQKTHLTRHMRTHSGEKPFKCDQCSYVASNQHEVTRHARQVHNGPKPLNCPHCDYKTADRSNFKKHVELHVNPRQFNCPVCDYAASKKCNLQYHFKSKHPTCPNKTMDVSKVKLKKTKKREADLPDNITNEKTEIEQTKIKGDVAGKKNEKSVKAEKRDVSKEKKPSNNVSVIQVTTRTRKSVTEVKEMDVHTGSNSEKFSKTKKSKRKLEVDSHSLHGPVNDEESSTKKKKKVESKSKNNSQEVPKGDSKVEENKKQNTCMKKSTKKKTLKNKSSKKSSKPPQKEPVEKGSAQMDPPQMGPAPTEAVQKGPVQVELPPPMEHAQMEGAQIRPAPDEPVQMEVVQEGPAQKELLPPVEPAQMVGAQIVLAHMELPPPMETAQTEVAQMGPAPMEPAQMEVAQVESAPMQVVQKEPVQMELSPPMEVVQKEPVQIELSPPMEVVQKEPVKIELSPPIEVVQKEPVQMELSPPMGVVQKEPAQREPPPPREPPLHMEPISKKPPLRKDKKEKSNMQSERARKEQVLIEVGLVPVKDSWLLKESVSTEDLSPPSPPLPKENLREEASGDQKLLNTGEGNKEAPLQKVGAEEADESLPGLAANINESTHISSSGQNLNTPEGETLNGKHQTDSIVCEMKMDTDQNTRENLTGINSTVEEPVSPMLPPSAVEEREAVSKTALASPPATMAANESQEIDEDEGIHSHEGSDLSDNMSEGSDDSGLHGARPVPQESSRKNAKEALAVKAAKGDFVCIFCDRSFRKGKDYSKHLNRHLVNVYYLEEAAQGQE

SEQ ID NO: 21 - DNA, 4754 bp, Homo sapiens
gaattctagaggcggcggagggtggcgaggagctctcgctttctctcgctccctccctctccgactccgtctctctctctctctctctctctcccctccctctctttccctctgttccattttttccccctctaaatcctccctgccctgcgcgcctggacacagatttaggaagcgaattcgctcacgttttaggacaaggaagagagagaggcacgggagaagagcccagcaagatttggattgaaaccgagacaccctccggaggctcggagcagaggaaggaggaggagggcggcgaacggaagccagtttgcaattcaagttttgatagcgctggtagaagggggtttaaatcagatttttttttttttaaaggagagagactttttccgctctctcgctccctgttaaagccgggtctagcacagctgcagacgccaccagcgagaaagagggagaggaagacagatagggggcgggggaagaagaaaaagaaaggtaaaaagtcttctaggagaacctttcacatttgcaacaaaagacctaggggctggagagagattcctgggacgcagggctggagtgtctatttcgagctcagcggcagggctcgggcgcgagtcgagaccctgctcgctcctctcgcttctgaaaccgacgttcaggagcggctttttaaaaacgcaaggcacaaggacggtcacccgcgcgactatgtttgctgatttttcgccttgccctctttaaaagcggcctcccattctccaaaagacacttcccctcctccctttgaagtgcattagttgtgatttctgcctccttttcttttttctttcttttttgttttgctttttccccccttttgaattatgtgctgctgttaaacaacaacaaaaaaacaacaaaacacagcagctgcggacttgtccccggctggagcccagcgccccgcctggagtggatgagcctctccatgagagatccggtcattcctgggacaagcatggcctaccatccgttcctacctcaccgggcgccggacttcgccatgagcgcggtgctgggtcaccagccgccgttcttccccgcgctgacgctgcctcccaacggcgcggcggcgctctcgctgccgggcgccctggccaagccgatcatggatcaattggtgggggcggccgagaccggcatcccgttctcctccctggggccccaggcgcatctgaggcctttgaagaccatggagcccgaagaagaggtggaggacgaccccaaggtgcacctggaggctaaagaactttgggatcagtttcacaagcggggcaccgagatggtcattaccaagtcgggaaggcgaatgtttcctccatttaaagtgagatgttctgggctggataaaaaagccaaatacattttattgatggacattatagctgctgatgactgtcgttataaatttcacaattctcggtggatggtggctggtaaggccgaccccgaaatgccaaagaggatgtacattcacccggacagccccgctactggggaacagtggatgtccaaagtcgtcactttccacaaactgaaactcaccaacaacatttcagacaaacatggatttactatattgaactccatgcacaaataccagccccggttccacattgtaagagccaatgacatcttgaaactcccttatagtacatttcggacatacttgttccccgaaactgaattcatcgctgtgactgcataccagaatgataagataacccagttaaaaatagacaacaacccttttgcaaaaggtttccgggacactggaaatggccgaagagaaaaaagaaaacagctcaccctgcagtccatgagggtgtttgatgaaagacacaaaaaggagaatgggacctctgatgagtcctccagtgaacaagcagctttcaactgcttcgcccaggcttcttctccagccgcctccactgtagggacatcgaacctcaaagatttatgtcccagcgagggtgagagcgacgccgaggccgagagcaaagaggagcatggccccgaggcctgcgacgcggccaagatctccaccaccacgtcggaggagccctgccgtgacaagggcagccccgcggtcaaggctcaccttttcgctgctgagcggccccgggacagcgggcggctggacaaagcgtcgcccgactcacgccatagccccgccaccatctcgtccagcactcgcggcctgggcgcggaggagcgcaggagcccggttcgcgagggcacagcgccggccaaggtggaagaggcgcgcgcgctcccgggcaaggaggccttcgcgccgctcacggtgcagacggacgcggccgccgcgcacctggcccagggccccctgcctggcctcggcttcgccccgggcctggcgggccaacagttcttcaacgggcacccgctcttcctgcaccccagccagtttgccatggggggcgccttctccagcatggcggccgctggcatgggtcccctcctggccacggtttctggggcctccaccggtgtctcgggcctggattccacggccatggcctctgccgctgcggcgcagggactgtccggggcgtccgcggccaccctgcccttccacctccagcagcacgtcctggcctctcagggcctggccatgtcccctttcggaagcctgttcccttacccctacacgtacatggccgcagcggcggccgcctcctctgcggcagcctccagctcggtgcaccgccaccccttcctcaatctgaacaccatgcgcccgcggctgcgctacagcccctactccatcccggtgccggtcccggacggcagcagtctgctcaccaccgccctgccctccatggcggcggccgcggggcccctggacggcaaagtcgccgccctggccgccagcccggcctcggtggcagtggactcgggctctgaactcaacagccgctcctccacgctctcctccagctccatgtccttgtcgcccaaactctgcgcggagaaagaggcggccaccagcgaactgcagagcatccagcggttggttagcggcttggaagccaagccggacaggtcccgcagcgcgtccccgtagacccgtcccagacacgtcttttcattccagtccagttcaggctgccgtgcactttgtcggatataaaataaaccacgggcccgccatggcgttagcccttccttttgcagttgcgtctgggaaggggccccggactccctcgagagaatgtgctagagacagcccctgtcttcttggcgtggtttatatgtccgggatctggatcagattctgggggctcagaaacgtcggttgcattgagctactgggggtaggagttccaacatttatgtccagagcaacttccagcaaggctggtctgggtctctgcccaccaggcggggaggtgttcaaagacatctccctcagtgcggatttatatatatatttttccttcactgtgtcaagtggaaacaaaaacaaaatctttcaaaaaaaaaatcgggacaagtgaacacattaacatgattctgtttgtgcagattaaaaactttatagggacttgcattatcggttctcaataaattactgagcagctttgtttggggagggaagtccctaccatccttgtttagtctatattaagaaaatctgtgtctttttaatattcttgtgatgttttcagagccgctgtaggtctcttcttgcatgtccacagtaatgtatttgtggtttttattttgaacgcttgcttttagagagaaaacaatatagccccctacccttttcccaatcctttgccctcaaatcagtgacccaagggagggggggatttaaagggaaggagtgggcaaaacacataaaatgaatttattatatctaagctctgtagcaggattcatgtcgttctttgacagttctttctctttcctgtatatgcaataacaaggttttaaaaaaataataaagaagtgagactattagacaaagtatttatgtaattatttgataactcttgtaaataggtggaatatgaatgcttggaaaattaaactttaatttattgacattgtacatagctctgtgtaaatagaattgcaactgtcaggttttgtgttcttgttttcctttagttgggtttatttccaggtcacagaattgctgttaacactagaaaacacacttcctgcaccaacaccaataccctttcaaaagagttgtctgcaacatttttgttttcttttttaatgtccaaaagtgggggaaagtgctatttcctattttcaccaaaattggggaaggagtgccactttccagctccacttcaaattccttaaaatataactgagattgctgtggggagggaggagggcagaggctgcggtttgactttttaatttttcttttgttatttgtatttgctagtctctgatttcctcaaaacgaagtggaatttactactgttgtcagtatcggtgttttgaattggtgcctgcctatagagatatattcacagttcaaaagtcaggtgctgagagatggtttaaagacaaattcatgaaggtatattttgtgttatagttgttgatgagttctttggttttctgtatttttccccctctctttaaaacatcactgaaatttcaataaatttttattgaaatgtctaaaaaaaaaaaaaaaaaaaaaaaaaaaaaaa

SEQ ID NO: 22 - protein, 723 aa, Homo sapiens
MSLSMRDPVIPGTSMAYHPFLPHRAPDFAMSAVLGHQPPFFPALTLPPNGAAALSLPGALAKPIMDQLVGAAETGIPFSSLGPQAHLRPLKTMEPEEEVEDDPKVHLEAKELWDQFHKRGTEMVITKSGRRMFPPFKVRCSGLDKKAKYILLMDIIAADDCRYKFHNSRWMVAGKADPEMPKRMYIHPDSPATGEQWMSKVVTFHKLKLTNNISDKHGFTILNSMHKYQPRFHIVRANDILKLPYSTFRTYLFPETEFIAVTAYQNDKITQLKIDNNPFAKGFRDTGNGRREKRKQLTLQSMRVFDERHKKENGTSDESSSEQAAFNCFAQASSPAASTVGTSNLKDLCPSEGESDAEAESKEEHGPEACDAAKISTTTSEEPCRDKGSPAVKAHLFAAERPRDSGRLDKASPDSRHSPATISSSTRGLGAEERRSPVREGTAPAKVEEARALPGKEAFAPLTVQTDAAAAHLAQGPLPGLGFAPGLAGQQFFNGHPLFLHPSQFAMGGAFSSMAAAGMGPLLATVSGASTGVSGLDSTAMASAAAAQGLSGASAATLPFHLQQHVLASQGLAMSPFGSLFPYPYTYMAAAAAASSAAASSSVHRHPFLNLNTMRPRLRYSPYSIPVPVPDGSSLLTTALPSMAAAAGPLDGKVAALAASPASVAVDSGSELNSRSSTLSSSSMSLSPKLCAEKEAATSELQSIQRLVSGLEAKPDRSRSASP

SEQ ID NO: 23 - DNA, 3452 bp, Homo sapiens
atgcaggcgcgctactccgtgtccagccccaactccctgggagtggtgccctacctcggcggcgagcagagctactaccgcgcggcggccgcggcggccgggggcggctacaccgccatgccggcccccatgagcgtgtactcgcaccctgcgcacgccgagcagtacccgggcggcatggcccgcgcctacgggccctacacgccgcagccgcagcccaaggacatggtgaagccgccctatagctacatcgcgctcatcaccatggccatccagaacgccccggacaagaagatcaccctgaacggcatctaccagttcatcatggaccgcttccccttctaccgggacaacaagcagggctggcagaacagcatccgccacaacctctcgctcaacgagtgcttcgtcaaggtgccgcgcgacgacaagaagccgggcaagggcagctactggacgctggacccggactcctacaacatgttcgagaacggcagcttcctgcggcggcggcggcgcttcaagaagaaggacgcggtgaaggacaaggaggagaaggacaggctgcacctcaaggagccgcccccgcccggccgccagcccccgcccgcgccgccggagcaggccgacggcaacgcgcccggtccgcagccgccgcccgtgcgcatccaggacatcaagaccgagaacggtacgtgcccctcgccgccccagcccctgtccccggccgccgccctgggcagcggcagcgccgccgcggtgcccaagatcgagagccccgacagcagcagcagcagcctgtccagcgggagcagccccccgggcagcctgccgtcggcgcggccgctcagcctggacggtgcggattccgcgccgccgccgcccgcgccctccgccccgccgccgcaccatagccagggcttcagcgtggacaacatcatgacgtcgctgcgggggtcgccgcagagcgcggccgcggagctcagctccggccttctggcctcggcggccgcgtcctcgcgcgcggggatcgcacccccgctggcgctcggcgcctactcgcccggccagagctccctctacagctccccctgcagccagacctccagcgcgggcagctcgggcggcggcggcggcggcgcgggggccgcggggggcgcgggcggcgccgggacctaccactgcaacctgcaagccatgagcctgtacgcggccggcgagcgcgggggccacttgcagggcgcgcccgggggcgcgggcggctcggccgtggacgaccccctgcccgactactctctgcctccggtcaccagcagcagctcgtcgtccctgagtcacggcggcggcggcggcggcggcgggggaggccaggaggccggccaccaccctgcggcccaccaaggccgcctcacctcgtggtacctgaaccaggcgggcggagacctgggccacttggcgagcgcggcggcggcggcggcggccgcaggctacccgggccagcagcagaacttccactcggtgcgggagatgttcgagtcacagaggatcggcttgaacaactctccagtgaacgggaatagtagctgtcaaatggccttcccttccagccagtctctgtaccgcacgtccggagctttcgtctacgactgtagcaagttttgacacaccctcaaagccgaactaaatcgaaccccaaagcaggaaaagctaaaggaacccatcaaggcaaaatcgaaactaaaaaaaaaaaatccaattaaaaaaaacccctgagaatattcaccacaccagcgaacagaatatccctccaaaaattcagctcaccagcaccagcacgaagaaaactctattttcttaaccgattaattcagagccacctccactttgccttgtctaaataaacaaacccgtaaactgttttatacagagacagcaaaatcttggtttattaaaggacagtgttactccagataacacgtaagtttcttcttgcttttcagagacctgctttcccctcctcccgtctcccctctcttgccttcttccttgcctctcacctgtaagatattattttatcctatgttgaagggagggggaaagtccccgtttatgaaagtcgctttctttttattcatggacttgttttaaaatgtaaattgcaacatagtaatttatttttaatttgtagttggatgtcgtggaccaaacgccagaaagtgttcccaaaacctgacgttaaattgcctgaaactttaaattgtgctttttttctcattataaaaagggaaactgtattaatcttattctatcctcttttctttctttttgttgaacatattcattgtttgtttattaataaattaccattcagtttgaatgagacctatatgtctggatactttaatagagctttaattattacgaaaaaagatttcagagataaaacactagaagttacctattctccacctaaatctctgaaaaatggagaaaccctctgactagtccatgtcaaattttactaaaagtctttttgtttagatttattttcctgcagcatcttctgcaaaatgtactatatagtcagcttgctttgaggctagtaaaaagatatttttctaaacagattggagttggcatataaacaaatacgttttctcactaatgacagtccatgattcggaaattttaagcccatgaatcagccgcggtcttaccacggtgatgcctgtgtgccgagagatgggactgtgcggccagatatgcacagataaatatttggcttgtgtattccatataaaattgcagtgcatattatacatccctgtgagccagatgctgaatagatattttcctattatttcagtcctttataaaaggaaaaataaaccagtttttaaatgtatgtatataattctcccccatttacaatccttcatgtattacatagaaggattgcttttttaaaaatatactgcgggttggaaagggatatttaatctttgagaaactattttagaaaatatgtttgtagaacaattatttttgaaaaagatttaaagcaataacaagaaggaaggcgagaggagcagaacattttggtctagggtggtttctttttaaaccattttttcttgttaatttacagttaaacctaggggacaatccggattggccctcccccttttgtaaataacccaggaaatgtaataaattcattatcttagggtgatctgccctgccaatcagactttggggagatggcgatttgattacagacgttcgggggggtggggggcttgcagtttgttttggagataatacagtttcctgctatctgccgctcctatctagaggcaacacttaagcagtaattgctgttgcttgttgtcaaaatttgatcattgttaaaggattgctgcaaataaatacactttaatttcagtcaaaaa

SEQ ID NO: 24 - protein, 553 aa, Homo sapiens
MQARYSVSSPNSLGVVPYLGGEQSYYRAAAAAAGGGYTAMPAPMSVYSHPAHAEQYPGGMARAYGPYTPQPQPKDMVKPPYSYIALITMAIQNAPDKKITLNGIYQFIMDRFPFYRDNKQGWQNSIRHNLSLNECFVKVPRDDKKPGKGSYWTLDPDSYNMFENGSFLRRRRRFKKKDAVKDKEEKDRLHLKEPPPPGRQPPPAPPEQADGNAPGPQPPPVRIQDIKTENGTCPSPPQPLSPAAALGSGSAAAVPKIESPDSSSSSLSSGSSPPGSLPSARPLSLDGADSAPPPPAPSAPPPHHSQGFSVDNIMTSLRGSPQSAAAELSSGLLASAAASSRAGIAPPLALGAYSPGQSSLYSSPCSQTSSAGSSGGGGGGAGAAGGAGGAGTYHCNLQAMSLYAAGERGGHLQGAPGGAGGSAVDDPLPDYSLPPVTSSSSSSLSHGGGGGGGGGGQEAGHHPAAHQGRLTSWYLNQAGGDLGHLASAAAAAAAAGYPGQQQNFHSVREMFESQRIGLNNSPVNGNSSCQMAFPSSQSLYRTSGAFVYDCSKF

SEQ ID NO: 25 - DNA, 1506 bp, Homo sapiens
atgcaggcgcgctactccgtgtccgaccccaacgccctgggagtggtgccctacctgagcgagcagaattactaccgggctgcgggcagctacggcggcatggccagccccatgggcgtctattccggccacccggagcagtacagcgcggggatgggccgctcctacgcgccctaccaccaccaccagcccgcggcgcctaaggacctggtgaagccgccctacagctacatcgcgctcatcaccatggccatccagaacgcgcccgagaagaagatcaccttgaacggcatctaccagttcatcatggaccgcttccccttctaccgggagaacaagcagggctggcagaacagcatccgccacaacctctcgctcaacgagtgcttcgtcaaggtgccccgcgacgacaagaagcccggcaagggcagttactggaccctggacccggactcctacaacatgttcgagaacggcagcttcctgcggcgccggcggcgcttcaaaaagaaggacgtgtccaaggagaaggaggagcgggcccacctcaaggagccgcccccggcggcgtccaagggcgccccggccaccccccacctagcggacgcccccaaggaggccgagaagaaggtggtgatcaagagcgaggcggcgtccccggcgctgccggtcatcaccaaggtggagacgctgagccccgagagcgcgctgcagggcagcccgcgcagcgcggcctccacgcccgccggctcccccgacggttcgctgccggagcaccacgccgcggcgcccaacgggctgcctggcttcagcgtggagaacatcatgaccctgcgaacgtcgccgccgggcggagagctgagcccgggggccggacgcgcgggcctggtggtgccgccgctggcgctgccatacgccgccgcgccgcccgccgcctacggccagccgtgcgctcagggcctggaggccggggccgccgggggctaccagtgcagcatgcgagcgatgagcctgtacaccggggccgagcggccggcgcacatgtgcgtcccgcccgccctggacgaggccctctcggaccacccgagcggccccacgtcgcccctgagcgctctcaacctcgccgccggccaggagggcgcgctcgccgccacgggccaccaccaccagcaccacggccaccaccacccgcaggcgccgccgcccccgccggctccccagccccagccgacgccgcagcccggggccgccgcggcgcaggcggcctcctggtatctcaaccacagcggggacctgaaccacctccccggccacacgttcgcggcccagcagcaaactttccccaacgtgcgggagatgttcaactcccaccggctggggattgagaactcgaccctcggggagtcccaggtgagtggcaatgccagctgccagctgccctacagatccacgccgcctctctatcgccacgcagccccctactcctacgactgcacgaaatactga

SEQ ID NO: 26 - protein, 501 aa, Homo sapiens
MQARYSVSDPNALGVVPYLSEQNYYRAAGSYGGMASPMGVYSGHPEQYSAGMGRSYAPYHHHQPAAPKDLVKPPYSYIALITMAIQNAPEKKITLNGIYQFIMDRFPFYRENKQGWQNSIRHNLSLNECFVKVPRDDKKPGKGSYWTLDPDSYNMFENGSFLRRRRRFKKKDVSKEKEERAHLKEPPPAASKGAPATPHLADAPKEAEKKVVIKSEAASPALPVITKVETLSPESALQGSPRSAASTPAGSPDGSLPEHHAAAPNGLPGFSVENIMTLRTSPPGGELSPGAGRAGLVVPPLALPYAAAPPAAYGQPCAQGLEAGAAGGYQCSMRAMSLYTGAERPAHMCVPPALDEALSDHPSGPTSPLSALNLAAGQEGALAATGHHHQHHGHHHPQAPPPPPAPQPQPTPQPGAAAAQAASWYLNHSGDLNHLPGHTFAAQQQTFPNVREMFNSHRLGIENSTLGESQVSGNASCQLPYRSTPPLYRHAAPYSYDCTKY

SEQ ID NO: 27 - DNA, 1217 bp, Homo sapiens
agggggcggggaggggcgcagggctgcgcgctcgccggcgctctctttcggtttggtcggcggctggaggagagtggacccccccactttaaggctctgtcctcggcgcgttcccgccgccccccggtcccgacgcggggctcggggatgcccgccagcatgttcagcatcgacaacatcctagccgcccggccgcgctgcaaggactcggtgttgccggtggcgcacagcgcggcggctcccgtcgtcttcccggccctgcacggggactcgctctacggcgccagcggcggcgcctcctcggactatggcgccttctacccgcgccccgtggcccccggcggcgcgggcctcccggccgcggtcagcggctcccgcctcggctacaacaactacttctacgggcagctgcacgtgcaggcggcgcccgtgggcccggcctgctgcggggccgtgccgccgctgggcgcccagcagtgctcctgcgtcccgacgcccccaggctacgagggccccggttcggtgctggtgtccccggtaccgcaccagatgctgccctacatgaacgtgggcacgctgtcgcgcaccgagctgcagcttctcaaccagctgcactgtcggcggaagcggcggcaccgcaccatcttcactgacgagcagctcgaagctctcgagaacctcttccaggagaccaagtacccggacgtgggcacgcgcgagcagctggcccggaaagtgcacctccgcgaggagaaagtggaggtctggtttaagaaccgccgcgccaaatggaggcggcagaagcggtcctcatcagaggagtcggagaacgcggagaagtggaacaagacgtcgtcgtcgaaggcgtcaccggagaagagggaagaggaaggtaaaagcgatttggactcggacagctgacggccgcgggacacttgcccgtattacttacctaactcgaaggacttgcacagacagacgatgctactttcttgcacacgcgctgccttgcgggagggggtcgagaaagaggaacgaggagctgtaaatagtgtacagagccgggagggtcggcgtctggggtcagggcgcgcacagcccagcagcccgaggccgcccgcgactagcccccaccgtagtatttatagttaaattaagggtgacagtacaataaagtgatggcgatgtaaaaaaaaaaaaaaaaaaaaaaaaaaaaa

SEQ ID NO: 28 - protein, 257 aa, Homo sapiens
MPASMFSIDNILAARPRCKDSVLPVAHSAAAPVVFPALHGDSLYGASGGASSDYGAFYPRPVAPGGAGLPAAVSGSRLGYNNYFYGQLHVQAAPVGPACCGAVPPLGAQQCSCVPTPPGYEGPGSVLVSPVPHQMLPYMNVGTLSRTELQLLNQLHCRRKRRHRTIFTDEQLEALENLFQETKYPDVGTREQLARKVHLREEKVEVWFKNRRAKWRRQKRSSSEESENAEKWNKTSSSKASPEKREEEGKSDLDSDS

SEQ ID NO: 29 - DNA, 1309 bp, Homo sapiens
cgctgtgacctagaatgggcgcatgcgccgagcggaactggctggtttgaaaaccatggcgtgggtaccagcggagtccgcagtggaagagttgatgcctcggctattgccggtagagccttgcgacttgacggaaggtttcgatccctcggtacccccgaggacgcctcaggaatacctgaggcgggtccagatcgaagcagctcaatgtccagatgttgtggtagctcaaattgacccaaagaagttgaaaaggaagcaaagtgtgaatatttctctttcaggatgccaacccgcccctgaaggttattccccaacacttcaatggcaacagcaacaagtggcacagttttcaactgttcgacagaatgtgaacaaacatagaagtcactggaaatcacaacagttggatagtaatgtgacaatgccaaaatctgaagatgaagaaggctggaagaaattttgtctgggtgaaaagttatgtgctgacggggctgttggaccagccacaaatgaaagtcctggaatagattatgtacaaattggttttcctcccttgcttagtattgttagcagaatgaatcaggcaacagtaactagtgtcttggaatatctgagtaattggtttggagaaagagactttactccagaattgggaagatggctttatgctttattggcttgtcttgaaaagcctttgttacctgaggctcattcactgattcggcagcttgcaagaaggtgctctgaagtgaggctcttagtggtattttgaccaacgtgatttagctgatgagccatcttgatgtagctgatctctcagggatagaagatatttctcatgaaggcagcctaactctgaggaaaacaatgccaattcaagtacagatttcaacacatcttcaacactatgtgaagggttcacatcttaacctgtgcaattcagattgatactcagaatatgggttgatttgaatatctgaaatatcaatggaaaatcccactcagtttttgatgaacagtttgaacagttttctgtaatcaagcagcttgcatagaaattgtatgatgaaattttacataggttcttggtgctgttttgttctttttttgttttttgttgttttgttatttacttatatacatataaaattttattgaaaatatgttttggttactaaaattttgtttgactcctaacaaaagacaatggatggccttagcatcagaattaaaataatctggattaaatggcaatgtgttcatagtcagcaataaaattaaacatttttccctttaaaaaaaaaaa

SEQ ID NO: 30 - protein, 250 aa, Homo sapiens
MRRAELAGLKTMAWVPAESAVEELMPRLLPVEPCDLTEGFDPSVPPRTPQEYLRRVQIEAAQCPDVVVAQIDPKKLKRKQSVNISLSGCQPAPEGYSPTLQWQQQQVAQFSTVRQNVNKHRSHWKSQQLDSNVTMPKSEDEEGWKKFCLGEKLCADGAVGPATNESPGIDYVQIGFPPLLSIVSRMNQATVTSVLEYLSNWFGERDFTPELGRWLYALLACLEKPLLPEAHSLIRQLARRCSEVRLLVVF

SEQ ID NO: 31 - DNA, 1708 bp, Homo sapiens
ggcacggcctagcgagtggttcttctgcgctactgctgcgcgaatcggcgaccccagtgcctcgaccactatgccgcgctctttcctcgtcaggaagccctccgaccccaatcggaagcctaactacagcgagctgcaggactctaatccagagtttaccttccagcagccctacgaccaggcccacctgctggcagccatcccacctccggagatcctcaaccccaccgcctcgctgccaatgctcatctgggactctgtcctggcgccccaagcccagccaattgcctgggcctcccttcggctccaggagagtcccagggtggcagagctgacctccctgtcagatgaggacagtgggaaaggctcccagccccccagcccaccctcaccggctccttcgtccttctcctctacttcagtctcttccttggaggccgaggcctatgctgccttcccaggcttgggccaagtgcccaagcagctggcccagctctctgaggccaaggatctccaggctcgaaaggccttcaactgcaaatactgcaacaaggaatacctcagcctgggtgccctcaagatgcacatccgaagccacacgctgccctgcgtctgcggaacctgcgggaaggccttctctaggccctggctgctacaaggccatgtccggacccacactggcgagaagcccttctcctgtccccactgcagccgtgccttcgctgaccgctccaacctgcgggcccacctccagacccactcagatgtcaagaagtaccagtgccaggcgtgtgctcggaccttctcccgaatgtccctgctccacaagcaccaagagtccggctgctcaggatgtccccgctgaccctcgaggctccctcttcctctccatacctgcccctgcctgacagccttccccagctccagcaggaaggaccccacatccttctcactgccatggaattccctcctgagtgccccacttctggccacatcagccccacaggactttgatgaagaccattttctggttctgtgtcctctgcctgggctctggaagaggccttcccatggccatttctgtggagggagggcagctggcccccagccctgggggattcctgagctggcctgtctgcgtgggtttttgtatccagagctgtttggatacagctgctttgagctacaggacaaaggctgacagactcactgggaagctcccaccccactcaggggaccccactcccctcacacacacccccccacaaggaaccctcaggccaccctccacgaggtgtgactaactatgcaataatccacccccaggtgcagccccagggcctgcggaggcggtggcagactagagtctgagatgccccgagcccaggcagctatttcagcctcctgtttggtggggtggcacctgtttcccgggcaatttaacaatgtctgaaaagggactgtgagtaatggctgtcacttgtcgggggcccaagtggggtgctctggtctgaccgatgtgtctcccagaactattctgggggcccgacaggtgggcctgggaggaagatgtttacatttttaaaggtacactggtatttatatttcaaacattttgtatcaaggaaacgttttgtatagttatatgtacagtttattgatattcaataaagcagttaatttatatattaaaaaaaaaaaaaaaaaa

SEQ ID NO: 32 - protein, 264 aa, Homo sapiens
MPRSFLVRKPSDPNRKPNYSELQDSNPEFTFQQPYDQAHLLAAIPPPEILNPTASLPMLIWDSVLAPQAQPIAWASLRLQESPRVAELTSLSDEDSGKGSQPPSPPSPAPSSFSSTSVSSLEAEAYAAFPGLGQVPKQLAQLSEAKDLQARKAFNCKYCNKEYLSLGALKMHIRSHTLPCVCGTCGKAFSRPWLLQGHVRTHTGEKPFSCPHCSRAFADRSNLRAHLQTHSDVKKYQCQACARTFSRMSLLHKHQESGCSGCPR

SEQ ID NO: 33 - DNA, 2101 bp, Homo sapiens
agttcgtaaaggagccgggtgacttcagaggcgccggcccgtccgtctgccgcacctgagcacggcccctgcccgagcctggcccgccgcgatgctgtagggaccgccgtgtcctcccgccggaccgttatccgcgccgggcgcccgccagacccgctggcaagatgccgcgctccttcctggtcaagaagcatttcaacgcctccaaaaagccaaactacagcgaactggacacacatacagtgattatttccccgtatctctatgagagttactccatgcctgtcataccacaaccagagatcctcagctcaggagcatacagccccatcactgtgtggactaccgctgctccattccacgcccagctacccaatggcctctctcctctttccggatactcctcatctttggggcgagtgagtccccctcctccatctgacacctcctccaaggaccacagtggctcagaaagccccattagtgatgaagaggaaagactacagtccaagctttcagacccccatgccattgaagctgaaaagtttcagtgcaatttatgcaataagacctattcaactttttctgggctggccaaacataagcagctgcactgcgatgcccagtctagaaaatctttcagctgtaaatactgtgacaaggaatatgtgagcctgggcgccctgaagatgcatattcggacccacacattaccttgtgtttgcaagatctgcggcaaggcgttttccagaccctggttgcttcaaggacacattagaactcacacgggggagaagcctttttcttgccctcactgcaacagagcatttgcagacaggtcaaatctgagggctcatctgcagacccattctgatgtaaagaaataccagtgcaaaaactgctccaaaaccttctccagaatgtctctcctgcacaaacatgaggaatctggctgctgtgtagcacactgagtgacgcaatcaatgtttactcgaacagaatgcatttcttcactccgaagccaaatgacaaataaagtccaaaggcattttctcctgtgctgaccaaccaaataatatgtatagacacacacacatatgcacacacacacacacacacccacagagagagagctgcaagagcatggaattcatgtgtttaaagataatcctttccatgtgaagtttaaaattactatatatttgctgatggctagattgagagaataaaagacagtaacctttctcttcaaagataaaatgaaaagcacattgcatcttttcttcctaaaaaaatgcaaagatttacattgctgccaaatcatttcaactgaaaagaacagtattgctttgtaatagagtctgtaataggatttcccataggaagagatctgccagacgcgaactcaggtgccttaaaaagtattccaagtttactccattacatgtcggttgtctggttgccattgttgaactaaagcctttttttgattacctgtagtgctttaaagtatatttttaaaagggaggaaaaaaataacaagaacaaaacacaggagaatgtattaaaagtatttttgttttgttttgtttttgccaattaacagtatgtgccttgggggaggagggaaagattagctttgaacattcctggcgcatgctccattgtcttactattttaaaacattttaataatttttgaaaattaattaaagatgggaataagtgcaaaagaggattcttacaaattcattaatgtacttaaactatttcaaatgcataccacaaatgcaataatacaataccccttccaagtgcctttttaaattgtatagttgatgagtcaatgtaaatttgtgtttatttttatatgattgaatgagttctgtatgaaactgagatgttgtctatagctatgtctataaacaacctgaagacttgtgaaatcaatgtttcttttttaaaaaacaattttcaagttttttttacaataaacagttttgatttaaaatctcgtttgtatactattttcagagactttacttgcttcatgattagtaccaaaccactgtacaaagaattgtttgttaacaagaaaaaaa

SEQ ID NO: 34 - protein, 268 aa, Homo sapiens
MPRSFLVKKHFNASKKPNYSELDTHTVIISPYLYESYSMPVIPQPEILSSGAYSPITVWTTAAPFHAQLPNGLSPLSGYSSSLGRVSPPPPSDTSSKDHSGSESPISDEEERLQSKLSDPHAIEAEKFQCNLCNKTYSTFSGLAKHKQLHCDAQSRKSFSCKYCDKEYVSLGALKMHIRTHTLPCVCKICGKAFSRPWLLQGHIRTHTGEKPFSCPHCNRAFADRSNLRAHLQTHSDVKKYQCKNCSKTFSRMSLLHKHEESGCCVAH

SEQ ID NO: 35 - DNA, 4396 bp, Homo sapiens
gcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcgctgggtgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaagccggactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaaggtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagccccccccgtccccccccccggaagccactgcacacagacagacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggccccagcggtaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactccttgccccagggacccaggctcatgctctgaactctggctgagaggagtctgctcaggagccagcacaggacaccccccaccccaccccaccatgtccccattacaccagagggccatcgtgacgtagacaggatgccaggggcctgaccagcctccccaatgctggggagcatccctggcctggggccacacctgctgccctccctctgtgtggtccaagggcaagagtggctggagccgggggactgtgctggtctgagccccacgaaggccttgggctgtggctccgaccctgctgcagaaccagcagggtgtcccctcgggcccatctgtgtcccatgtcccagcacccaggcctctctccaggtctccttttctggtcttttgccatgagggtaaccagctcttcccagctggctgggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctgcgccctgggagtttgatctcttaagggaacttgcctctccctcttgttttgctcctgccctgcccctaggtctgggtggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaattttttttaaagaaaataattgtatacgtgtcaacacagctggctggatgattgggactttaaaacgaccctctttcaggtggattcagagacctgtcctgtatataacagcactgtagcaataaacgtgacattttataaag

SEQ ID NO: 36 - protein, 654 aa, Homo sapiens
MNQPQRMAPVGTDKELSDLLDFSMMFPLPVTNGKGRPASLAGAQFGGSGLEDRPSSGSWGSGDQSSSSFDPSRTFSEGTHFTESHSSLSSSTFLGPGLGGKSGERGAYASFGRDAGVGGLTQAGFLSGELALNSPGPLSPSGMKGTSQYYPSYSGSSRRRAADGSLDTQPKKVRKVPPGLPSSVYPPSSGEDYGRDATAYPSAKTPSSTYPAPFYVADGSLHPSAELWSPPGQAGFGPMLGGGSSPLPLPPGSGPVGSSGSSSTFGGLHQHERMGYQLHGAEVNGGLPSASSFSSAPGATYGGVSSHTPPVSGADSLLGSRGTTAGSSGDALGKALASIYSPDHSSNNFSSSPSTPVGSPQGLAGTSQWPRAGAPGALSPSYDGGLHGLQSKIEDHLDEAIHVLRSHAVGTAGDMHTLLPGHGALASGFTGPMSLGGRHAGLVGGSHPEDGLAGSTSLMHNHAALPSQPGTLPDLSRPPDSYSGLGRAGATAAASEIKREEKEDEENTSAADHSEEEKKELKAPRARTSPDEDEDDLLPPEQKAEREKERRVANNARERLRVRDINEAFKELGRMCQLHLNSEKPQTKLLILHQAVSVILNLEQQVRERNLNPKAACLKRREEEKVSGVVGDPQMVLSAPHPGLSEAHNPAGHM

SEQ ID NO: 37 - DNA, 1669 bp, Homo sapiens
gaggtataagagcctccaagtctgcagctctcgcccaactcccagacacctcgcgggctctgcagcaccggcaccgtttccaggaggcctggcggggtgtgcgtccagccgttgggcgctttctttttggacctcggggccatccacaccgtcccctccccctcccgcctccctccccgcctcccccgcgcgccctccccgcggaggtccctcccgtccgtcctcctgctctctcctccgcgggccgcatcgcccgggccggcgccgcgcgcgggggaagctggcgggctgaggcgccccgctcttctcctctgccccgggcccgcgaggccacgcgtcgccgctcgagagatgatgcaggacgtgtccagctcgccagtctcgccggccgacgacagcctgagcaacagcgaggaagagccagaccggcagcagccgccgagcggcaagcgcgggggacgcaagcggcgcagcagcaggcgcagcgcgggcggcggcgcggggcccggcggagccgcgggtgggggcgtcggaggcggcgacgagccgggcagcccggcccagggcaagcgcggcaagaagtctgcgggctgtggcggcggcggcggcgcgggcggcggcggcggcagcagcagcggcggcgggagtccgcagtcttacgaggagctgcagacgcagcgggtcatggccaacgtgcgggagcgccagcgcacccagtcgctgaacgaggcgttcgccgcgctgcggaagatcatccccacgctgccctcggacaagctgagcaagattcagaccctcaagctggcggccaggtacatcgacttcctctaccaggtcctccagagcgacgagctggactccaagatggcaagctgcagctatgtggctcacgagcggctcagctacgccttctcggtctggaggatggagggggcctggtccatgtccgcgtcccactagcaggcggagccccccaccccctcagcagggccggagacctagatgtcattgtttccagagaaggagaaaatggacagtctagagactctggagctggataactaaaaataaaaatatatgccaaagattttcttggaaattagaagagcaaaatccaaattcaaagaaacagggcgtggggcgcacttttaaaagagaaagcgagacaggcccgtggacagtgattcccagacgggcagcggcaccatcctcacacctctgcattctgatagaagtctgaacagttgtttgtgttttttttttttttttttttgacgaagaatgtttttatttttatttttttcatgcatgcattctcaagaggtcgtgccaatcagccactgaaaggaaaggcatcactatggactttctctattttaaaatggtaacaatcagaggaactataagaacacctttagaaataaaaatactgggatcaaactggcctgcaaaaccatagtcagttaattctttttttcatccttcctctgaggggaaaaacaaaaaaaaacttaaaatacaaaaaacaacattctatttatttattgaggacccatggtaaaatgcaaatagatccggtgtctaaatgcattcatatttttatgattgttttgtaaatatctttgtatatttttctgcaataaataaatataaaaaatttagagaaaaa

SEQ ID NO: 38 - protein, 202 aa, Homo sapiens
MMQDVSSSPVSPADDSLSNSEEEPDRQQPPSGKRGGRKRRSSRRSAGGGAGPGGAAGGGVGGGDEPGSPAQGKRGKKSAGCGGGGGAGGGGGSSSGGGSPQSYEELQTQRVMANVRERQRTQSLNEAFAALRKIIPTLPSDKLSKIQTLKLAARYIDFLYQVLQSDELDSKMASCSYVAHERLSYAFSVWRMEGAWSMSASH

SEQ ID NO: 39 - RNA, 19 nt, Artificial Sequence
  Description of Artificial Sequence Synthetic oligonucleotide
aggagaagcuggagcaaaa

SEQ ID NO: 40 - RNA, 19 nt, Artificial Sequence
  Description of Artificial Sequence Synthetic oligonucleotide
ccgugaagcuggagaagga

SEQ ID NO: 41 - RNA, 19 nt, Artificial Sequence
  Description of Artificial Sequence Synthetic oligonucleotide
gagucgggguggagagcaa

SEQ ID NO: 42 - RNA, 19 nt, Artificial Sequence
  Description of Artificial Sequence Synthetic oligonucleotide
agaaggagaagcuggagca

SEQ ID NO: 43 - RNA, 19 nt, Artificial Sequence
  Description of Artificial Sequence Synthetic oligonucleotide
aaggagaagcuggagcaaa

SEQ ID NO: 44 - RNA, 19 nt, Artificial Sequence
  Description of Artificial Sequence Synthetic oligonucleotide
gugccgugaagcuggagaa

SEQ ID NO: 45 - RNA, 19 nt, Artificial Sequence
  Description of Artificial Sequence Synthetic oligonucleotide
gugaagcuggagaaggaga

SEQ ID NO: 46 - RNA, 19 nt, Artificial Sequence
  Description of Artificial Sequence Synthetic oligonucleotide
uggagaaggagaagcugga

SEQ ID NO: 47 - RNA, 19 nt, Artificial Sequence
  Description of Artificial Sequence Synthetic oligonucleotide
gaaggagaagcuggagcaa

SEQ ID NO: 48 - RNA, 19 nt, Artificial Sequence
  Description of Artificial Sequence Synthetic oligonucleotide
gagcaaaacccggaggagu

SEQ ID NO: 49 - RNA, 19 nt, Artificial Sequence
  Description of Artificial Sequence Synthetic oligonucleotide
agaaagaacucgagcaauu

SEQ ID NO: 50 - RNA, 19 nt, Artificial Sequence
  Description of Artificial Sequence Synthetic oligonucleotide
aggagaagcuggagcaaaa

SEQ ID NO: 51 - RNA, 19 nt, Artificial Sequence
  Description of Artificial Sequence Synthetic oligonucleotide
caucaaagcucugcagaaa

SEQ ID NO: 52 - RNA, 19 nt, Artificial Sequence
  Description of Artificial Sequence Synthetic oligonucleotide
gcagaaagaacucgagcaa

SEQ ID NO: 53 - RNA, 19 nt, Artificial Sequence
  Description of Artificial Sequence Synthetic oligonucleotide
ccgugaagcuggagaagga

SEQ ID NO: 54 - RNA, 19 nt, Artificial Sequence
  Description of Artificial Sequence Synthetic oligonucleotide
gaggcaaccuggagaauuu

SEQ ID NO: 55 - RNA, 19 nt, Artificial Sequence
  Description of Artificial Sequence Synthetic oligonucleotide
ggagauaugcaaagcagaa

SEQ ID NO: 56 - RNA, 19 nt, Artificial Sequence
  Description of Artificial Sequence Synthetic oligonucleotide
gcuucaagaacauguguaa

SEQ ID NO: 57 - RNA, 19 nt, Artificial Sequence
  Description of Artificial Sequence Synthetic oligonucleotide
cgaaagagaaagcgaacca

SEQ ID NO: 58 - RNA, 19 nt, Artificial Sequence
  Description of Artificial Sequence Synthetic oligonucleotide
gggaggagcuagggaaaga

SEQ ID NO: 59 - RNA, 19 nt, Artificial Sequence
  Description of Artificial Sequence Synthetic oligonucleotide
ggauuaaguucuucauuca

SEQ ID NO: 60 - RNA, 19 nt, Artificial Sequence
  Description of Artificial Sequence Synthetic oligonucleotide
cagaagggcaagcgaucaa

SEQ ID NO: 61 - RNA, 19 nt, Artificial Sequence
  Description of Artificial Sequence Synthetic oligonucleotide
gggacacaguagauagaca

SEQ ID NO: 62 - RNA, 19 nt, Artificial Sequence
  Description of Artificial Sequence Synthetic oligonucleotide
guagauagacacacuuaaa

SEQ ID NO: 63 - RNA, 19 nt, Artificial Sequence
  Description of Artificial Sequence Synthetic oligonucleotide
gagucgggguggagagcaa

SEQ ID NO: 64 - RNA, 19 nt, Artificial Sequence
  Description of Artificial Sequence Synthetic oligonucleotide
acaucaaagcucugcagaa

SEQ ID NO: 65 - RNA, 19 nt, Artificial Sequence
  Description of Artificial Sequence Synthetic oligonucleotide
ucaaagcucugcagaaaga

SEQ ID NO: 66 - RNA, 19 nt, Artificial Sequence
  Description of Artificial Sequence Synthetic oligonucleotide
ggguggaggaagcugacaa

SEQ ID NO: 67 - RNA, 19 nt, Artificial Sequence
  Description of Artificial Sequence Synthetic oligonucleotide
agagaaagcgaaccaguau

SEQ ID NO: 68 - RNA, 19 nt, Artificial Sequence
  Description of Artificial Sequence Synthetic oligonucleotide
caaugaugcucuugauuuu

SEQ ID NO: 69 - RNA, 19 nt, Artificial Sequence
  Description of Artificial Sequence Synthetic oligonucleotide
ccaagcuccugaagcagaa

SEQ ID NO: 70 - RNA, 19 nt, Artificial Sequence
  Description of Artificial Sequence Synthetic oligonucleotide
gagauaugcaaagcagaaa

SEQ ID NO: 71 - RNA, 19 nt, Artificial Sequence
  Description of Artificial Sequence Synthetic oligonucleotide
cuaaggaaggaauugggaa

SEQ ID NO: 72 - RNA, 19 nt, Artificial Sequence
  Description of Artificial Sequence Synthetic oligonucleotide
caguagauagacacacuua

SEQ ID NO: 73 - RNA, 19 nt, Artificial Sequence
  Description of Artificial Sequence Synthetic oligonucleotide
uugccaagcuccugaagca

SEQ ID NO: 74 - RNA, 19 nt, Artificial Sequence
  Description of Artificial Sequence Synthetic oligonucleotide
agaaguggguggaggaagc

SEQ ID NO: 75 - RNA, 19 nt, Artificial Sequence
  Description of Artificial Sequence Synthetic oligonucleotide
agaaggagaagcuggagca

SEQ ID NO: 76 - RNA, 19 nt, Artificial Sequence
  Description of Artificial Sequence Synthetic oligonucleotide
aaggagaagcuggagcaaa

SEQ ID NO: 77 - RNA, 19 nt, Artificial Sequence
  Description of Artificial Sequence Synthetic oligonucleotide
gcagaaguggguggaggaa

SEQ ID NO: 78 - RNA, 19 nt, Artificial Sequence
  Description of Artificial Sequence Synthetic oligonucleotide
gcccgaaagagaaagcgaa

SEQ ID NO: 79 - RNA, 19 nt, Artificial Sequence
  Description of Artificial Sequence Synthetic oligonucleotide
ugagaggcaaccuggagaa

SEQ ID NO: 80 - RNA, 19 nt, Artificial Sequence
  Description of Artificial Sequence Synthetic oligonucleotide
aggggaggagcuagggaaa

SEQ ID NO: 81 - RNA, 19 nt, Artificial Sequence
  Description of Artificial Sequence Synthetic oligonucleotide
gggauuaaguucuucauuc

SEQ ID NO: 82 - RNA, 19 nt, Artificial Sequence
  Description of Artificial Sequence Synthetic oligonucleotide
gugccgugaagcuggagaa

SEQ ID NO: 83 - RNA, 19 nt, Artificial Sequence
  Description of Artificial Sequence Synthetic oligonucleotide
gaaccgagugagaggcaac

SEQ ID NO: 84 - RNA, 19 nt, Artificial Sequence
  Description of Artificial Sequence Synthetic oligonucleotide
agaaggaugugguccgagu

SEQ ID NO: 85 - RNA, 19 nt, Artificial Sequence
  Description of Artificial Sequence Synthetic oligonucleotide
uaaggaaggaauugggaac

SEQ ID NO: 86 - RNA, 19 nt, Artificial Sequence
  Description of Artificial Sequence Synthetic oligonucleotide
gugaagcuggagaaggaga

SEQ ID NO: 87 - RNA, 19 nt, Artificial Sequence
  Description of Artificial Sequence Synthetic oligonucleotide
uggagaaggagaagcugga

SEQ ID NO: 88 - RNA, 19 nt, Artificial Sequence
  Description of Artificial Sequence Synthetic oligonucleotide
cugcagugcccgaaaccca

SEQ ID NO: 89 - RNA, 19 nt, Artificial Sequence
  Description of Artificial Sequence Synthetic oligonucleotide
gaaggagaagcuggagcaa

SEQ ID NO: 90 - RNA, 19 nt, Artificial Sequence
  Description of Artificial Sequence Synthetic oligonucleotide
agcuugggcucgagaagga

SEQ ID NO: 91 - RNA, 19 nt, Artificial Sequence
  Description of Artificial Sequence Synthetic oligonucleotide
gagcaaaacccggaggagu

SEQ ID NO: 92 - RNA, 19 nt, Artificial Sequence
  Description of Artificial Sequence Synthetic oligonucleotide
gaaagaacucgagcaauuu

SEQ ID NO: 93 - RNA, 19 nt, Artificial Sequence
  Description of Artificial Sequence Synthetic oligonucleotide
gccagaagggcaagcgauc

SEQ ID NO: 94 - RNA, 19 nt, Artificial Sequence
  Description of Artificial Sequence Synthetic oligonucleotide
ugguuggagggaaggugaa

SEQ ID NO: 95 - RNA, 19 nt, Artificial Sequence
  Description of Artificial Sequence Synthetic oligonucleotide
aguagauagacacacuuaa

SEQ ID NO: 96 - RNA, 19 nt, Artificial Sequence
  Description of Artificial Sequence Synthetic oligonucleotide
cagaaagaacucgagcaau

SEQ ID NO: 97 - RNA, 19 nt, Artificial Sequence
  Description of Artificial Sequence Synthetic oligonucleotide
agaaagaacucgagcaauu

SEQ ID NO: 98 - RNA, 19 nt, Artificial Sequence
  Description of Artificial Sequence Synthetic oligonucleotide
caucaaagcucugcagaaa

SEQ ID NO: 99 - RNA, 19 nt, Artificial Sequence
  Description of Artificial Sequence Synthetic oligonucleotide
gcagaaagaacucgagcaa

SEQ ID NO: 100 - RNA, 19 nt, Artificial Sequence
  Description of Artificial Sequence Synthetic oligonucleotide
gaggcaaccuggagaauuu

SEQ ID NO: 101 - RNA, 19 nt, Artificial Sequence
  Description of Artificial Sequence Synthetic oligonucleotide
gggaagguauucagccaaa

SEQ ID NO: 102 - RNA, 19 nt, Artificial Sequence
  Description of Artificial Sequence Synthetic oligonucleotide
ggagauaugcaaagcagaa

SEQ ID NO: 103 - RNA, 19 nt, Artificial Sequence
  Description of Artificial Sequence Synthetic oligonucleotide
gcuucaagaacauguguaa

SEQ ID NO: 104 - RNA, 19 nt, Artificial Sequence
  Description of Artificial Sequence Synthetic oligonucleotide
cgaaagagaaagcgaacca

SEQ ID NO: 105 - RNA, 19 nt, Artificial Sequence
  Description of Artificial Sequence Synthetic oligonucleotide
gggaggagcuagggaaaga

SEQ ID NO: 106 - RNA, 19 nt, Artificial Sequence
  Description of Artificial Sequence Synthetic oligonucleotide
ggauuaaguucuucauuca

SEQ ID NO: 107 - RNA, 19 nt, Artificial Sequence
  Description of Artificial Sequence Synthetic oligonucleotide
cagaagggcaagcgaucaa

SEQ ID NO: 108 - RNA, 19 nt, Artificial Sequence
  Description of Artificial Sequence Synthetic oligonucleotide
gggacacaguagauagaca

SEQ ID NO: 109 - RNA, 19 nt, Artificial Sequence
  Description of Artificial Sequence Synthetic oligonucleotide
guagauagacacacuuaaa

SEQ ID NO: 110 - RNA, 19 nt, Artificial Sequence
  Description of Artificial Sequence Synthetic oligonucleotide
acaucaaagcucugcagaa

SEQ ID NO: 111 - RNA, 19 nt, Artificial Sequence
  Description of Artificial Sequence Synthetic oligonucleotide
cugaagcagaagaggauca

SEQ ID NO: 112 - RNA, 19 nt, Artificial Sequence
  Description of Artificial Sequence Synthetic oligonucleotide
ucaaagcucugcagaaaga

SEQ ID NO: 113 - RNA, 19 nt, Artificial Sequence
  Description of Artificial Sequence Synthetic oligonucleotide
agaggaucacccugggaua

SEQ ID NO: 114 - RNA, 19 nt, Artificial Sequence
  Description of Artificial Sequence Synthetic oligonucleotide
ggguggaggaagcugacaa

SEQ ID NO: 115 - RNA, 19 nt, Artificial Sequence
  Description of Artificial Sequence Synthetic oligonucleotide
cgugcaggcccgaaagaga

SEQ ID NO: 116 - RNA, 19 nt, Artificial Sequence
  Description of Artificial Sequence Synthetic oligonucleotide
gugcaggcccgaaagagaa

SEQ ID NO: 117 - RNA, 19 nt, Artificial Sequence
  Description of Artificial Sequence Synthetic oligonucleotide
agagaaagcgaaccaguau

SEQ ID NO: 118 - RNA, 19 nt, Artificial Sequence
  Description of Artificial Sequence Synthetic oligonucleotide
caaugaugcucuugauuuu

SEQ ID NO: 119 - RNA, 19 nt, Artificial Sequence
  Description of Artificial Sequence Synthetic oligonucleotide
ccaagcuccugaagcagaa

SEQ ID NO: 120 - RNA, 19 nt, Artificial Sequence
  Description of Artificial Sequence Synthetic oligonucleotide
gagauaugcaaagcagaaa

SEQ ID NO: 121 - RNA, 19 nt, Artificial Sequence
  Description of Artificial Sequence Synthetic oligonucleotide
cuaaggaaggaauugggaa

SEQ ID NO: 122 - RNA, 19 nt, Artificial Sequence
  Description of Artificial Sequence Synthetic oligonucleotide
caguagauagacacacuua

SEQ ID NO: 123 - RNA, 19 nt, Artificial Sequence
  Description of Artificial Sequence Synthetic oligonucleotide
uugccaagcuccugaagca

SEQ ID NO: 124 - RNA, 19 nt, Artificial Sequence
  Description of Artificial Sequence Synthetic oligonucleotide
agaaguggguggaggaagc

SEQ ID NO: 125 - RNA, 19 nt, Artificial Sequence
  Description of Artificial Sequence Synthetic oligonucleotide
gcagaaguggguggaggaa

SEQ ID NO: 126 - RNA, 19 nt, Artificial Sequence
  Description of Artificial Sequence Synthetic oligonucleotide
gcccgaaagagaaagcgaa

SEQ ID NO: 127 - RNA, 19 nt, Artificial Sequence
  Description of Artificial Sequence Synthetic oligonucleotide
ugagaggcaaccuggagaa

SEQ ID NO: 128 - RNA, 19 nt, Artificial Sequence
  Description of Artificial Sequence Synthetic oligonucleotide
aggggaggagcuagggaaa

SEQ ID NO: 129 - RNA, 19 nt, Artificial Sequence
  Description of Artificial Sequence Synthetic oligonucleotide
gggauuaaguucuucauuc

SEQ ID NO: 130 - RNA, 19 nt, Artificial Sequence
  Description of Artificial Sequence Synthetic oligonucleotide
gguucuauuugggaaggua

SEQ ID NO: 131 - RNA, 19 nt, Artificial Sequence
  Description of Artificial Sequence Synthetic oligonucleotide
gaaccgagugagaggcaac

SEQ ID NO: 132 - RNA, 19 nt, Artificial Sequence
  Description of Artificial Sequence Synthetic oligonucleotide
agaaggaugugguccgagu

SEQ ID NO: 133 - RNA, 19 nt, Artificial Sequence
  Description of Artificial Sequence Synthetic oligonucleotide
uaaggaaggaauugggaac

SEQ ID NO: 134 - RNA, 19 nt, Artificial Sequence
  Description of Artificial Sequence Synthetic oligonucleotide
guucuauuugggaagguau

SEQ ID NO: 135 - RNA, 19 nt, Artificial Sequence
  Description of Artificial Sequence Synthetic oligonucleotide
cugcagugcccgaaaccca

SEQ ID NO: 136 - RNA, 19 nt, Artificial Sequence
  Description of Artificial Sequence Synthetic oligonucleotide
gaggaucacccugggauau

SEQ ID NO: 137 - RNA, 19 nt, Artificial Sequence
  Description of Artificial Sequence Synthetic oligonucleotide
aggaucacccugggauaua

SEQ ID NO: 138 - RNA, 19 nt, Artificial Sequence
  Description of Artificial Sequence Synthetic oligonucleotide
agcuugggcucgagaagga

SEQ ID NO: 139 - RNA, 19 nt, Artificial Sequence
  Description of Artificial Sequence Synthetic oligonucleotide
gccagaagggcaagcgauc

SEQ ID NO: 140 - RNA, 19 nt, Artificial Sequence
  Description of Artificial Sequence Synthetic oligonucleotide
gaaagaacucgagcaauuu

SEQ ID NO: 141 - RNA, 19 nt, Artificial Sequence
  Description of Artificial Sequence Synthetic oligonucleotide
ugguuggagggaaggugaa

SEQ ID NO: 142 - RNA, 19 nt, Artificial Sequence
  Description of Artificial Sequence Synthetic oligonucleotide
aguagauagacacacuuaa

SEQ ID NO: 143 - RNA, 19 nt, Artificial Sequence
  Description of Artificial Sequence Synthetic oligonucleotide
ugggauauacacaggccga

SEQ ID NO: 144 - RNA, 19 nt, Artificial Sequence
  Description of Artificial Sequence Synthetic oligonucleotide
uugggaagguauucagcca

SEQ ID NO: 145 - RNA, 19 nt, Artificial Sequence
  Description of Artificial Sequence Synthetic oligonucleotide
ucuucaggagauaugcaaa

SEQ ID NO: 146 - RNA, 19 nt, Artificial Sequence
  Description of Artificial Sequence Synthetic oligonucleotide
gggaaugggugaaugacau

SEQ ID NO: 147 - RNA, 19 nt, Artificial Sequence
  Description of Artificial Sequence Synthetic oligonucleotide
auugauaacugguguguuu

SEQ ID NO: 148 - RNA, 19 nt, Artificial Sequence
  Description of Artificial Sequence Synthetic oligonucleotide
ggaaaggggagauugauaa

SEQ ID NO: 149 - RNA, 19 nt, Artificial Sequence
  Description of Artificial Sequence Synthetic oligonucleotide
cuugaaucccgaauggaaa

SEQ ID NO: 150 - RNA, 19 nt, Artificial Sequence
  Description of Artificial Sequence Synthetic oligonucleotide
gugaacagggaauggguga

SEQ ID NO: 151 - RNA, 19 nt, Artificial Sequence
  Description of Artificial Sequence Synthetic oligonucleotide
gagucagugaacagggaau

SEQ ID NO: 152 - RNA, 19 nt, Artificial Sequence
  Description of Artificial Sequence Synthetic oligonucleotide
gaacagggaaugggugaau

SEQ ID NO: 153 - RNA, 19 nt, Artificial Sequence
  Description of Artificial Sequence Synthetic oligonucleotide
uggaaaggggagauugaua

SEQ ID NO: 154 - RNA, 19 nt, Artificial Sequence
  Description of Artificial Sequence Synthetic oligonucleotide
uuacaagucuucugccuuu

SEQ ID NO: 155 - RNA, 19 nt, Artificial Sequence
  Description of Artificial Sequence Synthetic oligonucleotide
acagggaaugggugaauga

SEQ ID NO: 156 - RNA, 19 nt, Artificial Sequence
  Description of Artificial Sequence Synthetic oligonucleotide
ucuugaaucccgaauggaa

SEQ ID NO: 157 - RNA, 19 nt, Artificial Sequence
  Description of Artificial Sequence Synthetic oligonucleotide
gguuauuucuagaaguuag

SEQ ID NO: 158 - RNA, 19 nt, Artificial Sequence
  Description of Artificial Sequence Synthetic oligonucleotide
gacauuuguggguagguua

SEQ ID NO: 159 - RNA, 19 nt, Artificial Sequence
  Description of Artificial Sequence Synthetic oligonucleotide
agggaaugggugaaugaca

SEQ ID NO: 160 - RNA, 19 nt, Artificial Sequence
  Description of Artificial Sequence Synthetic oligonucleotide
acacguagguucuugaauc

SEQ ID NO: 161 - RNA, 19 nt, Artificial Sequence
  Description of Artificial Sequence Synthetic oligonucleotide
ggagauugauaacuggugu

SEQ ID NO: 162 - RNA, 19 nt, Artificial Sequence
  Description of Artificial Sequence Synthetic oligonucleotide
agaaagaacucgagcaauu

SEQ ID NO: 163 - RNA, 19 nt, Artificial Sequence
  Description of Artificial Sequence Synthetic oligonucleotide
caucaaagcucugcagaaa

SEQ ID NO: 164 - RNA, 19 nt, Artificial Sequence
  Description of Artificial Sequence Synthetic oligonucleotide
gcagaaagaacucgagcaa

SEQ ID NO: 165 - RNA, 19 nt, Artificial Sequence
  Description of Artificial Sequence Synthetic oligonucleotide
gaggcaaccuggagaauuu

SEQ ID NO: 166 - RNA, 19 nt, Artificial Sequence
  Description of Artificial Sequence Synthetic oligonucleotide
gggaagguauucagccaaa

SEQ ID NO: 167 - RNA, 19 nt, Artificial Sequence
  Description of Artificial Sequence Synthetic oligonucleotide
ggagauaugcaaagcagaa

SEQ ID NO: 168 - RNA, 19 nt, Artificial Sequence
  Description of Artificial Sequence Synthetic oligonucleotide
gggaaugggugaaugacau

SEQ ID NO: 169 - RNA, 19 nt, Artificial Sequence
  Description of Artificial Sequence Synthetic oligonucleotide
gcuucaagaacauguguaa

SEQ ID NO: 170 - RNA, 19 nt, Artificial Sequence
  Description of Artificial Sequence Synthetic oligonucleotide
auugauaacugguguguuu

SEQ ID NO: 171 - RNA, 19 nt, Artificial Sequence
  Description of Artificial Sequence Synthetic oligonucleotide
cgaaagagaaagcgaacca

SEQ ID NO: 172 - RNA, 19 nt, Artificial Sequence
  Description of Artificial Sequence Synthetic oligonucleotide
gggaggagcuagggaaaga

SEQ ID NO: 173 - RNA, 19 nt, Artificial Sequence
  Description of Artificial Sequence Synthetic oligonucleotide
ggauuaaguucuucauuca

SEQ ID NO: 174 - RNA, 19 nt, Artificial Sequence
  Description of Artificial Sequence Synthetic oligonucleotide
cagaagggcaagcgaucaa

SEQ ID NO: 175 - RNA, 19 nt, Artificial Sequence
  Description of Artificial Sequence Synthetic oligonucleotide
gggacacaguagauagaca

SEQ ID NO: 176 - RNA, 19 nt, Artificial Sequence
  Description of Artificial Sequence Synthetic oligonucleotide
guagauagacacacuuaaa

SEQ ID NO: 177 - RNA, 19 nt, Artificial Sequence
  Description of Artificial Sequence Synthetic oligonucleotide
ggaaaggggagauugauaa

SEQ ID NO: 178 - RNA, 19 nt, Artificial Sequence
  Description of Artificial Sequence Synthetic oligonucleotide
cuugaaucccgaauggaaa

SEQ ID NO: 179 - RNA, 19 nt, Artificial Sequence
  Description of Artificial Sequence Synthetic oligonucleotide
acaucaaagcucugcagaa

SEQ ID NO: 180 - RNA, 19 nt, Artificial Sequence
  Description of Artificial Sequence Synthetic oligonucleotide
gugaacagggaauggguga

SEQ ID NO: 181 - RNA, 19 nt, Artificial Sequence
  Description of Artificial Sequence Synthetic oligonucleotide
ucaaagcucugcagaaaga

SEQ ID NO: 182 - RNA, 19 nt, Artificial Sequence
  Description of Artificial Sequence Synthetic oligonucleotide
cugaagcagaagaggauca

SEQ ID NO: 183 - RNA, 19 nt, Artificial Sequence
  Description of Artificial Sequence Synthetic oligonucleotide
gagucagugaacagggaau

SEQ ID NO: 184 - RNA, 19 nt, Artificial Sequence
  Description of Artificial Sequence Synthetic oligonucleotide
agaggaucacccugggaua

SEQ ID NO: 185 - RNA, 19 nt, Artificial Sequence
  Description of Artificial Sequence Synthetic oligonucleotide
ggguggaggaagcugacaa

SEQ ID NO: 186 - RNA, 19 nt, Artificial Sequence
  Description of Artificial Sequence Synthetic oligonucleotide
cgugcaggcccgaaagaga

SEQ ID NO: 187 - RNA, 19 nt, Artificial Sequence
  Description of Artificial Sequence Synthetic oligonucleotide
gugcaggcccgaaagagaa

SEQ ID NO: 188 - RNA, 19 nt, Artificial Sequence
  Description of Artificial Sequence Synthetic oligonucleotide
gaacagggaaugggugaau

SEQ ID NO: 189 - RNA, 19 nt, Artificial Sequence
  Description of Artificial Sequence Synthetic oligonucleotide
agagaaagcgaaccaguau

SEQ ID NO: 190 - RNA, 19 nt, Artificial Sequence
  Description of Artificial Sequence Synthetic oligonucleotide
caaugaugcucuugauuuu

SEQ ID NO: 191 - RNA, 19 nt, Artificial Sequence
  Description of Artificial Sequence Synthetic oligonucleotide
uggaaaggggagauugaua

SEQ ID NO: 192 - RNA, 19 nt, Artificial Sequence
  Description of Artificial Sequence Synthetic oligonucleotide
ccaagcuccugaagcagaa

SEQ ID NO: 193 - RNA, 19 nt, Artificial Sequence
  Description of Artificial Sequence Synthetic oligonucleotide
gagauaugcaaagcagaaa

SEQ ID NO: 194 - RNA, 19 nt, Artificial Sequence
  Description of Artificial Sequence Synthetic oligonucleotide
cuaaggaaggaauugggaa

SEQ ID NO: 195 - RNA, 19 nt, Artificial Sequence
  Description of Artificial Sequence Synthetic oligonucleotide
caguagauagacacacuua

SEQ ID NO: 196 - RNA, 19 nt, Artificial Sequence
  Description of Artificial Sequence Synthetic oligonucleotide
uuacaagucuucugccuuu

SEQ ID NO: 197 - RNA, 19 nt, Artificial Sequence
  Description of Artificial Sequence Synthetic oligonucleotide
uugccaagcuccugaagca

SEQ ID NO: 198 - RNA, 19 nt, Artificial Sequence
  Description of Artificial Sequence Synthetic oligonucleotide
agaaguggguggaggaagc

SEQ ID NO: 199 - RNA, 19 nt, Artificial Sequence
  Description of Artificial Sequence Synthetic oligonucleotide
acagggaaugggugaauga

SEQ ID NO: 200 - RNA, 19 nt, Artificial Sequence
  Description of Artificial Sequence Synthetic oligonucleotide
ucuugaaucccgaauggaa

SEQ ID NO: 201 - RNA, 19 nt, Artificial Sequence
  Description of Artificial Sequence Synthetic oligonucleotide
gguuauuucuagaaguuag

SEQ ID NO: 202 - RNA, 19 nt, Artificial Sequence
  Description of Artificial Sequence Synthetic oligonucleotide
gacauuuguggguagguua

SEQ ID NO: 203 - RNA, 19 nt, Artificial Sequence
  Description of Artificial Sequence Synthetic oligonucleotide
gcagaaguggguggaggaa

SEQ ID NO: 204 - RNA, 19 nt, Artificial Sequence
  Description of Artificial Sequence Synthetic oligonucleotide
gcccgaaagagaaagcgaa

SEQ ID NO: 205 - RNA, 19 nt, Artificial Sequence
  Description of Artificial Sequence Synthetic oligonucleotide
ugagaggcaaccuggagaa

SEQ ID NO: 206 - RNA, 19 nt, Artificial Sequence
  Description of Artificial Sequence Synthetic oligonucleotide
aggggaggagcuagggaaa

SEQ ID NO: 207 - RNA, 19 nt, Artificial Sequence
  Description of Artificial Sequence Synthetic oligonucleotide
gggauuaaguucuucauuc

SEQ ID NO: 208 - RNA, 19 nt, Artificial Sequence
  Description of Artificial Sequence Synthetic oligonucleotide
agggaaugggugaaugaca

SEQ ID NO: 209 - RNA, 19 nt, Artificial Sequence
  Description of Artificial Sequence Synthetic oligonucleotide
acacguagguucuugaauc

SEQ ID NO: 210 - RNA, 19 nt, Artificial Sequence
  Description of Artificial Sequence Synthetic oligonucleotide
ggagauugauaacuggugu

SEQ ID NO: 211 - RNA, 19 nt, Artificial Sequence
  Description of Artificial Sequence Synthetic oligonucleotide
ccaagacgcucaugaagaa

SEQ ID NO: 212 - RNA, 19 nt, Artificial Sequence
  Description of Artificial Sequence Synthetic oligonucleotide
cguucaucgacgaggcuaa

SEQ ID NO: 213 - RNA, 19 nt, Artificial Sequence
  Description of Artificial Sequence Synthetic oligonucleotide
ucaugaagaaggauaagua

SEQ ID NO: 214 - RNA, 19 nt, Artificial Sequence
  Description of Artificial Sequence Synthetic oligonucleotide
ugauggagacggagcugaa

SEQ ID NO: 215 - RNA, 19 nt, Artificial Sequence
  Description of Artificial Sequence Synthetic oligonucleotide
cgcucaugaagaaggauaa

SEQ ID NO: 216 - RNA, 19 nt, Artificial Sequence
  Description of Artificial Sequence Synthetic oligonucleotide
acgcucaugaagaaggaua

SEQ ID NO: 217 - RNA, 19 nt, Artificial Sequence
  Description of Artificial Sequence Synthetic oligonucleotide
augaagaaggauaaguaca

SEQ ID NO: 218 - RNA, 19 nt, Artificial Sequence
  Description of Artificial Sequence Synthetic oligonucleotide
caguacaacuccaugacca

SEQ ID NO: 219 - RNA, 19 nt, Artificial Sequence
  Description of Artificial Sequence Synthetic oligonucleotide
gcucuuggcuccauggguu

SEQ ID NO: 220 - RNA, 19 nt, Artificial Sequence
  Description of Artificial Sequence Synthetic oligonucleotide
cggaaaaccaagacgcuca

SEQ ID NO: 221 - RNA, 19 nt, Artificial Sequence
  Description of Artificial Sequence Synthetic oligonucleotide
aggagcacccggauuauaa

SEQ ID NO: 222 - RNA, 19 nt, Artificial Sequence
  Description of Artificial Sequence Synthetic oligonucleotide
ccauggguucgguggucaa

SEQ ID NO: 223 - RNA, 19 nt, Artificial Sequence
  Description of Artificial Sequence Synthetic oligonucleotide
acaugaacggcuggagcaa

SEQ ID NO: 224 - RNA, 19 nt, Artificial Sequence
  Description of Artificial Sequence Synthetic oligonucleotide
ugaccagcucgcagaccua

SEQ ID NO: 225 - RNA, 19 nt, Artificial Sequence
  Description of Artificial Sequence Synthetic oligonucleotide
gcucgcagaccuacaugaa

SEQ ID NO: 226 - RNA, 19 nt, Artificial Sequence
  Description of Artificial Sequence Synthetic oligonucleotide
accaagacgcucaugaaga

SEQ ID NO: 227 - RNA, 19 nt, Artificial Sequence
  Description of Artificial Sequence Synthetic oligonucleotide
ugaagaaggauaaguacac

SEQ ID NO: 228 - RNA, 19 nt, Artificial Sequence
  Description of Artificial Sequence Synthetic oligonucleotide
ugcaggaccagcugggcua

SEQ ID NO: 229 - RNA, 19 nt, Artificial Sequence
  Description of Artificial Sequence Synthetic oligonucleotide
ccaccuacagcauguccua

SEQ ID NO: 230 - RNA, 19 nt, Artificial Sequence
  Description of Artificial Sequence Synthetic oligonucleotide
cagcgcagaugcagcccau

SEQ ID NO: 231 - RNA, 19 nt, Artificial Sequence
  Description of Artificial Sequence Synthetic oligonucleotide
acaguuacgcgcacaugaa

SEQ ID NO: 232 - RNA, 19 nt, Artificial Sequence
  Description of Artificial Sequence Synthetic oligonucleotide
uggaaacuuuugucggaga

SEQ ID NO: 233 - RNA, 19 nt, Artificial Sequence
  Description of Artificial Sequence Synthetic oligonucleotide
gugaaccagcgcauggaca

SEQ ID NO: 234 - RNA, 19 nt, Artificial Sequence
  Description of Artificial Sequence Synthetic oligonucleotide
cugcaguacaacuccauga

SEQ ID NO: 235 - RNA, 19 nt, Artificial Sequence
  Description of Artificial Sequence Synthetic oligonucleotide
ggagcacccggauuauaaa

SEQ ID NO: 236 - RNA, 19 nt, Artificial Sequence
  Description of Artificial Sequence Synthetic oligonucleotide
agacgcucaugaagaagga

SEQ ID NO: 237 - RNA, 19 nt, Artificial Sequence
  Description of Artificial Sequence Synthetic oligonucleotide
gcaacggcagcuacagcau

SEQ ID NO: 238 - RNA, 19 nt, Artificial Sequence
  Description of Artificial Sequence Synthetic oligonucleotide
uggcauggcucuuggcucc

SEQ ID NO: 239 - RNA, 19 nt, Artificial Sequence
  Description of Artificial Sequence Synthetic oligonucleotide
accagcgcauggacaguua

SEQ ID NO: 240 - RNA, 19 nt, Artificial Sequence
  Description of Artificial Sequence Synthetic oligonucleotide
ugagcgcccugcaguacaa

SEQ ID NO: 241 - RNA, 19 nt, Artificial Sequence
  Description of Artificial Sequence Synthetic oligonucleotide
caugaagaaggauaaguac

SEQ ID NO: 242 - RNA, 19 nt, Artificial Sequence
  Description of Artificial Sequence Synthetic oligonucleotide
gcacaugaacggcuggagc

SEQ ID NO: 243 - RNA, 19 nt, Artificial Sequence
  Description of Artificial Sequence Synthetic oligonucleotide
cacaugaacggcuggagca

SEQ ID NO: 244 - RNA, 19 nt, Artificial Sequence
  Description of Artificial Sequence Synthetic oligonucleotide
uggagcaacggcagcuaca

SEQ ID NO: 245 - RNA, 19 nt, Artificial Sequence
  Description of Artificial Sequence Synthetic oligonucleotide
agaccuacaugaacggcuc

SEQ ID NO: 246 - RNA, 19 nt, Artificial Sequence
  Description of Artificial Sequence Synthetic oligonucleotide
uggucaaguccgaggccag

SEQ ID NO: 247 - RNA, 19 nt, Artificial Sequence
  Description of Artificial Sequence Synthetic oligonucleotide
ucgacgaggcuaagcggcu

SEQ ID NO: 248 - RNA, 19 nt, Artificial Sequence
  Description of Artificial Sequence Synthetic oligonucleotide
gcacccggauuauaaauac

SEQ ID NO: 249 - RNA, 19 nt, Artificial Sequence
  Description of Artificial Sequence Synthetic oligonucleotide
aguggaaacuuuugucgga

SEQ ID NO: 250 - RNA, 19 nt, Artificial Sequence
  Description of Artificial Sequence Synthetic oligonucleotide
cugcgagcgcugcacauga

SEQ ID NO: 251 - RNA, 19 nt, Artificial Sequence
  Description of Artificial Sequence Synthetic oligonucleotide
agaaagaagaggagagaga

SEQ ID NO: 252 - RNA, 19 nt, Artificial Sequence
  Description of Artificial Sequence Synthetic oligonucleotide
gugcaaaagaggagaguaa

SEQ ID NO: 253 - RNA, 19 nt, Artificial Sequence
  Description of Artificial Sequence Synthetic oligonucleotide
agacuaggacugagagaaa

SEQ ID NO: 254 - RNA, 19 nt, Artificial Sequence
  Description of Artificial Sequence Synthetic oligonucleotide
aaagaagaggagagagaaa

SEQ ID NO: 255 - RNA, 19 nt, Artificial Sequence
  Description of Artificial Sequence Synthetic oligonucleotide
augcacaguuugagauaaa

SEQ ID NO: 256 - RNA, 19 nt, Artificial Sequence
  Description of Artificial Sequence Synthetic oligonucleotide
ggaaagaaagcuacgaaaa

SEQ ID NO: 257 - RNA, 19 nt, Artificial Sequence
  Description of Artificial Sequence Synthetic oligonucleotide
uagaauaaguacuggcgaa

SEQ ID NO: 258 - RNA, 19 nt, Artificial Sequence
  Description of Artificial Sequence Synthetic oligonucleotide
ccaagacgcucaugaagaa

SEQ ID NO: 259 - RNA, 19 nt, Artificial Sequence
  Description of Artificial Sequence Synthetic oligonucleotide
guauagaucuggaggaaag

SEQ ID NO: 260 - RNA, 19 nt, Artificial Sequence
  Description of Artificial Sequence Synthetic oligonucleotide
ccaugaaauuacuguguuu

SEQ ID NO: 261 - RNA, 19 nt, Artificial Sequence
  Description of Artificial Sequence Synthetic oligonucleotide
agaagagaguguuugcaaa

SEQ ID NO: 262 - RNA, 19 nt, Artificial Sequence
  Description of Artificial Sequence Synthetic oligonucleotide
aaagaaagggagagaaguu

SEQ ID NO: 263 - RNA, 19 nt, Artificial Sequence
  Description of Artificial Sequence Synthetic oligonucleotide
gcaaaugacagcugcaaaa

SEQ ID NO: 264 - RNA, 19 nt, Artificial Sequence
  Description of Artificial Sequence Synthetic oligonucleotide
agauaaacauggcaaucaa

SEQ ID NO: 265 - RNA, 19 nt, Artificial Sequence
  Description of Artificial Sequence Synthetic oligonucleotide
aagaggagagagaaagaaa

SEQ ID NO: 266 - RNA, 19 nt, Artificial Sequence
  Description of Artificial Sequence Synthetic oligonucleotide
gcacaguuugagauaaaua

SEQ ID NO: 267 - RNA, 19 nt, Artificial Sequence
  Description of Artificial Sequence Synthetic oligonucleotide
gagaagagaguguuugcaa

SEQ ID NO: 268 - RNA, 19 nt, Artificial Sequence
  Description of Artificial Sequence Synthetic oligonucleotide
ggagagagaaagaaaggga

SEQ ID NO: 269 - RNA, 19 nt, Artificial Sequence
  Description of Artificial Sequence Synthetic oligonucleotide
agaaagaaagggagagaag

SEQ ID NO: 270 - RNA, 19 nt, Artificial Sequence
  Description of Artificial Sequence Synthetic oligonucleotide
ugagagagauccuggacuu

SEQ ID NO: 271 - RNA, 19 nt, Artificial Sequence
  Description of Artificial Sequence Synthetic oligonucleotide
aggaaagaaagcuacgaaa

SEQ ID NO: 272 - RNA, 19 nt, Artificial Sequence
  Description of Artificial Sequence Synthetic oligonucleotide
gcugagaauuugccaauau

SEQ ID NO: 273 - RNA, 19 nt, Artificial Sequence
  Description of Artificial Sequence Synthetic oligonucleotide
ccuuauaacagguacauuu

SEQ ID NO: 274 - RNA, 19 nt, Artificial Sequence
  Description of Artificial Sequence Synthetic oligonucleotide
gaagagaguguuugcaaaa

SEQ ID NO: 275 - RNA, 19 nt, Artificial Sequence
  Description of Artificial Sequence Synthetic oligonucleotide
agaagaggagagagaaaga

SEQ ID NO: 276 - RNA, 19 nt, Artificial Sequence
  Description of Artificial Sequence Synthetic oligonucleotide
gcaaaagaggagaguaaga

SEQ ID NO: 277 - RNA, 19 nt, Artificial Sequence
  Description of Artificial Sequence Synthetic oligonucleotide
ugaaauauggacacugaaa

SEQ ID NO: 278 - RNA, 19 nt, Artificial Sequence
  Description of Artificial Sequence Synthetic oligonucleotide
cguucaucgacgaggcuaa

SEQ ID NO: 279 - RNA, 19 nt, Artificial Sequence
  Description of Artificial Sequence Synthetic oligonucleotide
agagaaagaaagggagaga

SEQ ID NO: 280 - RNA, 19 nt, Artificial Sequence
  Description of Artificial Sequence Synthetic oligonucleotide
ucaugaagaaggauaagua

SEQ ID NO: 281 - RNA, 19 nt, Artificial Sequence
  Description of Artificial Sequence Synthetic oligonucleotide
aagaaacagcauggagaaa

SEQ ID NO: 282 - RNA, 19 nt, Artificial Sequence
  Description of Artificial Sequence Synthetic oligonucleotide
ccgcgaugccgacaagaaa

SEQ ID NO: 283 - RNA, 19 nt, Artificial Sequence
  Description of Artificial Sequence Synthetic oligonucleotide
ggagaggcuucuugcugaa

SEQ ID NO: 284 - RNA, 19 nt, Artificial Sequence
  Description of Artificial Sequence Synthetic oligonucleotide
gaaucagucugccgagaau

SEQ ID NO: 285 - RNA, 19 nt, Artificial Sequence
  Description of Artificial Sequence Synthetic oligonucleotide
uaagaaacagcauggagaa

SEQ ID NO: 286 - RNA, 19 nt, Artificial Sequence
  Description of Artificial Sequence Synthetic oligonucleotide
uuguauagaucuggaggaa

SEQ ID NO: 287 - RNA, 19 nt, Artificial Sequence
  Description of Artificial Sequence Synthetic oligonucleotide
ugauggagacggagcugaa

SEQ ID NO: 288 - RNA, 19 nt, Artificial Sequence
  Description of Artificial Sequence Synthetic oligonucleotide
gguaggagcuuugcaggaa

SEQ ID NO: 289 - RNA, 19 nt, Artificial Sequence
  Description of Artificial Sequence Synthetic oligonucleotide
ggacaguugcaaacgugaa

SEQ ID NO: 290 - RNA, 19 nt, Artificial Sequence
  Description of Artificial Sequence Synthetic oligonucleotide
aauaaguacuggcgaacca

SEQ ID NO: 291 - RNA, 19 nt, Artificial Sequence
  Description of Artificial Sequence Synthetic oligonucleotide
agguugacaccguugguaa

SEQ ID NO: 292 - RNA, 19 nt, Artificial Sequence
  Description of Artificial Sequence Synthetic oligonucleotide
gagaaagaaagggagagaa

SEQ ID NO: 293 - RNA, 19 nt, Artificial Sequence
  Description of Artificial Sequence Synthetic oligonucleotide
caggaguugucaaggcaga

SEQ ID NO: 294 - RNA, 19 nt, Artificial Sequence
  Description of Artificial Sequence Synthetic oligonucleotide
cgcucaugaagaaggauaa

SEQ ID NO: 295 - RNA, 19 nt, Artificial Sequence
  Description of Artificial Sequence Synthetic oligonucleotide
aagaggagaguaagaaaca

SEQ ID NO: 296 - RNA, 19 nt, Artificial Sequence
  Description of Artificial Sequence Synthetic oligonucleotide
ggagagagaccgaggaguu

SEQ ID NO: 297 - RNA, 19 nt, Artificial Sequence
  Description of Artificial Sequence Synthetic oligonucleotide
cagaggagcccaagccaaa

SEQ ID NO: 298 - RNA, 19 nt, Artificial Sequence
  Description of Artificial Sequence Synthetic oligonucleotide
ggacggcuguggauggaaa

SEQ ID NO: 299 - RNA, 19 nt, Artificial Sequence
  Description of Artificial Sequence Synthetic oligonucleotide
gggagaagacacugcguca

SEQ ID NO: 300 - RNA, 19 nt, Artificial Sequence
  Description of Artificial Sequence Synthetic oligonucleotide
ccuucaaccuggcggacau

SEQ ID NO: 301 - RNA, 19 nt, Artificial Sequence
  Description of Artificial Sequence Synthetic oligonucleotide
cagaauuggacccggugua

SEQ ID NO: 302 - RNA, 19 nt, Artificial Sequence
  Description of Artificial Sequence Synthetic oligonucleotide
ugggcaaguucgugcugaa

SEQ ID NO: 303 - RNA, 19 nt, Artificial Sequence
  Description of Artificial Sequence Synthetic oligonucleotide
ggucaucagcgucagcaaa

SEQ ID NO: 304 - RNA, 19 nt, Artificial Sequence
  Description of Artificial Sequence Synthetic oligonucleotide
ggcaaaaccuacacaaaga

SEQ ID NO: 305 - RNA, 19 nt, Artificial Sequence
  Description of Artificial Sequence Synthetic oligonucleotide
ugaccaggcacuaccguaa

SEQ ID NO: 306 - RNA, 19 nt, Artificial Sequence
  Description of Artificial Sequence Synthetic oligonucleotide
ccagaggagcccaagccaa

SEQ ID NO: 307 - RNA, 19 nt, Artificial Sequence
  Description of Artificial Sequence Synthetic oligonucleotide
ccuuacacaugaagaggca

SEQ ID NO: 308 - RNA, 19 nt, Artificial Sequence
  Description of Artificial Sequence Synthetic oligonucleotide
cgggaagggagaagacacu

SEQ ID NO: 309 - RNA, 19 nt, Artificial Sequence
  Description of Artificial Sequence Synthetic oligonucleotide
ccaaagaggggaagacgau

SEQ ID NO: 310 - RNA, 19 nt, Artificial Sequence
  Description of Artificial Sequence Synthetic oligonucleotide
uuacacaugaagaggcauu

SEQ ID NO: 311 - RNA, 19 nt, Artificial Sequence
  Description of Artificial Sequence Synthetic oligonucleotide
ccgaggaguucaacgaucu

SEQ ID NO: 312 - RNA, 19 nt, Artificial Sequence
  Description of Artificial Sequence Synthetic oligonucleotide
gagagaccgaggaguucaa

SEQ ID NO: 313 - RNA, 19 nt, Artificial Sequence
  Description of Artificial Sequence Synthetic oligonucleotide
gcggcaaaaccuacacaaa

SEQ ID NO: 314 - RNA, 19 nt, Artificial Sequence
  Description of Artificial Sequence Synthetic oligonucleotide
aacccacacaggugagaaa

SEQ ID NO: 315 - RNA, 19 nt, Artificial Sequence
  Description of Artificial Sequence Synthetic oligonucleotide
ggacuuuauucucuccaau

SEQ ID NO: 316 - RNA, 19 nt, Artificial Sequence
  Description of Artificial Sequence Synthetic oligonucleotide
gcacgugccccaagaucaa

SEQ ID NO: 317 - RNA, 19 nt, Artificial Sequence
  Description of Artificial Sequence Synthetic oligonucleotide
ggagaagacacugcgucaa

SEQ ID NO: 318 - RNA, 19 nt, Artificial Sequence
  Description of Artificial Sequence Synthetic oligonucleotide
agaucaagcaggaggcggu

SEQ ID NO: 319 - RNA, 19 nt, Artificial Sequence
  Description of Artificial Sequence Synthetic oligonucleotide
guucccaucucaaggcaca

SEQ ID NO: 320 - RNA, 19 nt, Artificial Sequence
  Description of Artificial Sequence Synthetic oligonucleotide
cagaugaacugaccaggca

SEQ ID NO: 321 - RNA, 19 nt, Artificial Sequence
  Description of Artificial Sequence Synthetic oligonucleotide
agaccgaggaguucaacga

SEQ ID NO: 322 - RNA, 19 nt, Artificial Sequence
  Description of Artificial Sequence Synthetic oligonucleotide
gugcugaaggcgucgcuga

SEQ ID NO: 323 - RNA, 19 nt, Artificial Sequence
  Description of Artificial Sequence Synthetic oligonucleotide
cggucaucagcgucagcaa

SEQ ID NO: 324 - RNA, 19 nt, Artificial Sequence
  Description of Artificial Sequence Synthetic oligonucleotide
aagcaggugccccgaauaa

SEQ ID NO: 325 - RNA, 19 nt, Artificial Sequence
  Description of Artificial Sequence Synthetic oligonucleotide
aauuggacccgguguacau

SEQ ID NO: 326 - RNA, 19 nt, Artificial Sequence
  Description of Artificial Sequence Synthetic oligonucleotide
aaaccuacacaaagaguuc

SEQ ID NO: 327 - RNA, 19 nt, Artificial Sequence
  Description of Artificial Sequence Synthetic oligonucleotide
aggcacuaccguaaacaca

SEQ ID NO: 328 - RNA, 19 nt, Artificial Sequence
  Description of Artificial Sequence Synthetic oligonucleotide
gagaagacacugcgucaag

SEQ ID NO: 329 - RNA, 19 nt, Artificial Sequence
  Description of Artificial Sequence Synthetic oligonucleotide
ggugagaaaccuuaccacu

SEQ ID NO: 330 - RNA, 19 nt, Artificial Sequence
  Description of Artificial Sequence Synthetic oligonucleotide
ucaacgaucuccuggaccu

SEQ ID NO: 331 - RNA, 19 nt, Artificial Sequence
  Description of Artificial Sequence Synthetic oligonucleotide
gcgggaagggagaagacac

SEQ ID NO: 332 - RNA, 19 nt, Artificial Sequence
  Description of Artificial Sequence Synthetic oligonucleotide
cccugggucuugaggaagu

SEQ ID NO: 333 - RNA, 19 nt, Artificial Sequence
  Description of Artificial Sequence Synthetic oligonucleotide
ccgaucagaugcagccgca

SEQ ID NO: 334 - RNA, 19 nt, Artificial Sequence
  Description of Artificial Sequence Synthetic oligonucleotide
gcaugccagaggagcccaa

SEQ ID NO: 335 - RNA, 19 nt, Artificial Sequence
  Description of Artificial Sequence Synthetic oligonucleotide
caaagaguucccaucucaa

SEQ ID NO: 336 - RNA, 19 nt, Artificial Sequence
  Description of Artificial Sequence Synthetic oligonucleotide
ucaaccuggcggacaucaa

SEQ ID NO: 337 - RNA, 19 nt, Artificial Sequence
  Description of Artificial Sequence Synthetic oligonucleotide
ggaaaaggaccgccaccca

SEQ ID NO: 338 - RNA, 19 nt, Artificial Sequence
  Description of Artificial Sequence Synthetic oligonucleotide
acacaaagaguucccaucu

SEQ ID NO: 339 - RNA, 19 nt, Artificial Sequence
  Description of Artificial Sequence Synthetic oligonucleotide
ugagaaaccuuaccacugu

SEQ ID NO: 340 - RNA, 19 nt, Artificial Sequence
  Description of Artificial Sequence Synthetic oligonucleotide
gaccaggcacuaccguaaa

SEQ ID NO: 341 - RNA, 19 nt, Artificial Sequence
  Description of Artificial Sequence Synthetic oligonucleotide
ggccagaauuggacccggu

SEQ ID NO: 342 - RNA, 19 nt, Artificial Sequence
  Description of Artificial Sequence Synthetic oligonucleotide
ccgucggucaucagcguca

SEQ ID NO: 343 - RNA, 19 nt, Artificial Sequence
  Description of Artificial Sequence Synthetic oligonucleotide
gccccaagaucaagcagga

SEQ ID NO: 344 - RNA, 19 nt, Artificial Sequence
  Description of Artificial Sequence Synthetic oligonucleotide
gccaaagaggggaagacga

SEQ ID NO: 345 - RNA, 19 nt, Artificial Sequence
  Description of Artificial Sequence Synthetic oligonucleotide
gugagaaaccuuaccacug

SEQ ID NO: 346 - RNA, 19 nt, Artificial Sequence
  Description of Artificial Sequence Synthetic oligonucleotide
ggagagagaccgaggaguu

SEQ ID NO: 347 - RNA, 19 nt, Artificial Sequence
  Description of Artificial Sequence Synthetic oligonucleotide
uguuagaagaagaggaaga

SEQ ID NO: 348 - RNA, 19 nt, Artificial Sequence
  Description of Artificial Sequence Synthetic oligonucleotide
aggaagaaauucagguaca

SEQ ID NO: 349 - RNA, 19 nt, Artificial Sequence
  Description of Artificial Sequence Synthetic oligonucleotide
uagaagaagaggaagaaau

SEQ ID NO: 350 - RNA, 19 nt, Artificial Sequence
  Description of Artificial Sequence Synthetic oligonucleotide
agaagaagaggaagaaauu

SEQ ID NO: 351 - RNA, 19 nt, Artificial Sequence
  Description of Artificial Sequence Synthetic oligonucleotide
cagaggagcccaagccaaa

SEQ ID NO: 352 - RNA, 19 nt, Artificial Sequence
  Description of Artificial Sequence Synthetic oligonucleotide
gaagaaggaucucggccaa

SEQ ID NO: 353 - RNA, 19 nt, Artificial Sequence
  Description of Artificial Sequence Synthetic oligonucleotide
ggacggcuguggauggaaa

SEQ ID NO: 354 - RNA, 19 nt, Artificial Sequence
  Description of Artificial Sequence Synthetic oligonucleotide
gacuggaaguuguggauau

SEQ ID NO: 355 - RNA, 19 nt, Artificial Sequence
  Description of Artificial Sequence Synthetic oligonucleotide
gauguuagaagaagaggaa

SEQ ID NO: 356 - RNA, 19 nt, Artificial Sequence
  Description of Artificial Sequence Synthetic oligonucleotide
agaaauucagguacagaaa

SEQ ID NO: 357 - RNA, 19 nt, Artificial Sequence
  Description of Artificial Sequence Synthetic oligonucleotide
gaucaacauuuaugaccua

SEQ ID NO: 358 - RNA, 19 nt, Artificial Sequence
  Description of Artificial Sequence Synthetic oligonucleotide
gggagaagacacugcguca

SEQ ID NO: 359 - RNA, 19 nt, Artificial Sequence
  Description of Artificial Sequence Synthetic oligonucleotide
gcacuacaaucauggucaa

SEQ ID NO: 360 - RNA, 19 nt, Artificial Sequence
  Description of Artificial Sequence Synthetic oligonucleotide
ccacacugccagaagagaa

SEQ ID NO: 361 - RNA, 19 nt, Artificial Sequence
  Description of Artificial Sequence Synthetic oligonucleotide
ccagaagagaauucaguau

SEQ ID NO: 362 - RNA, 19 nt, Artificial Sequence
  Description of Artificial Sequence Synthetic oligonucleotide
aagaagaggaagaaauuca

SEQ ID NO: 363 - RNA, 19 nt, Artificial Sequence
  Description of Artificial Sequence Synthetic oligonucleotide
aaguaugccuuaagcagaa

SEQ ID NO: 364 - RNA, 19 nt, Artificial Sequence
  Description of Artificial Sequence Synthetic oligonucleotide
ggauaucaggguauaaauu

SEQ ID NO: 365 - RNA, 19 nt, Artificial Sequence
  Description of Artificial Sequence Synthetic oligonucleotide
agucuugguucuaaaggua

SEQ ID NO: 366 - RNA, 19 nt, Artificial Sequence
  Description of Artificial Sequence Synthetic oligonucleotide
cugcauacuuugacaagga

SEQ ID NO: 367 - RNA, 19 nt, Artificial Sequence
  Description of Artificial Sequence Synthetic oligonucleotide
ccuucaaccuggcggacau

SEQ ID NO: 368 - RNA, 19 nt, Artificial Sequence
  Description of Artificial Sequence Synthetic oligonucleotide
cuaaauccgacuugaauau

SEQ ID NO: 369 - RNA, 19 nt, Artificial Sequence
  Description of Artificial Sequence Synthetic oligonucleotide
gaauauuccuggacuuaca

SEQ ID NO: 370 - RNA, 19 nt, Artificial Sequence
  Description of Artificial Sequence Synthetic oligonucleotide
cagaauuggacccggugua

SEQ ID NO: 371 - RNA, 19 nt, Artificial Sequence
  Description of Artificial Sequence Synthetic oligonucleotide
ugggcaaguucgugcugaa

SEQ ID NO: 372 - RNA, 19 nt, Artificial Sequence
  Description of Artificial Sequence Synthetic oligonucleotide
ggucaucagcgucagcaaa

SEQ ID NO: 373 - RNA, 19 nt, Artificial Sequence
  Description of Artificial Sequence Synthetic oligonucleotide
cagaagagaauucaguauu

SEQ ID NO: 374 - RNA, 19 nt, Artificial Sequence
  Description of Artificial Sequence Synthetic oligonucleotide
cuacaaucauggucaaguu

SEQ ID NO: 375 - RNA, 19 nt, Artificial Sequence
  Description of Artificial Sequence Synthetic oligonucleotide
ucaucuugugaguggauaa

SEQ ID NO: 376 - RNA, 19 nt, Artificial Sequence
  Description of Artificial Sequence Synthetic oligonucleotide
gugaguggauaaucaggaa

SEQ ID NO: 377 - RNA, 19 nt, Artificial Sequence
  Description of Artificial Sequence Synthetic oligonucleotide
gaggaauccaaaagacaaa

SEQ ID NO: 378 - RNA, 19 nt, Artificial Sequence
  Description of Artificial Sequence Synthetic oligonucleotide
cuugaauauuccuggacuu

SEQ ID NO: 379 - RNA, 19 nt, Artificial Sequence
  Description of Artificial Sequence Synthetic oligonucleotide
ggugagucuugguucuaaa

SEQ ID NO: 380 - RNA, 19 nt, Artificial Sequence
  Description of Artificial Sequence Synthetic oligonucleotide
ggcaaaaccuacacaaaga

SEQ ID NO: 381 - RNA, 19 nt, Artificial Sequence
  Description of Artificial Sequence Synthetic oligonucleotide
ugaccaggcacuaccguaa

SEQ ID NO: 382 - RNA, 19 nt, Artificial Sequence
  Description of Artificial Sequence Synthetic oligonucleotide
gaaggagcccagccagaaa

SEQ ID NO: 383 - RNA, 19 nt, Artificial Sequence
  Description of Artificial Sequence Synthetic oligonucleotide
gaguggauaaucaggaaaa

SEQ ID NO: 384 - RNA, 19 nt, Artificial Sequence
  Description of Artificial Sequence Synthetic oligonucleotide
cuauauaguuccuugccuu

SEQ ID NO: 385 - RNA, 19 nt, Artificial Sequence
  Description of Artificial Sequence Synthetic oligonucleotide
ccagaggagcccaagccaa

SEQ ID NO: 386 - RNA, 19 nt, Artificial Sequence
  Description of Artificial Sequence Synthetic oligonucleotide
ccuuacacaugaagaggca

SEQ ID NO: 387 - RNA, 19 nt, Artificial Sequence
  Description of Artificial Sequence Synthetic oligonucleotide
ucuaaauccgacuugaaua

SEQ ID NO: 388 - RNA, 19 nt, Artificial Sequence
  Description of Artificial Sequence Synthetic oligonucleotide
agaggaagaaauucaggua

SEQ ID NO: 389 - RNA, 19 nt, Artificial Sequence
  Description of Artificial Sequence Synthetic oligonucleotide
cgggaagggagaagacacu

SEQ ID NO: 390 - RNA, 19 nt, Artificial Sequence
  Description of Artificial Sequence Synthetic oligonucleotide
ccaaagaggggaagacgau

SEQ ID NO: 391 - RNA, 19 nt, Artificial Sequence
  Description of Artificial Sequence Synthetic oligonucleotide
uuacacaugaagaggcauu

SEQ ID NO: 392 - RNA, 19 nt, Artificial Sequence
  Description of Artificial Sequence Synthetic oligonucleotide
ggagggaagaccagaauuc

SEQ ID NO: 393 - RNA, 19 nt, Artificial Sequence
  Description of Artificial Sequence Synthetic oligonucleotide
guuagaagaagaggaagaa

SEQ ID NO: 394 - RNA, 19 nt, Artificial Sequence
  Description of Artificial Sequence Synthetic oligonucleotide
aagaaauucagguacagaa

SEQ ID NO: 395 - RNA, 19 nt, Artificial Sequence
  Description of Artificial Sequence Synthetic oligonucleotide
gcauacuuugacaaggaaa

SEQ ID NO: 396 - RNA, 19 nt, Artificial Sequence
  Description of Artificial Sequence Synthetic oligonucleotide
cuauugagguaaaggguua

SEQ ID NO: 397 - RNA, 19 nt, Artificial Sequence
  Description of Artificial Sequence Synthetic oligonucleotide
gaguaugguuggagccuaa

SEQ ID NO: 398 - RNA, 19 nt, Artificial Sequence
  Description of Artificial Sequence Synthetic oligonucleotide
gguaaaggguuaagcugua

SEQ ID NO: 399 - RNA, 19 nt, Artificial Sequence
  Description of Artificial Sequence Synthetic oligonucleotide
gaaucuaaccucaagaaua

SEQ ID NO: 400 - RNA, 19 nt, Artificial Sequence
  Description of Artificial Sequence Synthetic oligonucleotide
agaaagaggucucguauuu

SEQ ID NO: 401 - RNA, 19 nt, Artificial Sequence
  Description of Artificial Sequence Synthetic oligonucleotide
cuauaacuguggagaggaa

SEQ ID NO: 402 - RNA, 19 nt, Artificial Sequence
  Description of Artificial Sequence Synthetic oligonucleotide
ugacaugaguacugcuuua

SEQ ID NO: 403 - RNA, 19 nt, Artificial Sequence
  Description of Artificial Sequence Synthetic oligonucleotide
cagcagaccacuagguauu

SEQ ID NO: 404 - RNA, 19 nt, Artificial Sequence
  Description of Artificial Sequence Synthetic oligonucleotide
ucuaagagguggcagaaaa

SEQ ID NO: 405 - RNA, 19 nt, Artificial Sequence
  Description of Artificial Sequence Synthetic oligonucleotide
gcaugcaguuccagccaaa

SEQ ID NO: 406 - RNA, 19 nt, Artificial Sequence
  Description of Artificial Sequence Synthetic oligonucleotide
gggaaggccuuaauguaau

SEQ ID NO: 407 - RNA, 19 nt, Artificial Sequence
  Description of Artificial Sequence Synthetic oligonucleotide
uuggauaucuuuaggguuu

SEQ ID NO: 408 - RNA, 19 nt, Artificial Sequence
  Description of Artificial Sequence Synthetic oligonucleotide
cguauuugcugcaucguaa

SEQ ID NO: 409 - RNA, 19 nt, Artificial Sequence
  Description of Artificial Sequence Synthetic oligonucleotide
ucuagagacuccaggauuu

SEQ ID NO: 410 - RNA, 19 nt, Artificial Sequence
  Description of Artificial Sequence Synthetic oligonucleotide
cagagaagagugucgcaaa

SEQ ID NO: 411 - RNA, 19 nt, Artificial Sequence
  Description of Artificial Sequence Synthetic oligonucleotide
ggaucuuccuggagaaaau

SEQ ID NO: 412 - RNA, 19 nt, Artificial Sequence
  Description of Artificial Sequence Synthetic oligonucleotide
agagaagagugucgcaaaa

SEQ ID NO: 413 - RNA, 19 nt, Artificial Sequence
  Description of Artificial Sequence Synthetic oligonucleotide
aagacaaggucccggucaa

SEQ ID NO: 414 - RNA, 19 nt, Artificial Sequence
  Description of Artificial Sequence Synthetic oligonucleotide
augauagauuucagagaca

SEQ ID NO: 415 - RNA, 19 nt, Artificial Sequence
  Description of Artificial Sequence Synthetic oligonucleotide
ggggaaggccuuaauguaa

SEQ ID NO: 416 - RNA, 19 nt, Artificial Sequence
  Description of Artificial Sequence Synthetic oligonucleotide
ggaaggccuuaauguaaua

SEQ ID NO: 417 - RNA, 19 nt, Artificial Sequence
  Description of Artificial Sequence Synthetic oligonucleotide
gugcuaaucuuuguagaaa

SEQ ID NO: 418 - RNA, 19 nt, Artificial Sequence
  Description of Artificial Sequence Synthetic oligonucleotide
ggaacagucccuucuauaa

SEQ ID NO: 419 - RNA, 19 nt, Artificial Sequence
  Description of Artificial Sequence Synthetic oligonucleotide
ucucauggaggguggagua

SEQ ID NO: 420 - RNA, 19 nt, Artificial Sequence
  Description of Artificial Sequence Synthetic oligonucleotide
gcauccgacuguaaagaau

SEQ ID NO: 421 - RNA, 19 nt, Artificial Sequence
  Description of Artificial Sequence Synthetic oligonucleotide
uuccagaaccagagaauga

SEQ ID NO: 422 - RNA, 19 nt, Artificial Sequence
  Description of Artificial Sequence Synthetic oligonucleotide
aaaucuaagagguggcaga

SEQ ID NO: 423 - RNA, 19 nt, Artificial Sequence
  Description of Artificial Sequence Synthetic oligonucleotide
ccugaagacgugugaagau

SEQ ID NO: 424 - RNA, 19 nt, Artificial Sequence
  Description of Artificial Sequence Synthetic oligonucleotide
cgaguguuucaaugaguaa

SEQ ID NO: 425 - RNA, 19 nt, Artificial Sequence
  Description of Artificial Sequence Synthetic oligonucleotide
ccaccagucccaaaggcaa

SEQ ID NO: 426 - RNA, 19 nt, Artificial Sequence
  Description of Artificial Sequence Synthetic oligonucleotide
caccagucccaaaggcaaa

SEQ ID NO: 427 - RNA, 19 nt, Artificial Sequence
  Description of Artificial Sequence Synthetic oligonucleotide
gauagauuucagagacaga

SEQ ID NO: 428 - RNA, 19 nt, Artificial Sequence
  Description of Artificial Sequence Synthetic oligonucleotide
gcaaccagacccagaacau

SEQ ID NO: 429 - RNA, 19 nt, Artificial Sequence
  Description of Artificial Sequence Synthetic oligonucleotide
cuaaacuacuccaugaaca

SEQ ID NO: 430 - RNA, 19 nt, Artificial Sequence
  Description of Artificial Sequence Synthetic oligonucleotide
gagccuaaucagcgagguu

SEQ ID NO: 431 - RNA, 19 nt, Artificial Sequence
  Description of Artificial Sequence Synthetic oligonucleotide
caaugauagauuucagaga

SEQ ID NO: 432 - RNA, 19 nt, Artificial Sequence
  Description of Artificial Sequence Synthetic oligonucleotide
gcuacaaacaggugaagac

SEQ ID NO: 433 - RNA, 19 nt, Artificial Sequence
  Description of Artificial Sequence Synthetic oligonucleotide
gcaauggugugacgcagaa

SEQ ID NO: 434 - RNA, 19 nt, Artificial Sequence
  Description of Artificial Sequence Synthetic oligonucleotide
ggaacaaucaggccuggaa

SEQ ID NO: 435 - RNA, 19 nt, Artificial Sequence
  Description of Artificial Sequence Synthetic oligonucleotide
cuuggaagcugcuggggaa

SEQ ID NO: 436 - RNA, 19 nt, Artificial Sequence
  Description of Artificial Sequence Synthetic oligonucleotide
gauuugugggccugaagaa

SEQ ID NO: 437 - RNA, 19 nt, Artificial Sequence
  Description of Artificial Sequence Synthetic oligonucleotide
aagaaacagaagaccagaa

SEQ ID NO: 438 - RNA, 19 nt, Artificial Sequence
  Description of Artificial Sequence Synthetic oligonucleotide
ccagaaccagagaaugaaa

SEQ ID NO: 439 - RNA, 19 nt, Artificial Sequence
  Description of Artificial Sequence Synthetic oligonucleotide
aaccagagaaugaaaucua

SEQ ID NO: 440 - RNA, 19 nt, Artificial Sequence
  Description of Artificial Sequence Synthetic oligonucleotide
aacaacuggccgaagaaua

SEQ ID NO: 441 - RNA, 19 nt, Artificial Sequence
  Description of Artificial Sequence Synthetic oligonucleotide
guaauacagcagaccacua

SEQ ID NO: 442 - RNA, 19 nt, Artificial Sequence
  Description of Artificial Sequence Synthetic oligonucleotide
ucuuuaggguuuagaaucu

SEQ ID NO: 443 - RNA, 19 nt, Artificial Sequence
  Description of Artificial Sequence Synthetic oligonucleotide
guaaaggguuaagcuguaa

SEQ ID NO: 444 - RNA, 19 nt, Artificial Sequence
  Description of Artificial Sequence Synthetic oligonucleotide
cccaauuucuugauacuuu

SEQ ID NO: 445 - RNA, 19 nt, Artificial Sequence
  Description of Artificial Sequence Synthetic oligonucleotide
gucaagaaacagaagacca

SEQ ID NO: 446 - RNA, 19 nt, Artificial Sequence
  Description of Artificial Sequence Synthetic oligonucleotide
ggaacaagaagaugaggaa

SEQ ID NO: 447 - RNA, 19 nt, Artificial Sequence
  Description of Artificial Sequence Synthetic oligonucleotide
gaggauaucuggaagaaau

SEQ ID NO: 448 - RNA, 19 nt, Artificial Sequence
  Description of Artificial Sequence Synthetic oligonucleotide
acacaaacuugaacagcua

SEQ ID NO: 449 - RNA, 19 nt, Artificial Sequence
  Description of Artificial Sequence Synthetic oligonucleotide
gcgacgaggaggagaacuu

SEQ ID NO: 450 - RNA, 19 nt, Artificial Sequence
  Description of Artificial Sequence Synthetic oligonucleotide
gagaaugucaagaggcgaa

SEQ ID NO: 451 - RNA, 19 nt, Artificial Sequence
  Description of Artificial Sequence Synthetic oligonucleotide
gagaacaguugaaacacaa

SEQ ID NO: 452 - RNA, 19 nt, Artificial Sequence
  Description of Artificial Sequence Synthetic oligonucleotide
acacaauguuucucuguaa

SEQ ID NO: 453 - RNA, 19 nt, Artificial Sequence
  Description of Artificial Sequence Synthetic oligonucleotide
aacaagaagaugaggaaga

SEQ ID NO: 454 - RNA, 19 nt, Artificial Sequence
  Description of Artificial Sequence Synthetic oligonucleotide
aagaagaugaggaagaaau

SEQ ID NO: 455 - RNA, 19 nt, Artificial Sequence
  Description of Artificial Sequence Synthetic oligonucleotide
ucagaggcuuggcgggaaa

SEQ ID NO: 456 - RNA, 19 nt, Artificial Sequence
  Description of Artificial Sequence Synthetic oligonucleotide
uguaguaauuccagcgaga

SEQ ID NO: 457 - RNA, 19 nt, Artificial Sequence
  Description of Artificial Sequence Synthetic oligonucleotide
agggagauccggagcgaau

SEQ ID NO: 458 - RNA, 19 nt, Artificial Sequence
  Description of Artificial Sequence Synthetic oligonucleotide
gggucaaguuggacagugu

SEQ ID NO: 459 - RNA, 19 nt, Artificial Sequence
  Description of Artificial Sequence Synthetic oligonucleotide
cgagaacaguugaaacaca

SEQ ID NO: 460 - RNA, 19 nt, Artificial Sequence
  Description of Artificial Sequence Synthetic oligonucleotide
ggaagaaauucgagcugcu

SEQ ID NO: 461 - RNA, 19 nt, Artificial Sequence
  Description of Artificial Sequence Synthetic oligonucleotide
acaagaagaugaggaagaa

SEQ ID NO: 462 - RNA, 19 nt, Artificial Sequence
  Description of Artificial Sequence Synthetic oligonucleotide
cgauguuguuucuguggaa

SEQ ID NO: 463 - RNA, 19 nt, Artificial Sequence
  Description of Artificial Sequence Synthetic oligonucleotide
acacagaauuucaauccua

SEQ ID NO: 464 - RNA, 19 nt, Artificial Sequence
  Description of Artificial Sequence Synthetic oligonucleotide
gggaucgcgcugaguauaa

SEQ ID NO: 465 - RNA, 19 nt, Artificial Sequence
  Description of Artificial Sequence Synthetic oligonucleotide
cugcuuagacgcuggauuu

SEQ ID NO: 466 - RNA, 19 nt, Artificial Sequence
  Description of Artificial Sequence Synthetic oligonucleotide
aggaggaacaagaagauga

SEQ ID NO: 467 - RNA, 19 nt, Artificial Sequence
  Description of Artificial Sequence Synthetic oligonucleotide
aggaagaaaucgauguugu

SEQ ID NO: 468 - RNA, 19 nt, Artificial Sequence
  Description of Artificial Sequence Synthetic oligonucleotide
agaggaggaacgagcuaaa

SEQ ID NO: 469 - RNA, 19 nt, Artificial Sequence
  Description of Artificial Sequence Synthetic oligonucleotide
ggaacuaugaccucgacua

SEQ ID NO: 470 - RNA, 19 nt, Artificial Sequence
  Description of Artificial Sequence Synthetic oligonucleotide
aagaggacuuguugcggaa

SEQ ID NO: 471 - RNA, 19 nt, Artificial Sequence
  Description of Artificial Sequence Synthetic oligonucleotide
gacgagaacaguugaaaca

SEQ ID NO: 472 - RNA, 19 nt, Artificial Sequence
  Description of Artificial Sequence Synthetic oligonucleotide
cuaacucgcuguaguaauu

SEQ ID NO: 473 - RNA, 19 nt, Artificial Sequence
  Description of Artificial Sequence Synthetic oligonucleotide
gcgaggauaucuggaagaa

SEQ ID NO: 474 - RNA, 19 nt, Artificial Sequence
  Description of Artificial Sequence Synthetic oligonucleotide
gcuuguaccugcaggaucu

SEQ ID NO: 475 - RNA, 19 nt, Artificial Sequence
  Description of Artificial Sequence Synthetic oligonucleotide
ggaagaaaucgauguuguu

SEQ ID NO: 476 - RNA, 19 nt, Artificial Sequence
  Description of Artificial Sequence Synthetic oligonucleotide
cguccaagcagaggagcaa

SEQ ID NO: 477 - RNA, 19 nt, Artificial Sequence
  Description of Artificial Sequence Synthetic oligonucleotide
ccacgaaacuuugcccaua

SEQ ID NO: 478 - RNA, 19 nt, Artificial Sequence
  Description of Artificial Sequence Synthetic oligonucleotide
ccgccaagcucgucucaga

SEQ ID NO: 479 - RNA, 19 nt, Artificial Sequence
  Description of Artificial Sequence Synthetic oligonucleotide
cagagaagcuggccuccua

SEQ ID NO: 480 - RNA, 19 nt, Artificial Sequence
  Description of Artificial Sequence Synthetic oligonucleotide
caagaagaugaggaagaaa

SEQ ID NO: 481 - RNA, 19 nt, Artificial Sequence
  Description of Artificial Sequence Synthetic oligonucleotide
ccacacaucagcacaacua

SEQ ID NO: 482 - RNA, 19 nt, Artificial Sequence
  Description of Artificial Sequence Synthetic oligonucleotide
ccagaggaggaacgagcua

SEQ ID NO: 483 - RNA, 19 nt, Artificial Sequence
  Description of Artificial Sequence Synthetic oligonucleotide
gcggaaacgacgagaacag

SEQ ID NO: 484 - RNA, 19 nt, Artificial Sequence
  Description of Artificial Sequence Synthetic oligonucleotide
guuucaaaugcaugaucaa

SEQ ID NO: 485 - RNA, 19 nt, Artificial Sequence
  Description of Artificial Sequence Synthetic oligonucleotide
acuuacaacacccgagcaa

SEQ ID NO: 486 - RNA, 19 nt, Artificial Sequence
  Description of Artificial Sequence Synthetic oligonucleotide
cugaggaggaacaagaaga

SEQ ID NO: 487 - RNA, 19 nt, Artificial Sequence
  Description of Artificial Sequence Synthetic oligonucleotide
acugcgacgaggaggagaa

SEQ ID NO: 488 - RNA, 19 nt, Artificial Sequence
  Description of Artificial Sequence Synthetic oligonucleotide
cgaggauaucuggaagaaa

SEQ ID NO: 489 - RNA, 19 nt, Artificial Sequence
  Description of Artificial Sequence Synthetic oligonucleotide
aggauaucuggaagaaauu

SEQ ID NO: 490 - RNA, 19 nt, Artificial Sequence
  Description of Artificial Sequence Synthetic oligonucleotide
cgacgagaccuucaucaaa

SEQ ID NO: 491 - RNA, 19 nt, Artificial Sequence
  Description of Artificial Sequence Synthetic oligonucleotide
acucugaggaggaacaaga

SEQ ID NO: 492 - RNA, 19 nt, Artificial Sequence
  Description of Artificial Sequence Synthetic oligonucleotide
gagggaucgcgcugaguau

SEQ ID NO: 493 - RNA, 19 nt, Artificial Sequence
  Description of Artificial Sequence Synthetic oligonucleotide
gcucauuucugaagaggac

SEQ ID NO: 494 - RNA, 19 nt, Artificial Sequence
  Description of Artificial Sequence Synthetic oligonucleotide
gcagcgacucugaggagga

SEQ ID NO: 495 - RNA, 19 nt, Artificial Sequence
  Description of Artificial Sequence Synthetic oligonucleotide
gcgacucugaggaggaaca

SEQ ID NO: 496 - RNA, 19 nt, Artificial Sequence
  Description of Artificial Sequence Synthetic oligonucleotide
acaaauagccauugaacaa

SEQ ID NO: 497 - RNA, 19 nt, Artificial Sequence
  Description of Artificial Sequence Synthetic oligonucleotide
agguaauuccuuagagaua

SEQ ID NO: 498 - RNA, 19 nt, Artificial Sequence
  Description of Artificial Sequence Synthetic oligonucleotide
gugcaguacuguugguuaa

SEQ ID NO: 499 - RNA, 19 nt, Artificial Sequence
  Description of Artificial Sequence Synthetic oligonucleotide
ccaaagggcagaauaaaua

SEQ ID NO: 500 - RNA, 19 nt, Artificial Sequence
  Description of Artificial Sequence Synthetic oligonucleotide
gaugugaaauggagaagua

SEQ ID NO: 501 - RNA, 19 nt, Artificial Sequence
  Description of Artificial Sequence Synthetic oligonucleotide
cuauaauuccagagcauaa

SEQ ID NO: 502 - RNA, 19 nt, Artificial Sequence
  Description of Artificial Sequence Synthetic oligonucleotide
gcacaaaaguuuauaccaa

SEQ ID NO: 503 - RNA, 19 nt, Artificial Sequence
  Description of Artificial Sequence Synthetic oligonucleotide
gggcagaauaaauaagcaa

SEQ ID NO: 504 - RNA, 19 nt, Artificial Sequence
  Description of Artificial Sequence Synthetic oligonucleotide
cagagaugcuccagaauuu

SEQ ID NO: 505 - RNA, 19 nt, Artificial Sequence
  Description of Artificial Sequence Synthetic oligonucleotide
uggaagagcggaagaguuu

SEQ ID NO: 506 - RNA, 19 nt, Artificial Sequence
  Description of Artificial Sequence Synthetic oligonucleotide
uaaccaaagggcagaauaa

SEQ ID NO: 507 - RNA, 19 nt, Artificial Sequence
  Description of Artificial Sequence Synthetic oligonucleotide
gaagaagaauggauuguau

SEQ ID NO: 508 - RNA, 19 nt, Artificial Sequence
  Description of Artificial Sequence Synthetic oligonucleotide
acugaagagcuuaaagaua

SEQ ID NO: 509 - RNA, 19 nt, Artificial Sequence
  Description of Artificial Sequence Synthetic oligonucleotide
ugaaacaauuccagggcau

SEQ ID NO: 510 - RNA, 19 nt, Artificial Sequence
  Description of Artificial Sequence Synthetic oligonucleotide
acaauaagcuaaacgcaau

SEQ ID NO: 511 - RNA, 19 nt, Artificial Sequence
  Description of Artificial Sequence Synthetic oligonucleotide
ccuaacuauuccuguguaa

SEQ ID NO: 512 - RNA, 19 nt, Artificial Sequence
  Description of Artificial Sequence Synthetic oligonucleotide
ugaacaaauguguggguuu

SEQ ID NO: 513 - RNA, 19 nt, Artificial Sequence
  Description of Artificial Sequence Synthetic oligonucleotide
gcuguauaguuguagaauu

SEQ ID NO: 514 - RNA, 19 nt, Artificial Sequence
  Description of Artificial Sequence Synthetic oligonucleotide
ucccagagaccgugcguaa

SEQ ID NO: 515 - RNA, 19 nt, Artificial Sequence
  Description of Artificial Sequence Synthetic oligonucleotide
agauacaauagaaggagua

SEQ ID NO: 516 - RNA, 19 nt, Artificial Sequence
  Description of Artificial Sequence Synthetic oligonucleotide
gggagugugugcagcguuu

SEQ ID NO: 517 - RNA, 19 nt, Artificial Sequence
  Description of Artificial Sequence Synthetic oligonucleotide
aguucaaccucuuacaaua

SEQ ID NO: 518 - RNA, 19 nt, Artificial Sequence
  Description of Artificial Sequence Synthetic oligonucleotide
guaaauagaugacaaacga

SEQ ID NO: 519 - RNA, 19 nt, Artificial Sequence
  Description of Artificial Sequence Synthetic oligonucleotide
gcuccagaggugaacaaua

SEQ ID NO: 520 - RNA, 19 nt, Artificial Sequence
  Description of Artificial Sequence Synthetic oligonucleotide
gggucuuaauugaaaugaa

SEQ ID NO: 521 - RNA, 19 nt, Artificial Sequence
  Description of Artificial Sequence Synthetic oligonucleotide
cuccagaggugaacaauau

SEQ ID NO: 522 - RNA, 19 nt, Artificial Sequence
  Description of Artificial Sequence Synthetic oligonucleotide
caagaaaaccacaacuaaa

SEQ ID NO: 523 - RNA, 19 nt, Artificial Sequence
  Description of Artificial Sequence Synthetic oligonucleotide
ucuuuagagggaaggaaua

SEQ ID NO: 524 - RNA, 19 nt, Artificial Sequence
  Description of Artificial Sequence Synthetic oligonucleotide
cugaagagcuuaaagauag

SEQ ID NO: 525 - RNA, 19 nt, Artificial Sequence
  Description of Artificial Sequence Synthetic oligonucleotide
acacagugagacacaguaa

SEQ ID NO: 526 - RNA, 19 nt, Artificial Sequence
  Description of Artificial Sequence Synthetic oligonucleotide
ggaaacacaccuacaugaa

SEQ ID NO: 527 - RNA, 19 nt, Artificial Sequence
  Description of Artificial Sequence Synthetic oligonucleotide
gcaaacagcuguauaguug

SEQ ID NO: 528 - RNA, 19 nt, Artificial Sequence
  Description of Artificial Sequence Synthetic oligonucleotide
ugagagaaugagauguuua

SEQ ID NO: 529 - RNA, 19 nt, Artificial Sequence
  Description of Artificial Sequence Synthetic oligonucleotide
guccagacaagaugugaaa

SEQ ID NO: 530 - RNA, 19 nt, Artificial Sequence
  Description of Artificial Sequence Synthetic oligonucleotide
caagaugugaaauggagaa

SEQ ID NO: 531 - RNA, 19 nt, Artificial Sequence
  Description of Artificial Sequence Synthetic oligonucleotide
uccuauaauuccagagcau

SEQ ID NO: 532 - RNA, 19 nt, Artificial Sequence
  Description of Artificial Sequence Synthetic oligonucleotide
cacugacacugaaggguua

SEQ ID NO: 533 - RNA, 19 nt, Artificial Sequence
  Description of Artificial Sequence Synthetic oligonucleotide
caguauaccuggcaauuca

SEQ ID NO: 534 - RNA, 19 nt, Artificial Sequence
  Description of Artificial Sequence Synthetic oligonucleotide
aaucauuucuuuagaggga

SEQ ID NO: 535 - RNA, 19 nt, Artificial Sequence
  Description of Artificial Sequence Synthetic oligonucleotide
guucaaccucuuacaauaa

SEQ ID NO: 536 - RNA, 19 nt, Artificial Sequence
  Description of Artificial Sequence Synthetic oligonucleotide
uuacagugcaguuuaguua

SEQ ID NO: 537 - RNA, 19 nt, Artificial Sequence
  Description of Artificial Sequence Synthetic oligonucleotide
gugucugccuuuaaauaua

SEQ ID NO: 538 - RNA, 19 nt, Artificial Sequence
  Description of Artificial Sequence Synthetic oligonucleotide
uaacacacaucaagacaga

SEQ ID NO: 539 - RNA, 19 nt, Artificial Sequence
  Description of Artificial Sequence Synthetic oligonucleotide
acauccaaccugucagaua

SEQ ID NO: 540 - RNA, 19 nt, Artificial Sequence
  Description of Artificial Sequence Synthetic oligonucleotide
aagaauggauuguauguca

SEQ ID NO: 541 - RNA, 19 nt, Artificial Sequence
  Description of Artificial Sequence Synthetic oligonucleotide
guagguaauuccuuagaga

SEQ ID NO: 542 - RNA, 19 nt, Artificial Sequence
  Description of Artificial Sequence Synthetic oligonucleotide
acauauaugaguugccuau

SEQ ID NO: 543 - RNA, 19 nt, Artificial Sequence
  Description of Artificial Sequence Synthetic oligonucleotide
caaaucagcuuuauagguu

SEQ ID NO: 544 - RNA, 19 nt, Artificial Sequence
  Description of Artificial Sequence Synthetic oligonucleotide
acacuuacaguuaggauuu

SEQ ID NO: 545 - RNA, 19 nt, Artificial Sequence
  Description of Artificial Sequence Synthetic oligonucleotide
cuuuagagggaaggaauaa

SEQ ID NO: 546 - RNA, 19 nt, Artificial Sequence
  Description of Artificial Sequence Synthetic oligonucleotide
ugugaugccuugugagaaa

SEQ ID NO: 547 - RNA, 19 nt, Artificial Sequence
  Description of Artificial Sequence Synthetic oligonucleotide
guauauagugacugacaaa

SEQ ID NO: 548 - RNA, 19 nt, Artificial Sequence
  Description of Artificial Sequence Synthetic oligonucleotide
ggcaagaccuacaccaaga

SEQ ID NO: 549 - RNA, 19 nt, Artificial Sequence
  Description of Artificial Sequence Synthetic oligonucleotide
uggagcugcuggaggccaa

SEQ ID NO: 550 - RNA, 19 nt, Artificial Sequence
  Description of Artificial Sequence Synthetic oligonucleotide
gcggcaagaccuacaccaa

SEQ ID NO: 551 - RNA, 19 nt, Artificial Sequence
  Description of Artificial Sequence Synthetic oligonucleotide
gguauuuauuggacccaga

SEQ ID NO: 552 - RNA, 19 nt, Artificial Sequence
  Description of Artificial Sequence Synthetic oligonucleotide
uagagagacaggugggcau

SEQ ID NO: 553 - RNA, 19 nt, Artificial Sequence
  Description of Artificial Sequence Synthetic oligonucleotide
gcaccgacgacgaccucaa

SEQ ID NO: 554 - RNA, 19 nt, Artificial Sequence
  Description of Artificial Sequence Synthetic oligonucleotide
uacuguacauagagagaca

SEQ ID NO: 555 - RNA, 19 nt, Artificial Sequence
  Description of Artificial Sequence Synthetic oligonucleotide
uguauauagugacugacaa

SEQ ID NO: 556 - RNA, 19 nt, Artificial Sequence
  Description of Artificial Sequence Synthetic oligonucleotide
ccaaacugugacugguauu

SEQ ID NO: 557 - RNA, 19 nt, Artificial Sequence
  Description of Artificial Sequence Synthetic oligonucleotide
ugcuggaggccaagccaaa

SEQ ID NO: 558 - RNA, 19 nt, Artificial Sequence
  Description of Artificial Sequence Synthetic oligonucleotide
cagcguggcuacagagggu

SEQ ID NO: 559 - RNA, 19 nt, Artificial Sequence
  Description of Artificial Sequence Synthetic oligonucleotide
agaccuacaccaagaguuc

SEQ ID NO: 560 - RNA, 19 nt, Artificial Sequence
  Description of Artificial Sequence Synthetic oligonucleotide
acuagaggaucgaggcuug

SEQ ID NO: 561 - RNA, 19 nt, Artificial Sequence
  Description of Artificial Sequence Synthetic oligonucleotide
guauuacuguacauagaga

SEQ ID NO: 562 - RNA, 19 nt, Artificial Sequence
  Description of Artificial Sequence Synthetic oligonucleotide
guacauagagagacaggug

SEQ ID NO: 563 - RNA, 19 nt, Artificial Sequence
  Description of Artificial Sequence Synthetic oligonucleotide
auuacuguacauagagaga

SEQ ID NO: 564 - RNA, 19 nt, Artificial Sequence
  Description of Artificial Sequence Synthetic oligonucleotide
ugggcuaccugguucguuu

SEQ ID NO: 565 - RNA, 19 nt, Artificial Sequence
  Description of Artificial Sequence Synthetic oligonucleotide
ggugagaagcccuaccacu

SEQ ID NO: 566 - RNA, 19 nt, Artificial Sequence
  Description of Artificial Sequence Synthetic oligonucleotide
gcuggaaguuugcgcgcuc

SEQ ID NO: 567 - RNA, 19 nt, Artificial Sequence
  Description of Artificial Sequence Synthetic oligonucleotide
auuuauuggacccagagaa

SEQ ID NO: 568 - RNA, 19 nt, Artificial Sequence
  Description of Artificial Sequence Synthetic oligonucleotide
gggucucccucgaugacga

SEQ ID NO: 569 - RNA, 19 nt, Artificial Sequence
  Description of Artificial Sequence Synthetic oligonucleotide
ucgaugacgacgacgacga

SEQ ID NO: 570 - RNA, 19 nt, Artificial Sequence
  Description of Artificial Sequence Synthetic oligonucleotide
gggaaaagaccacgauccu

SEQ ID NO: 571 - RNA, 19 nt, Artificial Sequence
  Description of Artificial Sequence Synthetic oligonucleotide
accgaaagcacacgggcca

SEQ ID NO: 572 - RNA, 19 nt, Artificial Sequence
  Description of Artificial Sequence Synthetic oligonucleotide
ucccaaacugugacuggua

SEQ ID NO: 573 - RNA, 19 nt, Artificial Sequence
  Description of Artificial Sequence Synthetic oligonucleotide
accaagaguucgcaucuga

SEQ ID NO: 574 - RNA, 19 nt, Artificial Sequence
  Description of Artificial Sequence Synthetic oligonucleotide
ccaagaguucgcaucugaa

SEQ ID NO: 575 - RNA, 19 nt, Artificial Sequence
  Description of Artificial Sequence Synthetic oligonucleotide
cccaaacugugacugguau

SEQ ID NO: 576 - RNA, 19 nt, Artificial Sequence
  Description of Artificial Sequence Synthetic oligonucleotide
ugaugccuugugagaaaua

SEQ ID NO: 577 - RNA, 19 nt, Artificial Sequence
  Description of Artificial Sequence Synthetic oligonucleotide
acgacgaccucaacagcgu

SEQ ID NO: 578 - RNA, 19 nt, Artificial Sequence
  Description of Artificial Sequence Synthetic oligonucleotide
cugcuggaggccaagccaa

SEQ ID NO: 579 - RNA, 19 nt, Artificial Sequence
  Description of Artificial Sequence Synthetic oligonucleotide
guucgcaucugaaggcgca

SEQ ID NO: 580 - RNA, 19 nt, Artificial Sequence
  Description of Artificial Sequence Synthetic oligonucleotide
gugagaagcccuaccacug

SEQ ID NO: 581 - RNA, 19 nt, Artificial Sequence
  Description of Artificial Sequence Synthetic oligonucleotide
ucacgcgccacuaccgaaa

SEQ ID NO: 582 - RNA, 19 nt, Artificial Sequence
  Description of Artificial Sequence Synthetic oligonucleotide
cugcacaugaaacggcaca

SEQ ID NO: 583 - RNA, 19 nt, Artificial Sequence
  Description of Artificial Sequence Synthetic oligonucleotide
uuuauuggacccagagaac

SEQ ID NO: 584 - RNA, 19 nt, Artificial Sequence
  Description of Artificial Sequence Synthetic oligonucleotide
agagagacaggugggcauu

SEQ ID NO: 585 - RNA, 19 nt, Artificial Sequence
  Description of Artificial Sequence Synthetic oligonucleotide
acaccaagaguucgcaucu

SEQ ID NO: 586 - RNA, 19 nt, Artificial Sequence
  Description of Artificial Sequence Synthetic oligonucleotide
aaacugugacugguauuua

SEQ ID NO: 587 - RNA, 19 nt, Artificial Sequence
  Description of Artificial Sequence Synthetic oligonucleotide
ggcacagcguggcuacaga

SEQ ID NO: 588 - RNA, 19 nt, Artificial Sequence
  Description of Artificial Sequence Synthetic oligonucleotide
ugucugagcugcugcgacc

SEQ ID NO: 589 - RNA, 19 nt, Artificial Sequence
  Description of Artificial Sequence Synthetic oligonucleotide
ccuucggucucuucgacga

SEQ ID NO: 590 - RNA, 19 nt, Artificial Sequence
  Description of Artificial Sequence Synthetic oligonucleotide
gcaaacgcaccgccacuca

SEQ ID NO: 591 - RNA, 19 nt, Artificial Sequence
  Description of Artificial Sequence Synthetic oligonucleotide
gcguggcuacagagggucu

SEQ ID NO: 592 - RNA, 19 nt, Artificial Sequence
  Description of Artificial Sequence Synthetic oligonucleotide
gaucgaggcuugugaugcc

SEQ ID NO: 593 - RNA, 19 nt, Artificial Sequence
  Description of Artificial Sequence Synthetic oligonucleotide
gccuuaauuuguacugucu

SEQ ID NO: 594 - RNA, 19 nt, Artificial Sequence
  Description of Artificial Sequence Synthetic oligonucleotide
uuguacugucugcggcauu

SEQ ID NO: 595 - RNA, 19 nt, Artificial Sequence
  Description of Artificial Sequence Synthetic oligonucleotide
gcgucaaacugcagggcaa

SEQ ID NO: 596 - RNA, 19 nt, Artificial Sequence
  Description of Artificial Sequence Synthetic oligonucleotide
cagagugccuggauggaaa

SEQ ID NO: 597 - RNA, 19 nt, Artificial Sequence
  Description of Artificial Sequence Synthetic oligonucleotide
uggagaugcuggaggccaa

SEQ ID NO: 598 - RNA, 19 nt, Artificial Sequence
  Description of Artificial Sequence Synthetic oligonucleotide
ugguguacgcugaggacua

SEQ ID NO: 599 - RNA, 19 nt, Artificial Sequence
  Description of Artificial Sequence Synthetic oligonucleotide
acaagaagcucaaggugga

SEQ ID NO: 600 - RNA, 19 nt, Artificial Sequence
  Description of Artificial Sequence Synthetic oligonucleotide
ugaccaagauugucucaua

SEQ ID NO: 601 - RNA, 19 nt, Artificial Sequence
  Description of Artificial Sequence Synthetic oligonucleotide
ccauguacaucgaggaucu

SEQ ID NO: 602 - RNA, 19 nt, Artificial Sequence
  Description of Artificial Sequence Synthetic oligonucleotide
caccaggaggccagggaaa

SEQ ID NO: 603 - RNA, 19 nt, Artificial Sequence
  Description of Artificial Sequence Synthetic oligonucleotide
cggacaagcucuaugccau

SEQ ID NO: 604 - RNA, 19 nt, Artificial Sequence
  Description of Artificial Sequence Synthetic oligonucleotide
caagcagggaucagagcaa

SEQ ID NO: 605 - RNA, 19 nt, Artificial Sequence
  Description of Artificial Sequence Synthetic oligonucleotide
ucccugggcuggugaauaa

SEQ ID NO: 606 - RNA, 19 nt, Artificial Sequence
  Description of Artificial Sequence Synthetic oligonucleotide
cagaggugauccagugauu

SEQ ID NO: 607 - RNA, 19 nt, Artificial Sequence
  Description of Artificial Sequence Synthetic oligonucleotide
guggaagagaaaugagcuu

SEQ ID NO: 608 - RNA, 19 nt, Artificial Sequence
  Description of Artificial Sequence Synthetic oligonucleotide
ccaucaagugcgaguacau

SEQ ID NO: 609 - RNA, 19 nt, Artificial Sequence
  Description of Artificial Sequence Synthetic oligonucleotide
cgucaaacugcagggcaaa

SEQ ID NO: 610 - RNA, 19 nt, Artificial Sequence
  Description of Artificial Sequence Synthetic oligonucleotide
ggacauugccucuggcuac

SEQ ID NO: 611 - RNA, 19 nt, Artificial Sequence
  Description of Artificial Sequence Synthetic oligonucleotide
agcucaagguggagaagga

SEQ ID NO: 612 - RNA, 19 nt, Artificial Sequence
  Description of Artificial Sequence Synthetic oligonucleotide
agguggagaaggaggaguu

SEQ ID NO: 613 - RNA, 19 nt, Artificial Sequence
  Description of Artificial Sequence Synthetic oligonucleotide
acgaggcacugcaggacua

SEQ ID NO: 614 - RNA, 19 nt, Artificial Sequence
  Description of Artificial Sequence Synthetic oligonucleotide
cucccaaggaugaaagaau

SEQ ID NO: 615 - RNA, 19 nt, Artificial Sequence
  Description of Artificial Sequence Synthetic oligonucleotide
caagagcagcuuagaggau

SEQ ID NO: 616 - RNA, 19 nt, Artificial Sequence
  Description of Artificial Sequence Synthetic oligonucleotide
ggaaagcaucucuggcuca

SEQ ID NO: 617 - RNA, 19 nt, Artificial Sequence
  Description of Artificial Sequence Synthetic oligonucleotide
accgagagcuuguggucau

SEQ ID NO: 618 - RNA, 19 nt, Artificial Sequence
  Description of Artificial Sequence Synthetic oligonucleotide
gcagguacaagaagcucaa

SEQ ID NO: 619 - RNA, 19 nt, Artificial Sequence
  Description of Artificial Sequence Synthetic oligonucleotide
gagaaggaggaguuuguga

SEQ ID NO: 620 - RNA, 19 nt, Artificial Sequence
  Description of Artificial Sequence Synthetic oligonucleotide
cagcacuucuauagcguca

SEQ ID NO: 621 - RNA, 19 nt, Artificial Sequence
  Description of Artificial Sequence Synthetic oligonucleotide
gggcggaaguccugauggu

SEQ ID NO: 622 - RNA, 19 nt, Artificial Sequence
  Description of Artificial Sequence Synthetic oligonucleotide
caagauugucucauaccua

SEQ ID NO: 623 - RNA, 19 nt, Artificial Sequence
  Description of Artificial Sequence Synthetic oligonucleotide
caucgaggaucuagaggcu

SEQ ID NO: 624 - RNA, 19 nt, Artificial Sequence
  Description of Artificial Sequence Synthetic oligonucleotide
gcacuucuauagcgucaaa

SEQ ID NO: 625 - RNA, 19 nt, Artificial Sequence
  Description of Artificial Sequence Synthetic oligonucleotide
cagcaugugcauuuccuaa

SEQ ID NO: 626 - RNA, 19 nt, Artificial Sequence
  Description of Artificial Sequence Synthetic oligonucleotide
gaggaucucccaaggauga

SEQ ID NO: 627 - RNA, 19 nt, Artificial Sequence
  Description of Artificial Sequence Synthetic oligonucleotide
agagaaaugagcuuggcuu

SEQ ID NO: 628 - RNA, 19 nt, Artificial Sequence
  Description of Artificial Sequence Synthetic oligonucleotide
gagcuuggcuugcaacuca

SEQ ID NO: 629 - RNA, 19 nt, Artificial Sequence
  Description of Artificial Sequence Synthetic oligonucleotide
cuuugaggccagaggugau

SEQ ID NO: 630 - RNA, 19 nt, Artificial Sequence
  Description of Artificial Sequence Synthetic oligonucleotide
uggagaaggaggaguuugu

SEQ ID NO: 631 - RNA, 19 nt, Artificial Sequence
  Description of Artificial Sequence Synthetic oligonucleotide
ucgaggaucuagaggcugu

SEQ ID NO: 632 - RNA, 19 nt, Artificial Sequence
  Description of Artificial Sequence Synthetic oligonucleotide
ugaaagaaugucaagccau

SEQ ID NO: 633 - RNA, 19 nt, Artificial Sequence
  Description of Artificial Sequence Synthetic oligonucleotide
aaugagagaggcaggcaga

SEQ ID NO: 634 - RNA, 19 nt, Artificial Sequence
  Description of Artificial Sequence Synthetic oligonucleotide
gggacauugccucuggcua

SEQ ID NO: 635 - RNA, 19 nt, Artificial Sequence
  Description of Artificial Sequence Synthetic oligonucleotide
ccaagggaacauugaguac

SEQ ID NO: 636 - RNA, 19 nt, Artificial Sequence
  Description of Artificial Sequence Synthetic oligonucleotide
gcgccuugaucgagugcgu

SEQ ID NO: 637 - RNA, 19 nt, Artificial Sequence
  Description of Artificial Sequence Synthetic oligonucleotide
auaccugagcuuacaaauu

SEQ ID NO: 638 - RNA, 19 nt, Artificial Sequence
  Description of Artificial Sequence Synthetic oligonucleotide
ccuggcagaccgagagcuu

SEQ ID NO: 639 - RNA, 19 nt, Artificial Sequence
  Description of Artificial Sequence Synthetic oligonucleotide
ccaccaagaggcagcaugu

SEQ ID NO: 640 - RNA, 19 nt, Artificial Sequence
  Description of Artificial Sequence Synthetic oligonucleotide
augaaagaaugucaagcca

SEQ ID NO: 641 - RNA, 19 nt, Artificial Sequence
  Description of Artificial Sequence Synthetic oligonucleotide
gagaaaugagcuuggcuug

SEQ ID NO: 642 - RNA, 19 nt, Artificial Sequence
  Description of Artificial Sequence Synthetic oligonucleotide
ccaaaaugguguccagaac

SEQ ID NO: 643 - RNA, 19 nt, Artificial Sequence
  Description of Artificial Sequence Synthetic oligonucleotide
ggacuauccaagggaacau

SEQ ID NO: 644 - RNA, 19 nt, Artificial Sequence
  Description of Artificial Sequence Synthetic oligonucleotide
cuucaugaaaugccucaaa

SEQ ID NO: 645 - RNA, 19 nt, Artificial Sequence
  Description of Artificial Sequence Synthetic oligonucleotide
gcaaaguggaggagaauaa

SEQ ID NO: 646 - RNA, 19 nt, Artificial Sequence
  Description of Artificial Sequence Synthetic oligonucleotide
ggauguggcuggaaagaaa

SEQ ID NO: 647 - RNA, 19 nt, Artificial Sequence
  Description of Artificial Sequence Synthetic oligonucleotide
ggaaauugaugaagaugaa

SEQ ID NO: 648 - RNA, 19 nt, Artificial Sequence
  Description of Artificial Sequence Synthetic oligonucleotide
caacacaggugaaggaaau

SEQ ID NO: 649 - RNA, 19 nt, Artificial Sequence
  Description of Artificial Sequence Synthetic oligonucleotide
ccacaagaaucuagcagaa

SEQ ID NO: 650 - RNA, 19 nt, Artificial Sequence
  Description of Artificial Sequence Synthetic oligonucleotide
ggaggaaacauuuaagaaa

SEQ ID NO: 651 - RNA, 19 nt, Artificial Sequence
  Description of Artificial Sequence Synthetic oligonucleotide
gaucagaacacaagagaga

SEQ ID NO: 652 - RNA, 19 nt, Artificial Sequence
  Description of Artificial Sequence Synthetic oligonucleotide
gugcagagaagcaggcaaa

SEQ ID NO: 653 - RNA, 19 nt, Artificial Sequence
  Description of Artificial Sequence Synthetic oligonucleotide
acagcaaaguggaggagaa

SEQ ID NO: 654 - RNA, 19 nt, Artificial Sequence
  Description of Artificial Sequence Synthetic oligonucleotide
ccauagaggugguccagaa

SEQ ID NO: 655 - RNA, 19 nt, Artificial Sequence
  Description of Artificial Sequence Synthetic oligonucleotide
ccaugaaggaagugaccua

SEQ ID NO: 656 - RNA, 19 nt, Artificial Sequence
  Description of Artificial Sequence Synthetic oligonucleotide
gggaaaagauuacagcaaa

SEQ ID NO: 657 - RNA, 19 nt, Artificial Sequence
  Description of Artificial Sequence Synthetic oligonucleotide
aaaagaagguagaaagcaa

SEQ ID NO: 658 - RNA, 19 nt, Artificial Sequence
  Description of Artificial Sequence Synthetic oligonucleotide
gguagaaagcaaauccaaa

SEQ ID NO: 659 - RNA, 19 nt, Artificial Sequence
  Description of Artificial Sequence Synthetic oligonucleotide
ccacagaggcgguucagaa

SEQ ID NO: 660 - RNA, 19 nt, Artificial Sequence
  Description of Artificial Sequence Synthetic oligonucleotide
ucagaaaguaggagcagaa

SEQ ID NO: 661 - RNA, 19 nt, Artificial Sequence
  Description of Artificial Sequence Synthetic oligonucleotide
gggcaggaguaaugaaacu

SEQ ID NO: 662 - RNA, 19 nt, Artificial Sequence
  Description of Artificial Sequence Synthetic oligonucleotide
ugaagagucugcugauaua

SEQ ID NO: 663 - RNA, 19 nt, Artificial Sequence
  Description of Artificial Sequence Synthetic oligonucleotide
ggcaagagcucgaagacca

SEQ ID NO: 664 - RNA, 19 nt, Artificial Sequence
  Description of Artificial Sequence Synthetic oligonucleotide
ggaagagagugcagagaag

SEQ ID NO: 665 - RNA, 19 nt, Artificial Sequence
  Description of Artificial Sequence Synthetic oligonucleotide
cuucuaaaggaaaguguaa

SEQ ID NO: 666 - RNA, 19 nt, Artificial Sequence
  Description of Artificial Sequence Synthetic oligonucleotide
gagaagaggcaucaggaga

SEQ ID NO: 667 - RNA, 19 nt, Artificial Sequence
  Description of Artificial Sequence Synthetic oligonucleotide
ggugaaacuuuaaauggua

SEQ ID NO: 668 - RNA, 19 nt, Artificial Sequence
  Description of Artificial Sequence Synthetic oligonucleotide
agauagugaagaaggagaa

SEQ ID NO: 669 - RNA, 19 nt, Artificial Sequence
  Description of Artificial Sequence Synthetic oligonucleotide
ugaagaaggagaaggacuu

SEQ ID NO: 670 - RNA, 19 nt, Artificial Sequence
  Description of Artificial Sequence Synthetic oligonucleotide
ccagauauuuacaguucaa

SEQ ID NO: 671 - RNA, 19 nt, Artificial Sequence
  Description of Artificial Sequence Synthetic oligonucleotide
gagcggaggacaaaggcaa

SEQ ID NO: 672 - RNA, 19 nt, Artificial Sequence
  Description of Artificial Sequence Synthetic oligonucleotide
uaacagaggugaaagagau

SEQ ID NO: 673 - RNA, 19 nt, Artificial Sequence
  Description of Artificial Sequence Synthetic oligonucleotide
acaggaagcaauucagaaa

SEQ ID NO: 674 - RNA, 19 nt, Artificial Sequence
  Description of Artificial Sequence Synthetic oligonucleotide
aggaagugccaaaggguga

SEQ ID NO: 675 - RNA, 19 nt, Artificial Sequence
  Description of Artificial Sequence Synthetic oligonucleotide
gaaggagccuguucagaua

SEQ ID NO: 676 - RNA, 19 nt, Artificial Sequence
  Description of Artificial Sequence Synthetic oligonucleotide
agucuaacaugcagaguga

SEQ ID NO: 677 - RNA, 19 nt, Artificial Sequence
  Description of Artificial Sequence Synthetic oligonucleotide
ucuaacaugcagagugaaa

SEQ ID NO: 678 - RNA, 19 nt, Artificial Sequence
  Description of Artificial Sequence Synthetic oligonucleotide
ccuuauugaaguuggcuua

SEQ ID NO: 679 - RNA, 19 nt, Artificial Sequence
  Description of Artificial Sequence Synthetic oligonucleotide
caguaacagaggugaaaga

SEQ ID NO: 680 - RNA, 19 nt, Artificial Sequence
  Description of Artificial Sequence Synthetic oligonucleotide
ggaagugaccuaagugaca

SEQ ID NO: 681 - RNA, 19 nt, Artificial Sequence
  Description of Artificial Sequence Synthetic oligonucleotide
gugauuaccuggucgguga

SEQ ID NO: 682 - RNA, 19 nt, Artificial Sequence
  Description of Artificial Sequence Synthetic oligonucleotide
gaguaucacuggaggaaac

SEQ ID NO: 683 - RNA, 19 nt, Artificial Sequence
  Description of Artificial Sequence Synthetic oligonucleotide
aggagaacgcccauauaaa

SEQ ID NO: 684 - RNA, 19 nt, Artificial Sequence
  Description of Artificial Sequence Synthetic oligonucleotide
gaugaggaaucuucaacaa

SEQ ID NO: 685 - RNA, 19 nt, Artificial Sequence
  Description of Artificial Sequence Synthetic oligonucleotide
gccaaagggugacagcaaa

SEQ ID NO: 686 - RNA, 19 nt, Artificial Sequence
  Description of Artificial Sequence Synthetic oligonucleotide
agaaggaaccuguugagaa

SEQ ID NO: 687 - RNA, 19 nt, Artificial Sequence
  Description of Artificial Sequence Synthetic oligonucleotide
gagcagaagaggcagauga

SEQ ID NO: 688 - RNA, 19 nt, Artificial Sequence
  Description of Artificial Sequence Synthetic oligonucleotide
aaagaaaaguagucggaga

SEQ ID NO: 689 - RNA, 19 nt, Artificial Sequence
  Description of Artificial Sequence Synthetic oligonucleotide
aagaacaguuugugcauca

SEQ ID NO: 690 - RNA, 19 nt, Artificial Sequence
  Description of Artificial Sequence Synthetic oligonucleotide
gcuacaauacuaaucgaua

SEQ ID NO: 691 - RNA, 19 nt, Artificial Sequence
  Description of Artificial Sequence Synthetic oligonucleotide
aaacaauggaugucucaaa

SEQ ID NO: 692 - RNA, 19 nt, Artificial Sequence
  Description of Artificial Sequence Synthetic oligonucleotide
aaucaguaacagaggugaa

SEQ ID NO: 693 - RNA, 19 nt, Artificial Sequence
  Description of Artificial Sequence Synthetic oligonucleotide
gugcauacaggaagcaauu

SEQ ID NO: 694 - RNA, 19 nt, Artificial Sequence
  Description of Artificial Sequence Synthetic oligonucleotide
ggaaauggccgaagagaaa

SEQ ID NO: 695 - RNA, 19 nt, Artificial Sequence
  Description of Artificial Sequence Synthetic oligonucleotide
cgagaaagagggagaggaa

SEQ ID NO: 696 - RNA, 19 nt, Artificial Sequence
  Description of Artificial Sequence Synthetic oligonucleotide
agaaagagggagaggaaga

SEQ ID NO: 697 - RNA, 19 nt, Artificial Sequence
  Description of Artificial Sequence Synthetic oligonucleotide
guaaauagguggaauauga

SEQ ID NO: 698 - RNA, 19 nt, Artificial Sequence
  Description of Artificial Sequence Synthetic oligonucleotide
caacaacauuucagacaaa

SEQ ID NO: 699 - RNA, 19 nt, Artificial Sequence
  Description of Artificial Sequence Synthetic oligonucleotide
gaauaugaaugcuuggaaa

SEQ ID NO: 700 - RNA, 19 nt, Artificial Sequence
  Description of Artificial Sequence Synthetic oligonucleotide
aagaagagguggaggacga

SEQ ID NO: 701 - RNA, 19 nt, Artificial Sequence
  Description of Artificial Sequence Synthetic oligonucleotide
aggacaaggaagagagaga

SEQ ID NO: 702 - RNA, 19 nt, Artificial Sequence
  Description of Artificial Sequence Synthetic oligonucleotide
agggagaggaagacagaua

SEQ ID NO: 703 - RNA, 19 nt, Artificial Sequence
  Description of Artificial Sequence Synthetic oligonucleotide
gugccugccuauagagaua

SEQ ID NO: 704 - RNA, 19 nt, Artificial Sequence
  Description of Artificial Sequence Synthetic oligonucleotide
ccgaaaugccaaagaggau

SEQ ID NO: 705 - RNA, 19 nt, Artificial Sequence
  Description of Artificial Sequence Synthetic oligonucleotide
uggaaauggccgaagagaa

SEQ ID NO: 706 - RNA, 19 nt, Artificial Sequence
  Description of Artificial Sequence Synthetic oligonucleotide
cuuguaaauagguggaaua

SEQ ID NO: 707 - RNA, 19 nt, Artificial Sequence
  Description of Artificial Sequence Synthetic oligonucleotide
gagagaugguuuaaagaca

SEQ ID NO: 708 - RNA, 19 nt, Artificial Sequence
  Description of Artificial Sequence Synthetic oligonucleotide
ggagaagagcccagcaaga

SEQ ID NO: 709 - RNA, 19 nt, Artificial Sequence
  Description of Artificial Sequence Synthetic oligonucleotide
ccgaagaagagguggagga

SEQ ID NO: 710 - RNA, 19 nt, Artificial Sequence
  Description of Artificial Sequence Synthetic oligonucleotide
cugcauaccagaaugauaa

SEQ ID NO: 711 - RNA, 19 nt, Artificial Sequence
  Description of Artificial Sequence Synthetic oligonucleotide
ggacaagugaacacauuaa

SEQ ID NO: 712 - RNA, 19 nt, Artificial Sequence
  Description of Artificial Sequence Synthetic oligonucleotide
gcacuuugucggauauaaa

SEQ ID NO: 713 - RNA, 19 nt, Artificial Sequence
  Description of Artificial Sequence Synthetic oligonucleotide
gagaugguuuaaagacaaa

SEQ ID NO: 714 - RNA, 19 nt, Artificial Sequence
  Description of Artificial Sequence Synthetic oligonucleotide
ccauggagcccgaagaaga

SEQ ID NO: 715 - RNA, 19 nt, Artificial Sequence
  Description of Artificial Sequence Synthetic oligonucleotide
gcugaugacugucguuaua

SEQ ID NO: 716 - RNA, 19 nt, Artificial Sequence
  Description of Artificial Sequence Synthetic oligonucleotide
caucgaaccucaaagauuu

SEQ ID NO: 717 - RNA, 19 nt, Artificial Sequence
  Description of Artificial Sequence Synthetic oligonucleotide
cggacucccucgagagaau

SEQ ID NO: 718 - RNA, 19 nt, Artificial Sequence
  Description of Artificial Sequence Synthetic oligonucleotide
agugagacuauuagacaaa

SEQ ID NO: 719 - RNA, 19 nt, Artificial Sequence
  Description of Artificial Sequence Synthetic oligonucleotide
agagaugguuuaaagacaa

SEQ ID NO: 720 - RNA, 19 nt, Artificial Sequence
  Description of Artificial Sequence Synthetic oligonucleotide
gguggaugguggcugguaa

SEQ ID NO: 721 - RNA, 19 nt, Artificial Sequence
  Description of Artificial Sequence Synthetic oligonucleotide
ccagcgaacugcagagcau

SEQ ID NO: 722 - RNA, 19 nt, Artificial Sequence
  Description of Artificial Sequence Synthetic oligonucleotide
gcgccuggacacagauuua

SEQ ID NO: 723 - RNA, 19 nt, Artificial Sequence
  Description of Artificial Sequence Synthetic oligonucleotide
ccagcgagaaagagggaga

SEQ ID NO: 724 - RNA, 19 nt, Artificial Sequence
  Description of Artificial Sequence Synthetic oligonucleotide
ccaacaacauuucagacaa

SEQ ID NO: 725 - RNA, 19 nt, Artificial Sequence
  Description of Artificial Sequence Synthetic oligonucleotide
gcaaaagguuuccgggaca

SEQ ID NO: 726 - RNA, 19 nt, Artificial Sequence
  Description of Artificial Sequence Synthetic oligonucleotide
agagaaugugcuagagaca

SEQ ID NO: 727 - RNA, 19 nt, Artificial Sequence
  Description of Artificial Sequence Synthetic oligonucleotide
gguaggaguuccaacauuu

SEQ ID NO: 728 - RNA, 19 nt, Artificial Sequence
  Description of Artificial Sequence Synthetic oligonucleotide
ccaaugacaucuugaaacu

SEQ ID NO: 729 - RNA, 19 nt, Artificial Sequence
  Description of Artificial Sequence Synthetic oligonucleotide
ggacacagauuuaggaagc

SEQ ID NO: 730 - RNA, 19 nt, Artificial Sequence
  Description of Artificial Sequence Synthetic oligonucleotide
cgacuauguuugcugauuu

SEQ ID NO: 731 - RNA, 19 nt, Artificial Sequence
  Description of Artificial Sequence Synthetic oligonucleotide
gugcauuaguugugauuuc

SEQ ID NO: 732 - RNA, 19 nt, Artificial Sequence
  Description of Artificial Sequence Synthetic oligonucleotide
aaagggaaggagugggcaa

SEQ ID NO: 733 - RNA, 19 nt, Artificial Sequence
  Description of Artificial Sequence Synthetic oligonucleotide
ccuggaggcuaaagaacuu

SEQ ID NO: 734 - RNA, 19 nt, Artificial Sequence
  Description of Artificial Sequence Synthetic oligonucleotide
ccaugaggguguuugauga

SEQ ID NO: 735 - RNA, 19 nt, Artificial Sequence
  Description of Artificial Sequence Synthetic oligonucleotide
ccgugcacuuugucggaua

SEQ ID NO: 736 - RNA, 19 nt, Artificial Sequence
  Description of Artificial Sequence Synthetic oligonucleotide
ggauuuaaagggaaggagu

SEQ ID NO: 737 - RNA, 19 nt, Artificial Sequence
  Description of Artificial Sequence Synthetic oligonucleotide
aagugagacuauuagacaa

SEQ ID NO: 738 - RNA, 19 nt, Artificial Sequence
  Description of Artificial Sequence Synthetic oligonucleotide
gacaaauucaugaagguau

SEQ ID NO: 739 - RNA, 19 nt, Artificial Sequence
  Description of Artificial Sequence Synthetic oligonucleotide
guguuauaguuguugauga

SEQ ID NO: 740 - RNA, 19 nt, Artificial Sequence
  Description of Artificial Sequence Synthetic oligonucleotide
acgcagggcuggagugucu

SEQ ID NO: 741 - RNA, 19 nt, Artificial Sequence
  Description of Artificial Sequence Synthetic oligonucleotide
ccauuuaaagugagauguu

SEQ ID NO: 742 - RNA, 19 nt, Artificial Sequence
  Description of Artificial Sequence Synthetic oligonucleotide
caaagaggauguacauuca

SEQ ID NO: 743 - RNA, 19 nt, Artificial Sequence
  Description of Artificial Sequence Synthetic oligonucleotide
acaucgaaccucaaagauu

SEQ ID NO: 744 - RNA, 19 nt, Artificial Sequence
  Description of Artificial Sequence Synthetic oligonucleotide
gggaauaguagcugucaaa

SEQ ID NO: 745 - RNA, 19 nt, Artificial Sequence
  Description of Artificial Sequence Synthetic oligonucleotide
ccagauaugcacagauaaa

SEQ ID NO: 746 - RNA, 19 nt, Artificial Sequence
  Description of Artificial Sequence Synthetic oligonucleotide
ccagauaacacguaaguuu

SEQ ID NO: 747 - RNA, 19 nt, Artificial Sequence
  Description of Artificial Sequence Synthetic oligonucleotide
ggccagauaugcacagaua

SEQ ID NO: 748 - RNA, 19 nt, Artificial Sequence
  Description of Artificial Sequence Synthetic oligonucleotide
uguaaauaacccaggaaau

SEQ ID NO: 749 - RNA, 19 nt, Artificial Sequence
  Description of Artificial Sequence Synthetic oligonucleotide
ccucaaagccgaacuaaau

SEQ ID NO: 750 - RNA, 19 nt, Artificial Sequence
  Description of Artificial Sequence Synthetic oligonucleotide
agaagaaggacgcggugaa

SEQ ID NO: 751 - RNA, 19 nt, Artificial Sequence
  Description of Artificial Sequence Synthetic oligonucleotide
acagauuggaguuggcaua

SEQ ID NO: 752 - RNA, 19 nt, Artificial Sequence
  Description of Artificial Sequence Synthetic oligonucleotide
ggagauggcgauuugauua

SEQ ID NO: 753 - RNA, 19 nt, Artificial Sequence
  Description of Artificial Sequence Synthetic oligonucleotide
aggcaacacuuaagcagua

SEQ ID NO: 754 - RNA, 19 nt, Artificial Sequence
  Description of Artificial Sequence Synthetic oligonucleotide
ugaaggacaaggaggagaa

SEQ ID NO: 755 - RNA, 19 nt, Artificial Sequence
  Description of Artificial Sequence Synthetic oligonucleotide
ggaccaaacgccagaaagu

SEQ ID NO: 756 - RNA, 19 nt, Artificial Sequence
  Description of Artificial Sequence Synthetic oligonucleotide
cggugaaggacaaggagga

SEQ ID NO: 757 - RNA, 19 nt, Artificial Sequence
  Description of Artificial Sequence Synthetic oligonucleotide
gccagaaaguguucccaaa

SEQ ID NO: 758 - RNA, 19 nt, Artificial Sequence
  Description of Artificial Sequence Synthetic oligonucleotide
gauuggaguuggcauauaa

SEQ ID NO: 759 - RNA, 19 nt, Artificial Sequence
  Description of Artificial Sequence Synthetic oligonucleotide
gguuggaaagggauauuua

SEQ ID NO: 760 - RNA, 19 nt, Artificial Sequence
  Description of Artificial Sequence Synthetic oligonucleotide
ggaaagggauauuuaaucu

SEQ ID NO: 761 - RNA, 19 nt, Artificial Sequence
  Description of Artificial Sequence Synthetic oligonucleotide
cgggaauaguagcugucaa

SEQ ID NO: 762 - RNA, 19 nt, Artificial Sequence
  Description of Artificial Sequence Synthetic oligonucleotide
cgagaggagcagaacauuu

SEQ ID NO: 763 - RNA, 19 nt, Artificial Sequence
  Description of Artificial Sequence Synthetic oligonucleotide
gaucauuguuaaaggauug

SEQ ID NO: 764 - RNA, 19 nt, Artificial Sequence
  Description of Artificial Sequence Synthetic oligonucleotide
aggcaaaaucgaaacuaaa

SEQ ID NO: 765 - RNA, 19 nt, Artificial Sequence
  Description of Artificial Sequence Synthetic oligonucleotide
gaguuggcauauaaacaaa

SEQ ID NO: 766 - RNA, 19 nt, Artificial Sequence
  Description of Artificial Sequence Synthetic oligonucleotide
auucauuaucuuaggguga

SEQ ID NO: 767 - RNA, 19 nt, Artificial Sequence
  Description of Artificial Sequence Synthetic oligonucleotide
aggacgcggugaaggacaa

SEQ ID NO: 768 - RNA, 19 nt, Artificial Sequence
  Description of Artificial Sequence Synthetic oligonucleotide
cuaaauaaacaaacccgua

SEQ ID NO: 769 - RNA, 19 nt, Artificial Sequence
  Description of Artificial Sequence Synthetic oligonucleotide
acagcaaaaucuugguuua

SEQ ID NO: 770 - RNA, 19 nt, Artificial Sequence
  Description of Artificial Sequence Synthetic oligonucleotide
ggaguuggcauauaaacaa

SEQ ID NO: 771 - RNA, 19 nt, Artificial Sequence
  Description of Artificial Sequence Synthetic oligonucleotide
gggacugugcggccagaua

SEQ ID NO: 772 - RNA, 19 nt, Artificial Sequence
  Description of Artificial Sequence Synthetic oligonucleotide
ggcgagaggagcagaacau

SEQ ID NO: 773 - RNA, 19 nt, Artificial Sequence
  Description of Artificial Sequence Synthetic oligonucleotide
cccucaaagccgaacuaaa

SEQ ID NO: 774 - RNA, 19 nt, Artificial Sequence
  Description of Artificial Sequence Synthetic oligonucleotide
aggaacccaucaaggcaaa

SEQ ID NO: 775 - RNA, 19 nt, Artificial Sequence
  Description of Artificial Sequence Synthetic oligonucleotide
caucaaggcaaaaucgaaa

SEQ ID NO: 776 - RNA, 19 nt, Artificial Sequence
  Description of Artificial Sequence Synthetic oligonucleotide
gggaaacuguauuaaucuu

SEQ ID NO: 777 - RNA, 19 nt, Artificial Sequence
  Description of Artificial Sequence Synthetic oligonucleotide
uggagaaacccucugacua

SEQ ID NO: 778 - RNA, 19 nt, Artificial Sequence
  Description of Artificial Sequence Synthetic oligonucleotide
aguuaaaccuaggggacaa

SEQ ID NO: 779 - RNA, 19 nt, Artificial Sequence
  Description of Artificial Sequence Synthetic oligonucleotide
gcuccuaucuagaggcaac

SEQ ID NO: 780 - RNA, 19 nt, Artificial Sequence
  Description of Artificial Sequence Synthetic oligonucleotide
gaacaacucuccagugaac

SEQ ID NO: 781 - RNA, 19 nt, Artificial Sequence
  Description of Artificial Sequence Synthetic oligonucleotide
ggacaguguuacuccagau

SEQ ID NO: 782 - RNA, 19 nt, Artificial Sequence
  Description of Artificial Sequence Synthetic oligonucleotide
ccucucaccuguaagauau

SEQ ID NO: 783 - RNA, 19 nt, Artificial Sequence
  Description of Artificial Sequence Synthetic oligonucleotide
aguuggaugucguggacca

SEQ ID NO: 784 - RNA, 19 nt, Artificial Sequence
  Description of Artificial Sequence Synthetic oligonucleotide
ggagaaacccucugacuag

SEQ ID NO: 785 - RNA, 19 nt, Artificial Sequence
  Description of Artificial Sequence Synthetic oligonucleotide
ggucuagggugguuucuuu

SEQ ID NO: 786 - RNA, 19 nt, Artificial Sequence
  Description of Artificial Sequence Synthetic oligonucleotide
uuguaaauaacccaggaaa

SEQ ID NO: 787 - RNA, 19 nt, Artificial Sequence
  Description of Artificial Sequence Synthetic oligonucleotide
gggagauggcgauuugauu

SEQ ID NO: 788 - RNA, 19 nt, Artificial Sequence
  Description of Artificial Sequence Synthetic oligonucleotide
cgauuugauuacagacguu

SEQ ID NO: 789 - RNA, 19 nt, Artificial Sequence
  Description of Artificial Sequence Synthetic oligonucleotide
aguaauugcuguugcuugu

SEQ ID NO: 790 - RNA, 19 nt, Artificial Sequence
  Description of Artificial Sequence Synthetic oligonucleotide
gcuguugcuuguugucaaa

SEQ ID NO: 791 - RNA, 19 nt, Artificial Sequence
  Description of Artificial Sequence Synthetic oligonucleotide
agaagaagguggugaucaa

SEQ ID NO: 792 - RNA, 19 nt, Artificial Sequence
  Description of Artificial Sequence Synthetic oligonucleotide
ccaaggaggccgagaagaa

SEQ ID NO: 793 - RNA, 19 nt, Artificial Sequence
  Description of Artificial Sequence Synthetic oligonucleotide
gcuucagcguggagaacau

SEQ ID NO: 794 - RNA, 19 nt, Artificial Sequence
  Description of Artificial Sequence Synthetic oligonucleotide
ccgagaagaagguggugau

SEQ ID NO: 795 - RNA, 19 nt, Artificial Sequence
  Description of Artificial Sequence Synthetic oligonucleotide
gagaagaagaucaccuuga

SEQ ID NO: 796 - RNA, 19 nt, Artificial Sequence
  Description of Artificial Sequence Synthetic oligonucleotide
cgccuaaggaccuggugaa

SEQ ID NO: 797 - RNA, 19 nt, Artificial Sequence
  Description of Artificial Sequence Synthetic oligonucleotide
ccuacgacugcacgaaaua

SEQ ID NO: 798 - RNA, 19 nt, Artificial Sequence
  Description of Artificial Sequence Synthetic oligonucleotide
uguccaaggagaaggagga

SEQ ID NO: 799 - RNA, 19 nt, Artificial Sequence
  Description of Artificial Sequence Synthetic oligonucleotide
agaagaagaucaccuugaa

SEQ ID NO: 800 - RNA, 19 nt, Artificial Sequence
  Description of Artificial Sequence Synthetic oligonucleotide
agguggugaucaagagcga

SEQ ID NO: 801 - RNA, 19 nt, Artificial Sequence
  Description of Artificial Sequence Synthetic oligonucleotide
gagaagaagguggugauca

SEQ ID NO: 802 - RNA, 19 nt, Artificial Sequence
  Description of Artificial Sequence Synthetic oligonucleotide
ccaacgugcgggagauguu

SEQ ID NO: 803 - RNA, 19 nt, Artificial Sequence
  Description of Artificial Sequence Synthetic oligonucleotide
cagaauuacuaccgggcug

SEQ ID NO: 804 - RNA, 19 nt, Artificial Sequence
  Description of Artificial Sequence Synthetic oligonucleotide
gggagaacaagcagggcug

SEQ ID NO: 805 - RNA, 19 nt, Artificial Sequence
  Description of Artificial Sequence Synthetic oligonucleotide
accugagcgagcagaauua

SEQ ID NO: 806 - RNA, 19 nt, Artificial Sequence
  Description of Artificial Sequence Synthetic oligonucleotide
ccgagaagaagaucaccuu

SEQ ID NO: 807 - RNA, 19 nt, Artificial Sequence
  Description of Artificial Sequence Synthetic oligonucleotide
ugagcgagcagaauuacua

SEQ ID NO: 808 - RNA, 19 nt, Artificial Sequence
  Description of Artificial Sequence Synthetic oligonucleotide
gcgccuaaggaccugguga

SEQ ID NO: 809 - RNA, 19 nt, Artificial Sequence
  Description of Artificial Sequence Synthetic oligonucleotide
ccuaccugagcgagcagaa

SEQ ID NO: 810 - RNA, 19 nt, Artificial Sequence
  Description of Artificial Sequence Synthetic oligonucleotide
aagaagguggugaucaaga

SEQ ID NO: 811 - RNA, 19 nt, Artificial Sequence
  Description of Artificial Sequence Synthetic oligonucleotide
cagugcagcaugcgagcga

SEQ ID NO: 812 - RNA, 19 nt, Artificial Sequence
  Description of Artificial Sequence Synthetic oligonucleotide
cggcccagcagcaaacuuu

SEQ ID NO: 813 - RNA, 19 nt, Artificial Sequence
  Description of Artificial Sequence Synthetic oligonucleotide
uggagaacaucaugacccu

SEQ ID NO: 814 - RNA, 19 nt, Artificial Sequence
  Description of Artificial Sequence Synthetic oligonucleotide
cgggagaacaagcagggcu

SEQ ID NO: 815 - RNA, 19 nt, Artificial Sequence
  Description of Artificial Sequence Synthetic oligonucleotide
cuggcuucagcguggagaa

SEQ ID NO: 816 - RNA, 19 nt, Artificial Sequence
  Description of Artificial Sequence Synthetic oligonucleotide
ggauugagaacucgacccu

SEQ ID NO: 817 - RNA, 19 nt, Artificial Sequence
  Description of Artificial Sequence Synthetic oligonucleotide
gucccaggugaguggcaau

SEQ ID NO: 818 - RNA, 19 nt, Artificial Sequence
  Description of Artificial Sequence Synthetic oligonucleotide
aagaucaccuugaacggca

SEQ ID NO: 819 - RNA, 19 nt, Artificial Sequence
  Description of Artificial Sequence Synthetic oligonucleotide
gugcagcaugcgagcgaug

SEQ ID NO: 820 - RNA, 19 nt, Artificial Sequence
  Description of Artificial Sequence Synthetic oligonucleotide
uccuacgacugcacgaaau

SEQ ID NO: 821 - RNA, 19 nt, Artificial Sequence
  Description of Artificial Sequence Synthetic oligonucleotide
cuaaggaccuggugaagcc

SEQ ID NO: 822 - RNA, 19 nt, Artificial Sequence
  Description of Artificial Sequence Synthetic oligonucleotide
agaucaccuugaacggcau

SEQ ID NO: 823 - RNA, 19 nt, Artificial Sequence
  Description of Artificial Sequence Synthetic oligonucleotide
ccaaggagaaggaggagcg

SEQ ID NO: 824 - RNA, 19 nt, Artificial Sequence
  Description of Artificial Sequence Synthetic oligonucleotide
gccgagaagaaggugguga

SEQ ID NO: 825 - RNA, 19 nt, Artificial Sequence
  Description of Artificial Sequence Synthetic oligonucleotide
cagcugcccuacagaucca

SEQ ID NO: 826 - RNA, 19 nt, Artificial Sequence
  Description of Artificial Sequence Synthetic oligonucleotide
acaucaugacccugcgaac

SEQ ID NO: 827 - RNA, 19 nt, Artificial Sequence
  Description of Artificial Sequence Synthetic oligonucleotide
agucccaggugaguggcaa

SEQ ID NO: 828 - RNA, 19 nt, Artificial Sequence
  Description of Artificial Sequence Synthetic oligonucleotide
cuaccugagcgagcagaau

SEQ ID NO: 829 - RNA, 19 nt, Artificial Sequence
  Description of Artificial Sequence Synthetic oligonucleotide
ggagaagagggaagaggaa

SEQ ID NO: 830 - RNA, 19 nt, Artificial Sequence
  Description of Artificial Sequence Synthetic oligonucleotide
gcggagaaguggaacaaga

SEQ ID NO: 831 - RNA, 19 nt, Artificial Sequence
  Description of Artificial Sequence Synthetic oligonucleotide
caucagaggagucggagaa

SEQ ID NO: 832 - RNA, 19 nt, Artificial Sequence
  Description of Artificial Sequence Synthetic oligonucleotide
agagggaagaggaagguaa

SEQ ID NO: 833 - RNA, 19 nt, Artificial Sequence
  Description of Artificial Sequence Synthetic oligonucleotide
gagggaagaggaagguaaa

SEQ ID NO: 834 - RNA, 19 nt, Artificial Sequence
  Description of Artificial Sequence Synthetic oligonucleotide
ggaacgaggagcuguaaau

SEQ ID NO: 835 - RNA, 19 nt, Artificial Sequence
  Description of Artificial Sequence Synthetic oligonucleotide
acaauaaagugauggcgau

SEQ ID NO: 836 - RNA, 19 nt, Artificial Sequence
  Description of Artificial Sequence Synthetic oligonucleotide
cgaaggacuugcacagaca

SEQ ID NO: 837 - RNA, 19 nt, Artificial Sequence
  Description of Artificial Sequence Synthetic oligonucleotide
auaaagugauggcgaugua

SEQ ID NO: 838 - RNA, 19 nt, Artificial Sequence
  Description of Artificial Sequence Synthetic oligonucleotide
ugacaguacaauaaaguga

SEQ ID NO: 839 - RNA, 19 nt, Artificial Sequence
  Description of Artificial Sequence Synthetic oligonucleotide
agucggagaacgcggagaa

SEQ ID NO: 840 - RNA, 19 nt, Artificial Sequence
  Description of Artificial Sequence Synthetic oligonucleotide
aggagaaaguggaggucug

SEQ ID NO: 841 - RNA, 19 nt, Artificial Sequence
  Description of Artificial Sequence Synthetic oligonucleotide
cggcagaagcgguccucau

SEQ ID NO: 842 - RNA, 19 nt, Artificial Sequence
  Description of Artificial Sequence Synthetic oligonucleotide
cggagaagagggaagagga

SEQ ID NO: 843 - RNA, 19 nt, Artificial Sequence
  Description of Artificial Sequence Synthetic oligonucleotide
gccaaauggaggcggcaga

SEQ ID NO: 844 - RNA, 19 nt, Artificial Sequence
  Description of Artificial Sequence Synthetic oligonucleotide
uuaccuaacucgaaggacu

SEQ ID NO: 845 - RNA, 19 nt, Artificial Sequence
  Description of Artificial Sequence Synthetic oligonucleotide
cgagaaagaggaacgagga

SEQ ID NO: 846 - RNA, 19 nt, Artificial Sequence
  Description of Artificial Sequence Synthetic oligonucleotide
agaggaacgaggagcugua

SEQ ID NO: 847 - RNA, 19 nt, Artificial Sequence
  Description of Artificial Sequence Synthetic oligonucleotide
gaaagaggaacgaggagcu

SEQ ID NO: 848 - RNA, 19 nt, Artificial Sequence
  Description of Artificial Sequence Synthetic oligonucleotide
acgaggagcuguaaauagu

SEQ ID NO: 849 - RNA, 19 nt, Artificial Sequence
  Description of Artificial Sequence Synthetic oligonucleotide
ggaaagugcaccuccgcga

SEQ ID NO: 850 - RNA, 19 nt, Artificial Sequence
  Description of Artificial Sequence Synthetic oligonucleotide
gcgaggagaaaguggaggu

SEQ ID NO: 851 - RNA, 19 nt, Artificial Sequence
  Description of Artificial Sequence Synthetic oligonucleotide
cggagaacgcggagaagug

SEQ ID NO: 852 - RNA, 19 nt, Artificial Sequence
  Description of Artificial Sequence Synthetic oligonucleotide
gaggaagguaaaagcgauu

SEQ ID NO: 853 - RNA, 19 nt, Artificial Sequence
  Description of Artificial Sequence Synthetic oligonucleotide
gguaaaagcgauuuggacu

SEQ ID NO: 854 - RNA, 19 nt, Artificial Sequence
  Description of Artificial Sequence Synthetic oligonucleotide
aaguggaggucugguuuaa

SEQ ID NO: 855 - RNA, 19 nt, Artificial Sequence
  Description of Artificial Sequence Synthetic oligonucleotide
agacagacgaugcuacuuu

SEQ ID NO: 856 - RNA, 19 nt, Artificial Sequence
  Description of Artificial Sequence Synthetic oligonucleotide
aauuaagggugacaguaca

SEQ ID NO: 857 - RNA, 19 nt, Artificial Sequence
  Description of Artificial Sequence Synthetic oligonucleotide
aagggugacaguacaauaa

SEQ ID NO: 858 - RNA, 19 nt, Artificial Sequence
  Description of Artificial Sequence Synthetic oligonucleotide
aaagugauggcgauguaaa

SEQ ID NO: 859 - RNA, 19 nt, Artificial Sequence
  Description of Artificial Sequence Synthetic oligonucleotide
gcuacaacaacuacuucua

SEQ ID NO: 860 - RNA, 19 nt, Artificial Sequence
  Description of Artificial Sequence Synthetic oligonucleotide
acaacuacuucuacgggca

SEQ ID NO: 861 - RNA, 19 nt, Artificial Sequence
  Description of Artificial Sequence Synthetic oligonucleotide
gaacgaggagcuguaaaua

SEQ ID NO: 862 - RNA, 19 nt, Artificial Sequence
  Description of Artificial Sequence Synthetic oligonucleotide
auuaagggugacaguacaa

SEQ ID NO: 863 - RNA, 19 nt, Artificial Sequence
  Description of Artificial Sequence Synthetic oligonucleotide
guggaggucugguuuaaga

SEQ ID NO: 864 - RNA, 19 nt, Artificial Sequence
  Description of Artificial Sequence Synthetic oligonucleotide
acgcggagaaguggaacaa

SEQ ID NO: 865 - RNA, 19 nt, Artificial Sequence
  Description of Artificial Sequence Synthetic oligonucleotide
ucgaaggcgucaccggaga

SEQ ID NO: 866 - RNA, 19 nt, Artificial Sequence
  Description of Artificial Sequence Synthetic oligonucleotide
aaauuaagggugacaguac

SEQ ID NO: 867 - RNA, 19 nt, Artificial Sequence
  Description of Artificial Sequence Synthetic oligonucleotide
aagugauggcgauguaaaa

SEQ ID NO: 868 - RNA, 19 nt, Artificial Sequence
  Description of Artificial Sequence Synthetic oligonucleotide
ccgccagcauguucagcau

SEQ ID NO: 869 - RNA, 19 nt, Artificial Sequence
  Description of Artificial Sequence Synthetic oligonucleotide
aaaguggaggucugguuua

SEQ ID NO: 870 - RNA, 19 nt, Artificial Sequence
  Description of Artificial Sequence Synthetic oligonucleotide
ccaaauggaggcggcagaa

SEQ ID NO: 871 - RNA, 19 nt, Artificial Sequence
  Description of Artificial Sequence Synthetic oligonucleotide
agaacgcggagaaguggaa

SEQ ID NO: 872 - RNA, 19 nt, Artificial Sequence
  Description of Artificial Sequence Synthetic oligonucleotide
gagaaguggaacaagacgu

SEQ ID NO: 873 - RNA, 19 nt, Artificial Sequence
  Description of Artificial Sequence Synthetic oligonucleotide
cgaaggcgucaccggagaa

SEQ ID NO: 874 - RNA, 19 nt, Artificial Sequence
  Description of Artificial Sequence Synthetic oligonucleotide
aggaacgaggagcuguaaa

SEQ ID NO: 875 - RNA, 19 nt, Artificial Sequence
  Description of Artificial Sequence Synthetic oligonucleotide
uaagggugacaguacaaua

SEQ ID NO: 876 - RNA, 19 nt, Artificial Sequence
  Description of Artificial Sequence Synthetic oligonucleotide
gcugcaaggacucgguguu

SEQ ID NO: 877 - RNA, 19 nt, Artificial Sequence
  Description of Artificial Sequence Synthetic oligonucleotide
aaaugaaaguccuggaaua

SEQ ID NO: 878 - RNA, 19 nt, Artificial Sequence
  Description of Artificial Sequence Synthetic oligonucleotide
gaagaaggcuggaagaaau

SEQ ID NO: 879 - RNA, 19 nt, Artificial Sequence
  Description of Artificial Sequence Synthetic oligonucleotide
acauagaagucacuggaaa

SEQ ID NO: 880 - RNA, 19 nt, Artificial Sequence
  Description of Artificial Sequence Synthetic oligonucleotide
ggaacuggcugguuugaaa

SEQ ID NO: 881 - RNA, 19 nt, Artificial Sequence
  Description of Artificial Sequence Synthetic oligonucleotide
aguaauugguuuggagaaa

SEQ ID NO: 882 - RNA, 19 nt, Artificial Sequence
  Description of Artificial Sequence Synthetic oligonucleotide
uaacuagugucuuggaaua

SEQ ID NO: 883 - RNA, 19 nt, Artificial Sequence
  Description of Artificial Sequence Synthetic oligonucleotide
ggccuuagcaucagaauua

SEQ ID NO: 884 - RNA, 19 nt, Artificial Sequence
  Description of Artificial Sequence Synthetic oligonucleotide
guucauagucagcaauaaa

SEQ ID NO: 885 - RNA, 19 nt, Artificial Sequence
  Description of Artificial Sequence Synthetic oligonucleotide
cauagaagucacuggaaau

SEQ ID NO: 886 - RNA, 19 nt, Artificial Sequence
  Description of Artificial Sequence Synthetic oligonucleotide
gaauauggguugauuugaa

SEQ ID NO: 887 - RNA, 19 nt, Artificial Sequence
  Description of Artificial Sequence Synthetic oligonucleotide
ccaaagaaguugaaaagga

SEQ ID NO: 888 - RNA, 19 nt, Artificial Sequence
  Description of Artificial Sequence Synthetic oligonucleotide
ugaagaaggcuggaagaaa

SEQ ID NO: 889 - RNA, 19 nt, Artificial Sequence
  Description of Artificial Sequence Synthetic oligonucleotide
caaagaaguugaaaaggaa

SEQ ID NO: 890 - RNA, 19 nt, Artificial Sequence
  Description of Artificial Sequence Synthetic oligonucleotide
ggaagcaaagugugaauau

SEQ ID NO: 891 - RNA, 19 nt, Artificial Sequence
  Description of Artificial Sequence Synthetic oligonucleotide
gaguaauugguuuggagaa

SEQ ID NO: 892 - RNA, 19 nt, Artificial Sequence
  Description of Artificial Sequence Synthetic oligonucleotide
gagcggaacuggcugguuu

SEQ ID NO: 893 - RNA, 19 nt, Artificial Sequence
  Description of Artificial Sequence Synthetic oligonucleotide
gaagauggcuuuaugcuuu

SEQ ID NO: 894 - RNA, 19 nt, Artificial Sequence
  Description of Artificial Sequence Synthetic oligonucleotide
ucucagggauagaagauau

SEQ ID NO: 895 - RNA, 19 nt, Artificial Sequence
  Description of Artificial Sequence Synthetic oligonucleotide
cagccuaacucugaggaaa

SEQ ID NO: 896 - RNA, 19 nt, Artificial Sequence
  Description of Artificial Sequence Synthetic oligonucleotide
gcaacaaguggcacaguuu

SEQ ID NO: 897 - RNA, 19 nt, Artificial Sequence
  Description of Artificial Sequence Synthetic oligonucleotide
ggaccagccacaaaugaaa

SEQ ID NO: 898 - RNA, 19 nt, Artificial Sequence
  Description of Artificial Sequence Synthetic oligonucleotide
ucuuggaauaucugaguaa

SEQ ID NO: 899 - RNA, 19 nt, Artificial Sequence
  Description of Artificial Sequence Synthetic oligonucleotide
caacacaucuucaacacua

SEQ ID NO: 900 - RNA, 19 nt, Artificial Sequence
  Description of Artificial Sequence Synthetic oligonucleotide
gcgacuugacggaagguuu

SEQ ID NO: 901 - RNA, 19 nt, Artificial Sequence
  Description of Artificial Sequence Synthetic oligonucleotide
gaacaaacauagaagucac

SEQ ID NO: 902 - RNA, 19 nt, Artificial Sequence
  Description of Artificial Sequence Synthetic oligonucleotide
gaugaagaaggcuggaaga

SEQ ID NO: 903 - RNA, 19 nt, Artificial Sequence
  Description of Artificial Sequence Synthetic oligonucleotide
cgacagaaugugaacaaac

SEQ ID NO: 904 - RNA, 19 nt, Artificial Sequence
  Description of Artificial Sequence Synthetic oligonucleotide
gacagaaugugaacaaaca

SEQ ID NO: 905 - RNA, 19 nt, Artificial Sequence
  Description of Artificial Sequence Synthetic oligonucleotide
uaauugguuuggagaaaga

SEQ ID NO: 906 - RNA, 19 nt, Artificial Sequence
  Description of Artificial Sequence Synthetic oligonucleotide
ucagauugauacucagaau

SEQ ID NO: 907 - RNA, 19 nt, Artificial Sequence
  Description of Artificial Sequence Synthetic oligonucleotide
cagauugauacucagaaua

SEQ ID NO: 908 - RNA, 19 nt, Artificial Sequence
  Description of Artificial Sequence Synthetic oligonucleotide
ccgcaguggaagaguugau

SEQ ID NO: 909 - RNA, 19 nt, Artificial Sequence
  Description of Artificial Sequence Synthetic oligonucleotide
aagaaggcuggaagaaauu

SEQ ID NO: 910 - RNA, 19 nt, Artificial Sequence
  Description of Artificial Sequence Synthetic oligonucleotide
ugaaaguccuggaauagau

SEQ ID NO: 911 - RNA, 19 nt, Artificial Sequence
  Description of Artificial Sequence Synthetic oligonucleotide
guaacuagugucuuggaau

SEQ ID NO: 912 - RNA, 19 nt, Artificial Sequence
  Description of Artificial Sequence Synthetic oligonucleotide
ggaagauggcuuuaugcuu

SEQ ID NO: 913 - RNA, 19 nt, Artificial Sequence
  Description of Artificial Sequence Synthetic oligonucleotide
gcaagaaggugcucugaag

SEQ ID NO: 914 - RNA, 19 nt, Artificial Sequence
  Description of Artificial Sequence Synthetic oligonucleotide
agccuaacucugaggaaaa

SEQ ID NO: 915 - RNA, 19 nt, Artificial Sequence
  Description of Artificial Sequence Synthetic oligonucleotide
ggaaaaucccacucaguuu

SEQ ID NO: 916 - RNA, 19 nt, Artificial Sequence
  Description of Artificial Sequence Synthetic oligonucleotide
ggcaauguguucauaguca

SEQ ID NO: 917 - RNA, 19 nt, Artificial Sequence
  Description of Artificial Sequence Synthetic oligonucleotide
aaggaagcaaagugugaau

SEQ ID NO: 918 - RNA, 19 nt, Artificial Sequence
  Description of Artificial Sequence Synthetic oligonucleotide
guuggauaguaaugugaca

SEQ ID NO: 919 - RNA, 19 nt, Artificial Sequence
  Description of Artificial Sequence Synthetic oligonucleotide
augaagaaggcuggaagaa

SEQ ID NO: 920 - RNA, 19 nt, Artificial Sequence
  Description of Artificial Sequence Synthetic oligonucleotide
agacuuuacuccagaauug

SEQ ID NO: 921 - RNA, 19 nt, Artificial Sequence
  Description of Artificial Sequence Synthetic oligonucleotide
agaauugggaagauggcuu

SEQ ID NO: 922 - RNA, 19 nt, Artificial Sequence
  Description of Artificial Sequence Synthetic oligonucleotide
ccuuagcaucagaauuaaa

SEQ ID NO: 923 - RNA, 19 nt, Artificial Sequence
  Description of Artificial Sequence Synthetic oligonucleotide
aaauugacccaaagaaguu

SEQ ID NO: 924 - RNA, 19 nt, Artificial Sequence
  Description of Artificial Sequence Synthetic oligonucleotide
gacccaaagaaguugaaaa

SEQ ID NO: 925 - RNA, 19 nt, Artificial Sequence
  Description of Artificial Sequence Synthetic oligonucleotide
gaagcaaagugugaauauu

SEQ ID NO: 926 - RNA, 19 nt, Artificial Sequence
  Description of Artificial Sequence Synthetic oligonucleotide
cccaacacuucaauggcaa

SEQ ID NO: 927 - RNA, 19 nt, Artificial Sequence
  Description of Artificial Sequence Synthetic oligonucleotide
gcuuugagcuacaggacaa

SEQ ID NO: 928 - RNA, 19 nt, Artificial Sequence
  Description of Artificial Sequence Synthetic oligonucleotide
ggacaaaggcugacagacu

SEQ ID NO: 929 - RNA, 19 nt, Artificial Sequence
  Description of Artificial Sequence Synthetic oligonucleotide
gaaaagggacugugaguaa

SEQ ID NO: 930 - RNA, 19 nt, Artificial Sequence
  Description of Artificial Sequence Synthetic oligonucleotide
agaugaggacagugggaaa

SEQ ID NO: 931 - RNA, 19 nt, Artificial Sequence
  Description of Artificial Sequence Synthetic oligonucleotide
acucagaugucaagaagua

SEQ ID NO: 932 - RNA, 19 nt, Artificial Sequence
  Description of Artificial Sequence Synthetic oligonucleotide
ggacuuugaugaagaccau

SEQ ID NO: 933 - RNA, 19 nt, Artificial Sequence
  Description of Artificial Sequence Synthetic oligonucleotide
gugacuaacuaugcaauaa

SEQ ID NO: 934 - RNA, 19 nt, Artificial Sequence
  Description of Artificial Sequence Synthetic oligonucleotide
ccugggaggaagauguuua

SEQ ID NO: 935 - RNA, 19 nt, Artificial Sequence
  Description of Artificial Sequence Synthetic oligonucleotide
gcaaauacugcaacaagga

SEQ ID NO: 936 - RNA, 19 nt, Artificial Sequence
  Description of Artificial Sequence Synthetic oligonucleotide
aauacugcaacaaggaaua

SEQ ID NO: 937 - RNA, 19 nt, Artificial Sequence
  Description of Artificial Sequence Synthetic oligonucleotide
gagugguucuucugcgcua

SEQ ID NO: 938 - RNA, 19 nt, Artificial Sequence
  Description of Artificial Sequence Synthetic oligonucleotide
gcuacaggacaaaggcuga

SEQ ID NO: 939 - RNA, 19 nt, Artificial Sequence
  Description of Artificial Sequence Synthetic oligonucleotide
aaauacugcaacaaggaau

SEQ ID NO: 940 - RNA, 19 nt, Artificial Sequence
  Description of Artificial Sequence Synthetic oligonucleotide
ucaagaaguaccagugcca

SEQ ID NO: 941 - RNA, 19 nt, Artificial Sequence
  Description of Artificial Sequence Synthetic oligonucleotide
cacaggacuuugaugaaga

SEQ ID NO: 942 - RNA, 19 nt, Artificial Sequence
  Description of Artificial Sequence Synthetic oligonucleotide
gcaauuuaacaaugucuga

SEQ ID NO: 943 - RNA, 19 nt, Artificial Sequence
  Description of Artificial Sequence Synthetic oligonucleotide
ucucugaggccaaggaucu

SEQ ID NO: 944 - RNA, 19 nt, Artificial Sequence
  Description of Artificial Sequence Synthetic oligonucleotide
cggccuagcgagugguucu

SEQ ID NO: 945 - RNA, 19 nt, Artificial Sequence
  Description of Artificial Sequence Synthetic oligonucleotide
gaugugucucccagaacua

SEQ ID NO: 946 - RNA, 19 nt, Artificial Sequence
  Description of Artificial Sequence Synthetic oligonucleotide
gggccugggaggaagaugu

SEQ ID NO: 947 - RNA, 19 nt, Artificial Sequence
  Description of Artificial Sequence Synthetic oligonucleotide
uuuuaaagguacacuggua

SEQ ID NO: 948 - RNA, 19 nt, Artificial Sequence
  Description of Artificial Sequence Synthetic oligonucleotide
cgaaaggccuucaacugca

SEQ ID NO: 949 - RNA, 19 nt, Artificial Sequence
  Description of Artificial Sequence Synthetic oligonucleotide
cccacaggacuuugaugaa

SEQ ID NO: 950 - RNA, 19 nt, Artificial Sequence
  Description of Artificial Sequence Synthetic oligonucleotide
uuaaagguacacugguauu

SEQ ID NO: 951 - RNA, 19 nt, Artificial Sequence
  Description of Artificial Sequence Synthetic oligonucleotide
gaaaggccuucaacugcaa

SEQ ID NO: 952 - RNA, 19 nt, Artificial Sequence
  Description of Artificial Sequence Synthetic oligonucleotide
acaaaggcugacagacuca

SEQ ID NO: 953 - RNA, 19 nt, Artificial Sequence
  Description of Artificial Sequence Synthetic oligonucleotide
cuccacgaggugugacuaa

SEQ ID NO: 954 - RNA, 19 nt, Artificial Sequence
  Description of Artificial Sequence Synthetic oligonucleotide
gaguaauggcugucacuug

SEQ ID NO: 955 - RNA, 19 nt, Artificial Sequence
  Description of Artificial Sequence Synthetic oligonucleotide
aaucggaagccuaacuaca

SEQ ID NO: 956 - RNA, 19 nt, Artificial Sequence
  Description of Artificial Sequence Synthetic oligonucleotide
gcgagcugcaggacucuaa

SEQ ID NO: 957 - RNA, 19 nt, Artificial Sequence
  Description of Artificial Sequence Synthetic oligonucleotide
ccacaagcaccaagagucc

SEQ ID NO: 958 - RNA, 19 nt, Artificial Sequence
  Description of Artificial Sequence Synthetic oligonucleotide
caggacaaaggcugacaga

SEQ ID NO: 959 - RNA, 19 nt, Artificial Sequence
  Description of Artificial Sequence Synthetic oligonucleotide
acaaggaacccucaggcca

SEQ ID NO: 960 - RNA, 19 nt, Artificial Sequence
  Description of Artificial Sequence Synthetic oligonucleotide
cagaugaggacagugggaa

SEQ ID NO: 961 - RNA, 19 nt, Artificial Sequence
  Description of Artificial Sequence Synthetic oligonucleotide
gaaugucccugcuccacaa

SEQ ID NO: 962 - RNA, 19 nt, Artificial Sequence
  Description of Artificial Sequence Synthetic oligonucleotide
acuuugaugaagaccauuu

SEQ ID NO: 963 - RNA, 19 nt, Artificial Sequence
  Description of Artificial Sequence Synthetic oligonucleotide
ggccugucugcguggguuu

SEQ ID NO: 964 - RNA, 19 nt, Artificial Sequence
  Description of Artificial Sequence Synthetic oligonucleotide
gggcaauuuaacaaugucu

SEQ ID NO: 965 - RNA, 19 nt, Artificial Sequence
  Description of Artificial Sequence Synthetic oligonucleotide
uuuaaagguacacugguau

SEQ ID NO: 966 - RNA, 19 nt, Artificial Sequence
  Description of Artificial Sequence Synthetic oligonucleotide
agguacacugguauuuaua

SEQ ID NO: 967 - RNA, 19 nt, Artificial Sequence
  Description of Artificial Sequence Synthetic oligonucleotide
caaauacugcaacaaggaa

SEQ ID NO: 968 - RNA, 19 nt, Artificial Sequence
  Description of Artificial Sequence Synthetic oligonucleotide
gaaccugcgggaaggccuu

SEQ ID NO: 969 - RNA, 19 nt, Artificial Sequence
  Description of Artificial Sequence Synthetic oligonucleotide
agacccacucagaugucaa

SEQ ID NO: 970 - RNA, 19 nt, Artificial Sequence
  Description of Artificial Sequence Synthetic oligonucleotide
aagccuaacuacagcgagc

SEQ ID NO: 971 - RNA, 19 nt, Artificial Sequence
  Description of Artificial Sequence Synthetic oligonucleotide
ucagaugaggacaguggga

SEQ ID NO: 972 - RNA, 19 nt, Artificial Sequence
  Description of Artificial Sequence Synthetic oligonucleotide
gcucgaaaggccuucaacu

SEQ ID NO: 973 - RNA, 19 nt, Artificial Sequence
  Description of Artificial Sequence Synthetic oligonucleotide
augcacauccgaagccaca

SEQ ID NO: 974 - RNA, 19 nt, Artificial Sequence
  Description of Artificial Sequence Synthetic oligonucleotide
ccacucagaugucaagaag

SEQ ID NO: 975 - RNA, 19 nt, Artificial Sequence
  Description of Artificial Sequence Synthetic oligonucleotide
ggccauuucuguggaggga

SEQ ID NO: 976 - RNA, 19 nt, Artificial Sequence
  Description of Artificial Sequence Synthetic oligonucleotide
aguaauggcugucacuugu

SEQ ID NO: 977 - RNA, 19 nt, Artificial Sequence
  Description of Artificial Sequence Synthetic oligonucleotide
cauuagugaugaagaggaa

SEQ ID NO: 978 - RNA, 19 nt, Artificial Sequence
  Description of Artificial Sequence Synthetic oligonucleotide
ggacacacauacagugauu

SEQ ID NO: 979 - RNA, 19 nt, Artificial Sequence
  Description of Artificial Sequence Synthetic oligonucleotide
ggcuagauugagagaauaa

SEQ ID NO: 980 - RNA, 19 nt, Artificial Sequence
  Description of Artificial Sequence Synthetic oligonucleotide
gaacaguauugcuuuguaa

SEQ ID NO: 981 - RNA, 19 nt, Artificial Sequence
  Description of Artificial Sequence Synthetic oligonucleotide
caaauaaaguccaaaggca

SEQ ID NO: 982 - RNA, 19 nt, Artificial Sequence
  Description of Artificial Sequence Synthetic oligonucleotide
cuguagugcuuuaaaguau

SEQ ID NO: 983 - RNA, 19 nt, Artificial Sequence
  Description of Artificial Sequence Synthetic oligonucleotide
aagaaauaccagugcaaaa

SEQ ID NO: 984 - RNA, 19 nt, Artificial Sequence
  Description of Artificial Sequence Synthetic oligonucleotide
auggcuagauugagagaau

SEQ ID NO: 985 - RNA, 19 nt, Artificial Sequence
  Description of Artificial Sequence Synthetic oligonucleotide
uuguauaguugaugaguca

SEQ ID NO: 986 - RNA, 19 nt, Artificial Sequence
  Description of Artificial Sequence Synthetic oligonucleotide
aaauaaaguccaaaggcau

SEQ ID NO: 987 - RNA, 19 nt, Artificial Sequence
  Description of Artificial Sequence Synthetic oligonucleotide
ccugaagacuugugaaauc

SEQ ID NO: 988 - RNA, 19 nt, Artificial Sequence
  Description of Artificial Sequence Synthetic oligonucleotide
cuucaugauuaguaccaaa

SEQ ID NO: 989 - RNA, 19 nt, Artificial Sequence
  Description of Artificial Sequence Synthetic oligonucleotide
uaaagaaauaccagugcaa

SEQ ID NO: 990 - RNA, 19 nt, Artificial Sequence
  Description of Artificial Sequence Synthetic oligonucleotide
guauagacacacacacaua

SEQ ID NO: 991 - RNA, 19 nt, Artificial Sequence
  Description of Artificial Sequence Synthetic oligonucleotide
gcugauggcuagauugaga

SEQ ID NO: 992 - RNA, 19 nt, Artificial Sequence
  Description of Artificial Sequence Synthetic oligonucleotide
uguaauaggauuucccaua

SEQ ID NO: 993 - RNA, 19 nt, Artificial Sequence
  Description of Artificial Sequence Synthetic oligonucleotide
ccacaaaugcaauaauaca

SEQ ID NO: 994 - RNA, 19 nt, Artificial Sequence
  Description of Artificial Sequence Synthetic oligonucleotide
gaacaaaacacaggagaau

SEQ ID NO: 995 - RNA, 19 nt, Artificial Sequence
  Description of Artificial Sequence Synthetic oligonucleotide
ucguaaaggagccggguga

SEQ ID NO: 996 - RNA, 19 nt, Artificial Sequence
  Description of Artificial Sequence Synthetic oligonucleotide
acacacacccacagagaga

SEQ ID NO: 997 - RNA, 19 nt, Artificial Sequence
  Description of Artificial Sequence Synthetic oligonucleotide
gagauguugucuauagcua

SEQ ID NO: 998 - RNA, 19 nt, Artificial Sequence
  Description of Artificial Sequence Synthetic oligonucleotide
cauugaagcugaaaaguuu

SEQ ID NO: 999 - RNA, 19 nt, Artificial Sequence
  Description of Artificial Sequence Synthetic oligonucleotide
aauaaaguccaaaggcauu

SEQ ID NO: 1000 - RNA, 19 nt, Artificial Sequence
  Description of Artificial Sequence Synthetic oligonucleotide
agagagagcugcaagagca

SEQ ID NO: 1001 - RNA, 19 nt, Artificial Sequence
  Description of Artificial Sequence Synthetic oligonucleotide
gcugcaagagcauggaauu

SEQ ID NO: 1002 - RNA, 19 nt, Artificial Sequence
  Description of Artificial Sequence Synthetic oligonucleotide
agaacaaaacacaggagaa

SEQ ID NO: 1003 - RNA, 19 nt, Artificial Sequence
  Description of Artificial Sequence Synthetic oligonucleotide
gaaugaguucuguaugaaa

SEQ ID NO: 1004 - RNA, 19 nt, Artificial Sequence
  Description of Artificial Sequence Synthetic oligonucleotide
ugaugaagaggaaagacua

SEQ ID NO: 1005 - RNA, 19 nt, Artificial Sequence
  Description of Artificial Sequence Synthetic oligonucleotide
aauacugugacaaggaaua

SEQ ID NO: 1006 - RNA, 19 nt, Artificial Sequence
  Description of Artificial Sequence Synthetic oligonucleotide
gcacaaacaugaggaaucu

SEQ ID NO: 1007 - RNA, 19 nt, Artificial Sequence
  Description of Artificial Sequence Synthetic oligonucleotide
uugaaugaguucuguauga

SEQ ID NO: 1008 - RNA, 19 nt, Artificial Sequence
  Description of Artificial Sequence Synthetic oligonucleotide
aaacugagauguugucuau

SEQ ID NO: 1009 - RNA, 19 nt, Artificial Sequence
  Description of Artificial Sequence Synthetic oligonucleotide
ccaaaccacuguacaaaga

SEQ ID NO: 1010 - RNA, 19 nt, Artificial Sequence
  Description of Artificial Sequence Synthetic oligonucleotide
acacacauacagugauuau

SEQ ID NO: 1011 - RNA, 19 nt, Artificial Sequence
  Description of Artificial Sequence Synthetic oligonucleotide
gugaugaagaggaaagacu

SEQ ID NO: 1012 - RNA, 19 nt, Artificial Sequence
  Description of Artificial Sequence Synthetic oligonucleotide
guaaauacugugacaagga

SEQ ID NO: 1013 - RNA, 19 nt, Artificial Sequence
  Description of Artificial Sequence Synthetic oligonucleotide
ccacagagagagagcugca

SEQ ID NO: 1014 - RNA, 19 nt, Artificial Sequence
  Description of Artificial Sequence Synthetic oligonucleotide
auauauuugcugauggcua

SEQ ID NO: 1015 - RNA, 19 nt, Artificial Sequence
  Description of Artificial Sequence Synthetic oligonucleotide
gcuccuuccuggucaagaa

SEQ ID NO: 1016 - RNA, 19 nt, Artificial Sequence
  Description of Artificial Sequence Synthetic oligonucleotide
gaaacugagauguugucua

SEQ ID NO: 1017 - RNA, 19 nt, Artificial Sequence
  Description of Artificial Sequence Synthetic oligonucleotide
auaaacaaccugaagacuu

SEQ ID NO: 1018 - RNA, 19 nt, Artificial Sequence
  Description of Artificial Sequence Synthetic oligonucleotide
aaccugaagacuugugaaa

SEQ ID NO: 1019 - RNA, 19 nt, Artificial Sequence
  Description of Artificial Sequence Synthetic oligonucleotide
aagccaaacuacagcgaac

SEQ ID NO: 1020 - RNA, 19 nt, Artificial Sequence
  Description of Artificial Sequence Synthetic oligonucleotide
cagagagagagcugcaaga

SEQ ID NO: 1021 - RNA, 19 nt, Artificial Sequence
  Description of Artificial Sequence Synthetic oligonucleotide
gaugggaauaagugcaaaa

SEQ ID NO: 1022 - RNA, 19 nt, Artificial Sequence
  Description of Artificial Sequence Synthetic oligonucleotide
uuucaaaugcauaccacaa

SEQ ID NO: 1023 - RNA, 19 nt, Artificial Sequence
  Description of Artificial Sequence Synthetic oligonucleotide
uguaugaaacugagauguu

SEQ ID NO: 1024 - RNA, 19 nt, Artificial Sequence
  Description of Artificial Sequence Synthetic oligonucleotide
ccucacugcaacagagcau

SEQ ID NO: 1025 - RNA, 19 nt, Artificial Sequence
  Description of Artificial Sequence Synthetic oligonucleotide
caaucaauguuuacucgaa

SEQ ID NO: 1026 - RNA, 19 nt, Artificial Sequence
  Description of Artificial Sequence Synthetic oligonucleotide
gaagccaaaugacaaauaa

SEQ ID NO: 1027 - RNA, 19 nt, Artificial Sequence
  Description of Artificial Sequence Synthetic oligonucleotide
agaaggaggacgaggagaa

SEQ ID NO: 1028 - RNA, 19 nt, Artificial Sequence
  Description of Artificial Sequence Synthetic oligonucleotide
gcauagaauucaaacgaga

SEQ ID NO: 1029 - RNA, 19 nt, Artificial Sequence
  Description of Artificial Sequence Synthetic oligonucleotide
ccggaucacucaagcaaua

SEQ ID NO: 1030 - RNA, 19 nt, Artificial Sequence
  Description of Artificial Sequence Synthetic oligonucleotide
agaucaagcgggaggagaa

SEQ ID NO: 1031 - RNA, 19 nt, Artificial Sequence
  Description of Artificial Sequence Synthetic oligonucleotide
aaacaaaaccugaaagcaa

SEQ ID NO: 1032 - RNA, 19 nt, Artificial Sequence
  Description of Artificial Sequence Synthetic oligonucleotide
acucggaggaggagaagaa

SEQ ID NO: 1033 - RNA, 19 nt, Artificial Sequence
  Description of Artificial Sequence Synthetic oligonucleotide
gggcacaugugaaagguau

SEQ ID NO: 1034 - RNA, 19 nt, Artificial Sequence
  Description of Artificial Sequence Synthetic oligonucleotide
ccugaaagcaagcaacaaa

SEQ ID NO: 1035 - RNA, 19 nt, Artificial Sequence
  Description of Artificial Sequence Synthetic oligonucleotide
gggaggagaaggaggacga

SEQ ID NO: 1036 - RNA, 19 nt, Artificial Sequence
  Description of Artificial Sequence Synthetic oligonucleotide
gcaccagccucaugcacaa

SEQ ID NO: 1037 - RNA, 19 nt, Artificial Sequence
  Description of Artificial Sequence Synthetic oligonucleotide
acacuuugucagagaagaa

SEQ ID NO: 1038 - RNA, 19 nt, Artificial Sequence
  Description of Artificial Sequence Synthetic oligonucleotide
aggagaagaaggagcugaa

SEQ ID NO: 1039 - RNA, 19 nt, Artificial Sequence
  Description of Artificial Sequence Synthetic oligonucleotide
aaauugugccuaagcgaaa

SEQ ID NO: 1040 - RNA, 19 nt, Artificial Sequence
  Description of Artificial Sequence Synthetic oligonucleotide
cagacgaggacgaggacga

SEQ ID NO: 1041 - RNA, 19 nt, Artificial Sequence
  Description of Artificial Sequence Synthetic oligonucleotide
uagcaauaaacgugacauu

SEQ ID NO: 1042 - RNA, 19 nt, Artificial Sequence
  Description of Artificial Sequence Synthetic oligonucleotide
guucggagguucaggucuu

SEQ ID NO: 1043 - RNA, 19 nt, Artificial Sequence
  Description of Artificial Sequence Synthetic oligonucleotide
cggaggaggagaagaagga

SEQ ID NO: 1044 - RNA, 19 nt, Artificial Sequence
  Description of Artificial Sequence Synthetic oligonucleotide
gaaacggcgagaagaggaa

SEQ ID NO: 1045 - RNA, 19 nt, Artificial Sequence
  Description of Artificial Sequence Synthetic oligonucleotide
gcauauguuuuguaagcaa

SEQ ID NO: 1046 - RNA, 19 nt, Artificial Sequence
  Description of Artificial Sequence Synthetic oligonucleotide
agaguaagauagaagacca

SEQ ID NO: 1047 - RNA, 19 nt, Artificial Sequence
  Description of Artificial Sequence Synthetic oligonucleotide
gcgcgaggaggaagaaaca

SEQ ID NO: 1048 - RNA, 19 nt, Artificial Sequence
  Description of Artificial Sequence Synthetic oligonucleotide
cuacagugggcuagggcga

SEQ ID NO: 1049 - RNA, 19 nt, Artificial Sequence
  Description of Artificial Sequence Synthetic oligonucleotide
acauacacuuugucagaga

SEQ ID NO: 1050 - RNA, 19 nt, Artificial Sequence
  Description of Artificial Sequence Synthetic oligonucleotide
ucuaaagccaccagcaaau

SEQ ID NO: 1051 - RNA, 19 nt, Artificial Sequence
  Description of Artificial Sequence Synthetic oligonucleotide
cugugugguccaagggcaa

SEQ ID NO: 1052 - RNA, 19 nt, Artificial Sequence
  Description of Artificial Sequence Synthetic oligonucleotide
ugucaggugugguuggaga

SEQ ID NO: 1053 - RNA, 19 nt, Artificial Sequence
  Description of Artificial Sequence Synthetic oligonucleotide
aaacauacacuuugucaga

SEQ ID NO: 1054 - RNA, 19 nt, Artificial Sequence
  Description of Artificial Sequence Synthetic oligonucleotide
cagacaaggagcucaguga

SEQ ID NO: 1055 - RNA, 19 nt, Artificial Sequence
  Description of Artificial Sequence Synthetic oligonucleotide
ggggaagggacgucagcaa

SEQ ID NO: 1056 - RNA, 19 nt, Artificial Sequence
  Description of Artificial Sequence Synthetic oligonucleotide
ggaggaagaaacagcaguu

SEQ ID NO: 1057 - RNA, 19 nt, Artificial Sequence
  Description of Artificial Sequence Synthetic oligonucleotide
gcaauaaacgugacauuuu

SEQ ID NO: 1058 - RNA, 19 nt, Artificial Sequence
  Description of Artificial Sequence Synthetic oligonucleotide
cggccugcagaguaagaua

SEQ ID NO: 1059 - RNA, 19 nt, Artificial Sequence
  Description of Artificial Sequence Synthetic oligonucleotide
aggagaaggaggacgagga

SEQ ID NO: 1060 - RNA, 19 nt, Artificial Sequence
  Description of Artificial Sequence Synthetic oligonucleotide
cuucuaaagccaccagcaa

SEQ ID NO: 1061 - RNA, 19 nt, Artificial Sequence
  Description of Artificial Sequence Synthetic oligonucleotide
ccauuacaccagagggcca

SEQ ID NO: 1062 - RNA, 19 nt, Artificial Sequence
  Description of Artificial Sequence Synthetic oligonucleotide
augguagaugcaagggaaa

SEQ ID NO: 1063 - RNA, 19 nt, Artificial Sequence
  Description of Artificial Sequence Synthetic oligonucleotide
uagaagaccaccuggacga

SEQ ID NO: 1064 - RNA, 19 nt, Artificial Sequence
  Description of Artificial Sequence Synthetic oligonucleotide
ccagcgagaucaagcggga

SEQ ID NO: 1065 - RNA, 19 nt, Artificial Sequence
  Description of Artificial Sequence Synthetic oligonucleotide
gcaaauugugccuaagcga

SEQ ID NO: 1066 - RNA, 19 nt, Artificial Sequence
  Description of Artificial Sequence Synthetic oligonucleotide
gugccuaagcgaaauauuu

SEQ ID NO: 1067 - RNA, 19 nt, Artificial Sequence
  Description of Artificial Sequence Synthetic oligonucleotide
gaugaaaauuagcaaggau

SEQ ID NO: 1068 - RNA, 19 nt, Artificial Sequence
  Description of Artificial Sequence Synthetic oligonucleotide
uccacggccugcagaguaa

SEQ ID NO: 1069 - RNA, 19 nt, Artificial Sequence
  Description of Artificial Sequence Synthetic oligonucleotide
cugcagaguaagauagaag

SEQ ID NO: 1070 - RNA, 19 nt, Artificial Sequence
  Description of Artificial Sequence Synthetic oligonucleotide
aggaaaaggugucaggugu

SEQ ID NO: 1071 - RNA, 19 nt, Artificial Sequence
  Description of Artificial Sequence Synthetic oligonucleotide
cauugcauuucuugaucaa

SEQ ID NO: 1072 - RNA, 19 nt, Artificial Sequence
  Description of Artificial Sequence Synthetic oligonucleotide
gggacugucuuggguuuaa

SEQ ID NO: 1073 - RNA, 19 nt, Artificial Sequence
  Description of Artificial Sequence Synthetic oligonucleotide
gagcagaggugaacggugg

SEQ ID NO: 1074 - RNA, 19 nt, Artificial Sequence
  Description of Artificial Sequence Synthetic oligonucleotide
ucagugaccuccuggacuu

SEQ ID NO: 1075 - RNA, 19 nt, Artificial Sequence
  Description of Artificial Sequence Synthetic oligonucleotide
ugaaccagccgcagaggau

SEQ ID NO: 1076 - RNA, 19 nt, Artificial Sequence
  Description of Artificial Sequence Synthetic oligonucleotide
gcaacaaaacauacacuuu

SEQ ID NO: 1077 - RNA, 19 nt, Artificial Sequence
  Description of Artificial Sequence Synthetic oligonucleotide
ggaaauuagaagagcaaaa

SEQ ID NO: 1078 - RNA, 19 nt, Artificial Sequence
  Description of Artificial Sequence Synthetic oligonucleotide
cagaggaacuauaagaaca

SEQ ID NO: 1079 - RNA, 19 nt, Artificial Sequence
  Description of Artificial Sequence Synthetic oligonucleotide
ggaucaaacuggccugcaa

SEQ ID NO: 1080 - RNA, 19 nt, Artificial Sequence
  Description of Artificial Sequence Synthetic oligonucleotide
gguaacaaucagaggaacu

SEQ ID NO: 1081 - RNA, 19 nt, Artificial Sequence
  Description of Artificial Sequence Synthetic oligonucleotide
gcaaaaccauagucaguua

SEQ ID NO: 1082 - RNA, 19 nt, Artificial Sequence
  Description of Artificial Sequence Synthetic oligonucleotide
ggacaagcugagcaagauu

SEQ ID NO: 1083 - RNA, 19 nt, Artificial Sequence
  Description of Artificial Sequence Synthetic oligonucleotide
uuggaaauuagaagagcaa

SEQ ID NO: 1084 - RNA, 19 nt, Artificial Sequence
  Description of Artificial Sequence Synthetic oligonucleotide
ccucggacaagcugagcaa

SEQ ID NO: 1085 - RNA, 19 nt, Artificial Sequence
  Description of Artificial Sequence Synthetic oligonucleotide
ccggagaccuagaugucau

SEQ ID NO: 1086 - RNA, 19 nt, Artificial Sequence
  Description of Artificial Sequence Synthetic oligonucleotide
gauagaagucugaacaguu

SEQ ID NO: 1087 - RNA, 19 nt, Artificial Sequence
  Description of Artificial Sequence Synthetic oligonucleotide
auugaggacccaugguaaa

SEQ ID NO: 1088 - RNA, 19 nt, Artificial Sequence
  Description of Artificial Sequence Synthetic oligonucleotide
ccgacgacagccugagcaa

SEQ ID NO: 1089 - RNA, 19 nt, Artificial Sequence
  Description of Artificial Sequence Synthetic oligonucleotide
aggaagagccagaccggca

SEQ ID NO: 1090 - RNA, 19 nt, Artificial Sequence
  Description of Artificial Sequence Synthetic oligonucleotide
gagcaaaauccaaauucaa

SEQ ID NO: 1091 - RNA, 19 nt, Artificial Sequence
  Description of Artificial Sequence Synthetic oligonucleotide
gaucaaacuggccugcaaa

SEQ ID NO: 1092 - RNA, 19 nt, Artificial Sequence
  Description of Artificial Sequence Synthetic oligonucleotide
gcaaauagauccggugucu

SEQ ID NO: 1093 - RNA, 19 nt, Artificial Sequence
  Description of Artificial Sequence Synthetic oligonucleotide
gugucuaaaugcauucaua

SEQ ID NO: 1094 - RNA, 19 nt, Artificial Sequence
  Description of Artificial Sequence Synthetic oligonucleotide
gagagaugaugcaggacgu

SEQ ID NO: 1095 - RNA, 19 nt, Artificial Sequence
  Description of Artificial Sequence Synthetic oligonucleotide
ugagcaacagcgaggaaga

SEQ ID NO: 1096 - RNA, 19 nt, Artificial Sequence
  Description of Artificial Sequence Synthetic oligonucleotide
ucggacaagcugagcaaga

SEQ ID NO: 1097 - RNA, 19 nt, Artificial Sequence
  Description of Artificial Sequence Synthetic oligonucleotide
agacucuggagcuggauaa

SEQ ID NO: 1098 - RNA, 19 nt, Artificial Sequence
  Description of Artificial Sequence Synthetic oligonucleotide
cucuggagcuggauaacua

SEQ ID NO: 1099 - RNA, 19 nt, Artificial Sequence
  Description of Artificial Sequence Synthetic oligonucleotide
uaaaagagaaagcgagaca

SEQ ID NO: 1100 - RNA, 19 nt, Artificial Sequence
  Description of Artificial Sequence Synthetic oligonucleotide
acgaggagcugcagacgca

SEQ ID NO: 1101 - RNA, 19 nt, Artificial Sequence
  Description of Artificial Sequence Synthetic oligonucleotide
ugucauuguuuccagagaa

SEQ ID NO: 1102 - RNA, 19 nt, Artificial Sequence
  Description of Artificial Sequence Synthetic oligonucleotide
gaaaggaaaggcaucacua

SEQ ID NO: 1103 - RNA, 19 nt, Artificial Sequence
  Description of Artificial Sequence Synthetic oligonucleotide
gacgacagccugagcaaca

SEQ ID NO: 1104 - RNA, 19 nt, Artificial Sequence
  Description of Artificial Sequence Synthetic oligonucleotide
gcaagaagucugcgggcug

SEQ ID NO: 1105 - RNA, 19 nt, Artificial Sequence
  Description of Artificial Sequence Synthetic oligonucleotide
cuuggaaauuagaagagca

SEQ ID NO: 1106 - RNA, 19 nt, Artificial Sequence
  Description of Artificial Sequence Synthetic oligonucleotide
auucaaagaaacagggcgu

SEQ ID NO: 1107 - RNA, 19 nt, Artificial Sequence
  Description of Artificial Sequence Synthetic oligonucleotide
ccacugaaaggaaaggcau

SEQ ID NO: 1108 - RNA, 19 nt, Artificial Sequence
  Description of Artificial Sequence Synthetic oligonucleotide
augguaacaaucagaggaa

SEQ ID NO: 1109 - RNA, 19 nt, Artificial Sequence
  Description of Artificial Sequence Synthetic oligonucleotide
guaacaaucagaggaacua

SEQ ID NO: 1110 - RNA, 19 nt, Artificial Sequence
  Description of Artificial Sequence Synthetic oligonucleotide
aaucagaggaacuauaaga

SEQ ID NO: 1111 - RNA, 19 nt, Artificial Sequence
  Description of Artificial Sequence Synthetic oligonucleotide
uauugaggacccaugguaa

SEQ ID NO: 1112 - RNA, 19 nt, Artificial Sequence
  Description of Artificial Sequence Synthetic oligonucleotide
ccugagcaacagcgaggaa

SEQ ID NO: 1113 - RNA, 19 nt, Artificial Sequence
  Description of Artificial Sequence Synthetic oligonucleotide
caacagcgaggaagagcca

SEQ ID NO: 1114 - RNA, 19 nt, Artificial Sequence
  Description of Artificial Sequence Synthetic oligonucleotide
acagcgaggaagagccaga

SEQ ID NO: 1115 - RNA, 19 nt, Artificial Sequence
  Description of Artificial Sequence Synthetic oligonucleotide
gagaaggagaaaauggaca

SEQ ID NO: 1116 - RNA, 19 nt, Artificial Sequence
  Description of Artificial Sequence Synthetic oligonucleotide
uagaagagcaaaauccaaa

SEQ ID NO: 1117 - RNA, 19 nt, Artificial Sequence
  Description of Artificial Sequence Synthetic oligonucleotide
uuuaaaagagaaagcgaga

SEQ ID NO: 1118 - RNA, 19 nt, Artificial Sequence
  Description of Artificial Sequence Synthetic oligonucleotide
gguaaaaugcaaauagauc

SEQ ID NO: 1119 - RNA, 19 nt, Artificial Sequence
  Description of Artificial Sequence Synthetic oligonucleotide
gcacccagucgcugaacga

SEQ ID NO: 1120 - RNA, 19 nt, Artificial Sequence
  Description of Artificial Sequence Synthetic oligonucleotide
cggacaagcugagcaagau

SEQ ID NO: 1121 - RNA, 19 nt, Artificial Sequence
  Description of Artificial Sequence Synthetic oligonucleotide
cauuguuuccagagaagga

SEQ ID NO: 1122 - RNA, 19 nt, Artificial Sequence
  Description of Artificial Sequence Synthetic oligonucleotide
uuccagagaaggagaaaau

SEQ ID NO: 1123 - RNA, 19 nt, Artificial Sequence
  Description of Artificial Sequence Synthetic oligonucleotide
aggagaaaauggacagucu

SEQ ID NO: 1124 - RNA, 19 nt, Artificial Sequence
  Description of Artificial Sequence Synthetic oligonucleotide
cugcaaaaccauagucagu

SEQ ID NO: 1125 - RNA, 19 nt, Artificial Sequence
  Description of Artificial Sequence Synthetic oligonucleotide
ggagaaaauggacagucua

SEQ ID NO: 1126 - RNA, 19 nt, Artificial Sequence
  Description of Artificial Sequence Synthetic oligonucleotide
aggcaucacuauggacuuu